(19) United States
(12) Patent Application Publication
(10) Pub. No.: US 2024/0200093 A1
(43) Pub. Date: Jun. 20, 2024

(54) ARTIFICIAL REGULATORY CASSETTES FOR MUSCLE-SPECIFIC GENE EXPRESSION

(71) Applicant: University of Washington, Seattle, WA (US)

(72) Inventors: Jeffrey S. Chamberlain, Seattle, WA (US); Stephen D. Hauschka, Seattle, WA (US)

(73) Assignee: University of Washington, Seattle, WA (US)

(21) Appl. No.: 18/286,215

(22) PCT Filed: Apr. 7, 2022
(86) PCT No.: PCT/US2022/023915

§ 371 Date: Oct. 9, 2023

Related U.S. Application Data
(60) Provisional application No. 63/173,295, filed on Apr. 9, 2021.

(51) Int. Cl. C12N 15/86

(52) U.S. Cl. CPC C12N 15/86; C12N 2750/14143; C12N 2830/008

(57) ABSTRACT

A library of artificial muscle-specific expression cassettes (MSECs) for muscle-specific gene expression is described. Different members of the library can be selected for varied transcription levels in different muscle cell types, for different research or therapeutic purposes. MSECs within the library can be used to develop treatments for muscle-related disorders.

### CROSS REFERENCE TO RELATED APPLICATIONS

This application is a U.S. National Phase Application based on International Patent Application No. PCT/US2022/023915 filed Apr. 7, 2022 which claims priority to U.S. Provisional Patent Application No. 63/173,295 filed Apr. 9, 2021, the entire contents of each of which are incorporated by reference herein.

### REFERENCE TO SEQUENCE LISTING

The Sequence Listing associated with this application is provided in text format in lieu of a paper copy, and is hereby incorporated by reference into the specification. The name of the text file containing the Sequence Listing is 2ZC2795_txt. The text is 321,979 bytes, was created on Oct. 9, 2023, and is being submitted electronically via EFS Web.

### FIELD OF THE DISCLOSURE

The current disclosure relates to the fields of molecular biology, medicine and genetics, and in particular describes regulatory cassettes that provide different levels of protein or RNA products within skeletal and cardiac muscle cells, and minimal levels in all other cell types. These artificial Muscle-Specific Expression Cassettes (MSECs) can be used to develop treatments for virtually any vertebrate muscle-related disorders, as well as many other human and vertebrate disorders in which skeletal muscle tissue can be used to produce beneficial secreted products such as hormones, clotting factors, antibodies, or other beneficial protein or metabolite.

### SUMMARY OF THE DISCLOSURE

Muscle-Specific Expression Cassettes (MSECs) are artificial expression constructs including DNA that can be used to selectively express operably linked coding sequences in skeletal and cardiac muscle cells, while expressing virtually none of the coding sequence in non-muscle cells. MSECs can be attached to cDNAs encoding any protein or RNA, and when these constructs are inserted (transduced) into cardiac or skeletal muscle cells, the cDNA-encoded protein or RNA product will be synthesized. Based on different designs, individual MSEC-mediated product levels vary over concentration ranges exceeding 1000-fold. MSECs within the disclosed library can thus be used to selectively express any selected protein or RNA in cardiac and/or skeletal muscle cells over very broad concentration ranges. This MSEC library capacity can be applied to develop gene therapy treatments for Neuromuscular and cardiac muscle diseases, cancer cachexia, and aging diseases, as well as for any other diseases for which factors produced and secreted by muscle cells would be beneficial. The latter can range from secreted polypeptide hormones and cytokines, extracellular matrix proteins, enzymes, clotting factors and antibodies to metabolites. MSECs could also be used for immunization against virtually any antigen, for a wide variety of veterinary and animal agricultural purposes, as well as for cell-based meat production.

### BRIEF DESCRIPTION OF THE SEVERAL VIEWS OF THE DRAWINGS

Some of the drawings may be better understood in color. Applicant considers the color versions of the drawings as part of the original submission and reserves the right to present them in later proceedings.

FIG. 1 shows a representative embodiment of a Muscle Specific Expression Cassette (MSEC). MSECs can be used to drive expression of product cDNA. The combination of an MSEC and a product cDNA can be inserted into a delivery vector, in some embodiments a viral vector such as an AAV, and delivered to a patient to achieve product expression in skeletal and cardiac muscle and not in non-muscle. In some embodiments, differential expression in cardiac and skeletal muscle can be achieved by selecting an MSEC with differential expression activity in skeletal and cardiac muscle. The MSEC library allows for specific tuning of the expression levels of a transgene with the ability to both enhance and lower expression in targeted tissues. MSECs have a compact size and can therefore be used in a variety of vectors, including base-pair limited delivery vectors such as viral vectors, including AAV.

FIG. 2 depicts skeletal muscle control elements (CEs), heart muscle CEs, a permissive promoter, and representative combinations thereof into multi-muscle therapeutic gene constructs. A specific combination of skeletal muscle CEs and heart muscle CEs is ligated to a permissive promoter. This basic form of an MSEC can then be ligated to a product cDNA. A particular MSEC can be chosen to drive differential expression in different target tissues. For example, in some embodiments, an MSEC comprising a combination of skeletal muscle CEs and heart muscle CEs may drive differential expression in skeletal and cardiac muscle. In particular embodiments, an MSEC may drive higher, lower, or equivalent expression of a transgene in skeletal muscle as compared to cardiac muscle. Designing MSECs with different component CEs allows tunable cDNA expression levels in targeted muscle tissues.

FIG. 3. In some embodiments, MSECs are based on different promoter and enhancer regions of muscle-specific genes such as the M-Creatine Kinase (CKM) gene. Native CKM exhibits very high expression in both skeletal and cardiac muscle and is only activated upon muscle differentiation. The current disclosure is based on discoveries regarding CKM's 5′- and intron-1 enhancers and proximal promoter regions, the E-box and its MyoD binding, and other discoveries indicating that these CEs play essential roles in the muscle-specific expression of CKM, as well as in many other muscle genes. Via quantitative proteomics, previously unrecognized transcription factors with novel DNA binding sites in the CKM enhancer and promoter that constitute other CEs (Six4/5, MAZ, and KLF3) that are also important for muscle gene expression were identified. Depending on vector packaging size capacity, CKM-mediated regulatory components can be used to express cDNA-encoded products in differentiated skeletal and cardiac muscle cells while limiting its expression in non-muscle cells. However, when used in conjunction with vectors such as AdenoAssociated Virus (AAV) that have limited packaging capacities, native CKM regulatory regions require miniaturization to accommodate the efficient co-packaging of cDNAs exceeding about 4 kb. This is achieved by removing genomic sequence regions that are not conserved based on sequence alignments between different mammalian and/or vertebrate species (see FIGS. 8-10). An additional consequence of such miniaturizations is that transcriptional activity of the smaller regulatory components can be either increased or decreased (see FIGS. 5 and 7).

FIG. 4. Standard cell culture assay method for evaluating MSEC expression activity in differentiated skeletal muscle cells. A permanent clonally-derived mouse skeletal muscle cell line (MM14) is seeded into culture wells in the presence of Fibroblast Growth Factor (FGF). This stimulates proliferation and represses differentiation. Cultures are transfected by adding MSEC-test and reference plasmids. MSEC-test plasmids contain MSECs ligated to Luciferase cDNAs and reference plasmids contain the native CKM, referred to herein as MSEC-1263a ligated to a Renilla cDNA. The reference plasmid serves as an assay standardization protocol for normalizing each assay to the number and percentage of transfected cells and extent of differentiation in each culture plate via calculating the ratio of Luciferase to Renilla levels. Four hours later cultures are aspirated, rinsed, and switched to differentiation medium containing low serum and no FGF; 48 hours later cultures are collected, frozen, and extracts are subsequently analyzed for relative levels of Luciferase and Renilla activity. As illustrated, some MSECs (t, u, v, w, x) produce higher levels of the Luciferase product than the MSEC-1263a control (c), while other MSECs (y and z) produce less Luciferase product. Typical experiments use 4 culture dishes for testing each MSEC and the mean relative Luciferase to Renilla levels are calculated and then compared to data from previous experiments. Mean expression values for the same MSEC are adjusted as repeated assays of the same MSEC are performed.

FIG. 5. Transcriptional activities for 59 different MSECs in mouse skeletal muscle cultures normalized to that of the native mouse CKM (−1256 to +7) MSEC-1263a plotted=1. Data shown includes MSECs containing modified mouse CKM enhancer and promoter regions, MSECs containing modified mouse CKM regions with multimerized small intronic enhancers (SIEs), MSECs containing modified human CKM regulatory regions and MSECs containing fully synthetic enhancers ligated to miniaturized CKM or human ACTA1 promoters. Data for a ubiquitously active (non-muscle-specific) Cytomegalovirus enhancer/promoter cassette (CMV) is shown for comparison. MSECs exhibit graded transcription rates over a nearly 20-fold activity range above native MCK, which is the second most active skeletal muscle gene. Human versions of many MSECs have activities similar to the analogous mouse CKM versions. Synthetic regulatory cassettes contain optimized arrays of CEs from diverse striated muscle genes. (CK7=MSEC-571, CK8=MSEC-438a, MHCK7=MSEC-770, Native Mouse=Native CKM=MSEC-1263a).

FIG. 6. mRNA transcript levels per million for various genes in adult human skeletal muscle (Broad Institute Gtexportal). Human diseases associated with mutations of these genes are bolded. Disregarding unknown parameters such as mRNA and protein half-lives, the 5000-fold range of normal mRNA levels implies that optimal gene replacement therapies for the indicated diseases will require MSECs with a wide range of transcriptional activities to provide optimal therapeutic product levels (see FIG. 7).

FIG. 7. Log-scale plot demonstrating that the transcriptional activities of current MSECs span a 1000-fold range. This MSEC activity range facilitates selecting MSECs that—depending on vector dose and transduction efficiency—are likely to be appropriate for different gene therapy targets.

FIG. 8. Sequence conservation of the CKM gene 5′-enhancer regions among 6 mammalian species aligned with the mouse (−1256 to −1051) sequence, as well as miniaturization strategies. Highly conserved sequences are boxed and named. Xs indicate non-conserved regions that were tested to determine whether their complete or partial deletion caused appreciable losses in transcriptional activity. MSECs exhibiting either increased, decreased, or no change in activity are included in the total MSEC sequence Library.

FIG. 9. Sequence conservation of the CKM gene proximal promoter regions among 5 mammalian species aligned with the mouse (−358 to +7) sequence, as well as MSEC miniaturization strategies. Identified highly conserved sequences are boxed and named, other highly conserved, but unidentified, sequences are unboxed. Deletion tests of the unboxed conserved regions resulted in decreased transcriptional activities. Xs indicate non-conserved regions that were tested to determine whether their complete or partial deletion caused appreciable losses in transcriptional activity. Some deleted sequences were also replaced with additional CEs. Sequence alignments of the CKM Exon-1 region also exhibit extensive conservation. Deletion tests of this region have indicated that most miniaturized CKM enhancer+promoter MSECs exhibit higher transcriptional activity when combined with the (+1 to +50) region than with the (+1 to +7 region), but the (+1 to +12) region is superior to either of the other Exon-1 regions in the context of certain MSECs. MSECs with either increased, decreased, or no change in activity are included in the total MSEC sequence Library. (CK7=MSEC-571, CK8=MSEC-538a, CK9=MSEC-336c.)

FIG. 10. Sequence alignment of the mouse and human cardiac troponin T (TNNT2) genomic sequences within the established 5′ portions of both genes. Identified CEs are boxed and unknown conserved regions are underlined. Deletion-mediated miniaturization designs of the human TNNT2 enhancer-promoter region were tested and a miniaturized 130-bp cardiac-specific enhancer located between the two triangles and a 170-bp miniaturized proximal promoter were created. MSEC-320 and MSEC-455c, containing the miniaturized promoter and either 1 or 2 miniaturized enhancers exhibit very high cardiac specific transcriptional activity. The 130-bp enhancer also provides greater skeletal and cardiac muscle transcriptional activity when ligated to CKM-based MSECs such as MSEC-571a (e.g., MSEC-725a and 875), see FIG. 16.

FIG. 11. Addition of one or more MCK small intronic enhancers (SIEs) confers stepwise activity increases to MSECs. In the experiment shown, multimerized (SIEs) were ligated to the 5′ end of MSEC-571 and expression activities were tested in skeletal muscle cultures using the dual luciferase reporter assay (FIG. 4). Conclusion: multimerized SIEs confer graded activity to MSECs and can achieve expression activity levels 15-fold higher than wildtype MSECs, and 7-fold higher than that of a strong CMV enhancer/promoter ubiquitously active regulatory cassette. Base pair lengths of the individual MSECs are indicated below the X-axis.

FIG. 12. Exemplary pGL3 plasmid map of MSEC-285. This MSEC contains a totally synthetic enhancer with its CEs indicated that is ligated to a miniaturized 99-bp promoter from human skeletal alpha-actin (ACTA1), and then to a Luciferase reporter cDNA.

FIG. 13. Exemplary pGL3 plasmid map of MSEC-429a. This MSEC contains a synthetic enhancer that differs from the MSEC-285 enhancer (FIG. 12), with respect to its CEs, their linear order and their spacing; and the synthetic enhancer is ligated to a miniaturized 129-bp promoter from human skeletal alpha-actin (ACTA1), and then to a Luciferase reporter cDNA.

FIG. 14. Exemplary pGL3 plasmid map of MSEC-393b. This MSEC contains a synthetic enhancer that differs from the MSEC-285 and MSEC-429a enhancers (FIGS. 12 and 13), with respect to its CEs, their linear order and their spacing. The synthetic enhancer is ligated to a miniaturized 134-bp basal promoter+Exon-1 region (−84 to +50) from mouse CKM, and then to a Luciferase reporter cDNA.

FIG. 15. DNA linkers of varying sizes and sequences are included in many MSECs as inserted sequences between enhancer and promoter regions, between multiple enhancers, and between multimerized miRNA target sites. Examples of short and long linker sequences are provided to illustrate the size range and sequence varieties that are present in different MSECs. Three fundamental aspects of linker inclusion within MSEC sequences are that: (1.) they introduce artificial sequences between the ligated components; (2.) they introduce different spatial distances between the ligated components; and (3.) these two factors can both affect each MSEC's transcriptional activity independently of the intrinsic activities of the MSEC's enhancer and promoter sequences. Linker sequences are thus considered as functional components within each MSEC as related to particular level of transcription within muscle cells. The linkers do not impact the selectivity of expression within muscle cells versus non-muscle cells.

FIG. 16. Transcriptional activities of 5 different MSECs in different adult mouse tissues following systemic administration of AdenoAssociatedVirus serotype-6 (AAV6) at 2×10¹² vector genomes per kg. Each MSEC was ligated to the human placental alkaline phosphatase (hPAP) cDNA, each cDNA contained the same poly-A sequence ligated to its 3′-end, and AAV LTR sequences were ligated to the 5′- and 3′-ends of each construct. Adult mice were injected retro-orbitally (N=6), euthanized 5 weeks later; different tissues were harvested, frozen and subsequently analyzed for hPAP/mg protein activity levels. Transcriptional activities of each MSEC were compared by normalizing all hPAP levels to those in the TA muscle of mice injected with MSEC-571a. Eight conclusions are supported by the overall data: (1.) Each MSEC expresses at much higher levels in muscle tissues than in non-muscle tissues; (2.) Each MSEC expresses at different levels in different anatomical muscles; (3.) MSEC-438a (CK8e). is more active in all skeletal muscles than any of the other MSECs; (4.) MSEC-455a is active in cardiac muscle, but essentially inactive in skeletal muscles; (5.) Addition of a 144-bp hTNNT2 miniaturized enhancer in a positive orientation to MSEC-571a to create MSEC-725a increases transcriptional activity in all skeletal muscles as well as in cardiac muscle (the 144-bp sequence is the 5′-most 144-bp of sequence shown for MSEC-725a in the MSEC library: (6.) Ligation of a second hTNNT2 enhancer in a positive orientation 5′- of the first enhancer (but lacking its 5′-most bp does not provide additional activity increases; (7.) Both MSECs containing additional hTNNT2 enhancers exhibit the highest activity in cardiac muscle; (8.) The very low expression of all of the MSECs in non-muscle tissues avoids deleterious effects of expressing a product in an unintended cell type, as well as stimulating immune system dendritic cell responses to the product.

FIG. 17. Use of microRNA (miR) target sites for limiting product expression in cell types in which the product is, or might be, deleterious; e.g., cardiac muscle. Although many MSECs exhibit different transcriptional activities in different muscle types, as well as much higher activities in muscle than in non-muscle cells (e.g., FIG. 16), some therapeutic products may be deleterious if expressed above particular levels in one or more tissue types following systemic vector delivery. This problem can be ameliorated by inserting binding site sequences that are complementary to whatever miRs are known to be present in one or more cell types in which the expressed product is undesirable. In this study 3 tandem miR208a target sites (see miR208aTSx3 in FIG. 27, page-1) were ligated into the 3′-portion of a Luciferase cDNA and targeted and control cDNAs were ligated to MSEC-438a (indicated as CK8e in the FIG.) constructs were transiently transfected into either differentiating MM14 skeletal muscle cultures or into neonatal rat cardiomyocyte cultures and culture extracts were collected 48 hours later. The presence of miR208a target sites repressed Luciferase product levels by 95% in cardiomyocytes; while miR208a target sites caused no product level repression in differentiated skeletal muscle cells. (MSEC-1263a was transfected as a positive control in both cultures, and as a means of illustrating the elevated transcriptional activity of MSEC-438a).

FIG. 18. Use of microRNA (miR) target sites for limiting product expression in skeletal muscle. Three tandem miR206 target sites (see miR206TS-3Gx3 in FIG. 27, page-1) were ligated into the 3′-portion of a Luciferase cDNA and targeted and control cDNAs were ligated to MSEC-438a (indicated as CK8e in the FIG.), or to MSEC-725a (indicated as h-cTnt(E)-CK7 in the FIG.) constructs were either electroporated into cultured intact mouse FDB muscle fibers isolated directly from adult mice; i.e., to mimic an in vivo differentiated muscle fiber environment) (left bars per group), or transiently transfected into neonatal rat cardiomyocyte cultures (right bars per group), and culture extracts were collected 48 hours later. The presence of miR206 target sites in both MSEC constructs repressed Luciferase product levels by 90% in skeletal muscle fibers; while miR206 target sites caused no product level repression in differentiated cardiomyocytes.

FIG. 19. Frequency plot at each base position in the identified TATA-box elements in all vertebrate genes queried in the Jaspar 2020 Transcription Factor DataBase (http://jaspar.genereg.net/search?q=TATA+box&collection=CORE&tax_group=vertebrates&tax_id=all&type=all&class=all&family=all&version=all). This is compared to the mouse CKM TATA-box sequence written below. The FIG. illustrates three concepts: (1.) the most frequent core TATA-box sequence is: TATAAAA; (2.) TATA-box sequences exhibit high variability; and (3.) The mouse CKM core TATA-box sequence differs from the “consensus” vertebrate TATA sequence by lacking the 2 3′-As, whereas the TATA box of the most highly transcribed human skeletal muscle gene (FIG. 6) ACTA1 is a perfect match to the vertebrate consensus TATA sequence (see MSEC-239 sequence). These comparisons are consistent with the hypothesis that the TATA-box sequence is an important determinant of MSEC transcriptional activities.

FIG. 20. Naturally occurring Adenovirus major late promoter TATA-box variants exhibit 20-fold activity differences. By analogy to the Adenovirus TATA-box sequence variations and transcriptional activity differences, in some embodiments, TATA modifications of selected MSECs can be used to reduce or increase activities with or without affecting the MSEC's response to the binding of other transcription factors (TFs) to its CEs.

FIG. 21. N-box CEs occur in the promoters and enhancers of muscle genes that are transcribed at high levels in muscle fiber myonuclei located near neuromuscularjunctions (NMJs). This FIG. illustrates that the naturally occurring locations of N-box elements vary widely, and that N-boxes function within Proximal promoter regions close to the transcription start site (TSS), as well as within enhancer regions located 5′- and 3′- from the TSS. This implies that N-boxes can function when inserted in many different MSEC locations, either as miniaturized N-box-containing enhancers isolated from muscle genes such as Acetylcholine esterase (ACHE) and Acetylycholine receptor subunits such as (CHRM1, CHRND, CHRNG), or as multimerized N-box clusters such as the 43-bp cluster of 3 N-boxes shown in the FIG. In vitro assays prove the function of such N-box-containing MSECs in differentiated skeletal muscle cells in response to the addition of specific ligands such as agrin and neuregulin to the culture media (PMID: 11498047; and Refs listed in the FIG). Optimal N-box locations within MSECs are thus determined by quantitative comparisons of expression levels of N-box-containing MSECs in response to externally added nerve-mediated factors that stimulate N-box-binding TFs such as GAPB to associate with the inserted to N-boxes (PMID: 11498047). N-box function in association with different MSECs ligated therapeutic cDNAs to is thus anticipated to provide therapeutic products to NMJ regions and much less product to non-NMJ regions of skeletal muscle fibers.

FIG. 22. MSEC design scheme for miniaturization of an alpha-MyHC enhancer for designing smaller versions of MSEC-770. Progressive deletions of unannotated and nonconserved regions reduces the human alpha-MyHC enhancer from 190-bp to 90-bp. Combining the most extensively miniaturized alpha-MyHC enhancer with MSEC-438a would increase activity while reducing the new MSC's length to 526-bp (relative to MSEC-770). Reductions of MSEC length facilitate packaging of MSECs in conjunction with large cDNAs in viral vectors or other gene delivery systems with limited space for nucleic acid cargos.

FIG. 23. Human cardiac Troponin T (TNNT2) enhancer and promoter regions (see FIG. 10) were used to design MSECs with graded expression levels in differentiated cardiomyocytes in vitro. MSEC-455a contains 2 miniaturized TNNT2 enhancers and exhibits high levels of cardiac muscle-specific activity in vivo (see MSEC-455a data in FIG. 16.

FIG. 24. Synthetic striated muscle-specific enhancers are designed by combining muscle gene control elements in 5′ to 3′ orders and spacings that provide different qualitative and quantitative transcriptional activities in different striated muscle types. Because any number of control elements can be linked together with different spacings this facilitates the design of both small and large enhancers. Synergistic transcription factor interactions can also be facilitated by placing CEs known to bind synergistic TFs adjacent to each other. Synthetic enhancers are then linked to a wide variety of muscle gene or ubiquitous gene promoters and cDNAs to produce desired product levels.

FIG. 25. All MSEC designs are tested for expression in non-muscle cell cultures to determine their muscle-specific properties. FIG. 25 illustrates the range of very low activities that typical MSEC exhibit in 3T3 mouse fibroblast and human foreskin fibroblast cultures in comparison to the activities of two CMV enhancer/promoter combinations that express at high levels in all cell types. Other examples of MSEC versus ubiquitous enhancer/promoter combinations in a variety of non-muscle cell types are published in PMID: 17235310.

FIG. 26. illustrates comparative in vivo data between two MSECs that contain multiple differences in their control elements as well as in the length of their CKM Exon-1 components (see Library sequences for MSEC-411 compared to MSEC-438a). Mice were euthanized 4 weeks post-injection and tissues were frozen, extracted and assayed as in FIG. 26. Mean hPAP activities (×10⁶ enzyme units) per mg extract protein were determined for each tissue type and the relative expression levels in each tissue were calculated by determining the ratios of MSEC-411 to MSEC-438a activities. The data shows that MSEC-411 has about 2 times greater activity than MSEC-438a in all skeletal muscles and equal activity in cardiac muscle; and both MSECs have equivalent very low expression in a non-muscle tissue (liver).

FIG. 27. Library of exemplary MSEC sequences supporting the disclosure. All sequences are printed from Left to Right in their 5′- to 3′-directions. Linker sequences are underlined in most sequences. Some exemplary MSECs sequences also show the sequences of introns and/or 3′-untranslated regions. miRNA target site sequences are shown at the beginning of the list; these would be inserted 3′- of cDNAs that are ligated to any of the MSECs. Sequences are ordered from smallest to largest.

### DETAILED DESCRIPTION

Skeletal and cardiac muscles are affected by hundreds of genetic diseases, are subject to many types of physical injury, undergo progressive functional weakness during disuse and aging, and skeletal muscle also undergoes debilitating catabolic degradation in conjunction with cancers. Since all skeletal muscle fibers are innervated, and since the function of muscle cell synaptic regions where neuronal axons stimulate muscle contraction depends on neuronal interactions, muscle cells also exhibit a variety of Neuromuscular Junction (NMJ) diseases. Additionally, since the maintenance of innervating neurons is partially dependent on muscle-mediated signals, muscles also play important roles in the normal function of their innervating neurons. Muscle-Specific Expression Cassette (MSECs) described herein can play major roles in therapeutic strategies to develop treatments to combat all of these medical issues, as well as analogous issues in veterinary medicine. Importantly, two previously developed MSECs are providing encouraging results in several on-going Duchenne Muscular Dystrophy clinical trials.

The DNA sequences described herein have been determined via iterative design and testing strategies that were originally based upon four decades of data from basic research studies focused exclusively toward understanding skeletal and cardiac muscle gene regulation. These basic studies examined the mouse M-creatine kinase (MCK/CKM) gene to identify its regulatory regions. As illustrated in the many basic research publications summarized below, none of the basic findings were described in terms of their applicability to gene therapy development or other commercial uses. Although the 1993 transgenic mouse publication (Cox, G A, et al: PMID: 8355788) demonstrated the potential feasibility of eventually treating Duchenne Muscular Dystrophy (DMD) via gene replacement therapy, the 6,300 bp regulatory sequence and 14,000 bp dystrophin cDNA sequences used would not have been applicable to any realistic therapeutic strategies of that era. Furthermore, while this and many subsequent basic research studies provided extensive publicly available information regarding the mouse CKM gene's regulatory components, no other investigators applied available research data toward these ends until the potential use in a miniaturized MSEC (“CK6”) in the 2000 publication, Hauser, M A, et al., 2000; PMID: 10899824.

An example of considerations regarding uses of MSEC disclosed herein is provided in relation to striated muscle and as compared between skeletal and cardiac striated muscle types. Striated muscles represent the largest tissue mass in all vertebrates. Humans and many vertebrates contain more than 600 anatomically different skeletal muscles as well as multiple cardiac muscle types. Striated muscles are composed of fibers and muscle cell types that exhibit type-specific gene expression. Each human anatomical skeletal muscle is unique and contains dozens to thousands of multinucleated fibers with varying proportions of each of 3 fiber types as well as mixed fiber types. Fiber lengths range from 1 mm in the inner ear to 60 cm in the thigh and contain ˜50 myonuclei per mm. The longest muscle fibers in tall individuals thus contain as many as 30,000 myonuclei each, and the entire muscle contains well more than 10 million myonuclei. With few exceptions, all myonuclei in each skeletal muscle fiber are presumed to express the same genes at the appropriate levels for each gene. Based on quantitative analysis of all skeletal muscle mRNA levels, the relative steady-state rates of gene expression vary over more than 5000-fold between the most active and least active genes. In contrast, human ventricular, atrial, and several other cardiac muscle types contain only 1-3 myonuclei/cell, but they also exhibit wide ranges of transcription rates for different genes.

Multinuclearity is potentially beneficial for skeletal muscle gene therapy, but less so for cardiac muscle gene therapy. Ideal striated muscle gene therapy would result in the transduction of each muscle cell nucleus with equal vector numbers so that all nuclei would produce equal amounts of therapeutic product. This goal has not been achieved by any viral mediated therapies, and current protocols in which the highest FDA-approved vector doses for humans have been tested at equivalent body-weight does in mice, indicate that not all skeletal and cardiac myonuclei are transduced. Furthermore, transduced nuclei appear to contain variable vector numbers. This has critical consequences for gene therapy efficacy, because if too few myonuclei are transduced some regions within long skeletal muscle fibers may receive suboptimal therapeutic product levels, and some cardiomyocytes may contain no transduced myonuclei and thus receive no direct therapeutic benefits. Variable transduction in skeletal muscle myonuclei can be largely overcome via the use of Muscle Specific Expression Cassettes (MSECs) with very high transcription activities, since the high product levels produced by transduced myonuclei can diffuse laterally into fiber regions lacking transduction and thus compensate for transduction deficits in these regions. In contrast, human cardiomyocytes typically have only 1-3 nuclei, thus at least one nucleus in each cell needs to be transduced to provide benefits. However, because random myonuclei may also be transduced by very high vector numbers, excessive product levels produced by these myonuclei could produce local toxicity. Although therapies for some muscle diseases may provide partial benefits to neighboring fibers and cardiomyocytes that lack sufficient therapeutic products, a necessity for optimal treatments is obtaining sufficient, yet non-toxic, levels of therapeutic product.

These fiber type differences are critical with respect to the design of MSECs for muscle gene therapy because fiber type transcription factor (TF) differences can cause the same MSEC to express excess therapeutic product in some fibers and insufficient product in others. It is thus advantageous to have a wide range of MSECs that can express equal high, medium, or low product levels in all fiber types, as well as MSECs whose transcription rates are high, medium, low, or even “off” in different fiber types (e.g., high in Type I, medium in Type IIa, and low or “off” in Type IIx, as well as all combinations of these activity levels). Analogous TF differences are responsible for differential gene expression in atrial, ventricular, and conducting cardiomyocytes in the heart. The wide range of MSEC transcriptional activities in the disclosed MSEC library has been created by modifying control element (CE) types, sequences, spacing and adjacent neighboring CEs to create MSECs that respond differently to the wide variety of physiological signals associated with different muscle types as well as changes in workloads. As described below, optimal MSECs for each therapeutic goal will differ due to unique attributes of each therapeutic product, as well as pathological differences between diseases.

An additional level of gene product control is exerted by the numerous microRNAs (miRNAs) expressed in proliferating skeletal muscle myoblasts, differentiated fibers types, and cardiomyocyte types. miRNAs typically affect the extent to which specific mRNAs are translated into proteins by enhancing degradation rates of the mRNAs to which they bind. The selectivity of which mRNAs are degraded is due to qualitative and concentration differences among the miRNAs produced by each cell type, and on the miRNA binding affinities for the slightly differing miR target site (miRTS) sequences that reside within different muscle gene mRNAs. By inserting different numbers of miRTs with high-to-low binding affinities into the non-coding regions of any desired product's cDNA, it is thus possible to adjust product levels in specific muscle subtypes with even greater precision than can be achieved via transcriptional controls alone. Finer tuning of product levels between muscle types can thus be obtained by selecting appropriate MSECs and miRTSs for each disease therapy.

As indicated, skeletal muscles contain mixtures of fast and slow fibers whose biochemistry and physiology is largely influenced by their initial embryonic cell lineages, subsequent innervation, and on-going workloads. The relative numbers and spatial distributions of each fiber type differ between and within sub-regions of individual anatomical muscles. Slow fibers (Type I) contract for relatively long time periods, use aerobic metabolism, and express myosin heavy chain type I, whereas Fast fibers contract and relax more rapidly, depend on greater levels of glycolytic metabolism, and express 2 or 3 different myosin heavy chain genes: Type IIa and IIx in humans, dogs, sheep and cattle, and Type IIa, IIx, and IIb in pigs, cats and rodents, and analogous fiber types in poultry. Each fiber type also contains unique subsets of contractile and other proteins that are specialized for their slow and fast muscle functions.

Muscle diseases often exhibit different severities among striated muscle types, or between skeletal and cardiac muscle. Age-related sarcopenia, cancer cachexia, and spinal cord injuries tend to affect Type II fibers more than Type I fibers. Duchenne (DMD) and Facioscapulohumeral (FSHD) muscular dystrophies also exhibit greater effects on Type II fibers, while FKRP-mediated Dystroglycanopathies (MDDGA5, MDDGB5 and MDDGC5), exhibit relative increases in Type I fibers, possibly due to gradual Type II- to Type I transitions. In contrast, Myotonic dystrophy and some Limb Girdle Muscular Dystrophies (e.g., LGMD2A due to Calpain-3 deficiency) are associated with reduced Type I fibers. NMJ diseases also exhibit skeletal muscle fiber type changes; e.g., infants with the most severe forms of Spinal Muscular Atrophy (SMA) have many fewer Type II fibers and an associated increase in Type I fibers; and patients with advanced Amyotrophic Lateral Sclerosis (ALS) exhibit a transition from Type II to Type I fibers. Some striated muscle diseases such as DMD affect both skeletal and cardiac muscles, whereas others primarily affect skeletal or cardiac muscle, and some cardiac muscle diseases have their most pronounced effects on either ventricular, atrial, or conduction components. These disease-specific muscle type differences underscore the importance of developing MSECs that are optimized for each disease type so as to focus gene therapies to the most affected muscles and fiber types.

Challenges for Achieving Product-level control in treating neuromuscular diseases. The natural gene expression levels of all striated muscle proteins and RNAs in different muscle types have evolved such that each has an optimal level. A critical consequence of this is that either under- or over-expression of any therapeutic product will—at the very least—be sub-optimal; and over-expression could be potentially deleterious due to either dominant-negative protein-protein interaction effects or to inappropriate activities of a therapeutic protein in a particular muscle type; (e.g., systemic AAV administration of the Calpain-3 protease under control of the relatively high-activity desmin gene promoter in skeletal muscles of a mouse LGMD2A model is beneficial, whereas the same mice exhibit Calpain-3 toxicity in their ventricular muscles due to Calpain-3 over-expression).

Normal levels of each muscle protein and regulatory RNA species are achieved via a combination of transcriptional, post transcriptional and translational mechanisms. Transcriptional controls are mediated by the interactions of ubiquitous and tissue-specific transcription factors with CE complexes within each gene's regulatory regions (proximal promoter and one or more enhancers). Post-transcriptional controls are mediated by miRNAs and miRNA target sites, and by protein-mRNA interactions, and translational controls are mediated via mRNA splicing mechanisms. Additional product level controls are regulated by mechanisms affecting protein half-lives, such ubiquitin-mediated degradation pathways. A standardized approach to determining optimal MSECs and vector doses is described elsewhere herein.

Vector Dose—MSEC Activity Relationships. Since therapeutic product levels obtained with different MSECs vary depending on vector dose and intrinsic differences in vector type-specific transduction efficiencies, it is important to recognize that each fiber or cardiomyocyte needs to be transduced with sufficient vectors to produce at least minimally beneficial therapeutic product levels. For many LGMDs that affect genes encoding enzymes the necessary product levels may be much lower than those encoding high-abundance contractile proteins such as actin. This is demonstrated by quantitative studies of relative mRNA transcript levels available on public websites such as the Broad Institute gtexportal.org site (e.g., -Skeletal muscle actin, ACTA1, mRNA levels in adult human skeletal muscle are 5,000 times greater than those of -Dystroglycan glycosylase (GMPPB)). Thus, while it might seem that the same high-expressing MSEC could be used to treat both Nemaline Myopathy (an ACTA1 muscle disease) and MDDGC14 (a GMPPB enzyme muscle disease) by simply reducing the vector dose by 5,000-fold, this assumption would be incorrect since dose reduction inevitably leads to some fibers receiving zero or too few vectors to provide therapeutic benefits. That said, relative mRNA levels on the gtexportal.org site were primarily obtained from older adults who died from cardiac disease, thus these values may not be fully accurate for younger patients with different disease phenotypes.

MSEC Characteristics. Basic studies have identified multiple skeletal and cardiac muscle gene enhancer and promoter regions, their CEs, and transcription factor interactions with these DNA components. Continually increasing knowledge of these components, the signal transduction pathways that affect them, and the identification of muscle type-specific miRNAs, and their diverse binding sites on mRNAs, provide the underlying strategies for selecting MSECs that exhibit desirable qualitative and quantitative product level differences in each muscle type.

The present disclosure describes more than 300 MSECs that can be used to express cDNAs encoding any protein or RNA in differentiated skeletal and cardiac muscle cells, while expressing at only background levels in non-muscle cells. All of these RCs have been tested for transcriptional activities in differentiated skeletal muscle cultures, and many have been evaluated in newborn rat cardiomyocyte and non-muscle cell cultures. Additionally, a subset has been tested for expression in vivo in either transgenic mice or via systemic vascular delivery of AAV vectors.

A general finding from the in vivo studies is that the relative transcriptional activities of different MSECs based on in vitro studies are suggestive, but not fully predictive of relative in vivo activities of the same MSECs. 4 points are important to highlight:

(1) All of the in vitro MSEC evaluations are fully relevant with respect to expressing graded levels of desirable protein or RNA products in differentiated skeletal and cardiac muscle cell cultures over a 1000-fold concentration range.

(2) The absence of MSEC expression in non-muscle cell cultures correlates extremely well with the absence of expression in all non-striated muscle tissues in vivo.

(3) Differences in MSEC expression levels in differentiated skeletal and cardiac muscle cell cultures are partially predictive of similar relative differences between the same MSECs in vivo, and thus serve as useful starting points from which to identify optimal MSECs for each in vivo therapeutic purpose.

(4) Since many MSECs differ from each other by only 1 or 2 subtle variables, in vivo data for one member of a related MSEC set is very likely to be predictive for most other members of the related MSEC set.

The MSECs presented in the library of the present disclosure have a wide range of transcriptional activities; and importantly, those tested in vivo exhibit differences in relative expression levels among individual anatomical muscles and muscle fiber types. Since many MSECs have been designed using regulatory components of the M-creatine kinase gene (CKM), and since the native gene exhibits higher transcriptional activity in skeletal than cardiac muscle, and in fast fibers than in slow fibers, it was anticipated that these attributes would be seen in CKM-derived MSECs. This is indeed the case; e.g., “MSEC-438a” one of the more active MSECs, exhibits relative expression levels in the fibers of mouse TA and Soleus muscles in the order: Type IIb>Type IIx>Type IIa>Type I in ratios of about: 8:4:2:1. Because humans do not have Type IIb fibers, the current unverified prediction is that therapeutic products expressed via MSEC-438a in humans would be about: 4:2:1 in Type IIx, IIa, and I fibers.

An important corollary of these fiber type transcriptional differences is using MSEC-438a for micro-dystrophin mediated DMD gene therapy is likely to produce 4:2:1 ratios of micro-dystrophin in patient Type IIx, IIa, and I fibers. Although previous mouse DMD studies suggest that higher than normal dystrophin levels are non-toxic, the fact remains that irrespective of vector dose, DMD patient Type IIx fibers may have 4-times higher micro-dystrophin levels than Type I fibers, and twice as much as Type IIa fibers. Biopsy data from on-going human clinical trials using MSEC-438a should indicate whether this is the case; and physiological studies of anatomical muscle with different fiber type distributions should indicate whether there are functional consequences.

Nevertheless, if a given vector dose indicates that Type IIx fibers have barely sufficient levels of micro-dystrophin, the levels in Type IIa and Type I fibers would likely be much lower. Type IIx fibers might thus appear “cured,” while type IIa and I fibers would have 2-4 times lower micro-dystrophin levels. Obtaining sufficient product levels in all fiber types is thus highly beneficial, and new MSECs containing alternative sets and arrangements of striated muscle CEs can provide these properties.

MSECs in Clinical Trials. Two previously developed MSECs (MSEC-770 and MSEC-438a) are functioning well in ongoing DMD Clinical Trials being carried out by Serepta and Solid Biosciences. For example, biopsy data from the Serepta Trial indicate that MSEC-770 is probably expressing micro-dystrophin in all fiber types (although relative levels are not yet known), and plasma creatine kinase levels in the treated boys are significantly reduced—an indication that body-wide dystrophic muscle fiber damage is being ameliorated. Based on these findings, these same MSECs could be used for pilot gene therapy studies for treating other Neuromuscular diseases. However, as detailed data becomes available from the ongoing trials, it will almost certainly suggest that either more or less transcriptional activity would be beneficial for DMD therapy, and thus that alternative MSECs should be selected in subsequent Clinical Trials. Furthermore, since the protein products involved in other NMDs will have different functions and be required at different levels than the Micro-Dystrophin used for DMD therapy, it is very likely that different MSECs will be optimal for these therapies. Identifying beneficial MSECs for each NMD will require several rounds of in vitro and then in vivo pilot studies to identify optimal MSECs. The availability of several hundred MSECs with transcriptional activities spanning a 1000-fold range will greatly facilitate this process. An example of this sequential approach for rapidly identifying optimal MSECs for different NMDs is provided below.

In assessing vector dose ranges for particular uses, this can depend on previously established vector dose safety studies, on transduction efficiencies of the viral vector, and knowledge concerning post-transcription diffusion/transport and half-life parameters of the NMD mRNA and protein. Although “ideal vector doses” might be envisioned to transduce all skeletal and cardiomyocyte myonuclei with only one transcriptionally active therapeutic vector genome that produces the same NMD product levels produced by normal myonuclei, this simplistic goal is not yet achievable. At therapeutically safe vector doses, some myonuclei invariably contain more vectors than others and some contain no vectors. At very high vector doses, some myonuclei might thus produce locally toxic therapeutic product levels, and at low vector doses some myonuclei will produce no therapeutic product. Current AAV dose levels are thus best guided by pragmatic experience related to the specific serotype, route of delivery, and infusion rate parameters, and knowledge regarding transduction efficiencies among different anatomical muscles.

Since human transduction efficiency data is essentially non-existent, vector doses are typically based on extrapolations from mouse and other animal studies. That said, Duchenne Muscular Dystrophy gene therapy trials in humans suggest that beneficial systemic micro-dystrophin delivery is obtained at doses of 2e14 AAVrh74 vg/kg in conjunction with the MHCK7 MSEC (Asher, et al. 2020. //doi.org/10.1080/14712598.2020.1725469). This dose is at least 3-fold lower than equivalent AAV doses in mice and nonhuman primates that are known to be tolerated without obvious vector toxicity. Since lower vector doses would be desirable from cost and safety perspectives, two reasonable AAV doses for initial MSEC selection studies in mice would thus be 2e14 and 2e13 vgs/kg, or if testing at a single dose 7e13 vg/kg (3.5e12 vg/20 g 5 wk old male mouse). Alternatively, intramuscular injections of 1e10 vg into mouse TA muscles could provide preliminary data regarding MSEC-mediated expression of the therapeutic product. Prior to making AAV vectors it is always cost- and time-effective to test MSEC-therapeutic cDNA constructs in skeletal and cardiac muscle cultures, as well as in non-muscle cell cultures to verify that reasonable levels of muscle-specific product expression are obtained. This avoids expression issues due to cloning errors and to unanticipated positive or negative effects of the cDNA sequence on transcription.

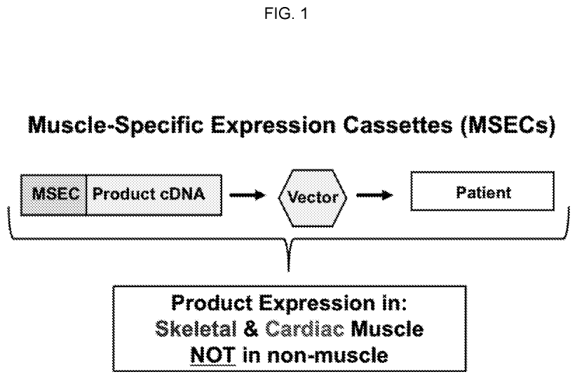

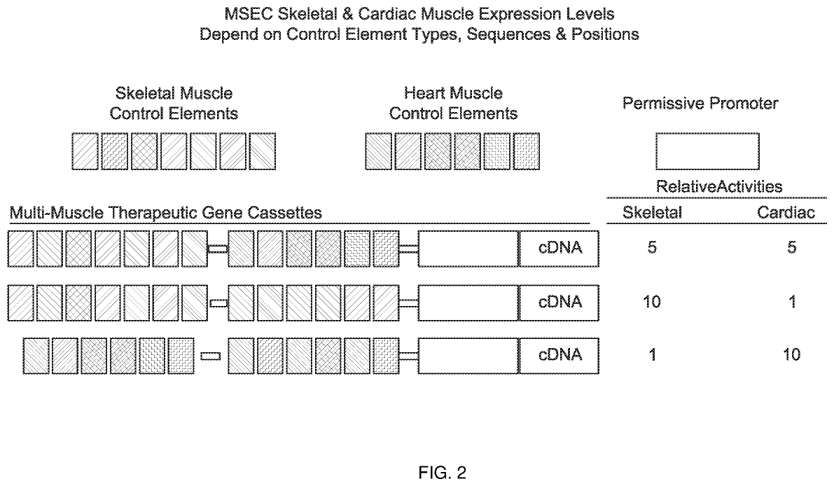

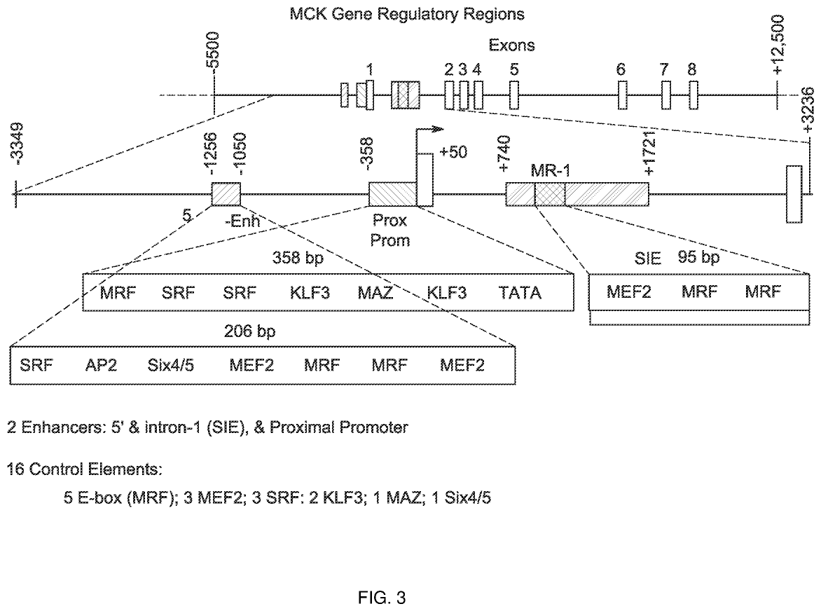

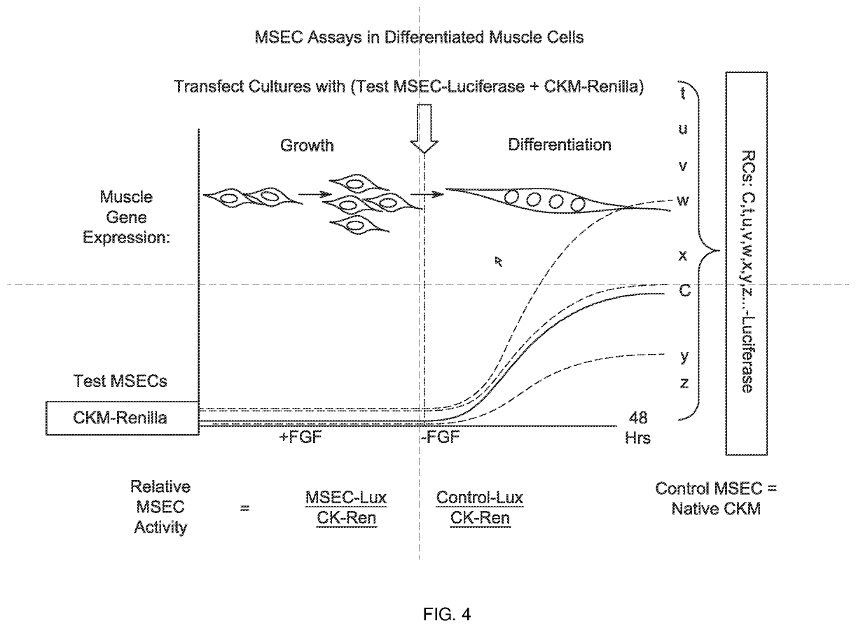

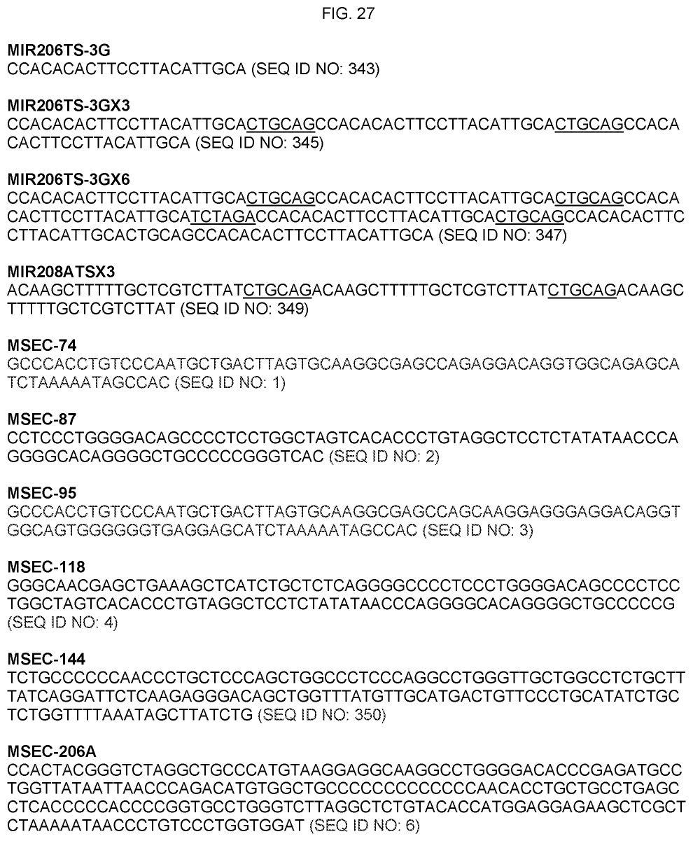

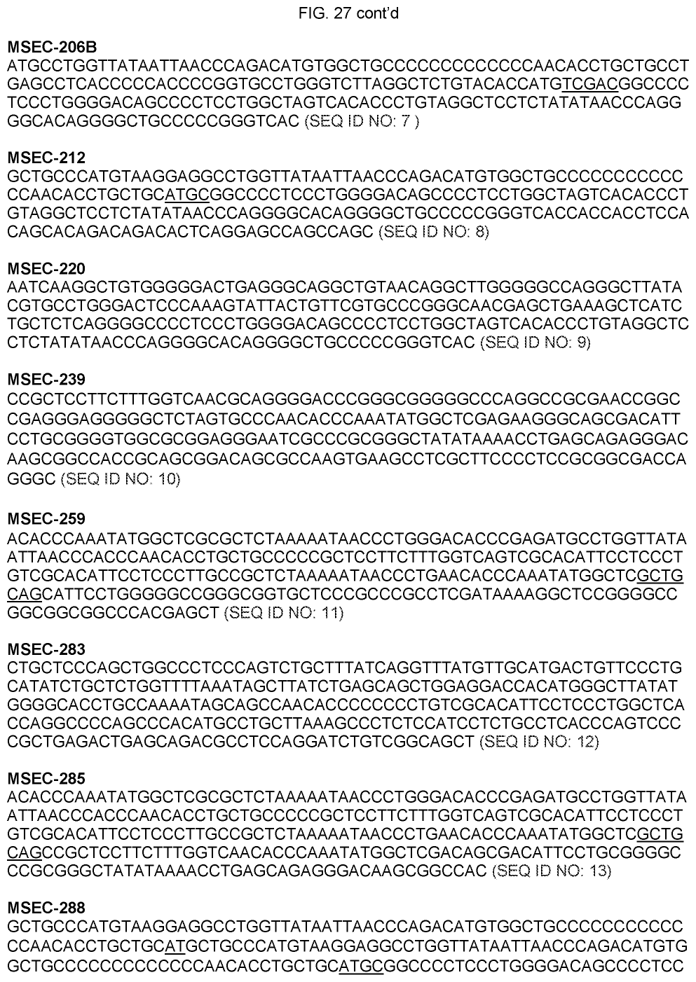

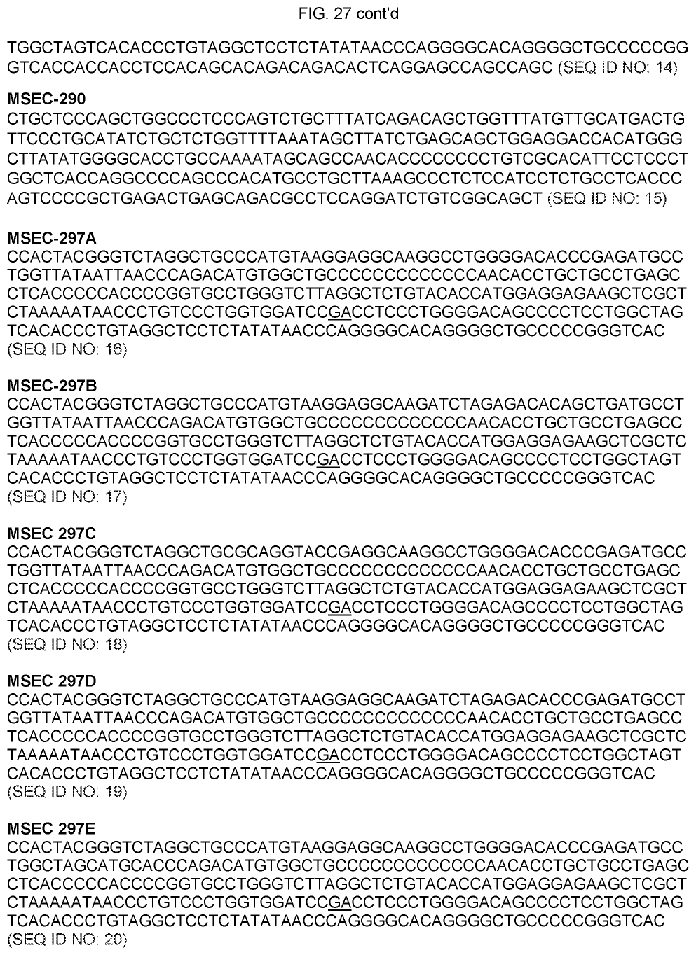

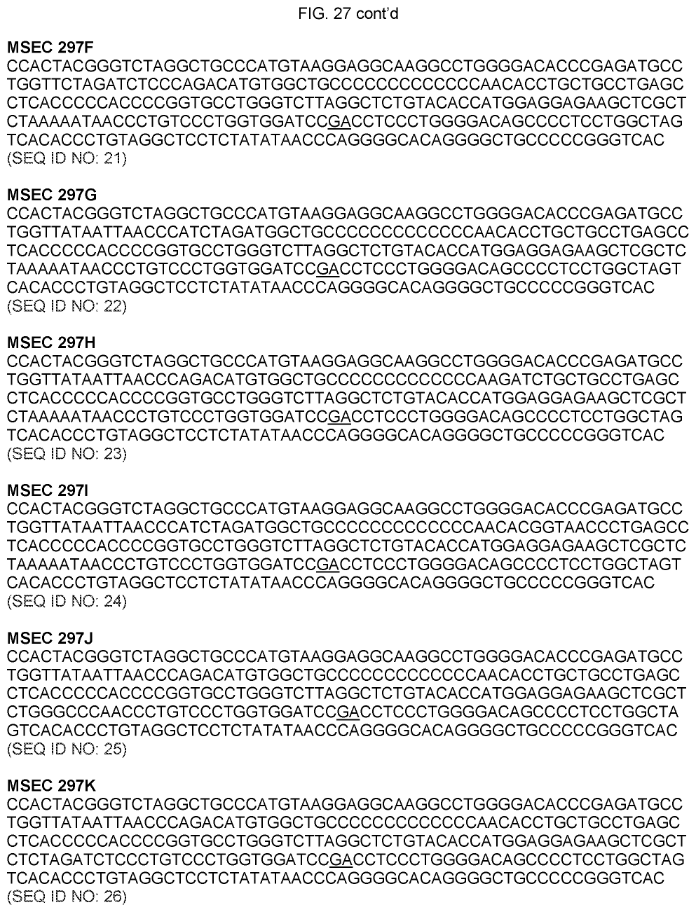

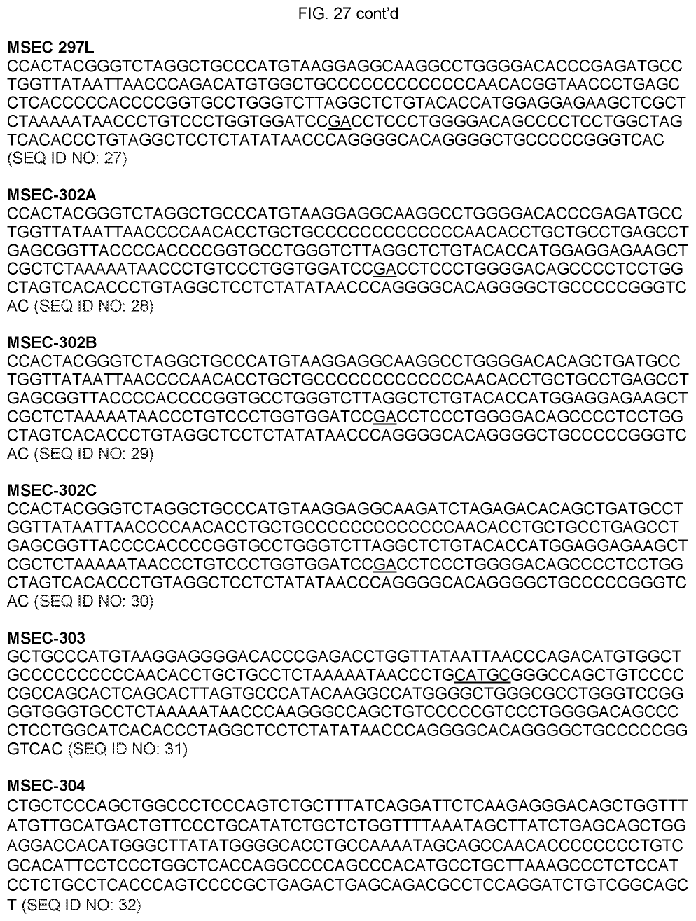

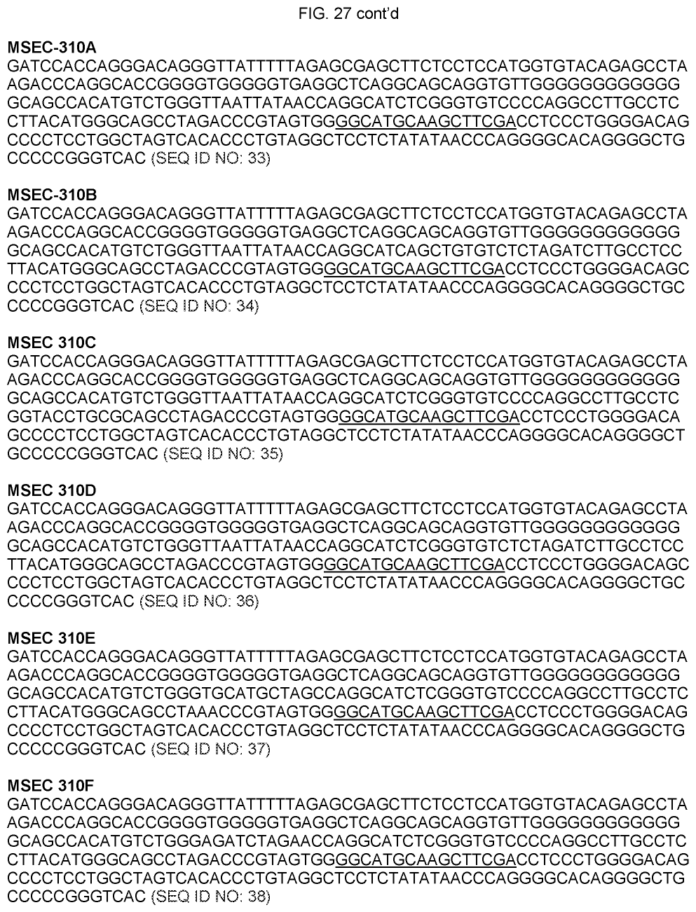

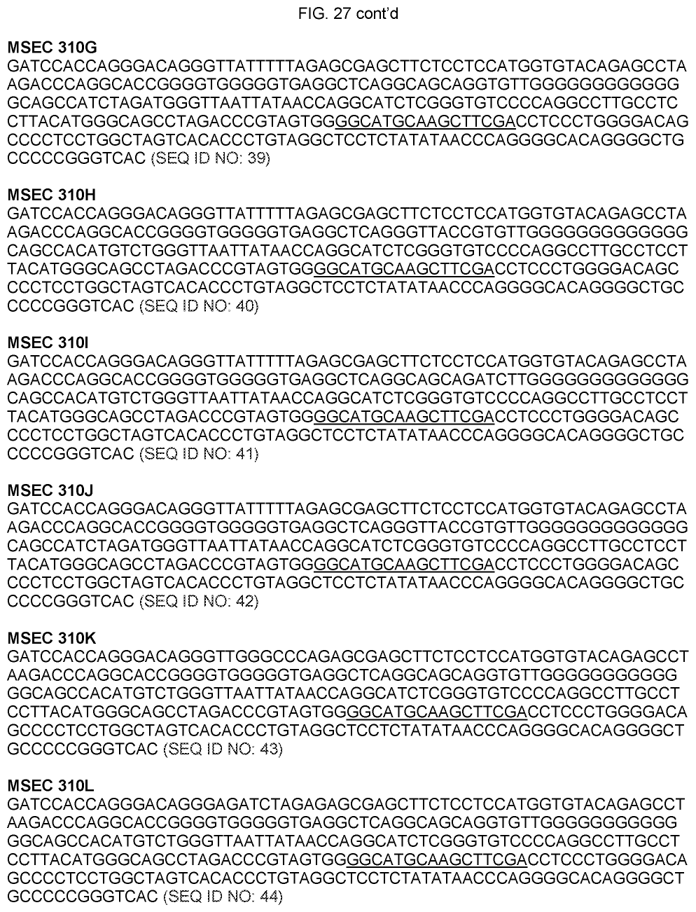

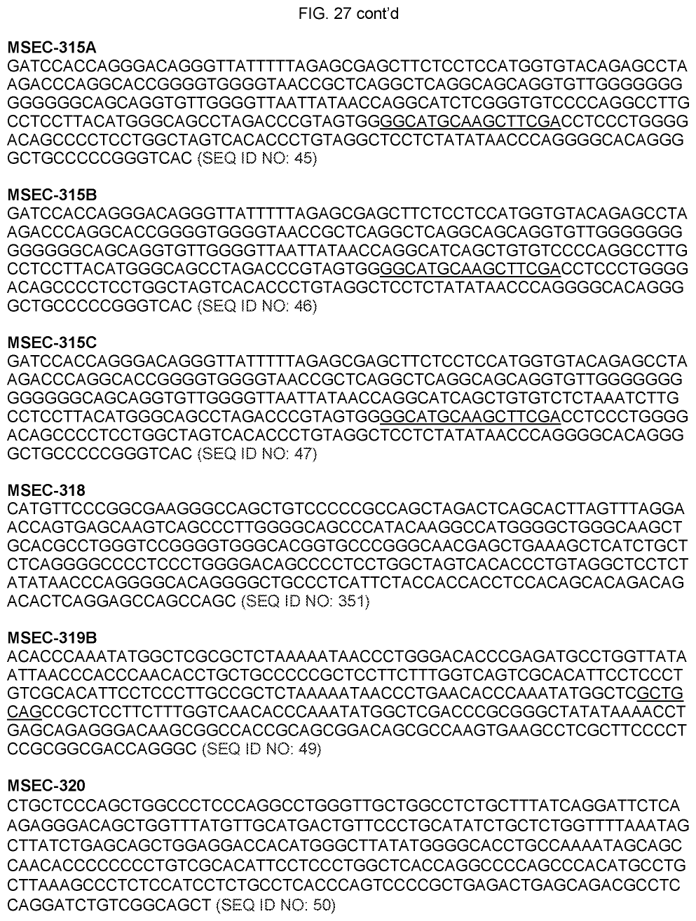

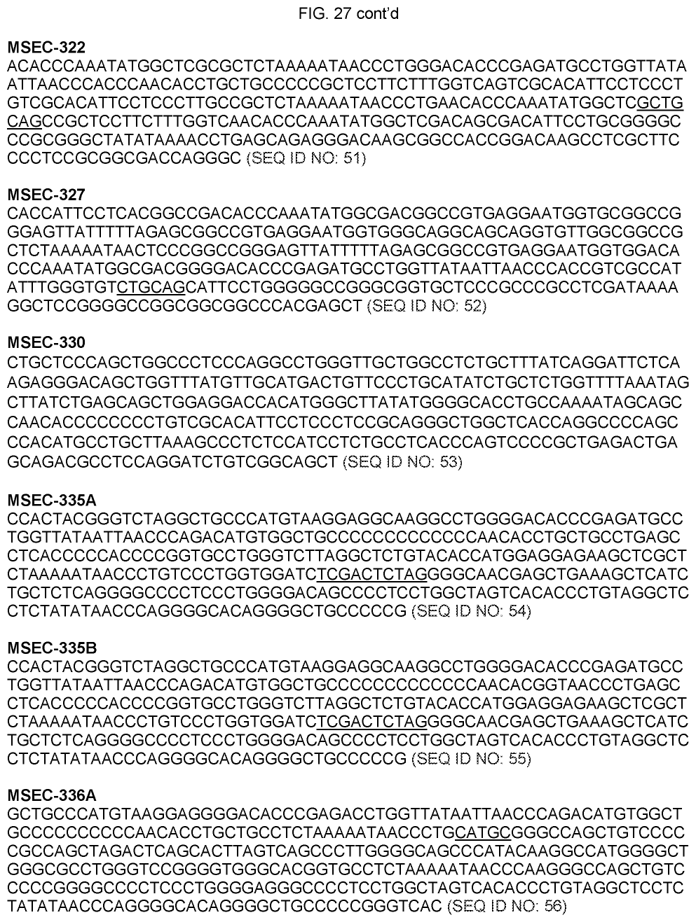

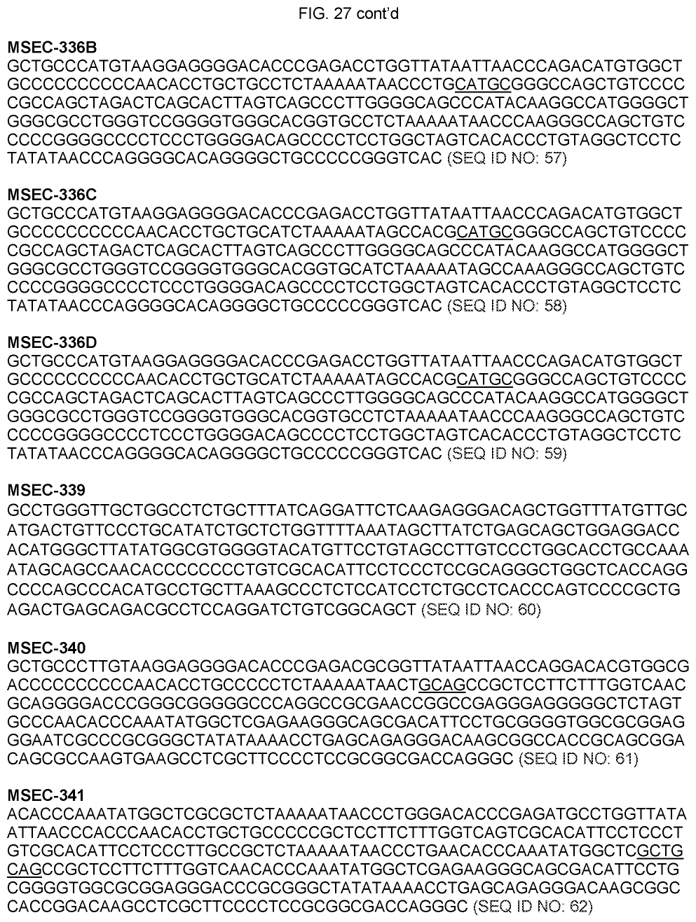

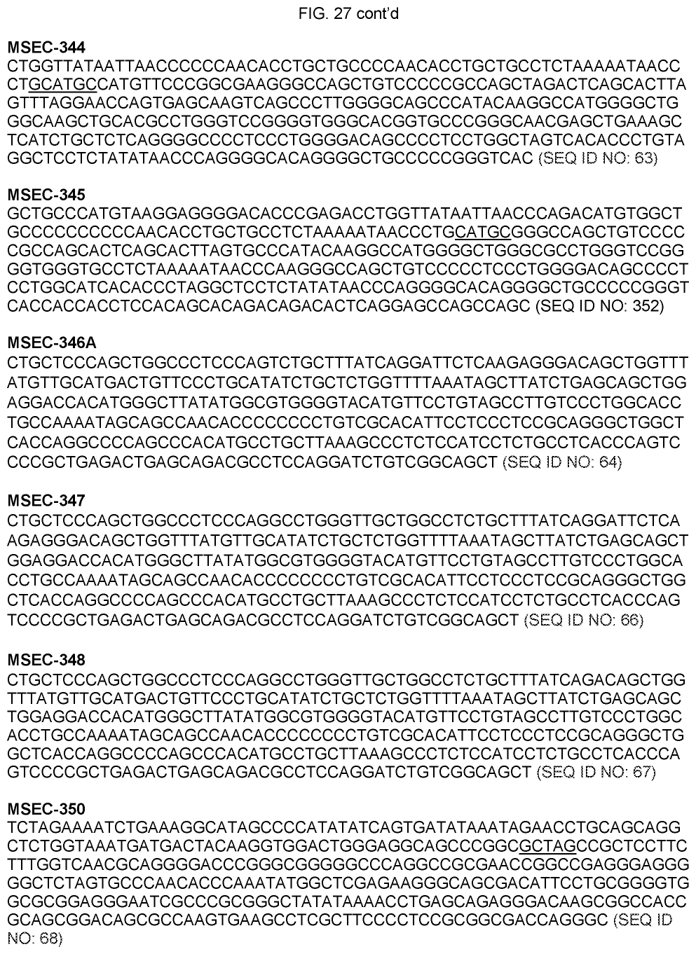

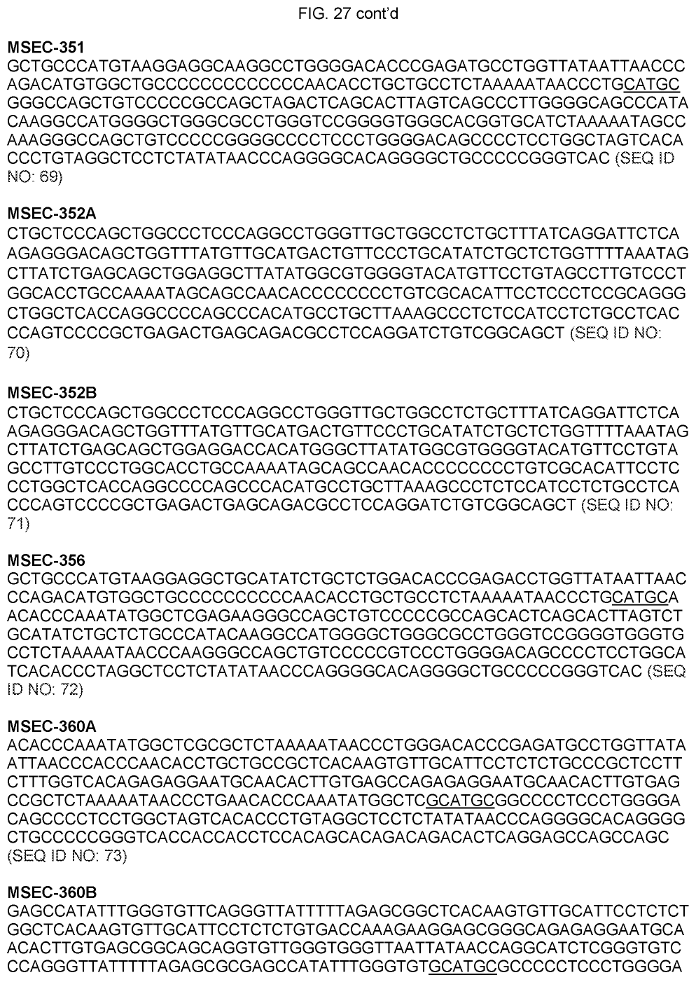

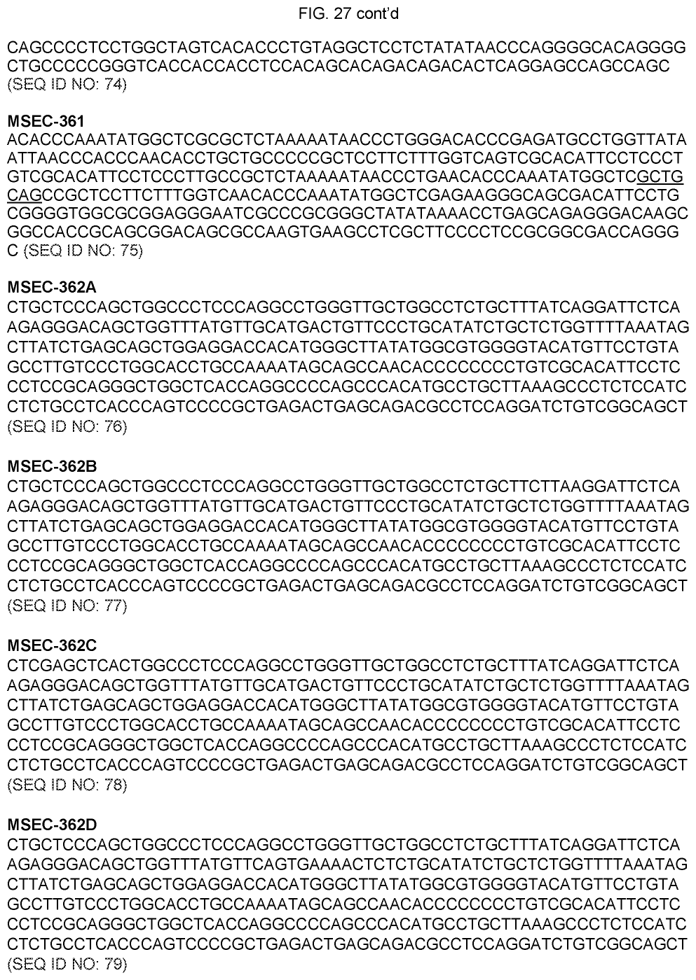

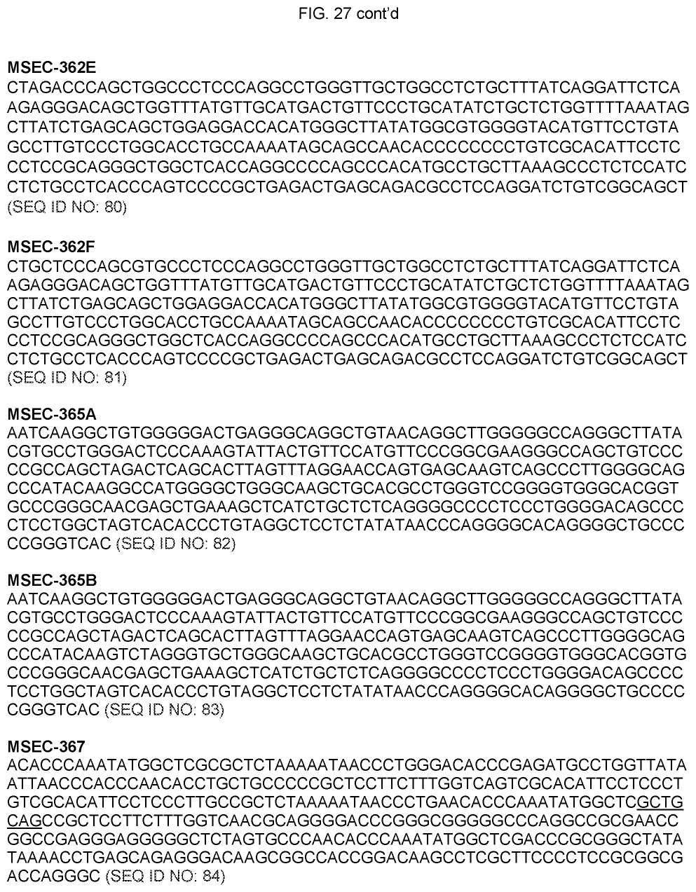

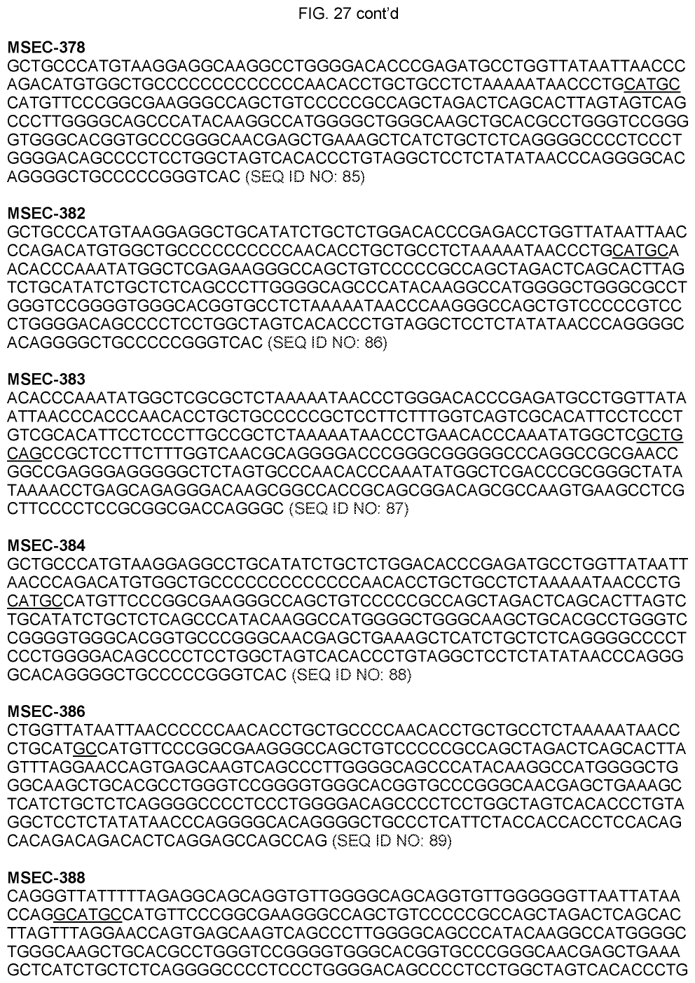

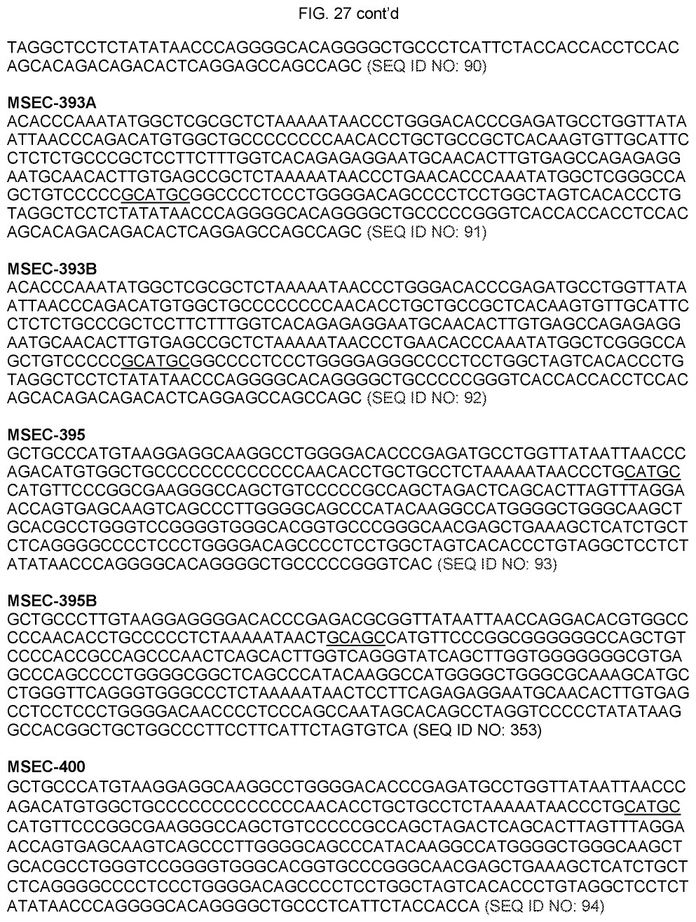

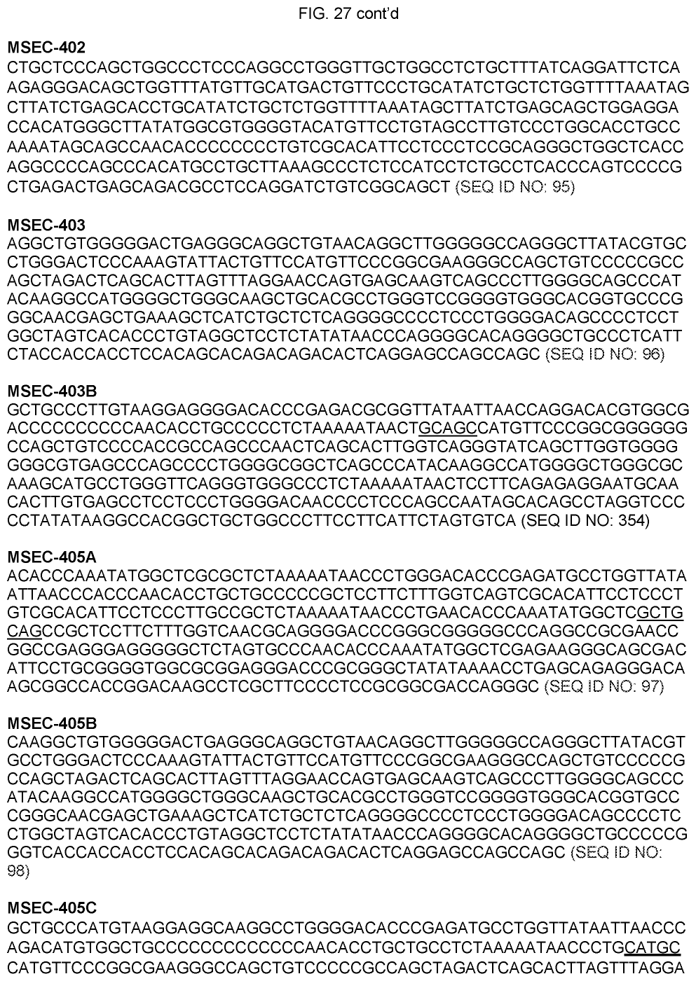

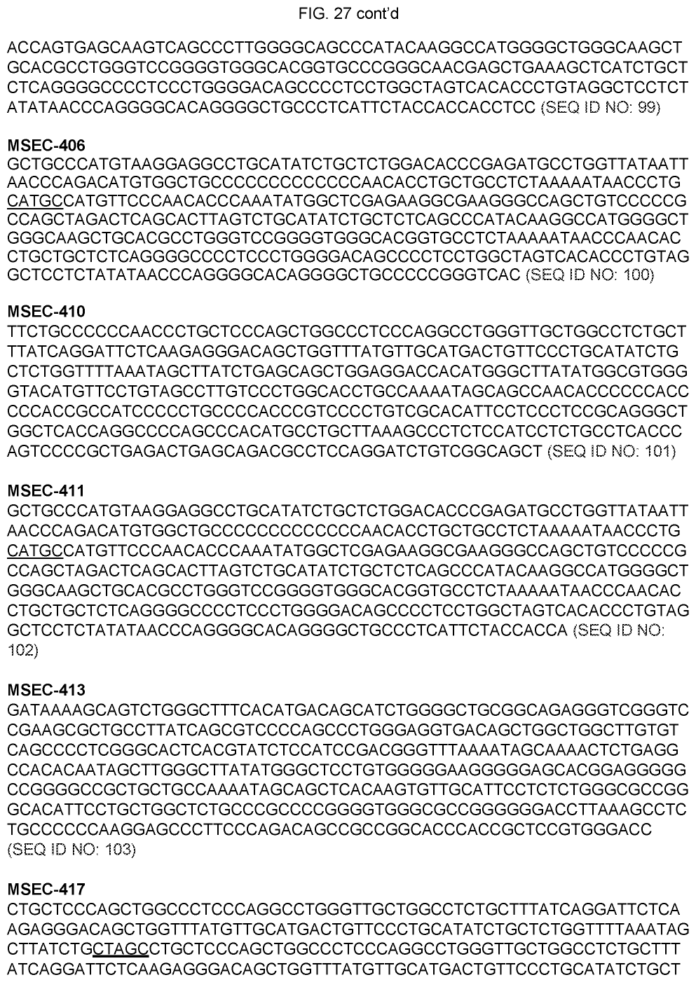

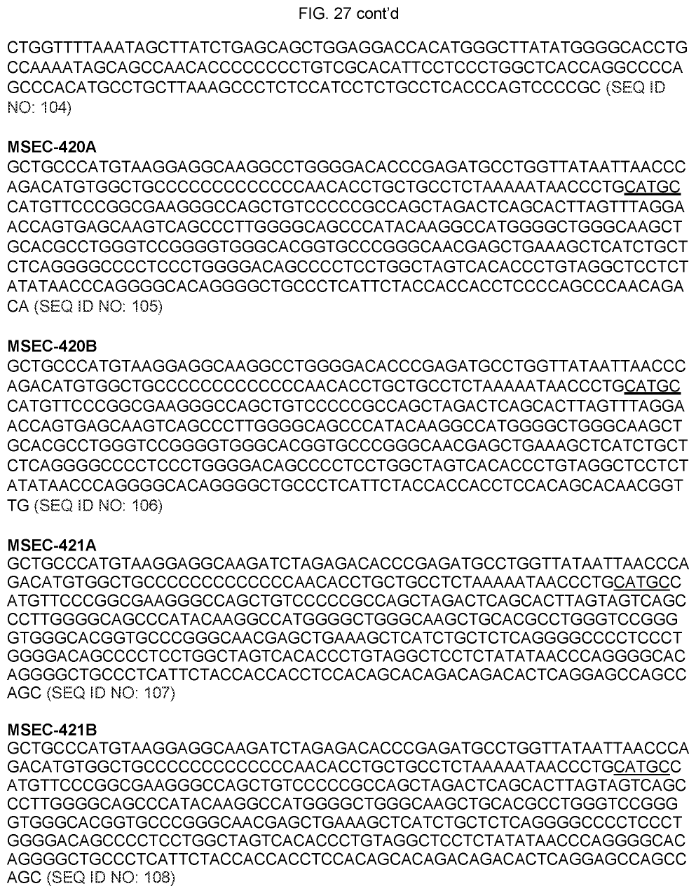

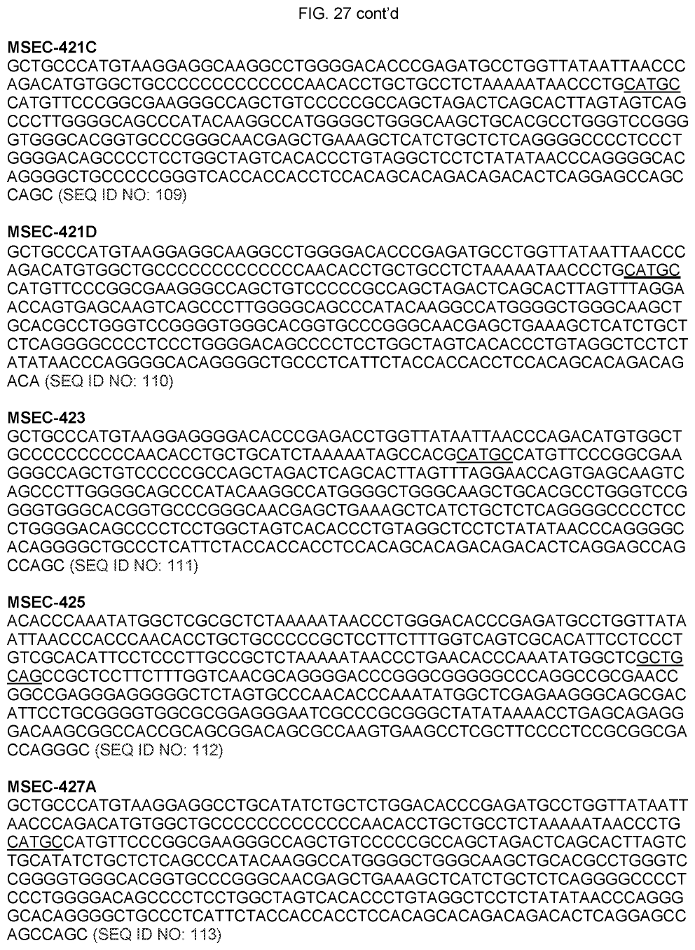

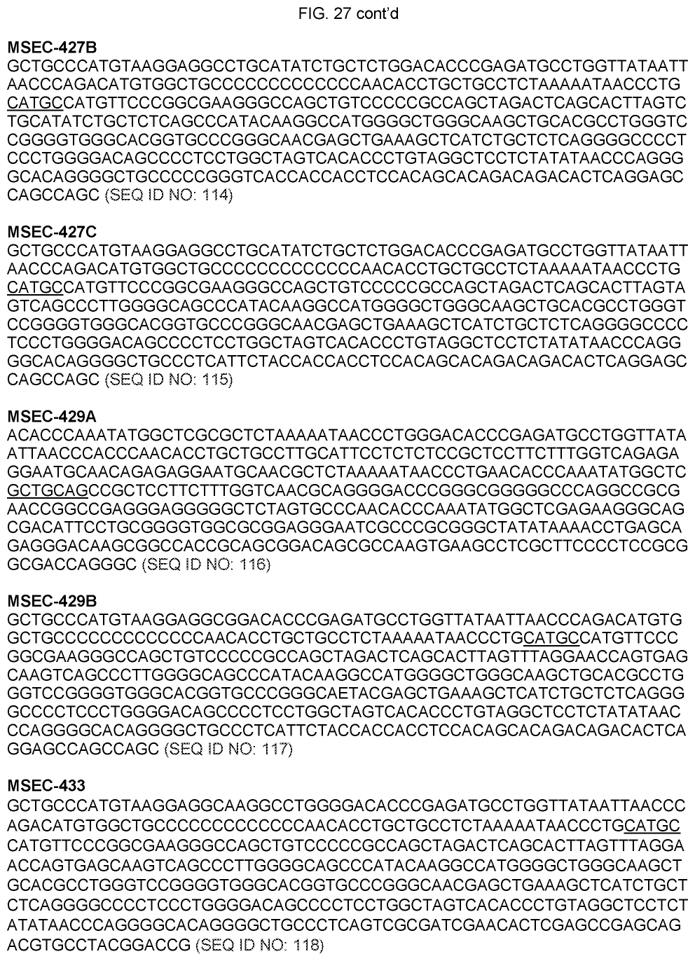

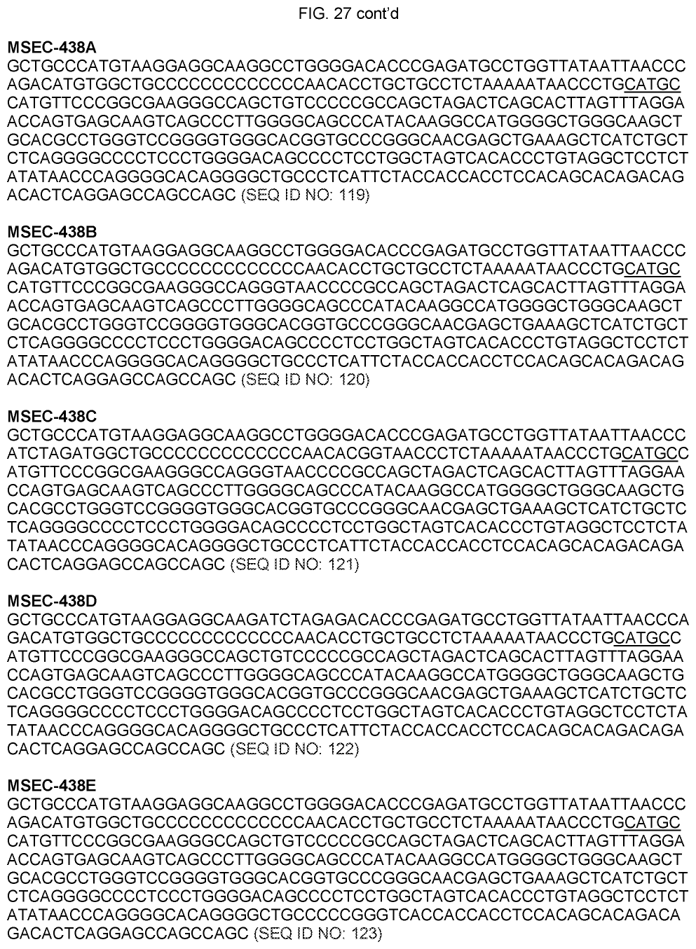

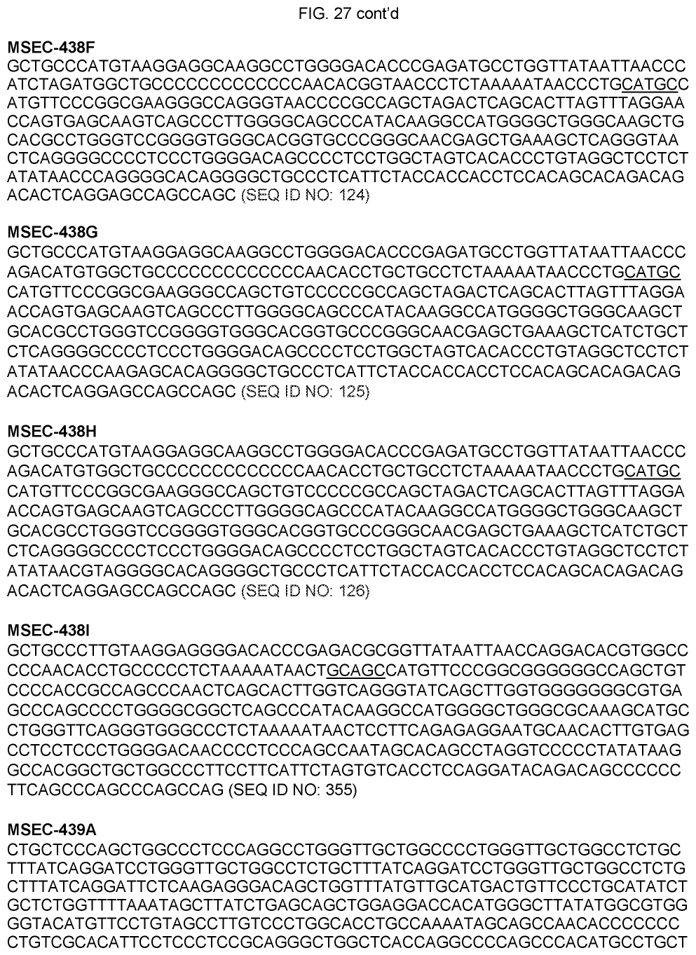

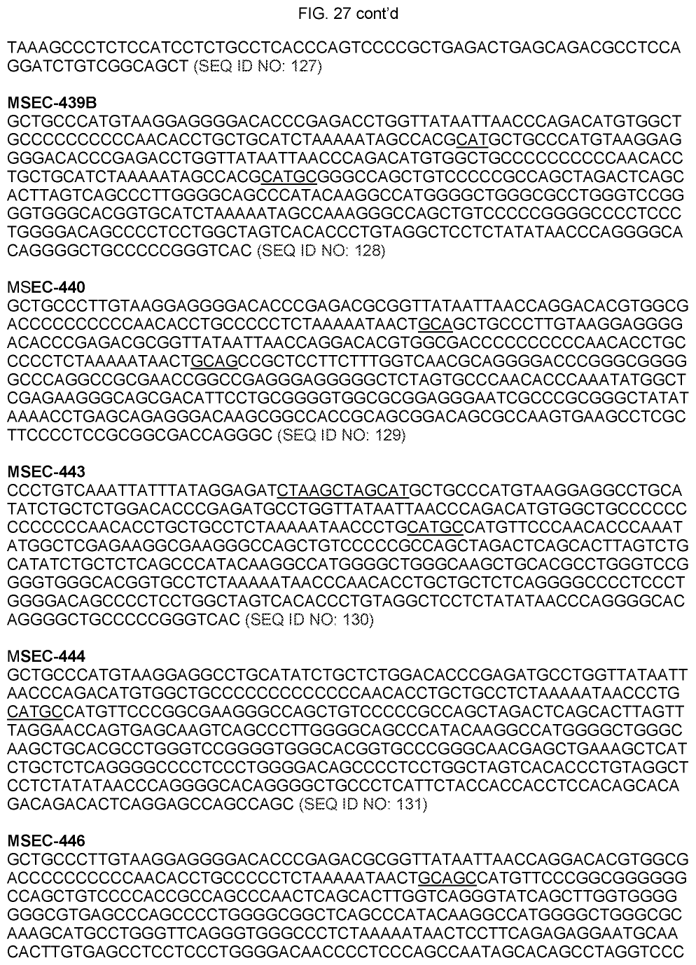

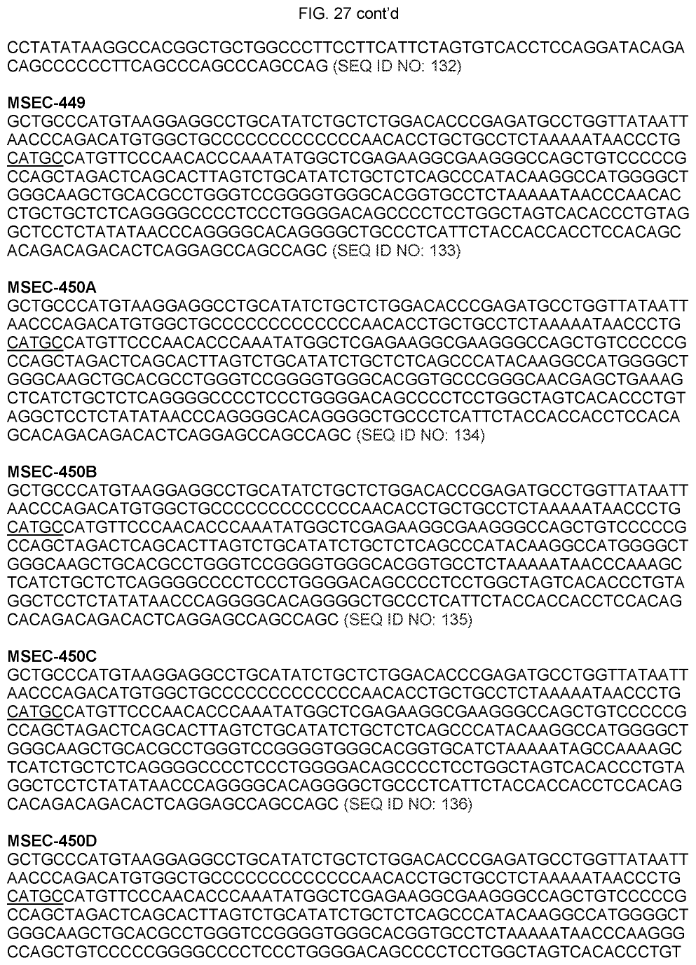

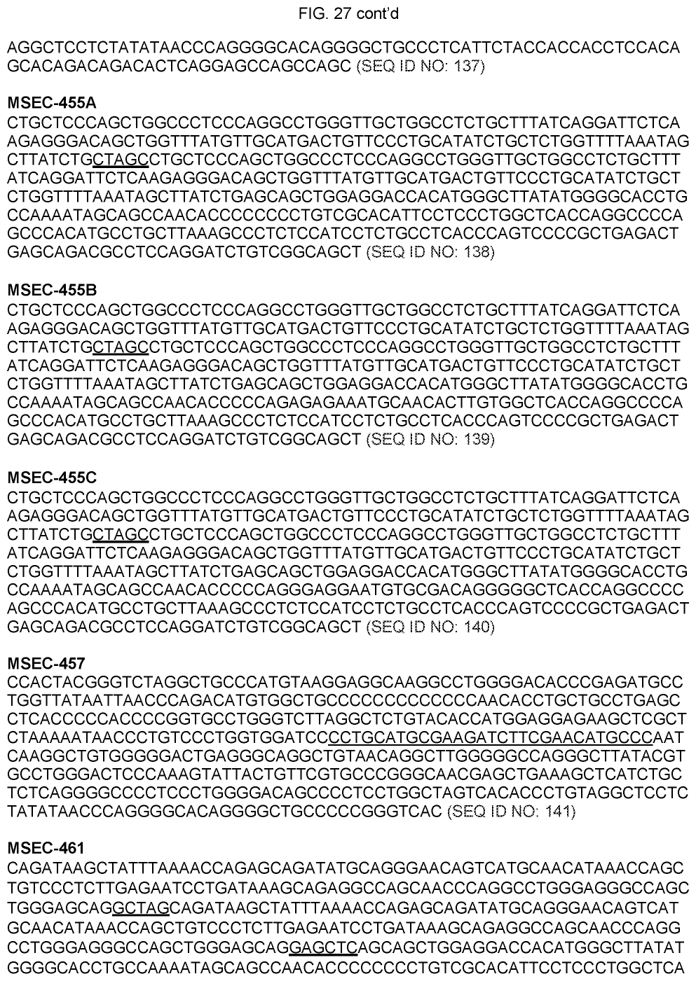

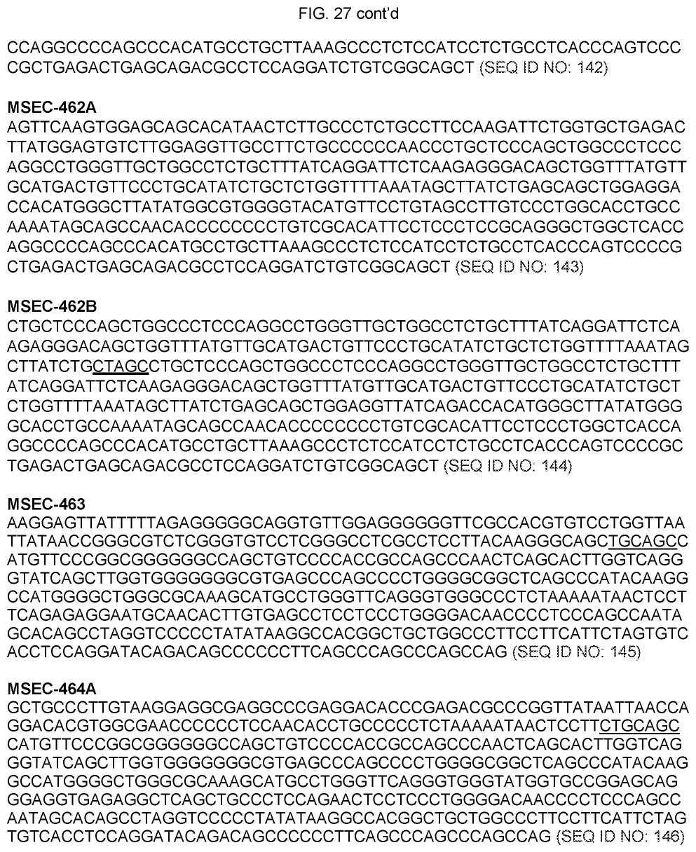

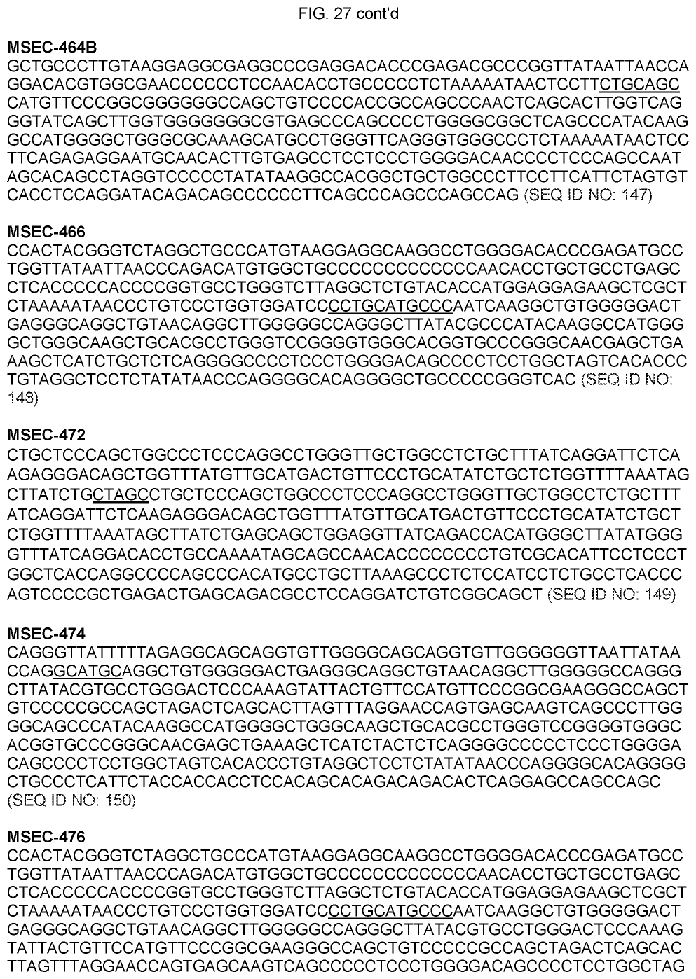

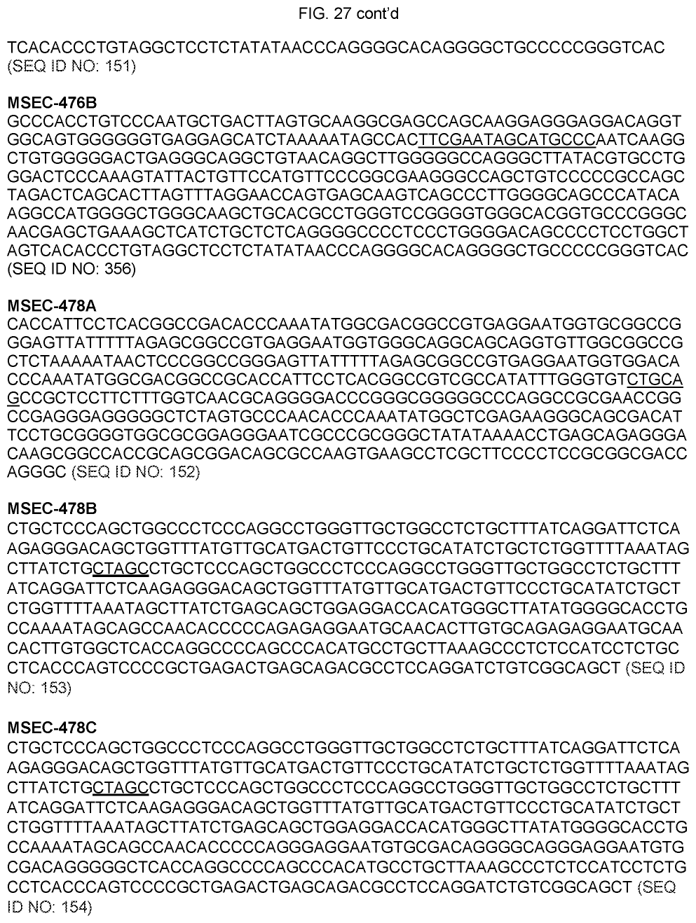

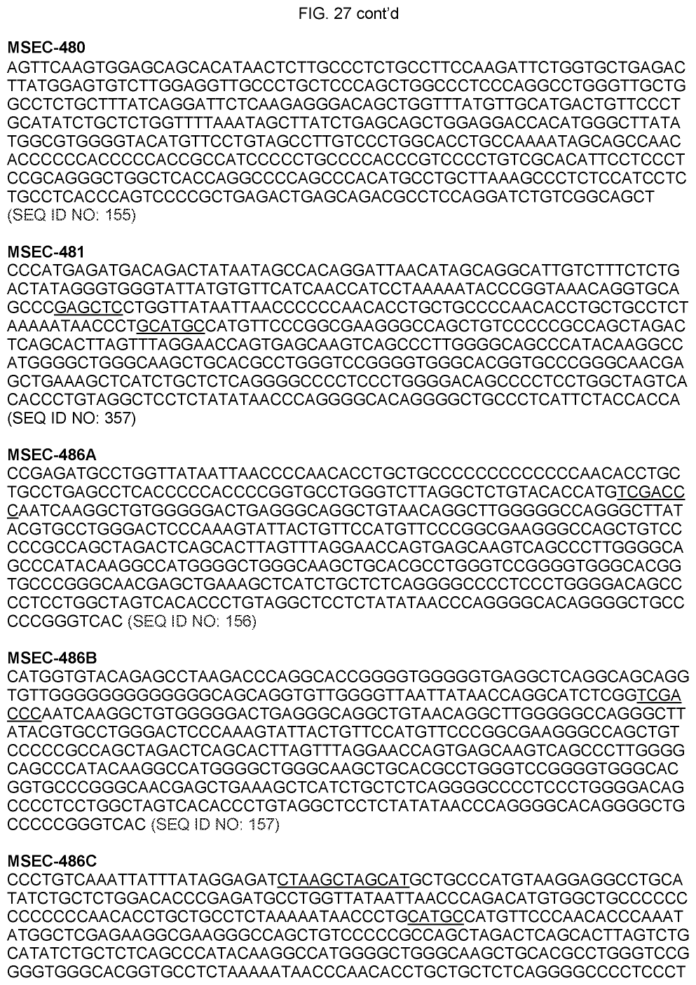

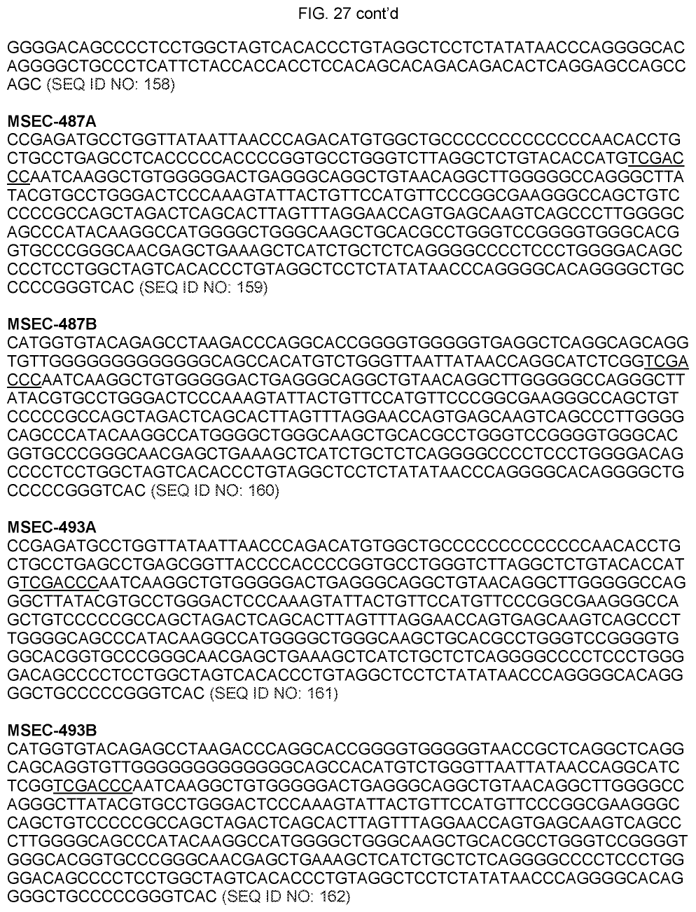

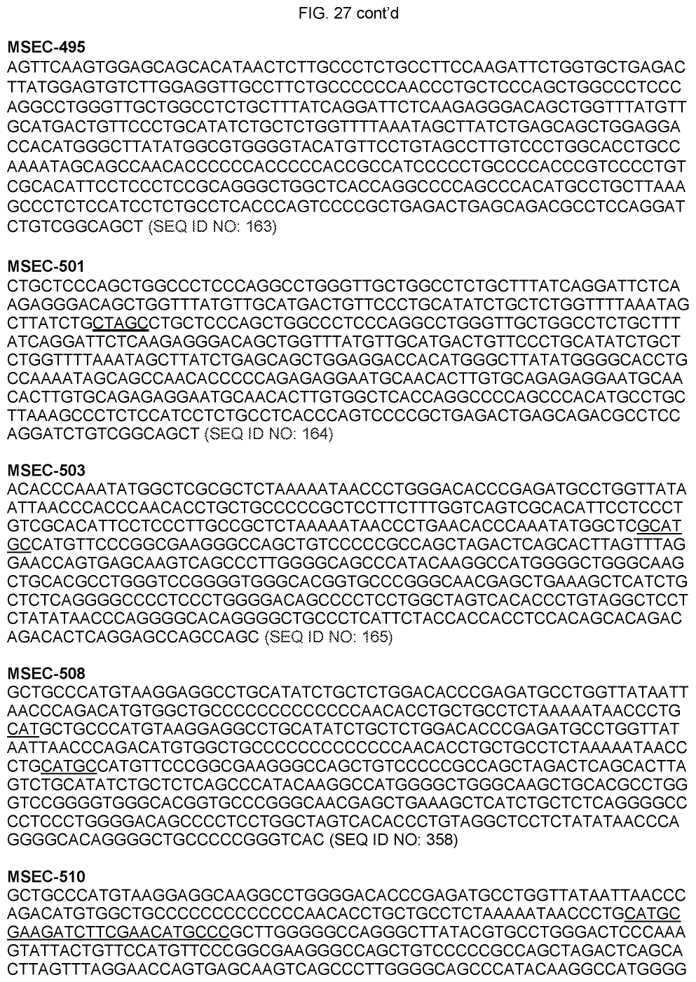

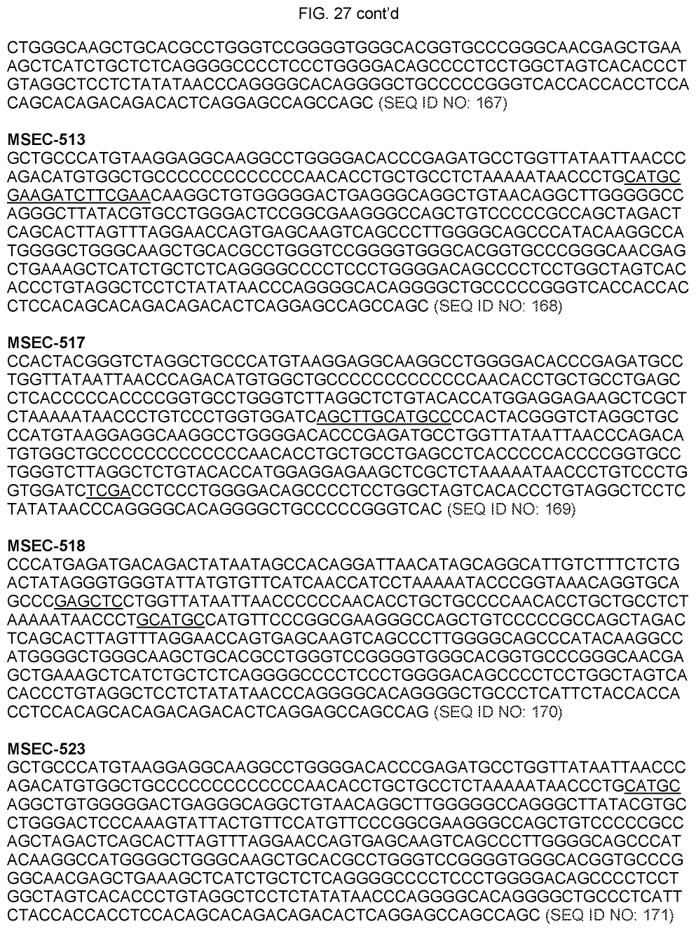

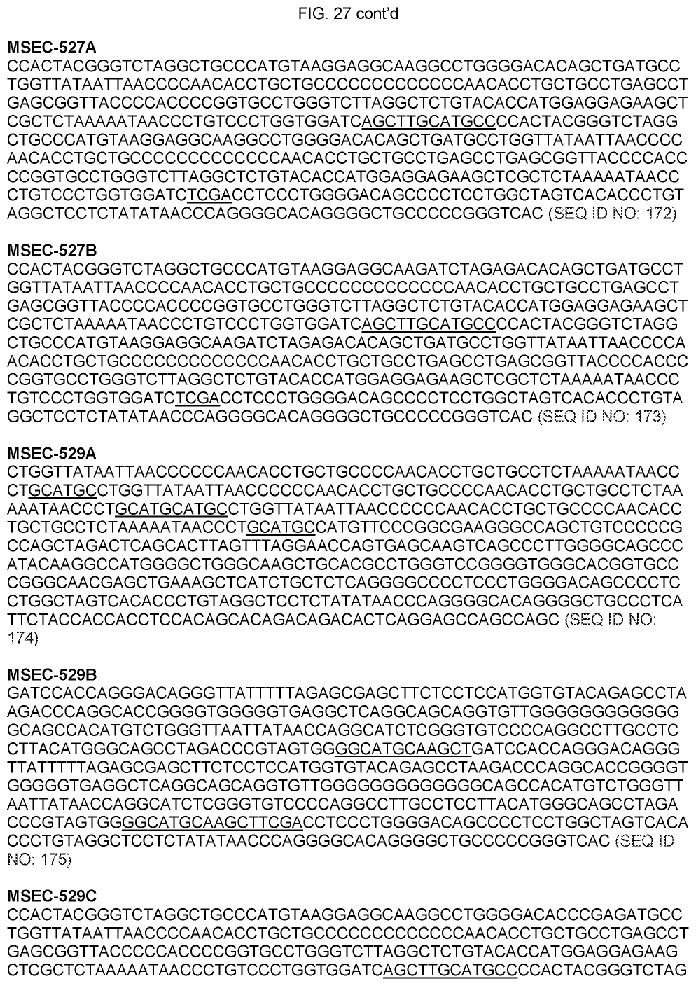

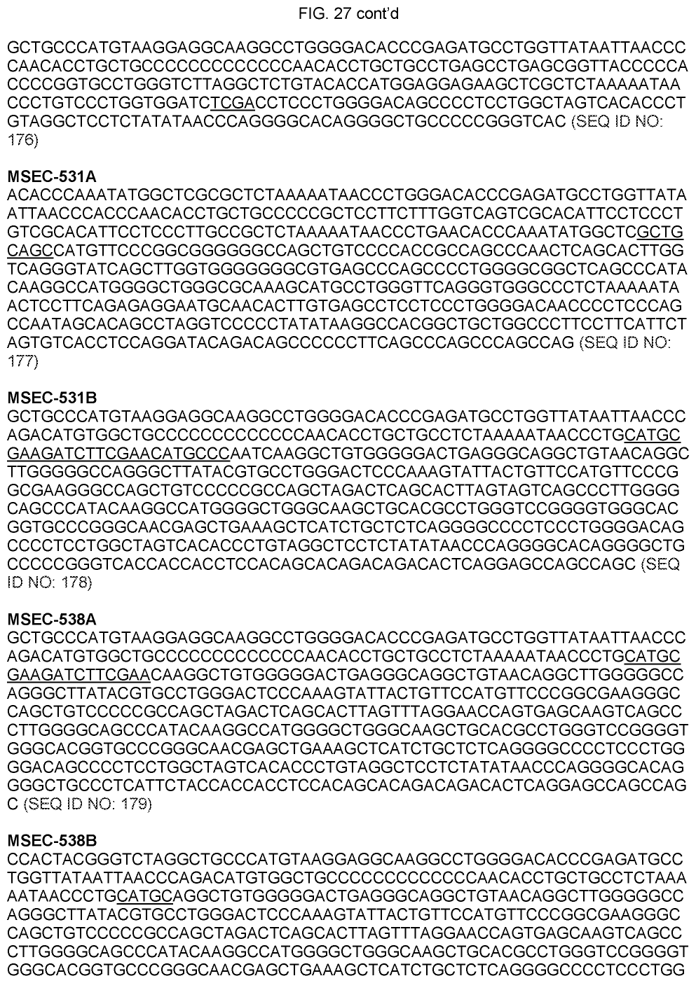

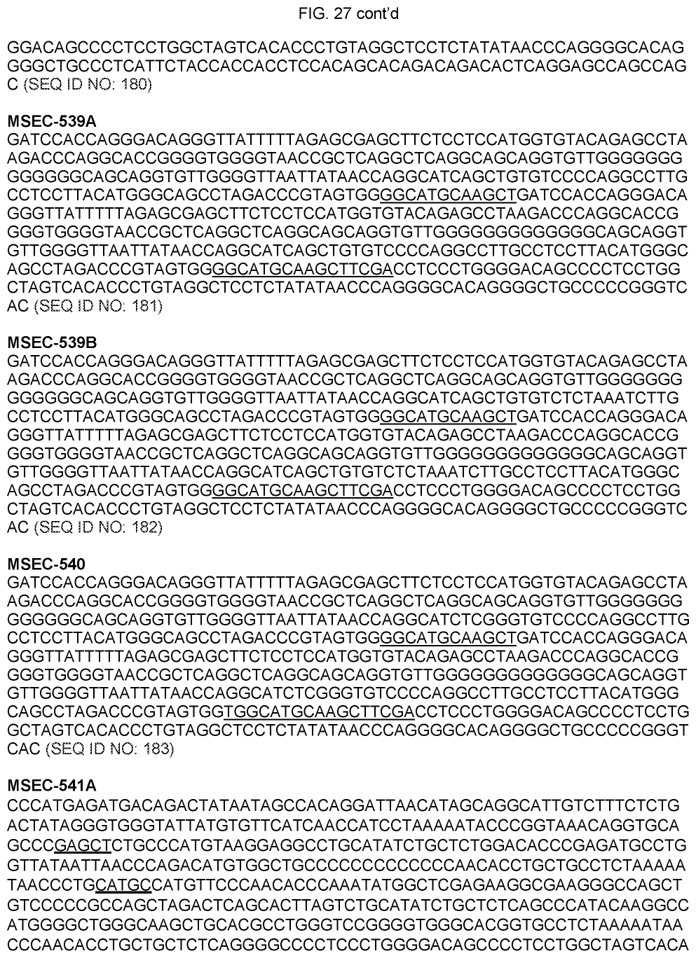

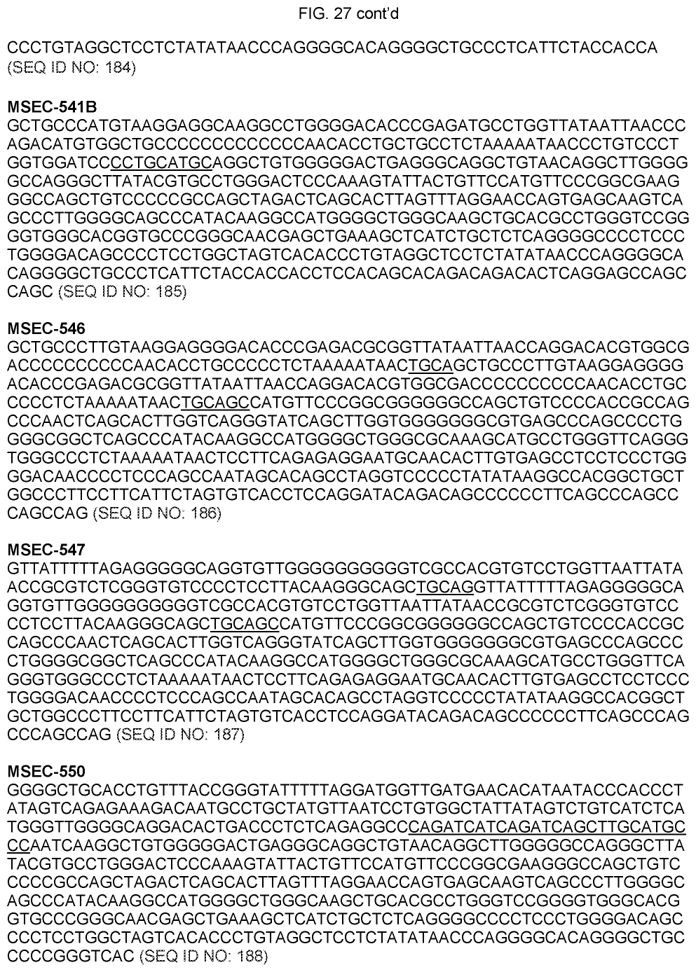

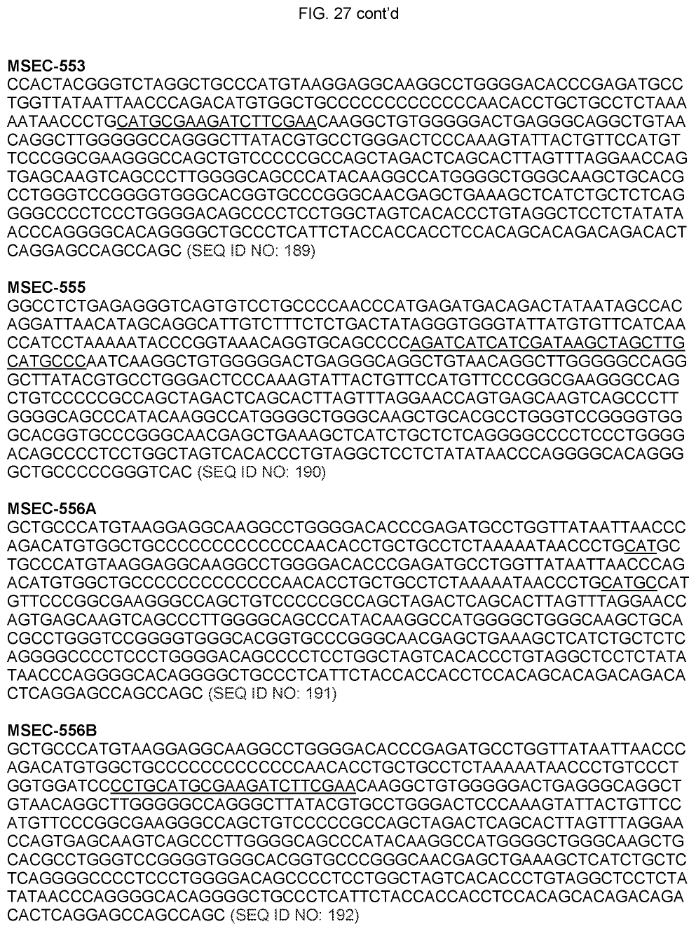

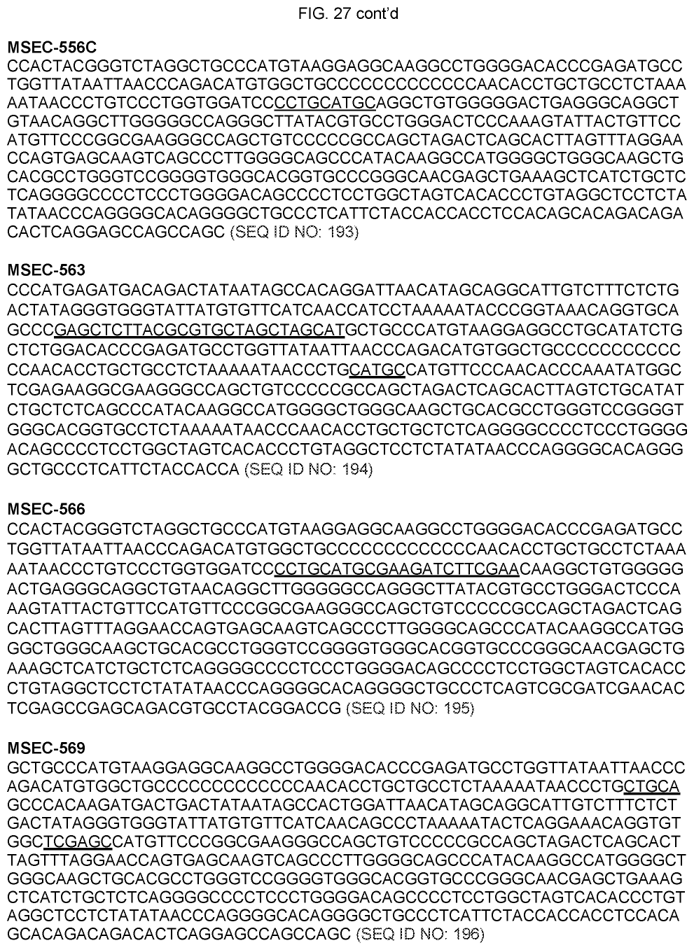

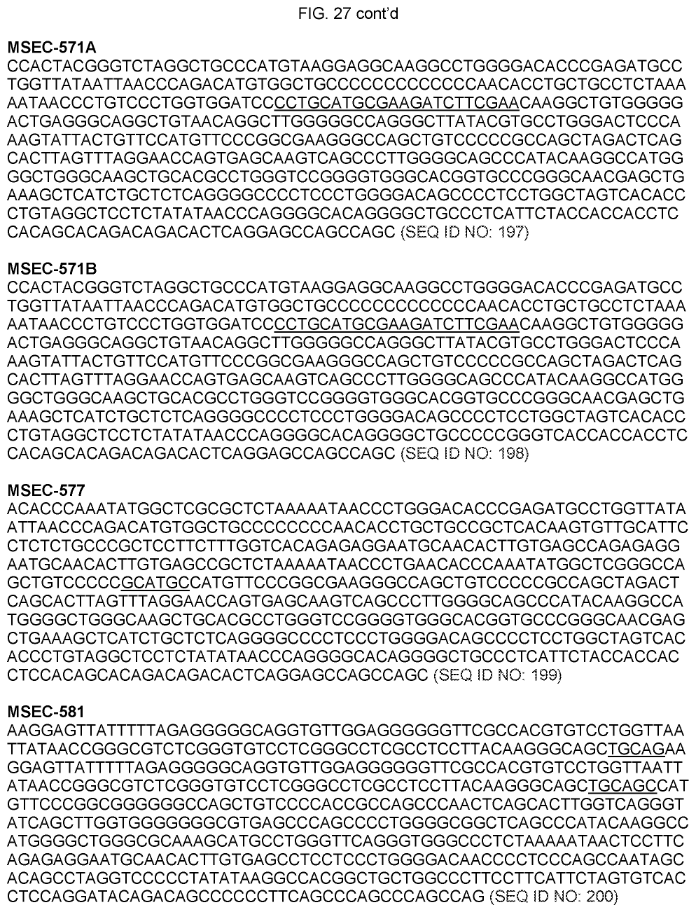

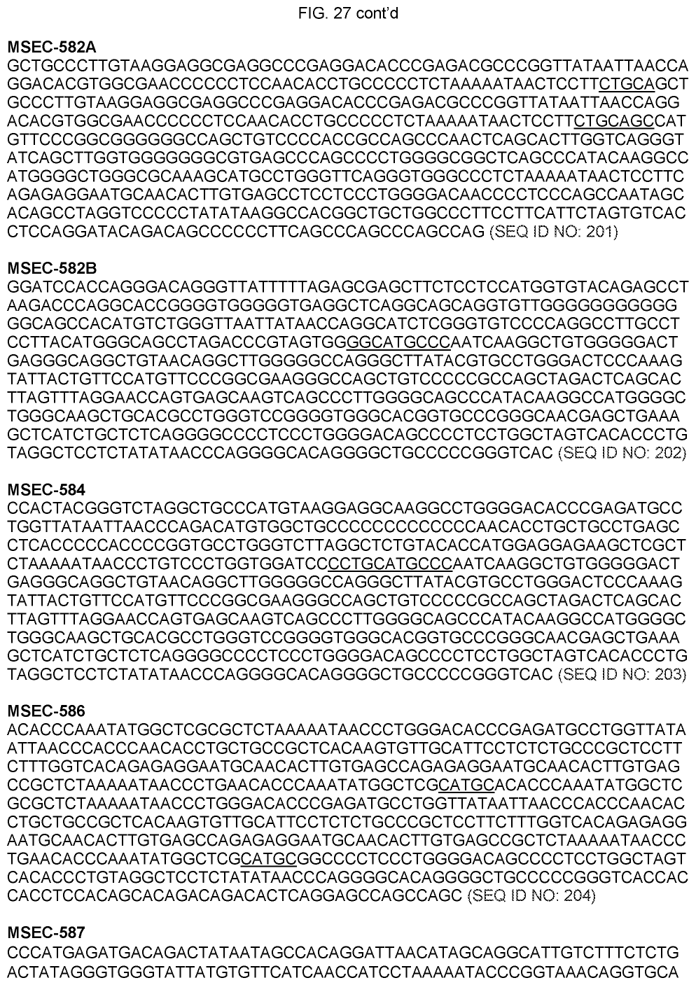

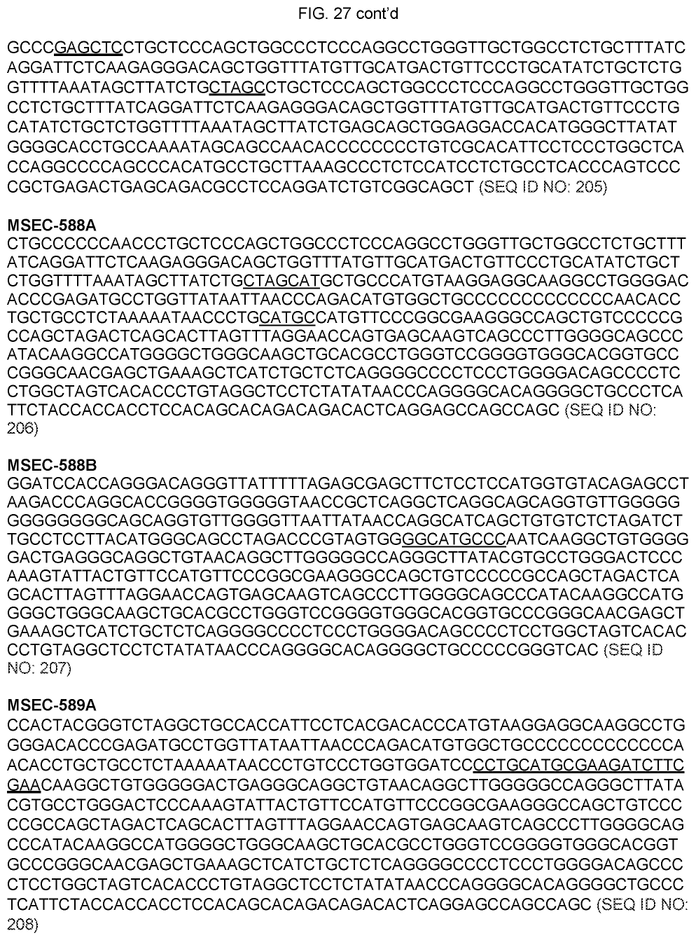

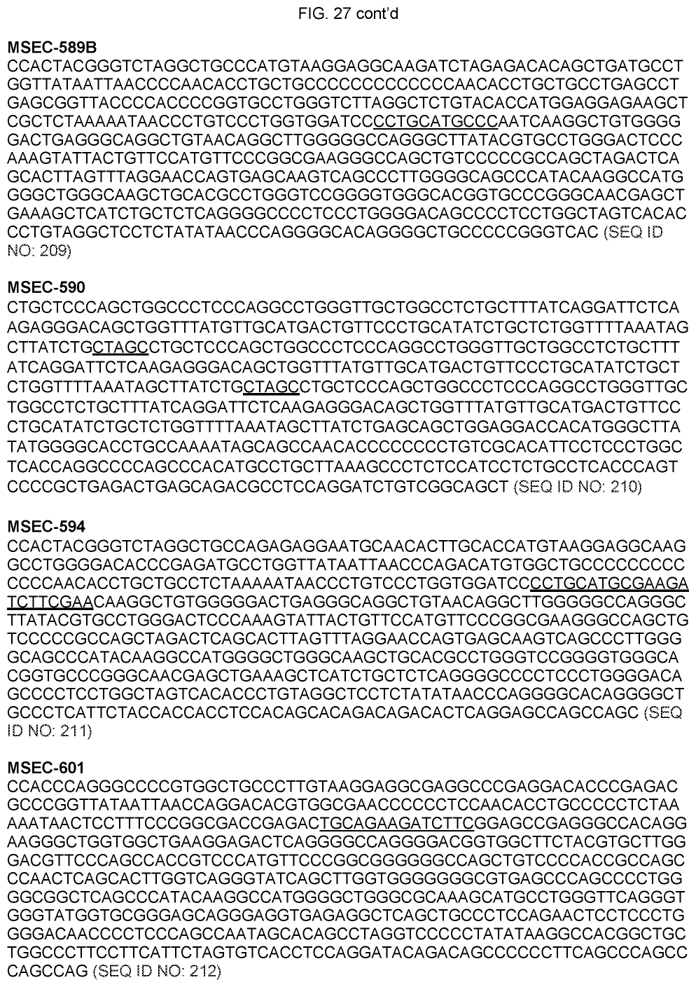

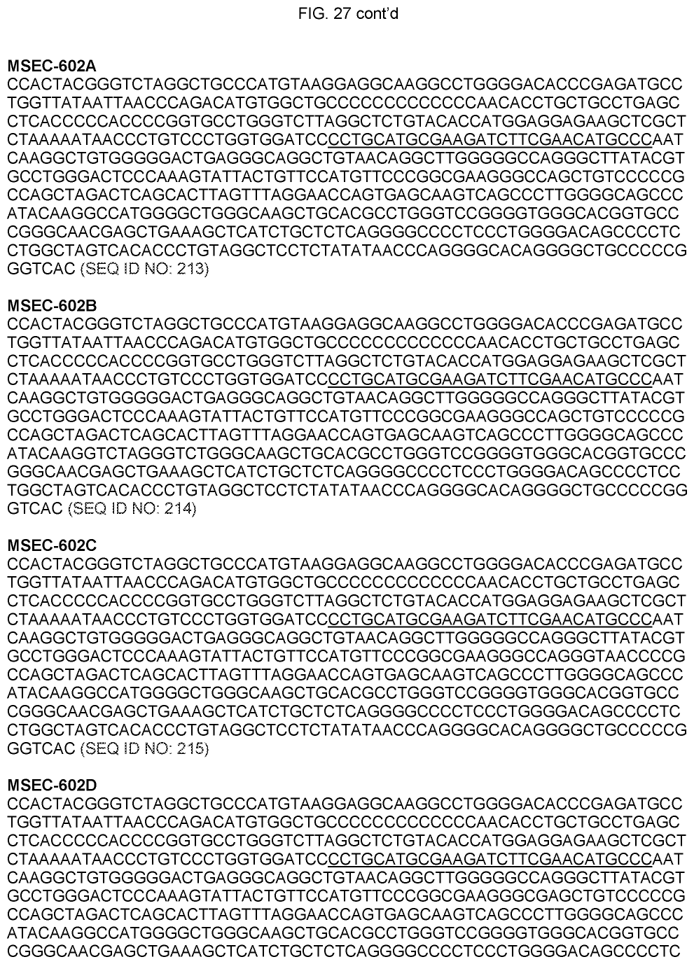

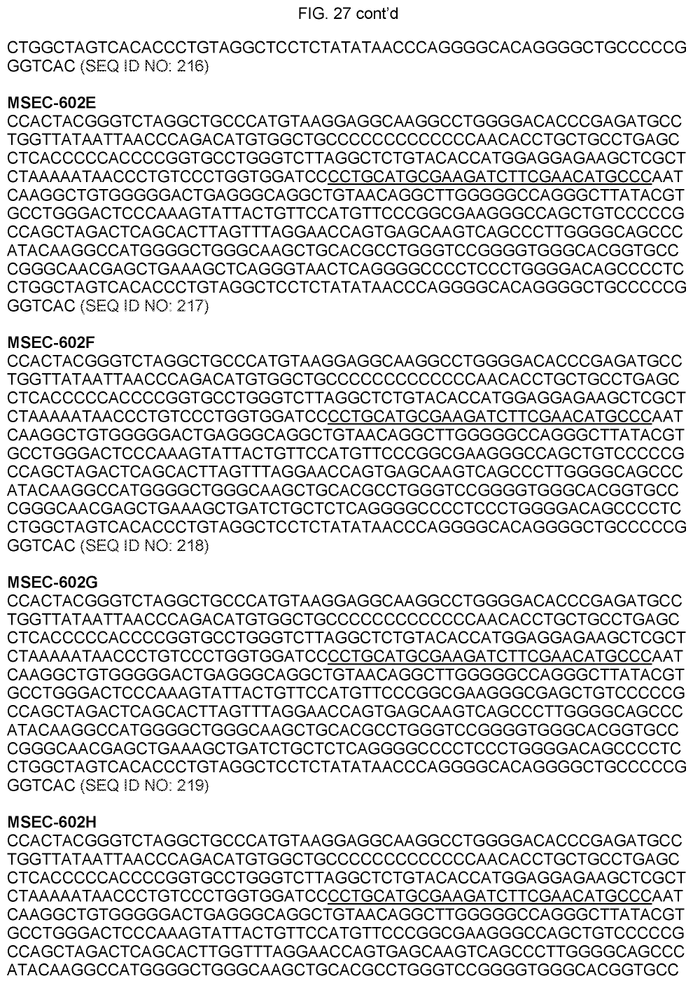

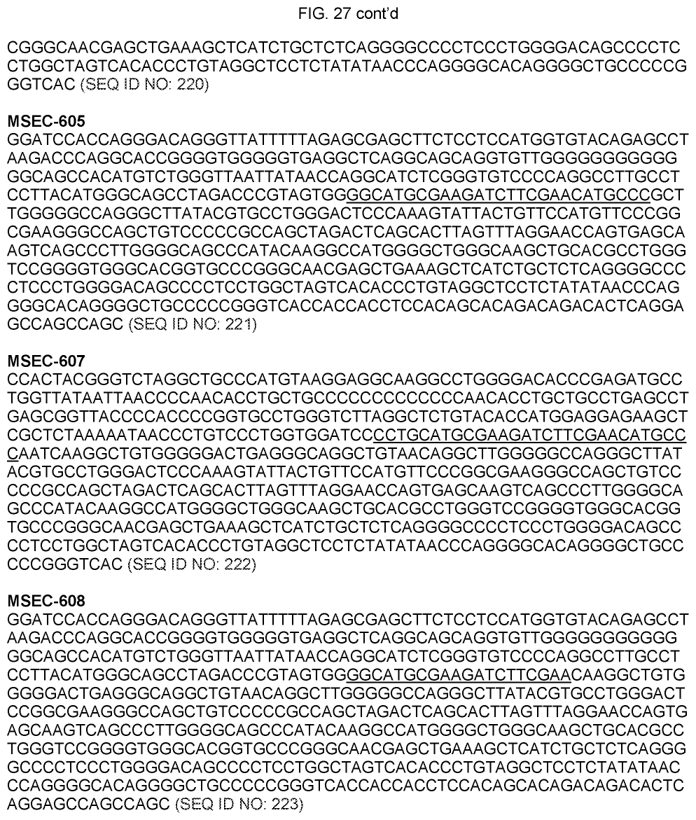

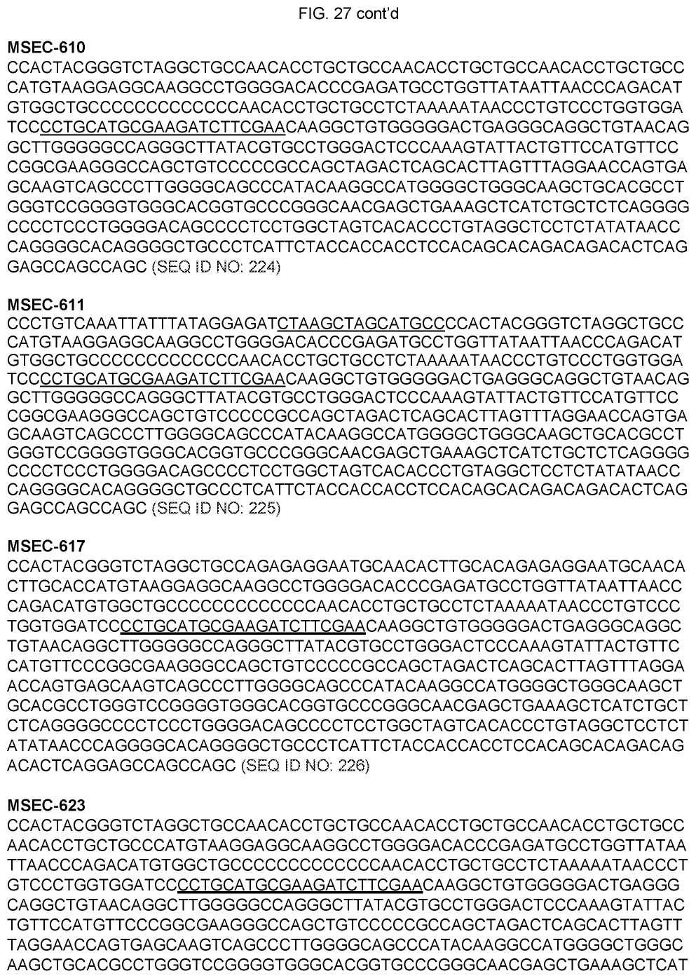

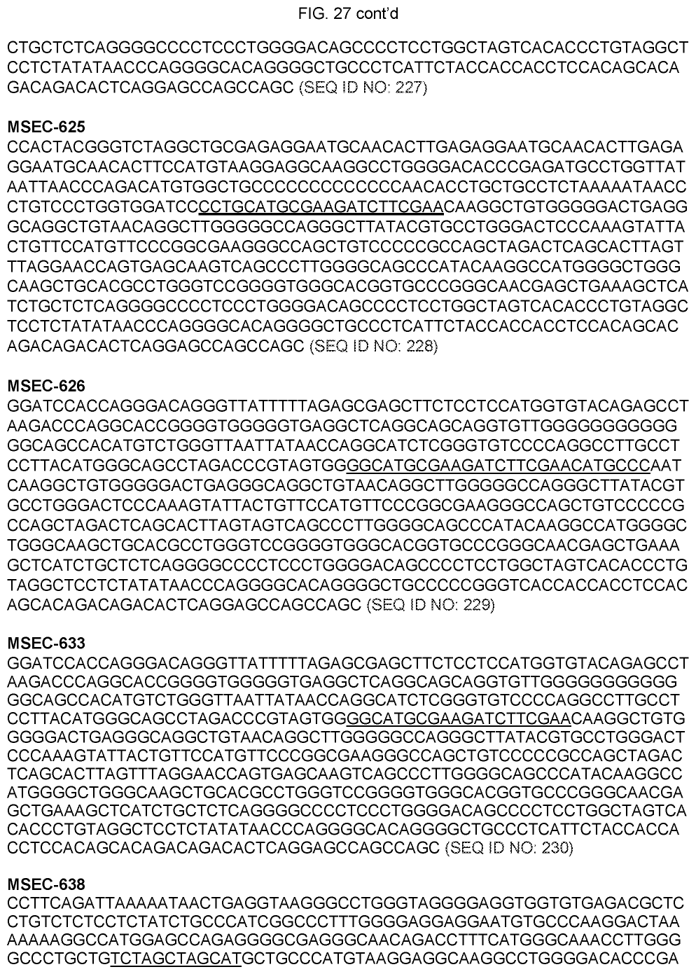

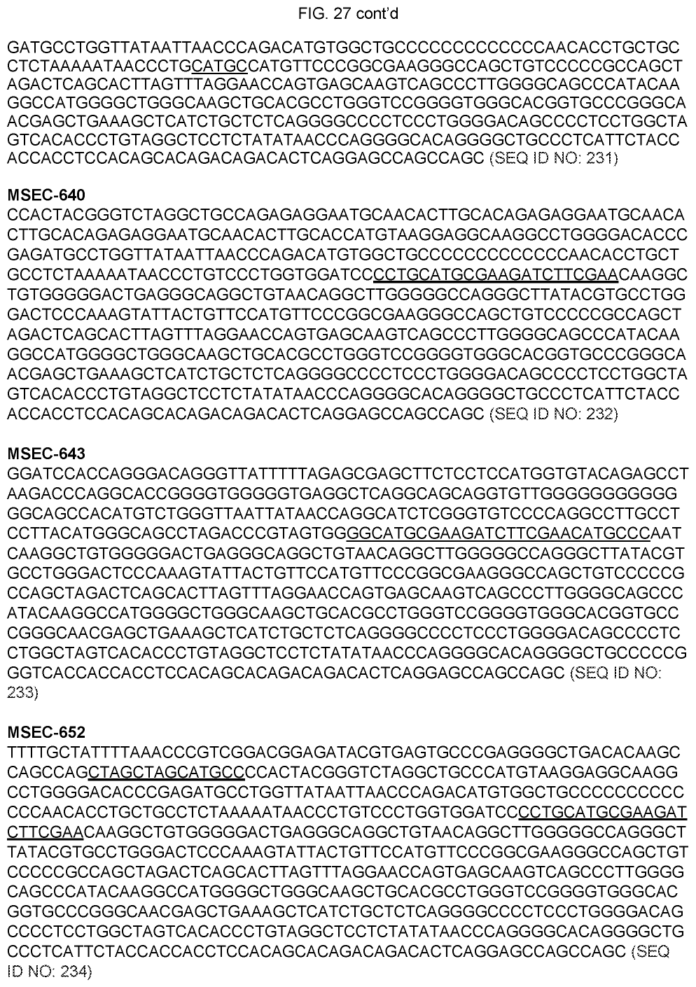

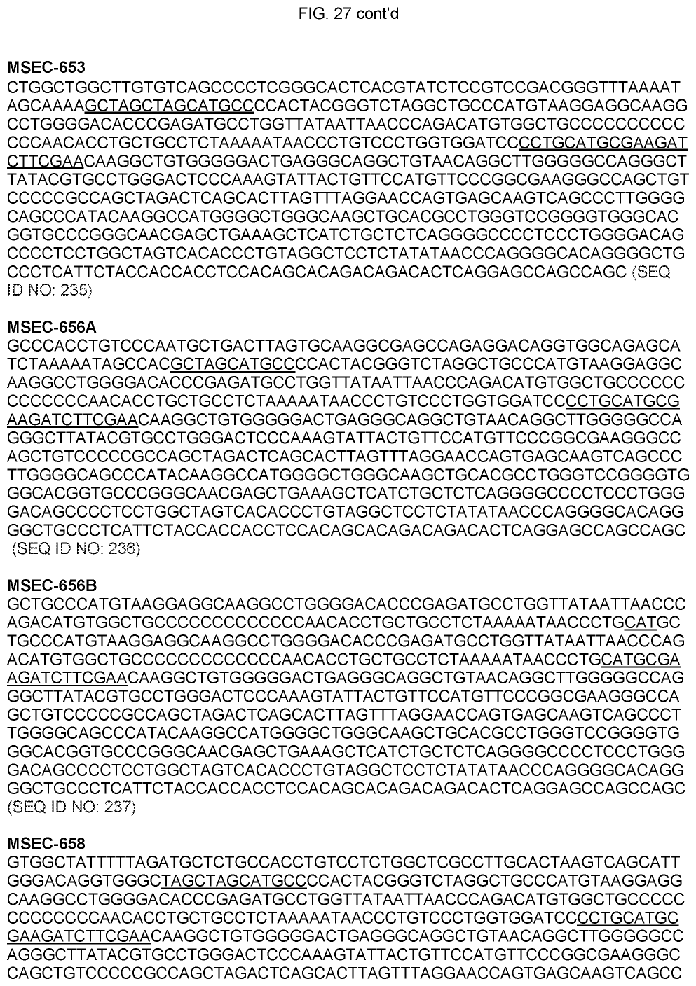

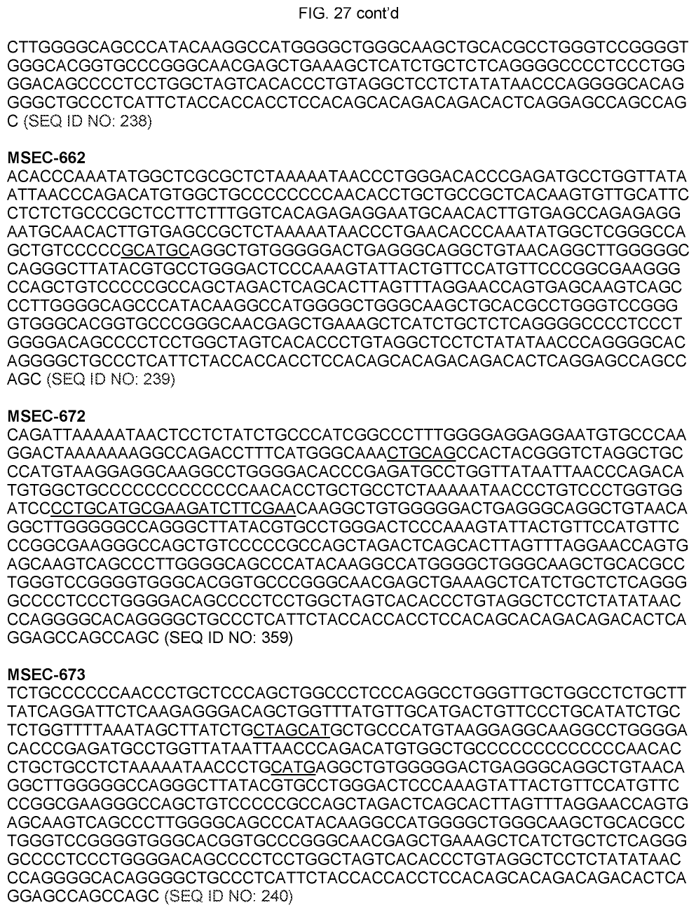

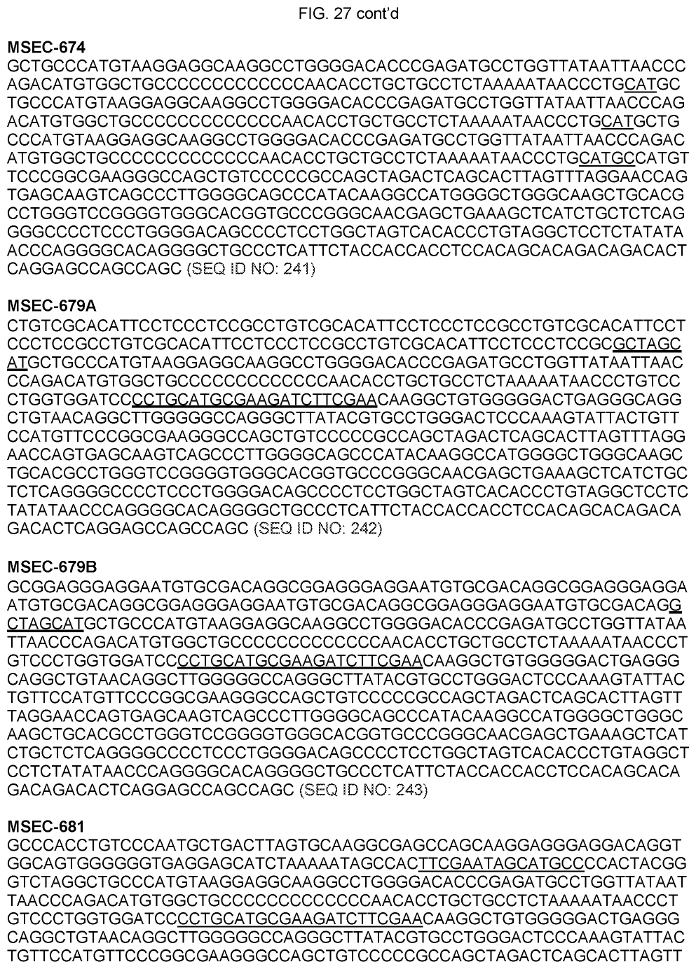

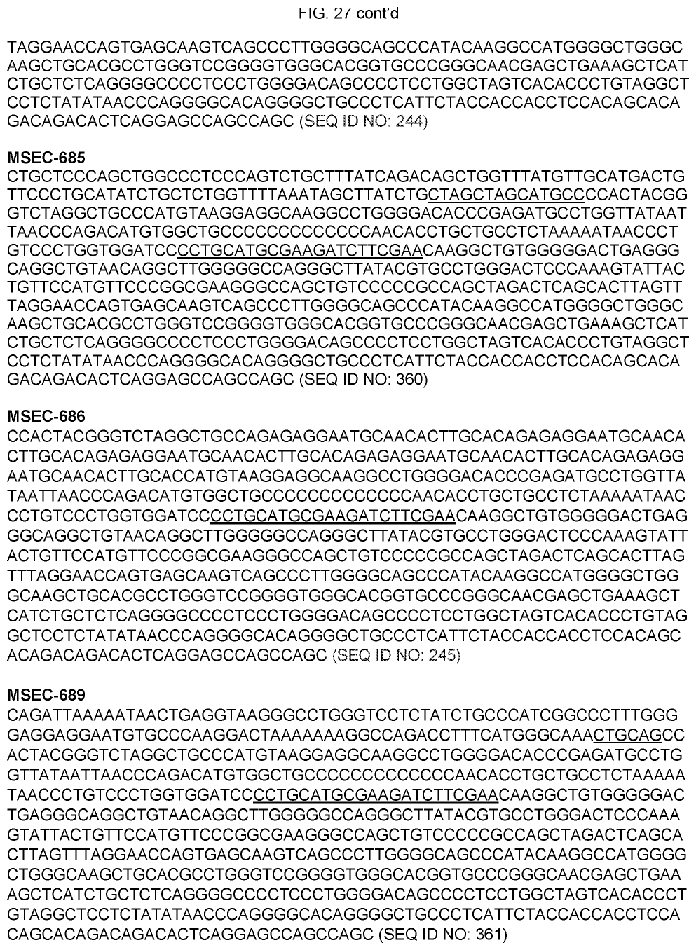

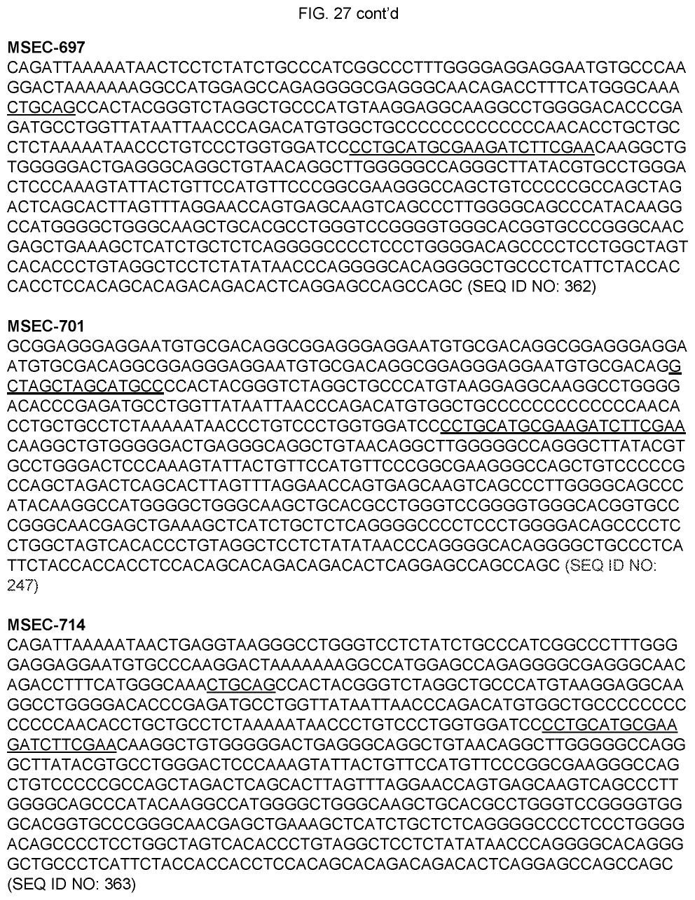

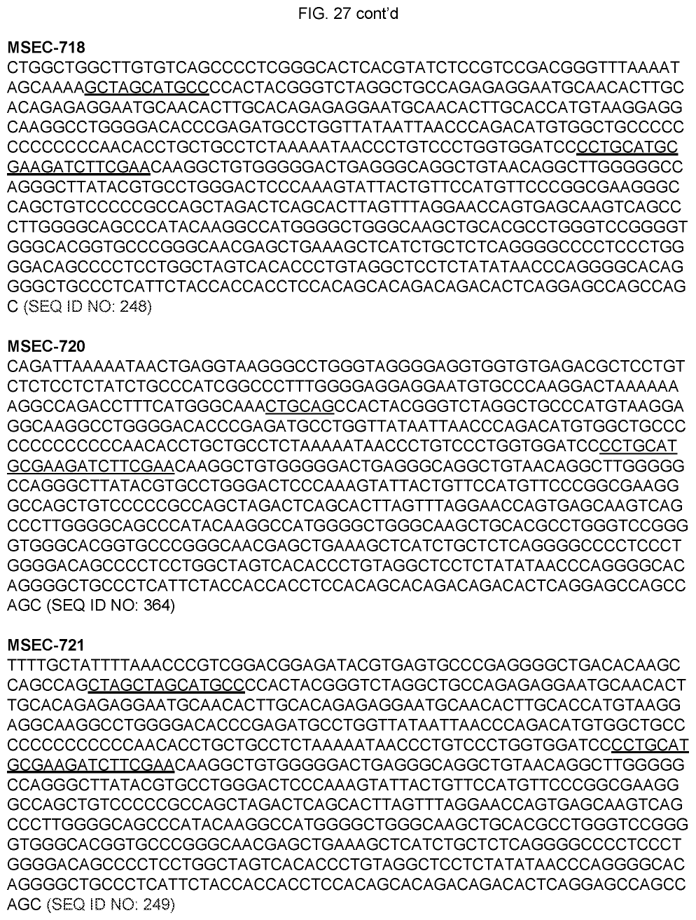

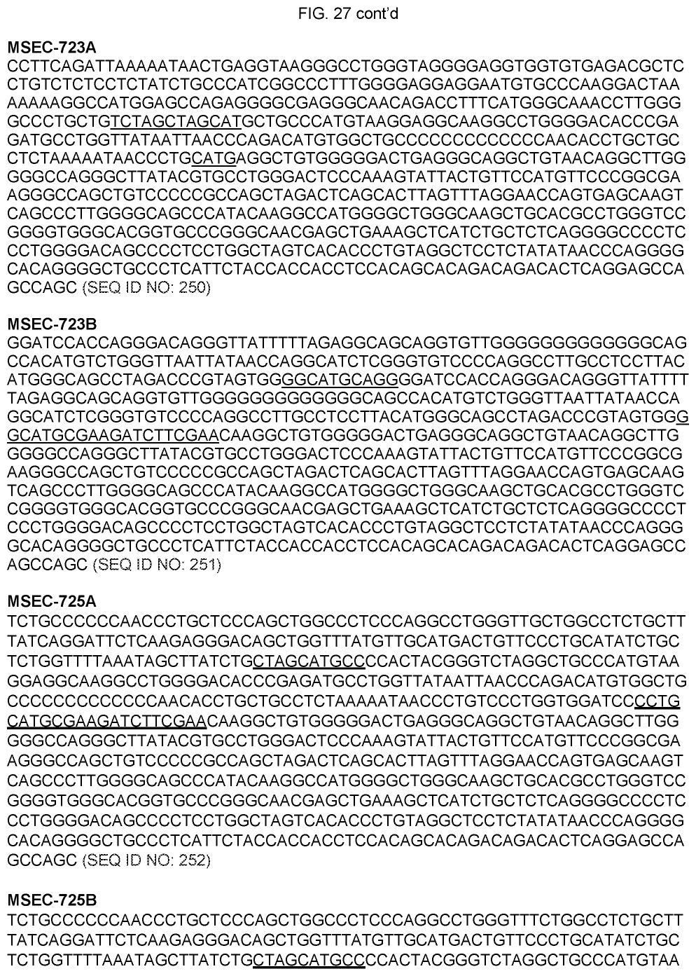

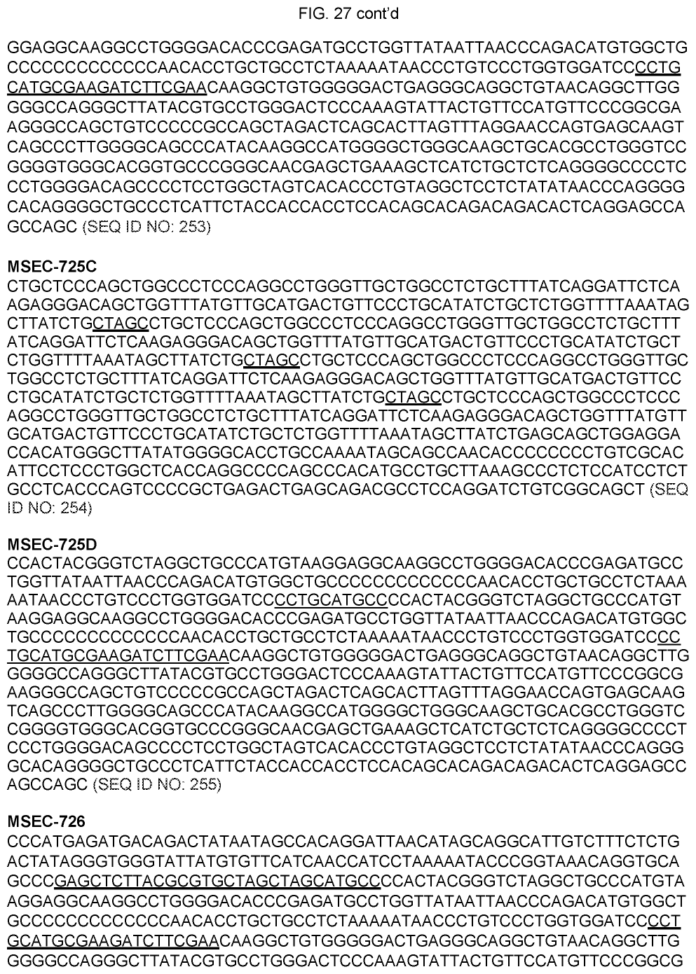

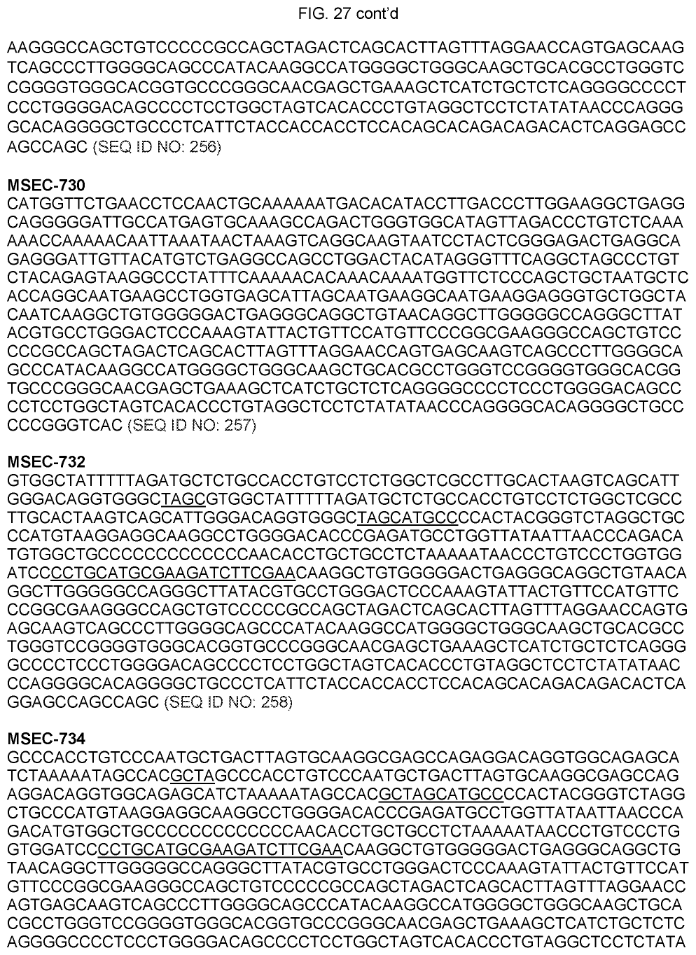

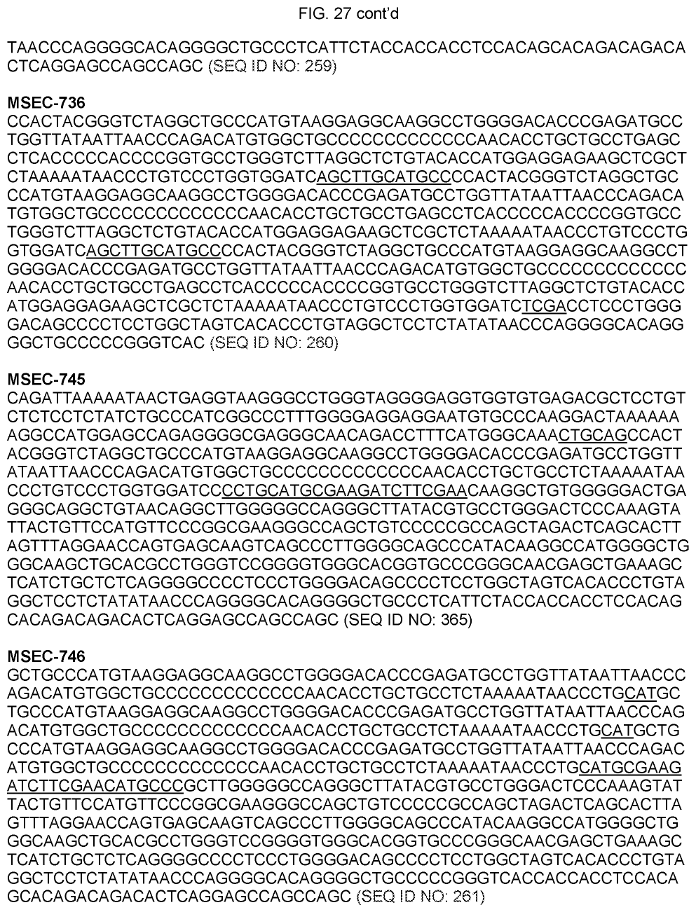

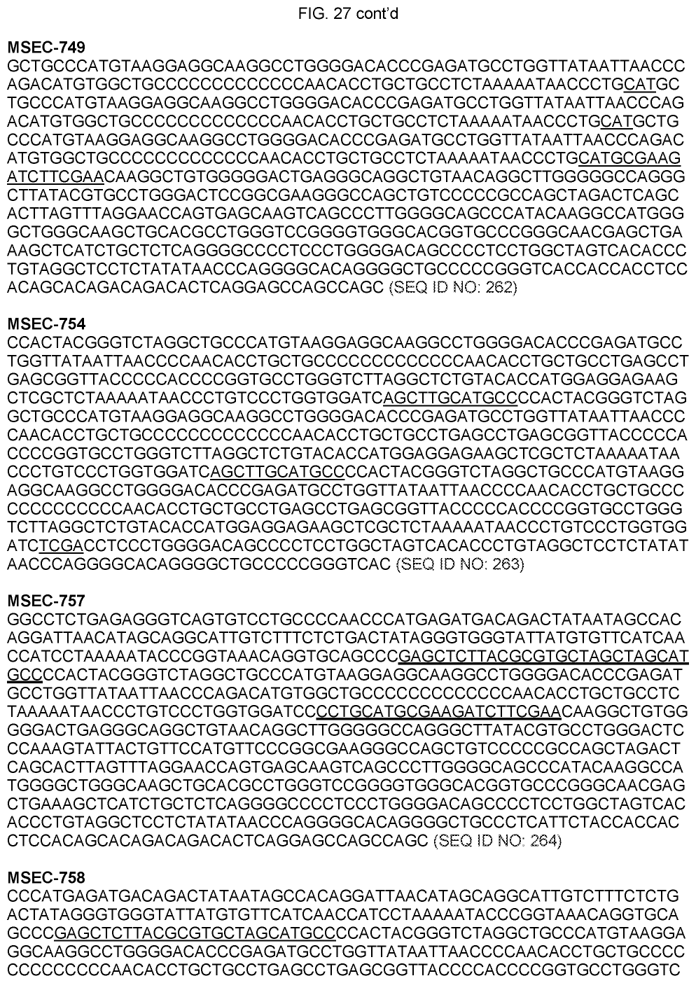

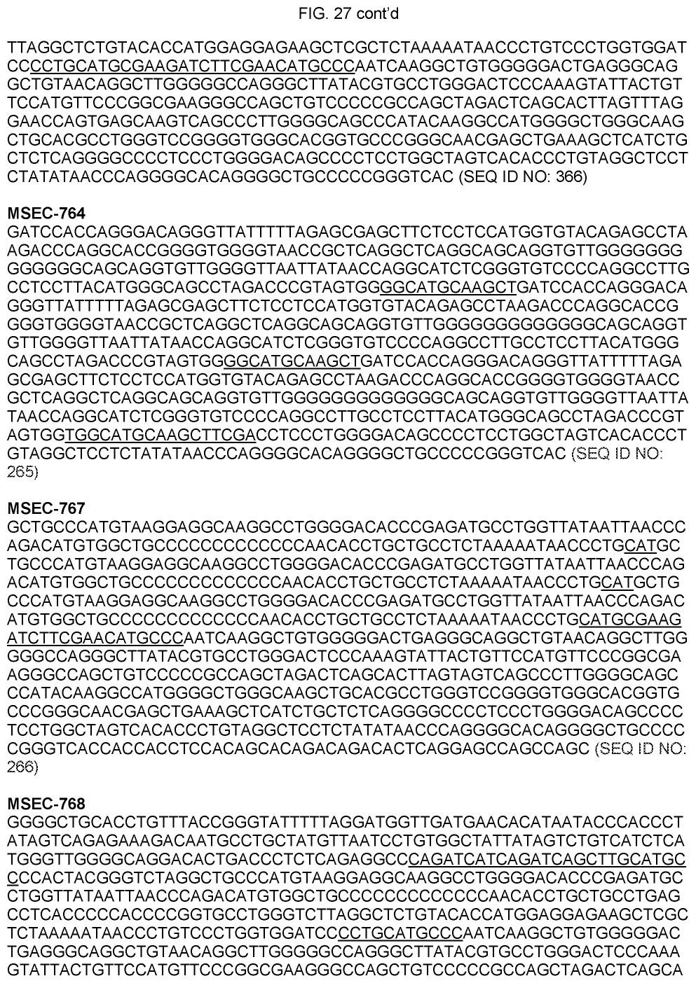

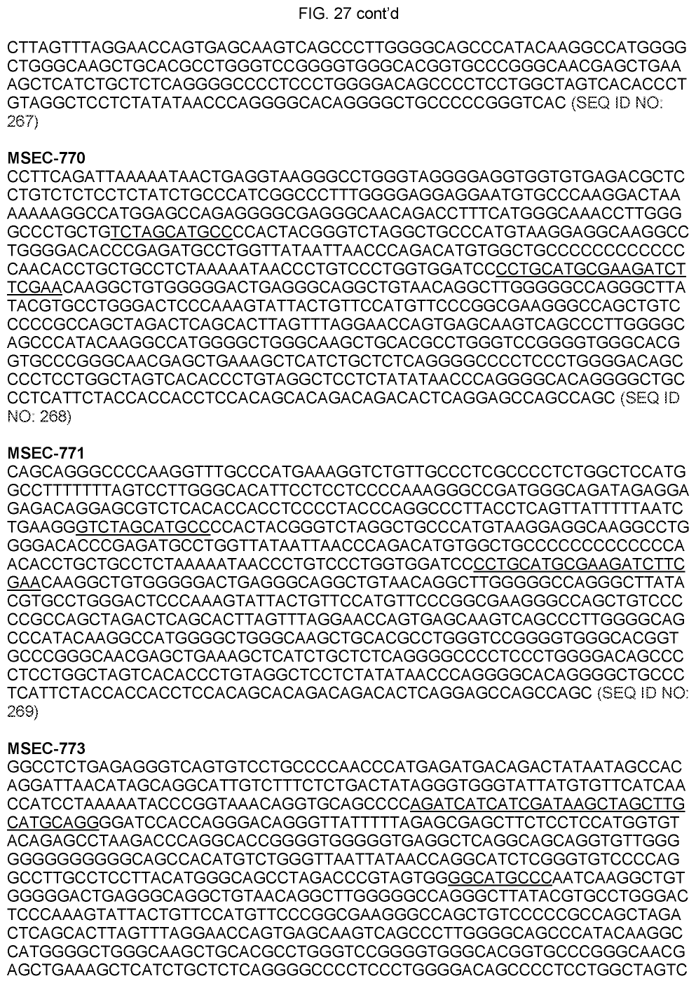

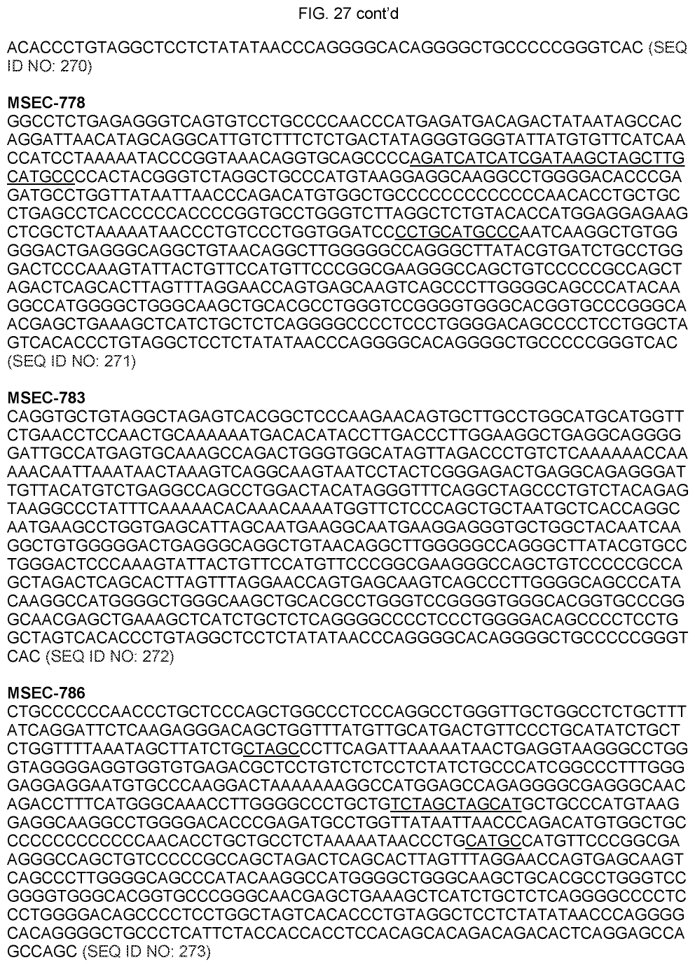

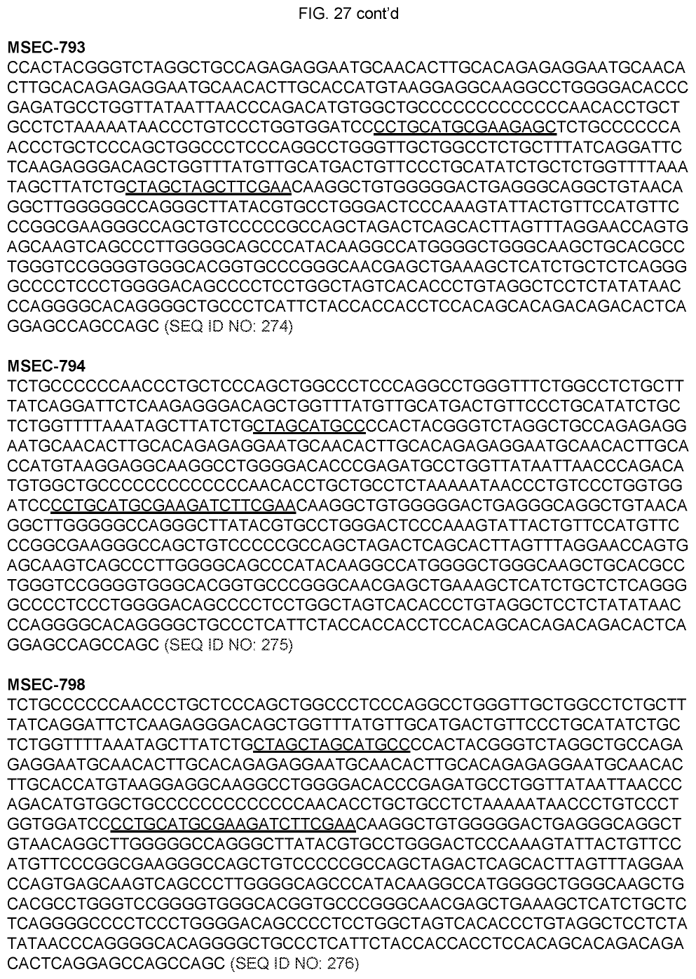

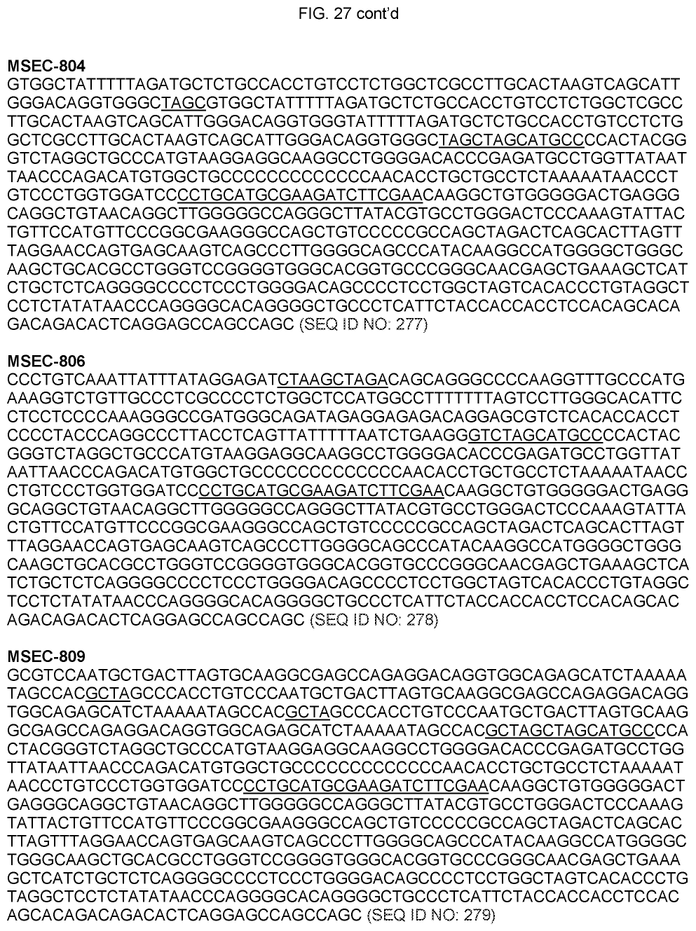

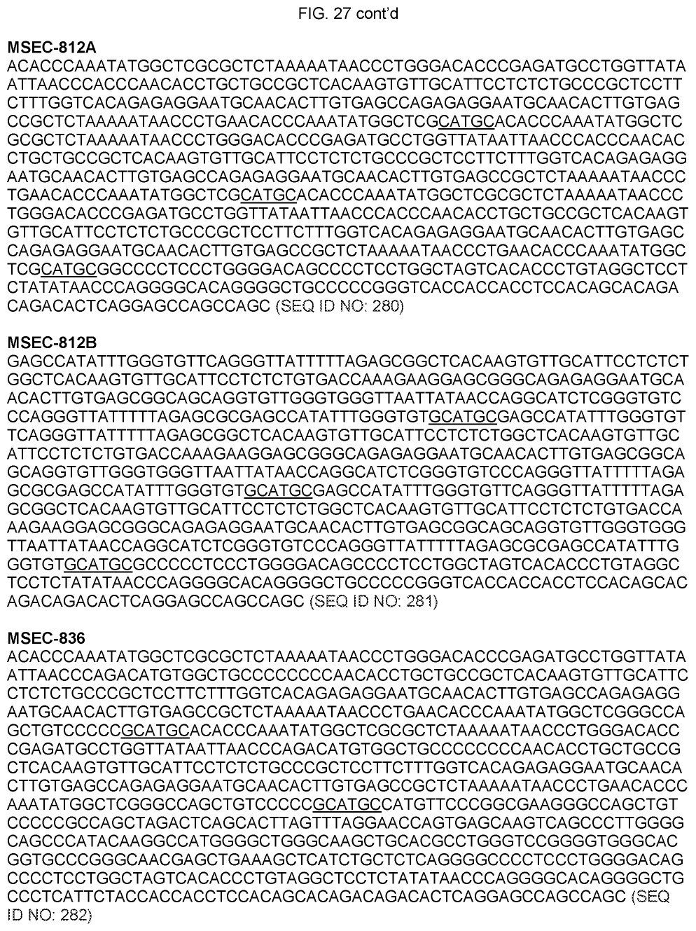

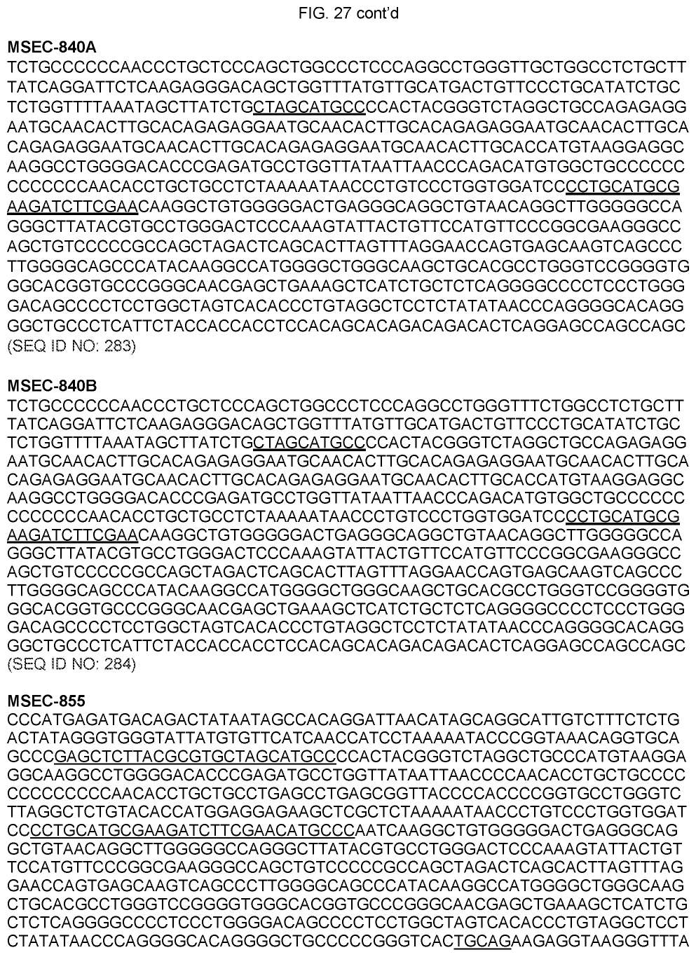

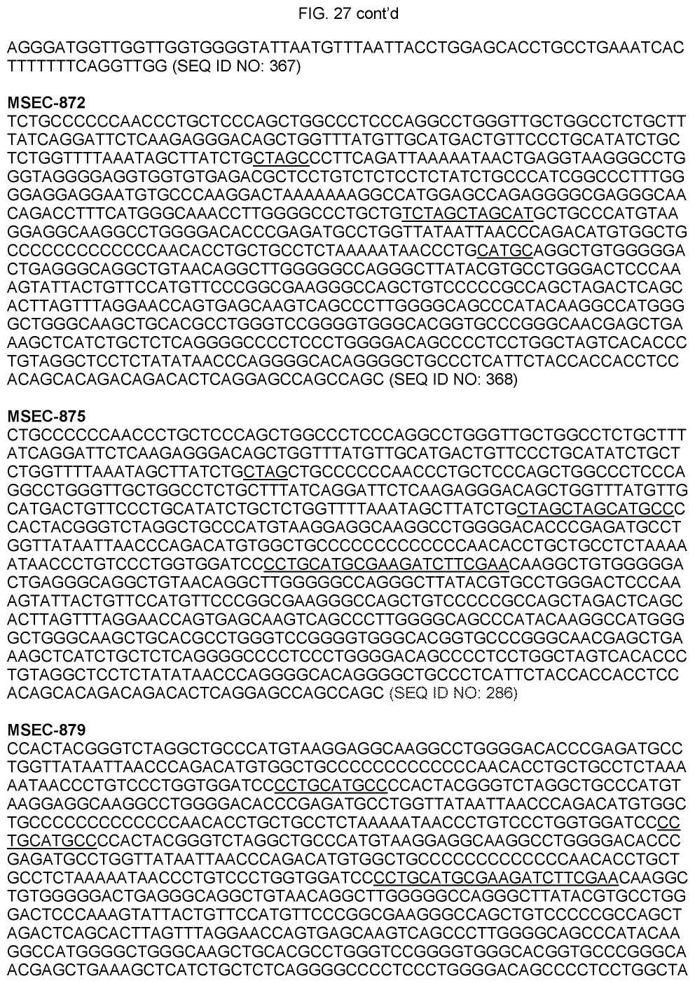

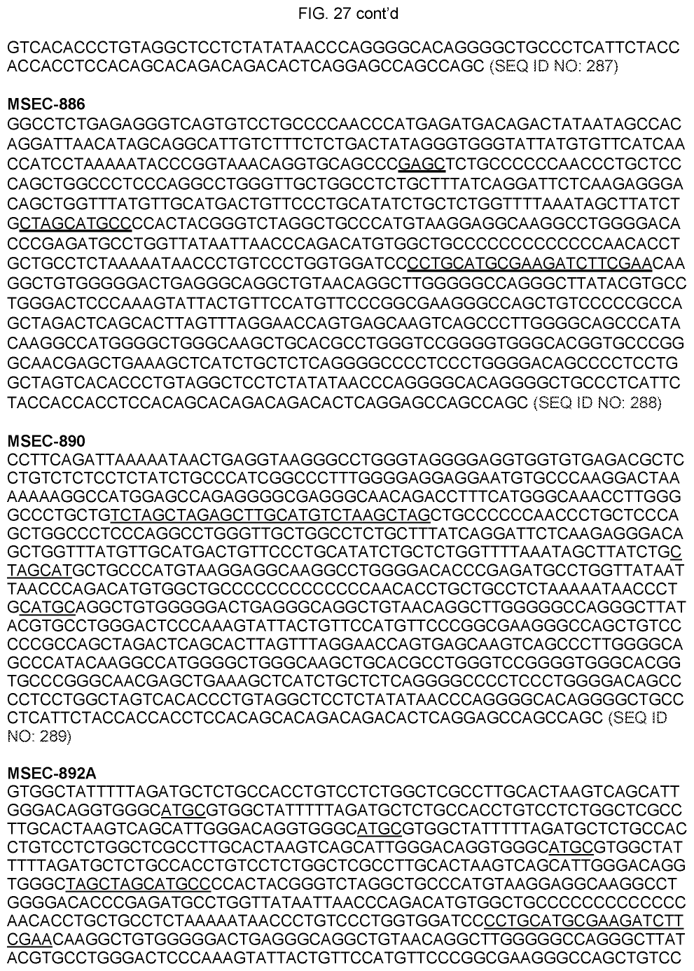

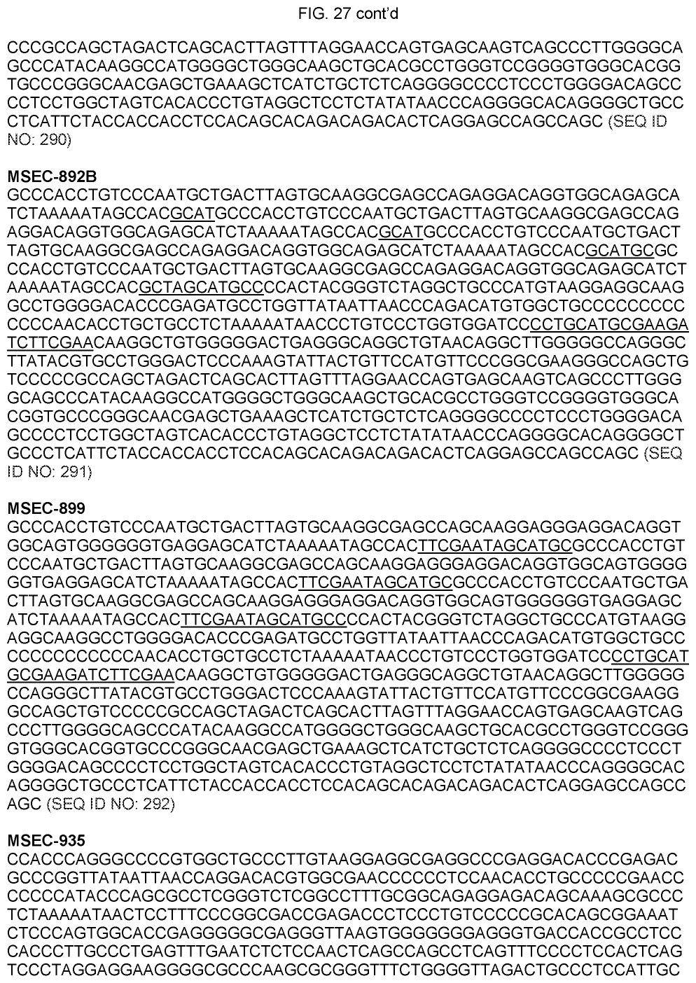

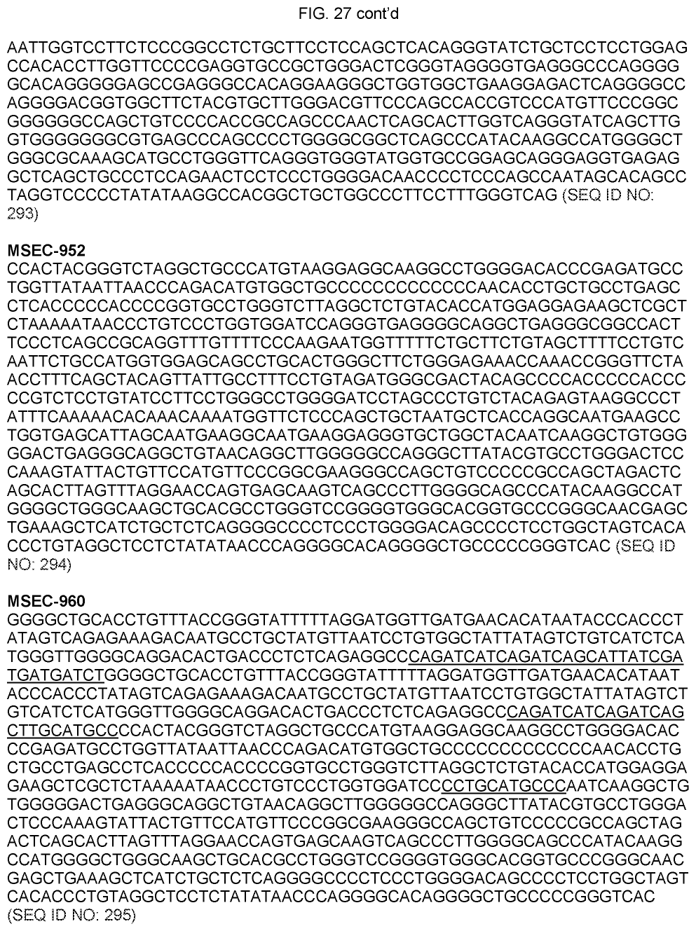

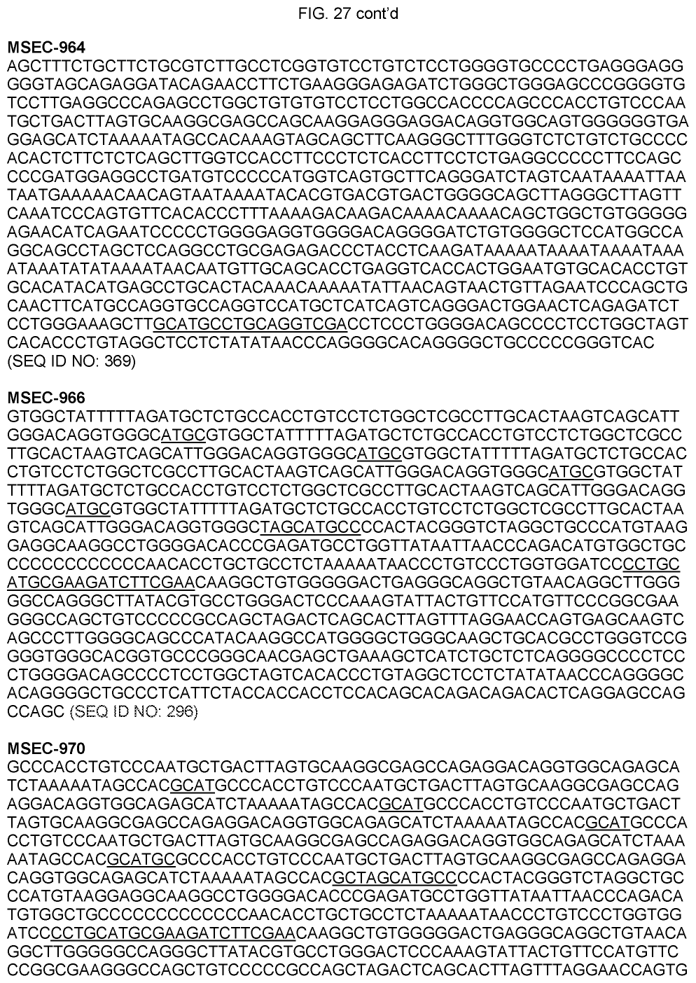

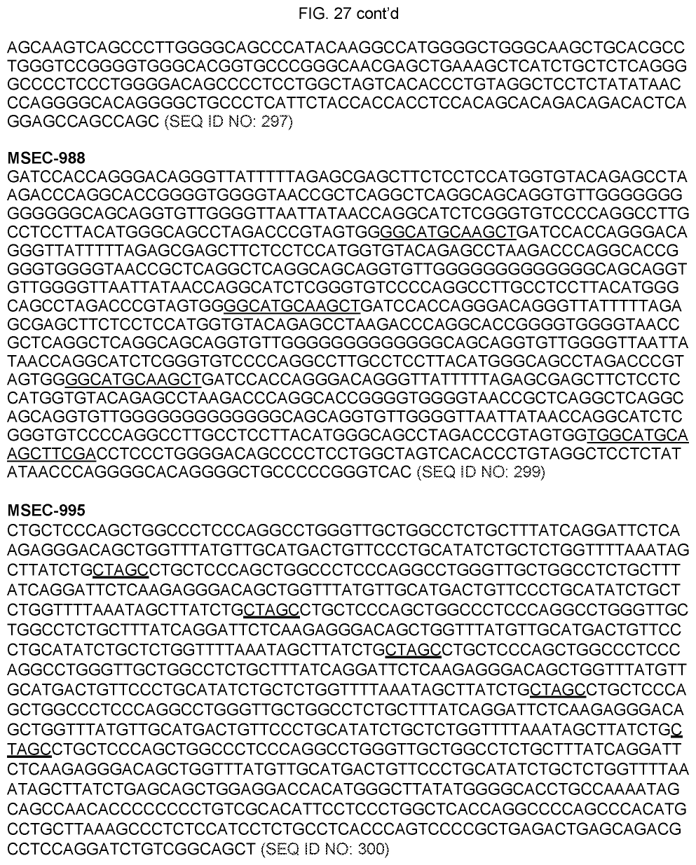

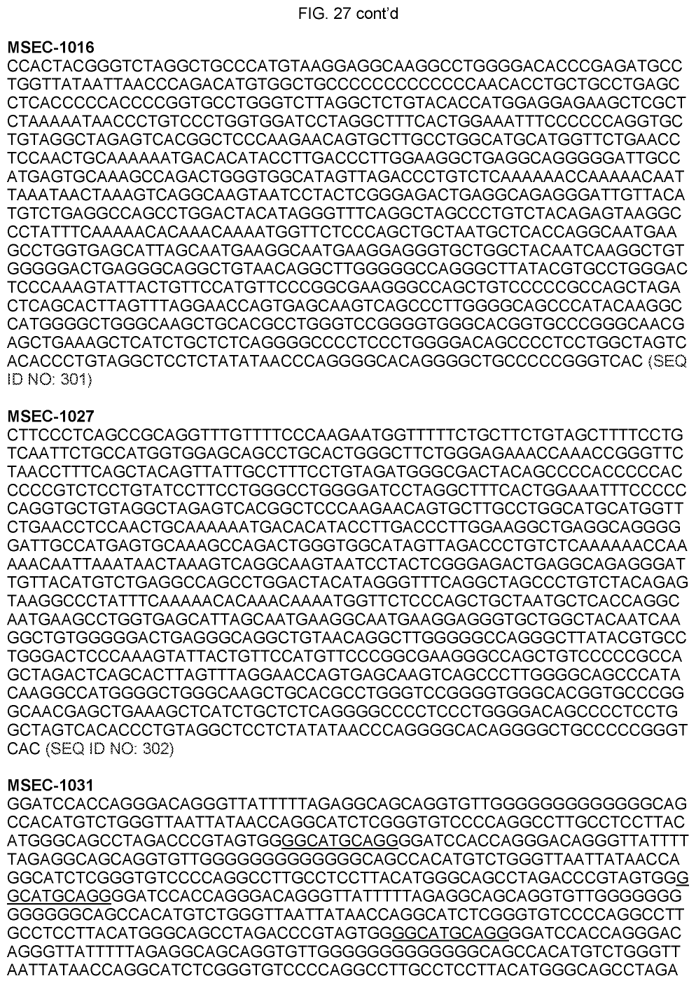

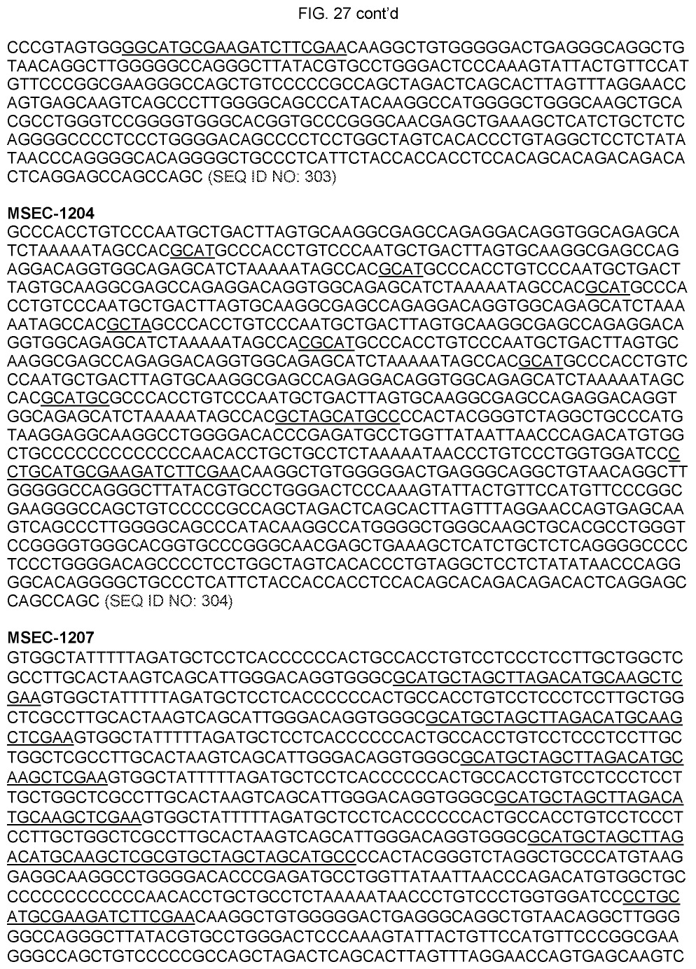

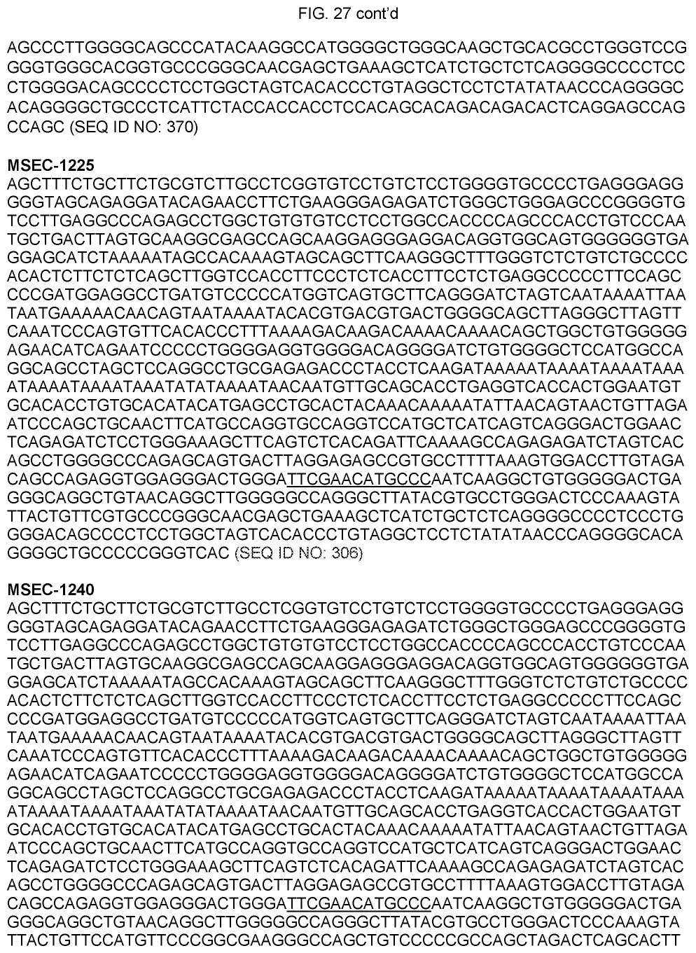

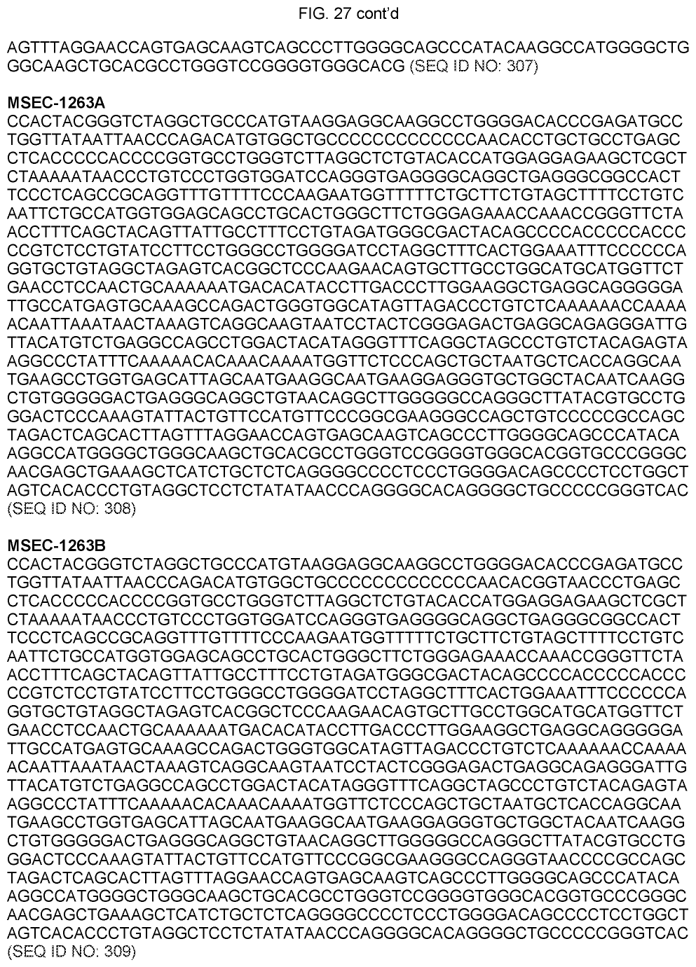

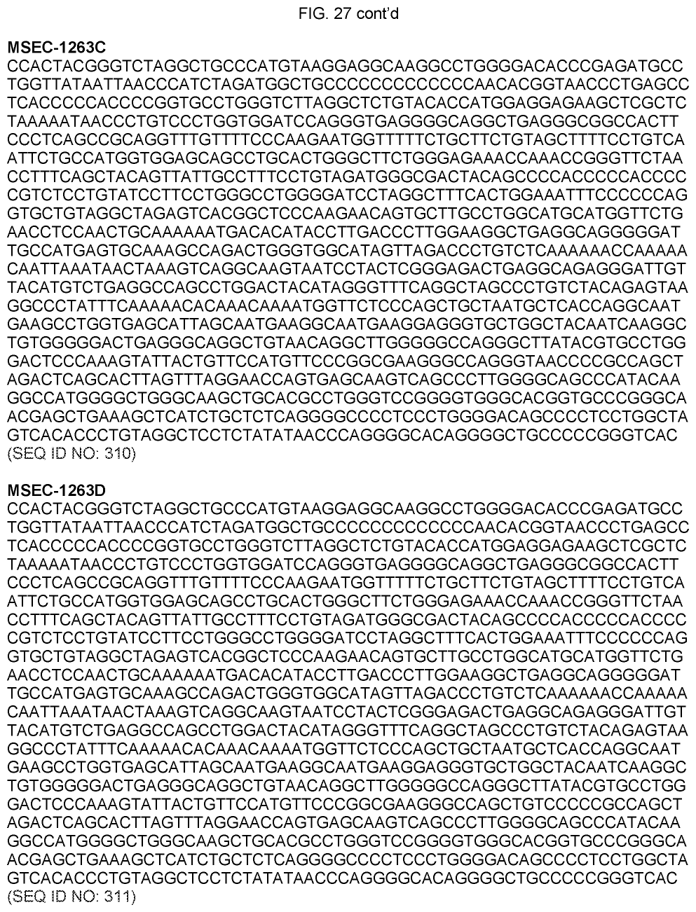

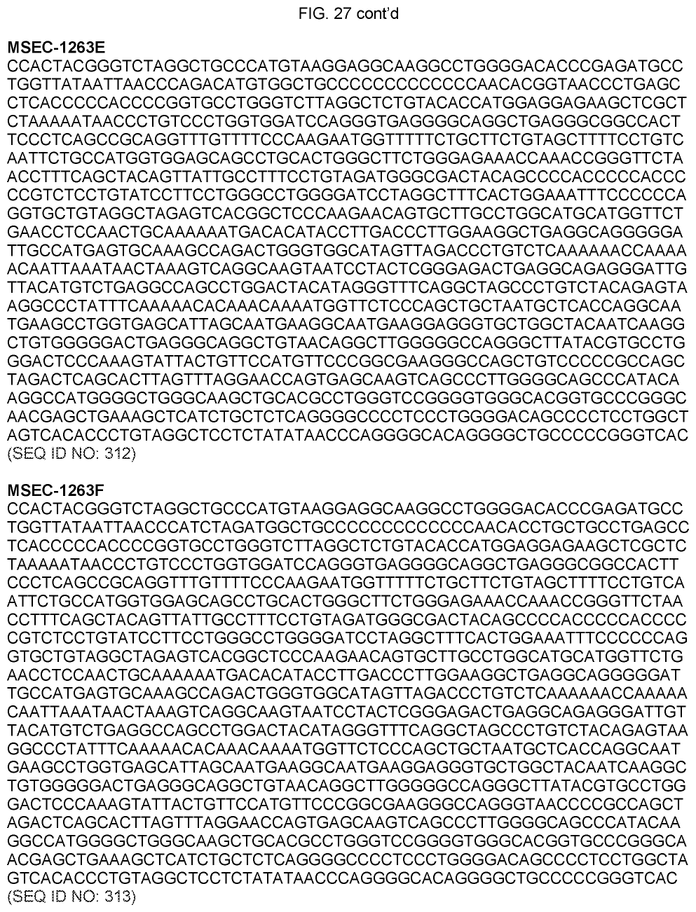

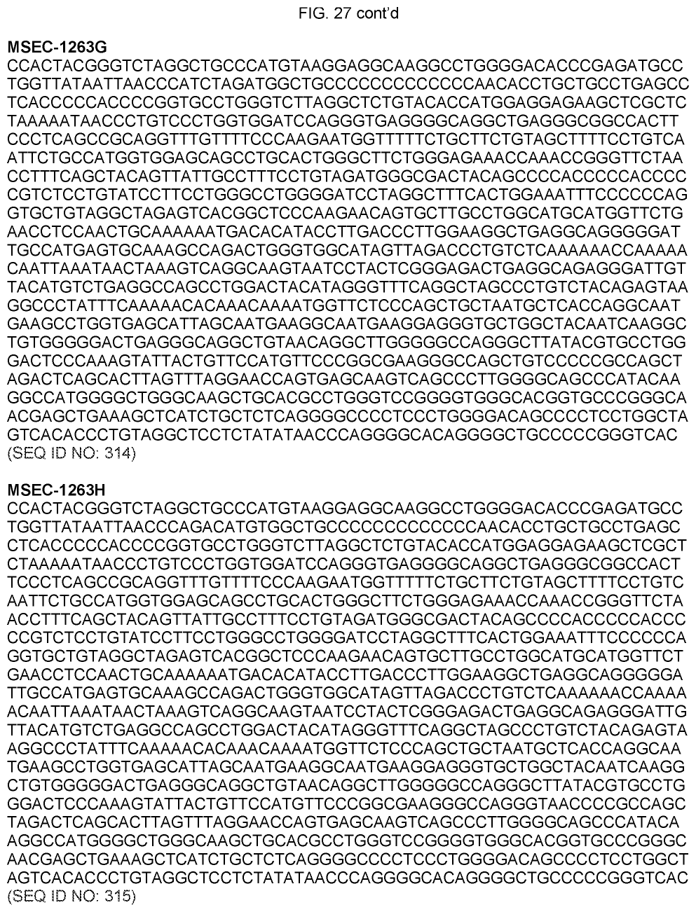

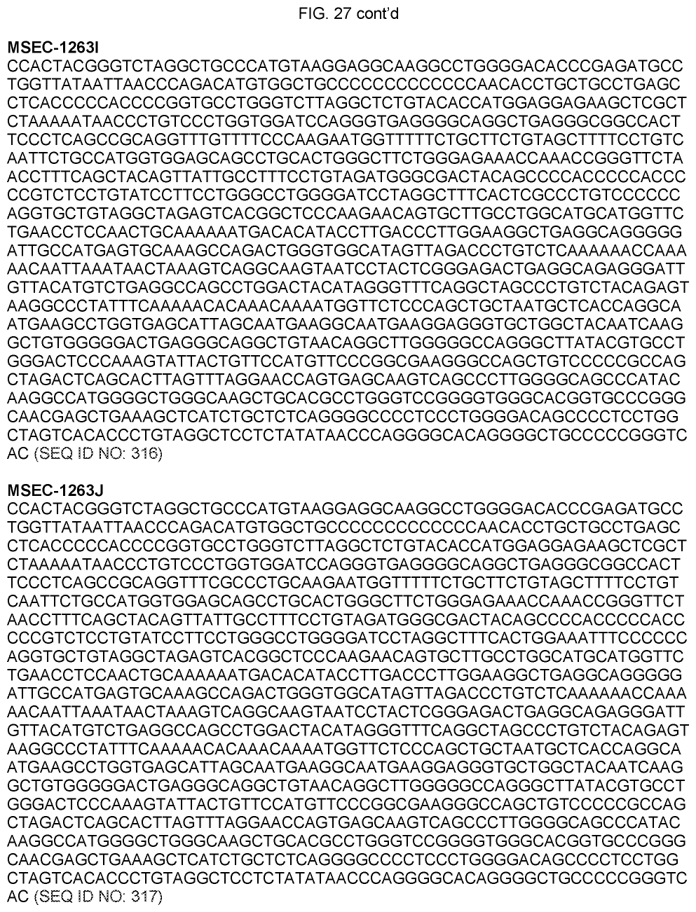

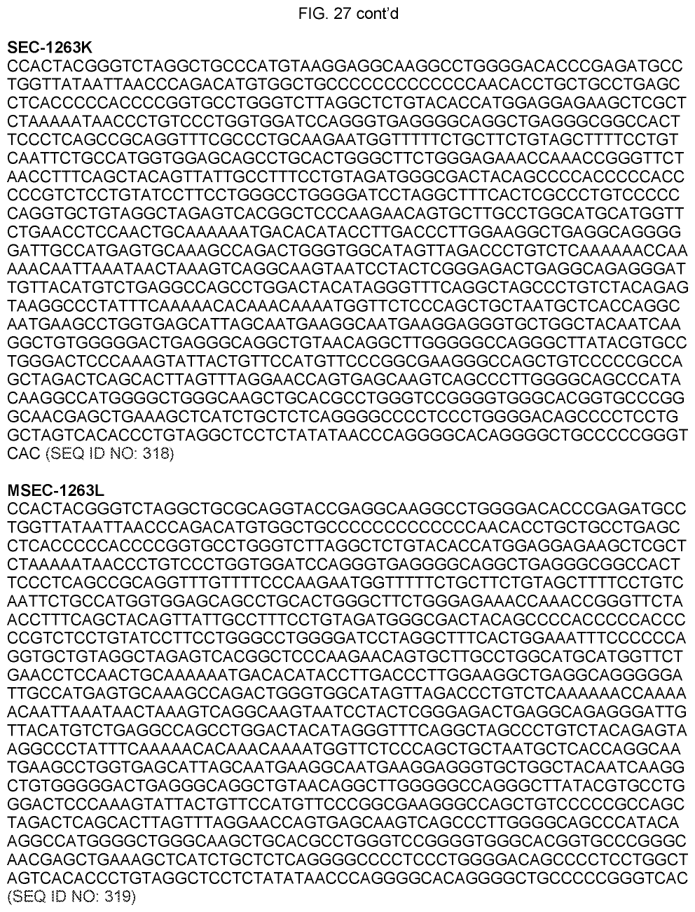

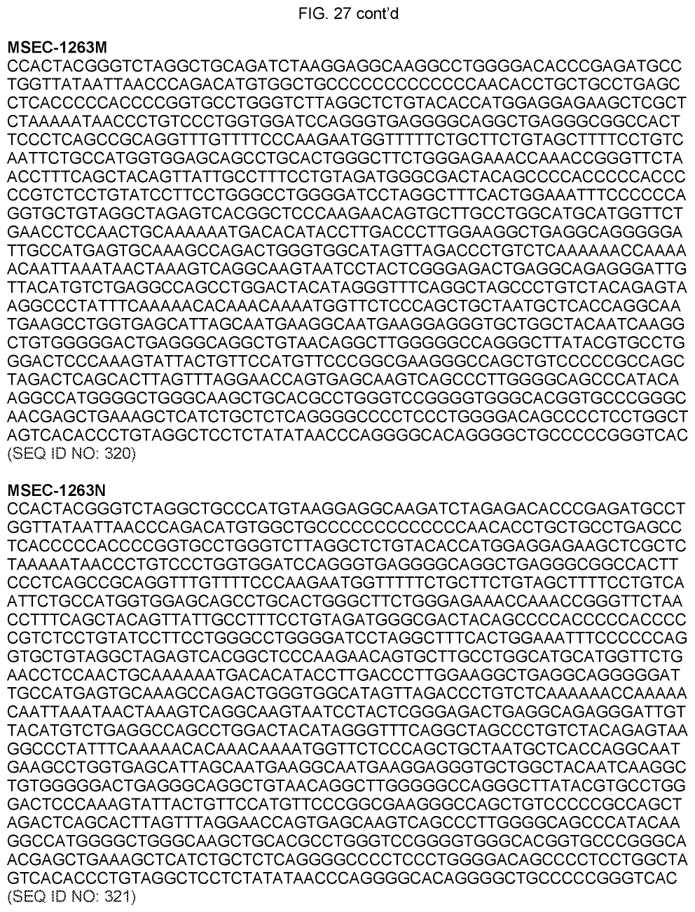

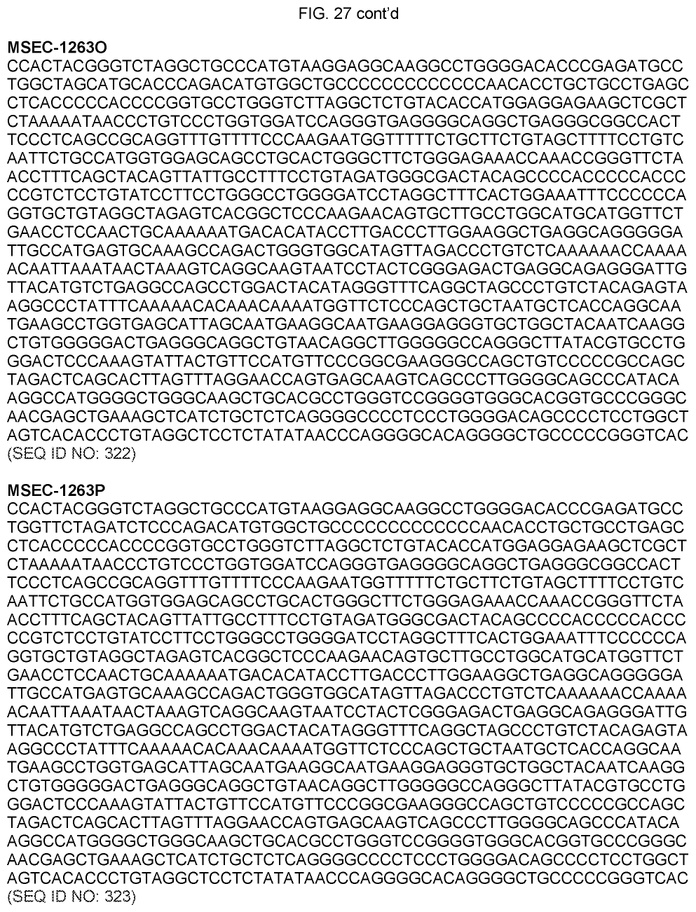

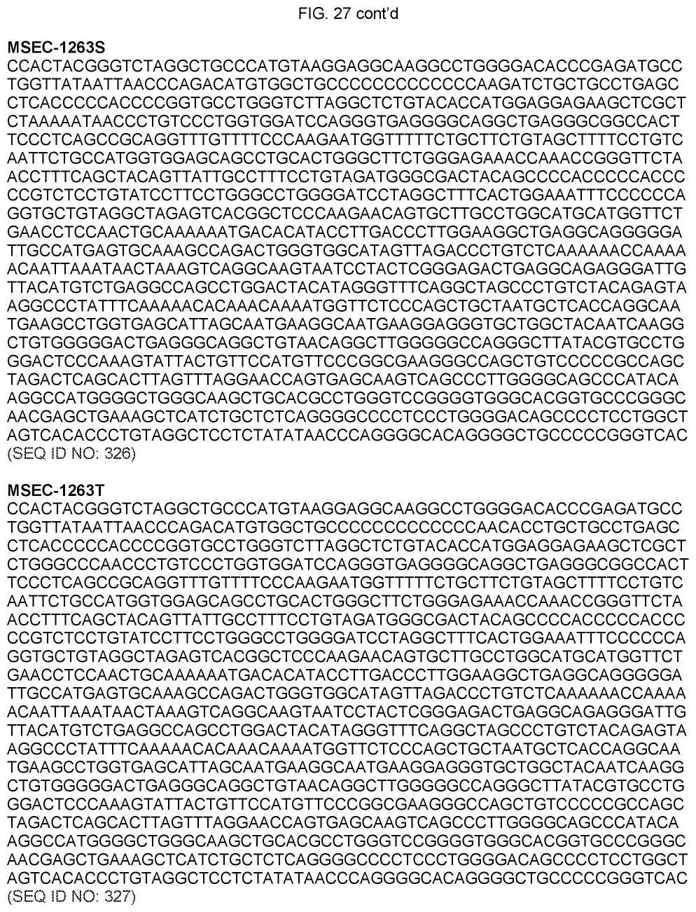

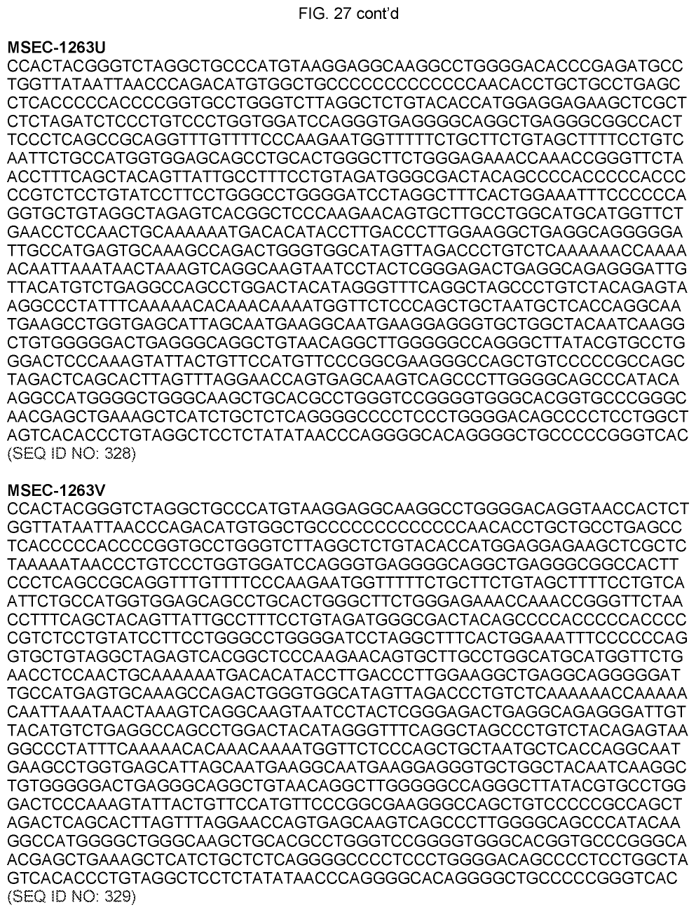

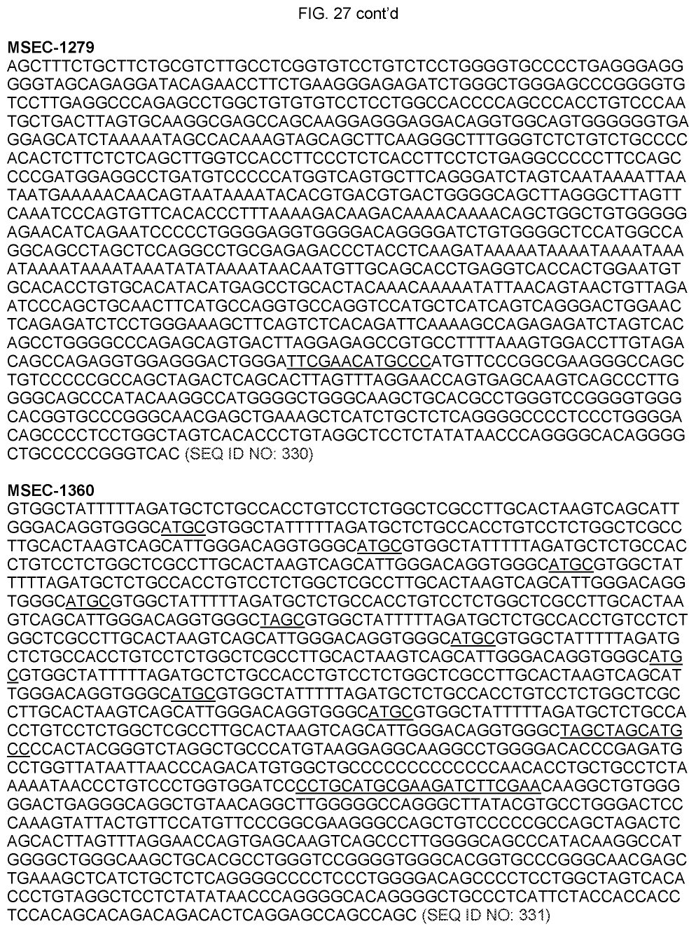

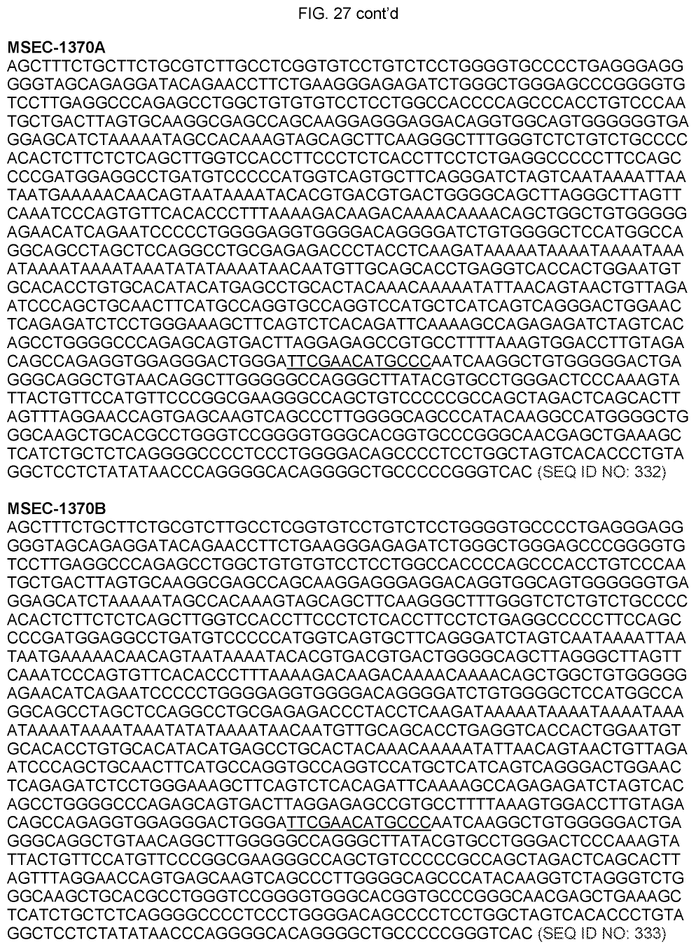

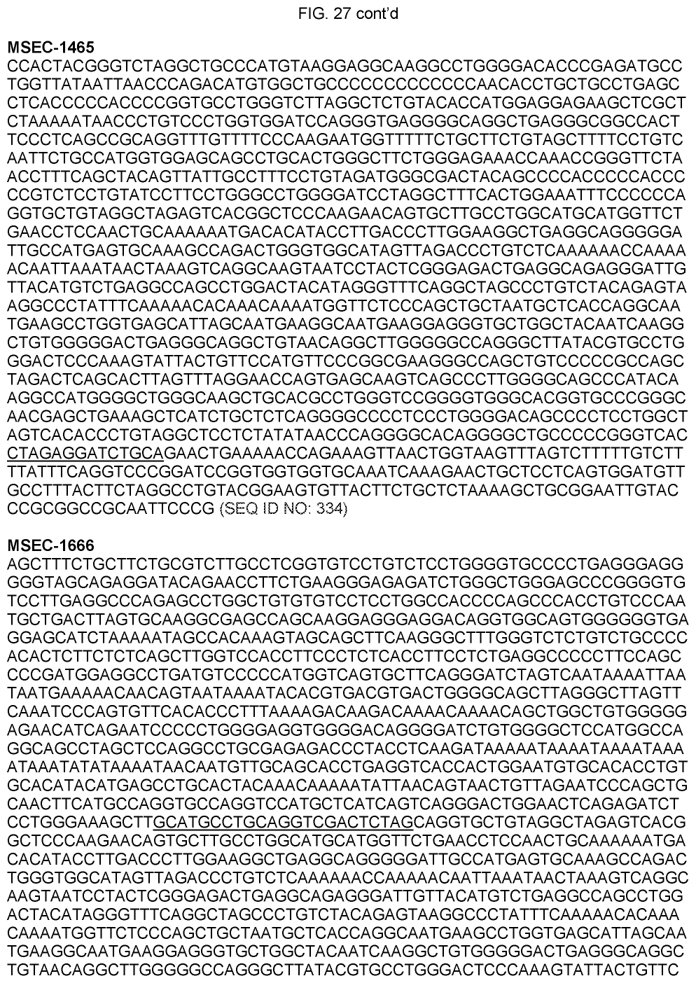

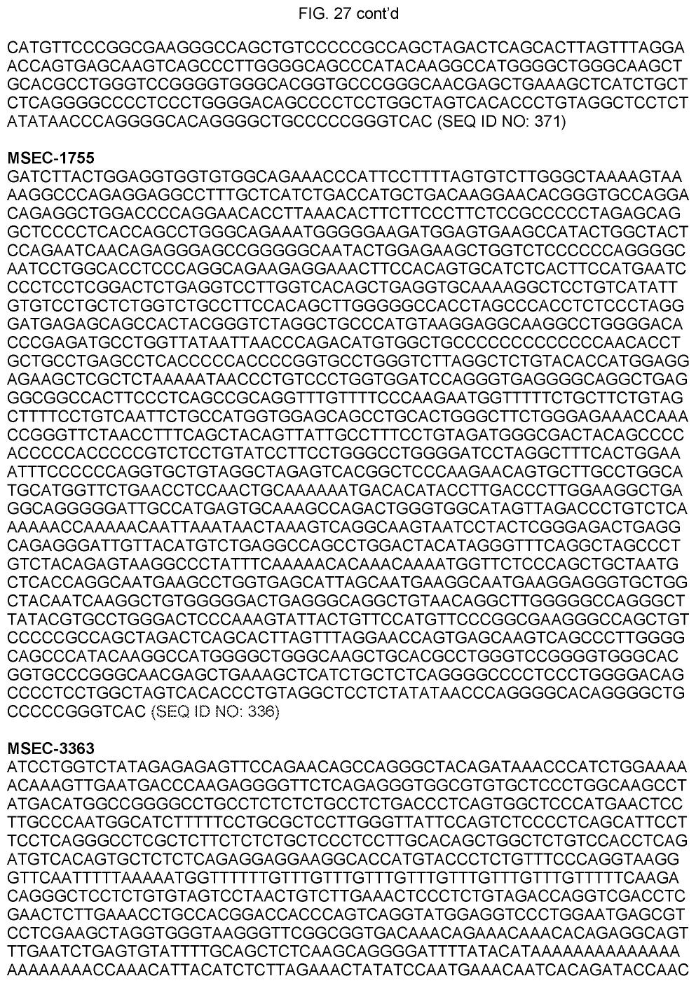

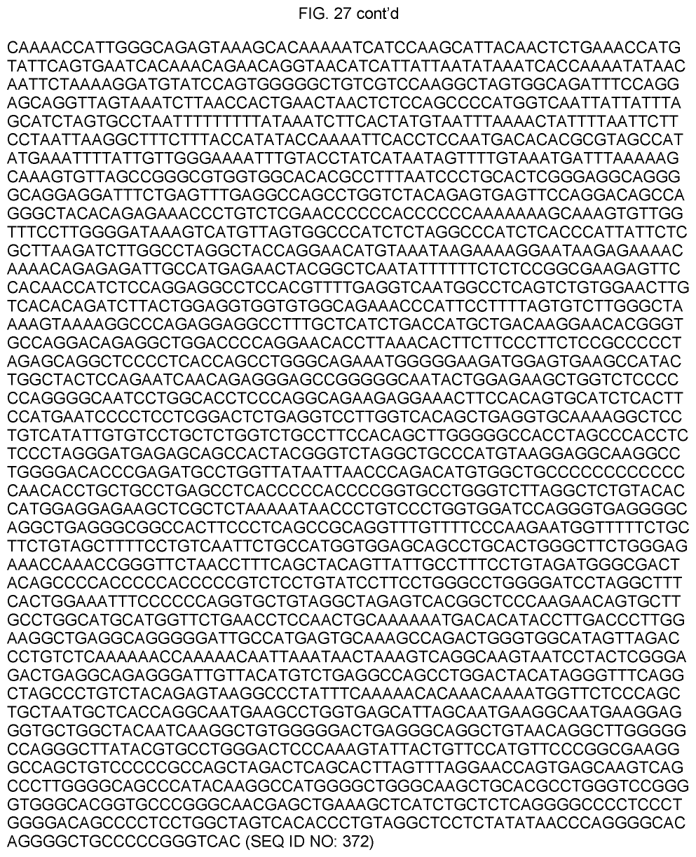

Identification of CKM Gene Regulatory Regions. Initial identification of the mouse CKM gene was based upon screening Lambda phage mouse genomic DNA libraries with a plasmid containing a partial mouse CKM cDNA (Chamberlain, J S, et al., 1985; PMID: 3990682; Jaynes, J B, et al., 1986). This strategy identified a 20 kb mouse genomic DNA fragment containing what was thought to be the “entire” mouse CKM gene (FIG. 3). This included 8 distinct exons spread over an 11 kb region, plus 3.3 kb of genomic DNA 5′ of Exon-1 and 6 kb of genomic DNA 3′ of Exon-8. This genomic and accompanying near full-length cDNA information was then used to identify the CKM transcription start site (TSS), and a typical TATA-box sequence (TATATAA) was identified 5′ of the TSS. Evidence that this genomic region contained CKM regulatory sequences was obtained via 2 complementary strategies. First, a unique 21-bp “mRNA-labeling sequence” was inserted into Exon-7 within a 16 kb genomic sequence extending from −3,300 bp to +12,700 bp, or into a putative “promoterless” genomic sequence lacking the (−3,300 to +7) region, and transfected into proliferating MM14 mouse myoblasts or differentiating MM14 mouse myocytes. Analysis of RNA indicated that the labeled mRNA was found only in differentiated cultures transfected with the entire 16 kb region. Second, a Chloramphenicol Acetyl Transferase (CAT) cDNA was ligated to the (−3,300 to +7) region and transfected into proliferating MM14 myoblasts or differentiating myocytes. Subsequent analysis showed extremely high CAT levels only in the differentiating cultures (FIG. 4 and other publications by Stephen D. Hauschka). Importantly, these two studies proved that the (−3,300 to +7) mouse CKM gene region contains regulatory information that exhibits preferential transcriptional activity in differentiated muscle cultures. Based on the common vocabulary of that era, and because an included enhancer region had not yet been delineated, the (−3,300 to +7) mouse CKM gene region was thus called the “MCK gene promoter.” This native mouse genomic DNA sequence is referred to as: MSEC-3363 in the FIG. 27 MSEC sequence library. Successful use of the CAT reporter gene cDNA in this study also showed that the 3.3 kb CKM promoter is capable of preferentially expressing heterologous proteins in differentiated muscle cells—a discovery with direct relevance to the subsequent use of CKM gene regulatory sequences for expressing essentially any protein of interest (FIGS. 1 and 2).

Delineation of the CKM gene 5′-enhancer and Promoter Regions. The native mouse CKM 5′-enhancer and proximal promoter regions were identified via basic research studies that removed sequential 5′-portions of the (−3,300 to +7) CKM region starting from the 5′end, ligating the remaining segment to a CAT cDNA, and then testing each segment for transcriptional activity in differentiated versus proliferating MM14 mouse muscle cultures (FIG. 1 in (Jaynes, J B et al., 1988; PMID: 3336366). This study disclosed that 100% of the activity exhibited by the (−3,300 to +7) region is maintained by genomic fragments as small as the (−1256 to +7) region. This native mouse genomic DNA sequence is referred to as: MSEC-1263a in FIG. 27, to indicate the MSEC's total bp number when the +7 bp 3′ of the TSS is included, and based on the common vocabulary of that era it was also referred to as the 1256 bp mouse MCK gene promoter. However, when an additional 243 bp deletion of 5′-DNA was made, the transcriptional activity of MSEC-1020 (−1020 to +7 region) in differentiated skeletal muscle cultures decreased by more than 95%, and when additional 5′-deletion fragments were tested, transcriptional activity decreased a further several percent (FIG. 1 in PMID: 3336366). These findings implied that a skeletal muscle gene enhancer might be present within the 243 bp deleted genomic DNA region, and also that a “proximal promoter” region MSEC-783 (−776 to +7) contained additional muscle gene regulatory components. However, when the proximal promoter region was reduced to a (−80 to +7) fragment, MSEC-87, this “basal promoter” and TATA-box-containing region had only background level activity in differentiated muscle cultures (FIG. 2 in PMID: 3336366). Further delineation of the enhancer-containing genomic fragment identified a 206-bp fragment, MSEC-206a, spanning the (−1256 to −1050) region that, when ligated to either the proximal or basal promoter fragments (MSEC-1263a, and MSEC-297a) exhibits strong transcriptional activity in differentiated muscle cultures, but not in undifferentiated myoblasts or in non-muscle cells, and high activity was also seen when the 206-bp fragment was ligated in an opposite 5′-to-3′ orientation, and even when it was ligated 3′ of the CAT cDNA reporter (FIG. 2 in PMID: 3336366). The ability of the 206-bp fragment to function at different 5′- and 3′-distances from the TSS as well as in either orientation proved that this DNA sequence is an enhancer; and this interpretation was strengthened by demonstrating that the 206-bp fragment could also respond to muscle differentiation signals when ligated to heterologous basal gene promoters such as those from Thymidine Kinase and from the SV40 virus (FIG. 4 in PMID: 3336366). These studies were the first publications to identify a muscle-specific enhancer sequence.

In vivo Analysis of CKM 5′-Enhancer and Promoter Regions: To determine whether these mouse CKM sequences were active in vivo, whether the 206-bp region exhibited enhancer properties, and whether genomic fragments of the types described in PMID: 3336366 exhibited analogous transcriptional activities in cardiac muscle and were inactive in tissues other than striated muscle, the genomic fragments were tested in transgenic mice (FIG. 1 in PMID: 2796990). These studies disclosed several important facts relevant to subsequent MSEC designs: (1.) CKM genomic fragments containing the 5′-enhancer (207-bp in this study) are transcriptionally active in both skeletal and cardiac muscle, and exhibit only background activities in tissues other than striated muscle (Tables 1 and 2 in PMID: 2796990). (2.) These enhancer-mediated activities occur with both proximal and basal mouse CKM gene promoter fragments, but are potentiated when combined with larger proximal promoter fragments; e.g., MSEC-1263a>MSEC-335a (Table 2 in PMID: 2796990). (3.) The proximal promoter region encompassed by the (−723 to +7) mouse CKM region (MSEC-730) was also shown to contain low level skeletal and cardiac muscle transcriptional activity in the absence of the 5′-enhancer (Table 2 in PMID: 2796990). (4.) An additional region (E2) of the mouse CKM gene (+738 to 1599) exhibits enhancer-like activity in skeletal and cardiac muscle, but not in other tissue types (Table 2 in PMID: 2796990). MSECs containing the E2 region also exhibit greater transcriptional activity when combined with larger proximal promoter fragments such as (−776 to +7) CKM gene region; e.g., (MSEC-1676) than when ligated to the (−80 to +7) proximal promoter fragment; e.g. (MSEC-974). The mouse CKM gene thus contains at least 2 enhancers. The E2 enhancer was analyzed further in subsequent studies in which it was renamed “Modulatory Region-1” (MR1), and in these studies the core enhancer region was further delineated to a 95-bp fragment named the “Small Intronic Enhancer” (SIE), see below (FIGS. 3, 5, 11). The SIE, and even smaller fragments are used in single and multimerized forms in several different MSECs.

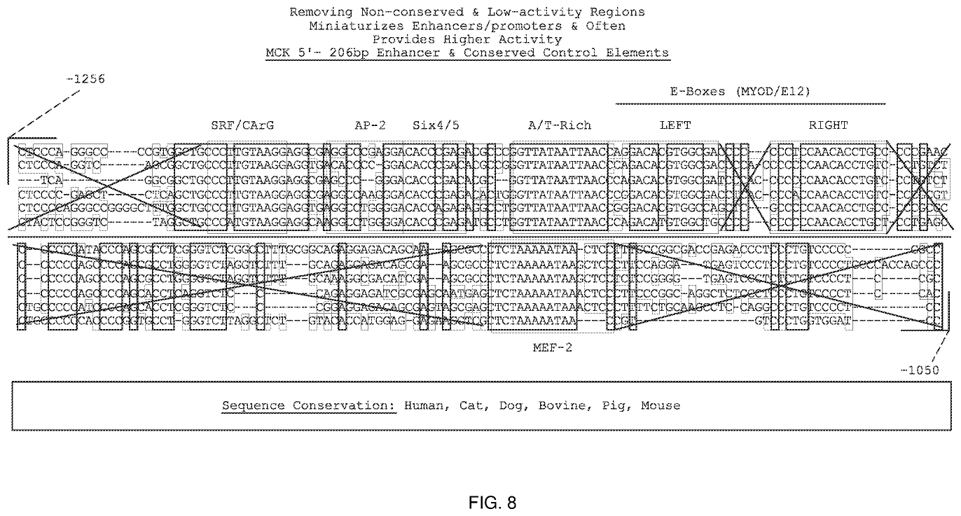

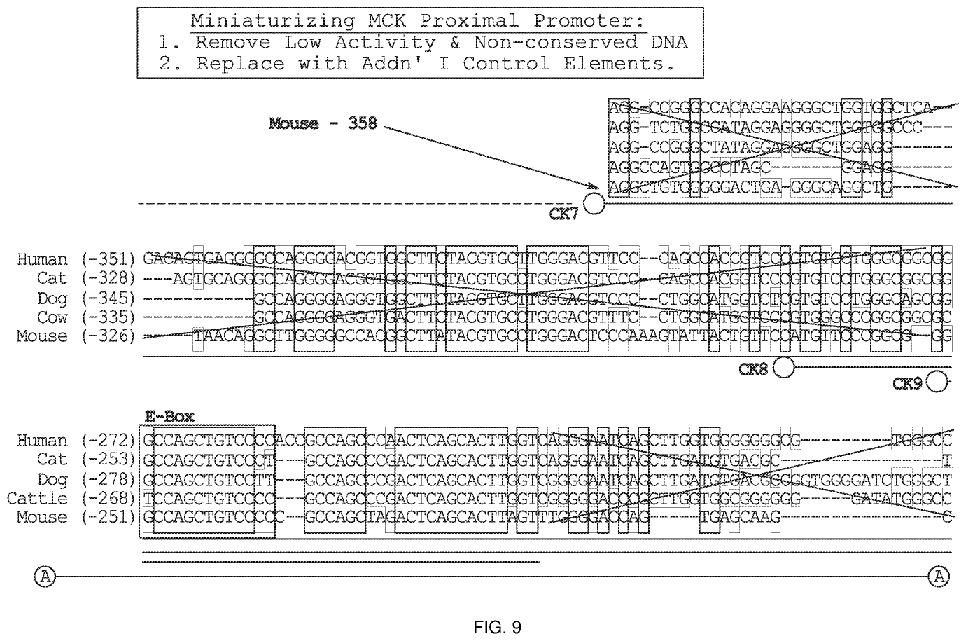

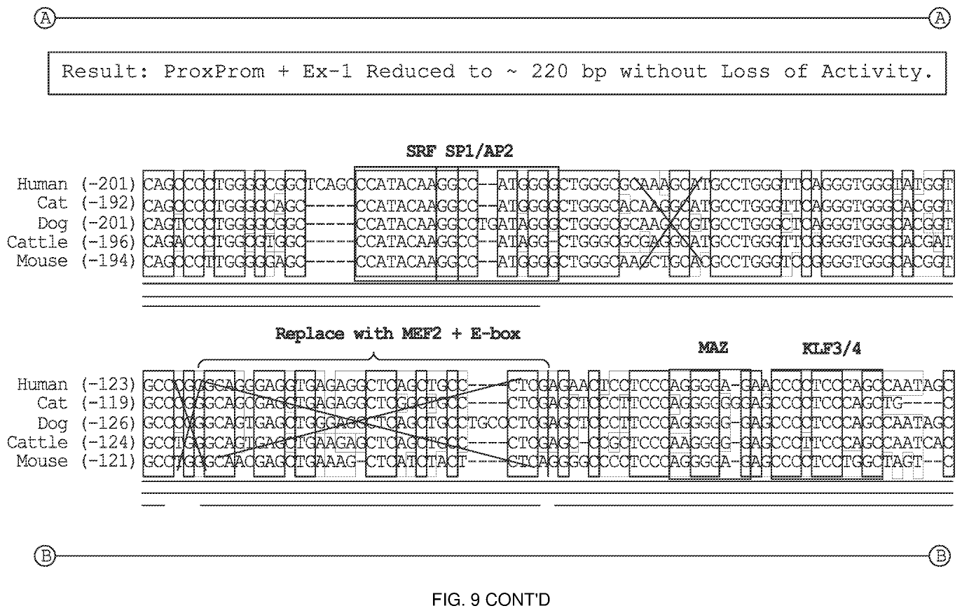

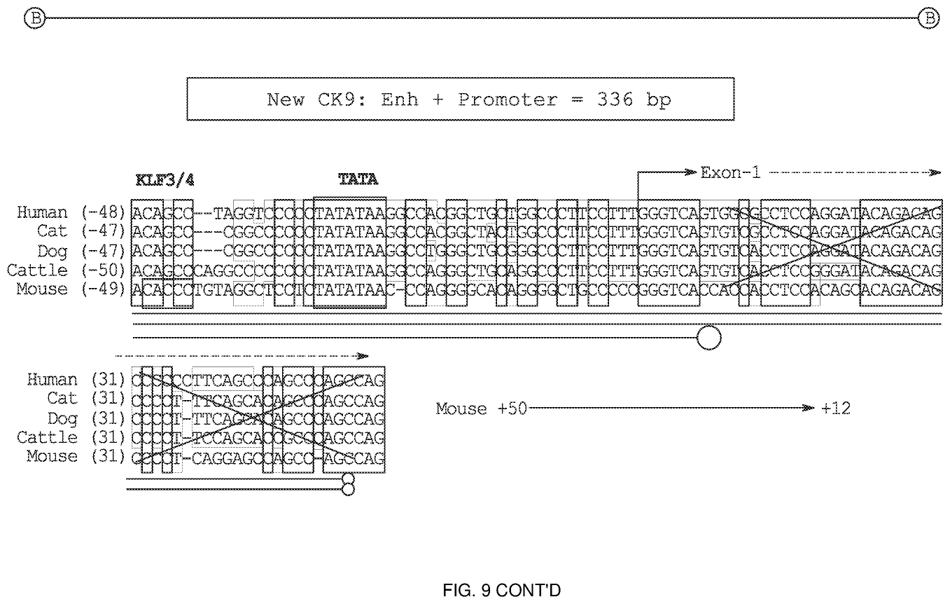

Identification of CKM 5′-enhancer Control Elements (CEs). Subsequent basic studies analyzed the mouse CKM 5′-enhancer to identify its CEs (FIG. 3). Since all vertebrates were known to contain CKM genes, it was hypothesized that the basic gene regulatory mechanisms would be similar, and that for this evolutionary reason, CKM genes in other species would have enhancers with very similar DNA sequences. Sequence searches of other vertebrate CKM genes confirmed this hypothesis, and when the putative enhancer sequences from different CKM genes were aligned so as to maximize their similarity, it was evident that they contained blocks of very highly conserved sequences that might be CEs (FIGS. 8 and 9). To verify this hypothesis, each putative CE region within mouse CKM enhancer regions located within either a (−1256 to +7) genomic fragment, or within a (−1256 to −1048; i.e., in this experimental case a 208-bp fragment) was subjected to two different mutations and then ligated to the (−80 to +7) basal promoter in either a positive or negative orientation, and each mutation was examined for its effect on transcriptional activity in differentiating skeletal muscle and cardiac muscle cells (FIGS. 3, 4, 5 in PMID: 8474439).

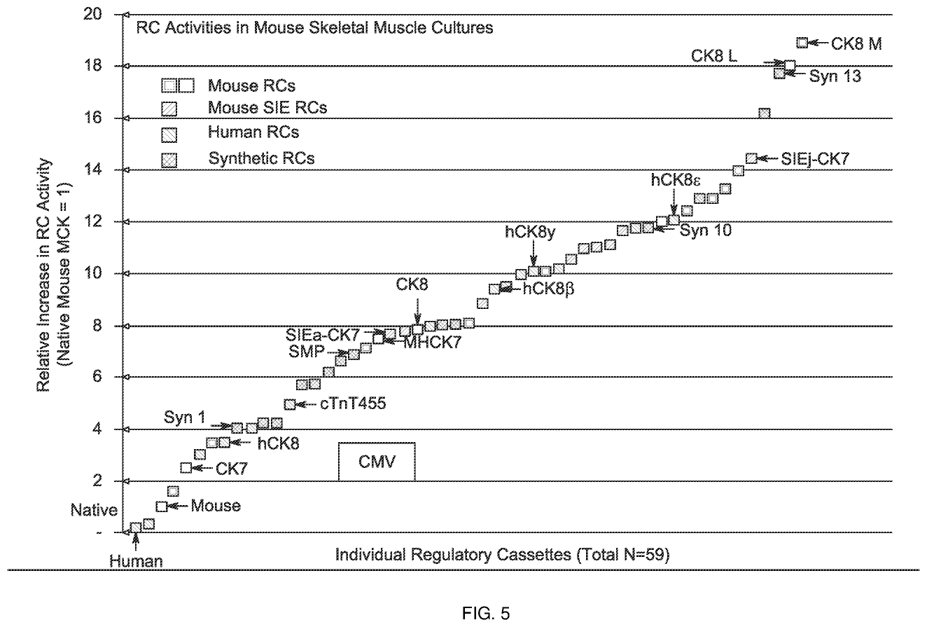

These studies substantiated the existence of at least 6 different CEs within the mouse CKM 5′-enhancer region, and by inference based on sequence similarity, existence of the same 6 CEs in the 5′-enhancers of other vertebrate CKM genes. As demonstrated by the data in FIG. 5 in PMID: 8474439 it is also evident that each mutation confers different transcriptional activities to the overall DNA construct, and that mutations of 5 CE regions: CArG, A/T, Left E-box, MEF1 (Right E-box), and MEF2, as well as mutation of both E-boxes decreased activity, while mutations of the AP2 CE increased activity. Later basic studies identified a 6th positively acting CE (Six4) within the CKM 5′-enhancer (see below) (PMID: 8617727; PMID: 14966291). The differential activities of CKM 5′-enhancer fragments containing each CE mutation were subsequently recognized as providing the basis for designing MSECs containing graded levels of transcriptional activities that would be potentially useful for gene therapy and other muscle-specific expression applications (see Exemplary Embodiments). Furthermore, the increased transcriptional activity of both AP2 mutations, shows that these modifications provide design strategies for increasing MSEC activities in both skeletal and cardiac muscle. Wildtype and all mutated versions of these 206-bp enhancer sequences are included in FIG. 27 MSECs (297a-297L) and (1263a-1263v). Other studies in this publication disclosed that a 110-bp genomic segment within the 206-bp enhancer that lacks 3 of the 7 CEs (CArG, AP2, and MEF2) also exhibits skeletal muscle-specific enhancer activity when combined with the (−80 to +7) basal CKM promoter, but is devoid of cardiac muscle activity in this context (FIG. 3 in PMID: 8474439); this construct (MSEC-206b) may be applicable to gene therapy situations in which skeletal muscle, but not cardiac muscle expression is desirable (see Exemplary Embodiments).

Identification of Transcription Factors Associated with Unknown CKM Gene Control Elements (CEs). As illustrated in (FIG. 8) the 5′-CKM enhancer regions from different vertebrate species contain many sequence regions of contiguous bp that are highly conserved. Most of these regions have sequences identified as corresponding to the consensus DNA binding sites for known transcription factors (PMID: 8474439). However, some conserved sequence regions do not correspond to known consensus binding sites or exhibit close similarities to several TF binding sites (PMID: 8617727; PMID: 14966291). To identify the specific TFs in differentiated skeletal muscle cells that bind to unknown conserved wildtype sequences, the individual sequence was mutated and the transcriptional activities of 5′-enhancers with native and mutated sequences were then compared. Significant decreases in activity were interpreted as meaning that the conserved region binds one or more TFs with positive transcriptional activity (PMID: 8617727). Differential quantitative proteomics were then applied to discriminate between the TFs capable of binding to only the wildtype DNA sequence from those capable of binding to both the wildtype and mutant sequence. As one example, this strategy identified Six/4 as the primary TF in skeletal muscle cells that binds to a conserved sequence region (called TREX in initial studies) located between the AP-2 and A/T-rich CEs in the mouse CKM 5′-enhancer (PMID: 14966291). The same CE-discovery approach was used to successfully identify two other conserved CEs within the mouse CKM gene proximal promoter, see below (PMID: 18710939; PMID: 20404088). Importantly with respect to MSEC designs, the CKM 5′-enhancer Six/4 CE is important for maximal transcriptional activity of both the native mouse (−1256 to +7) CKM genomic fragment (MSEC-1263a) and for the 5′-enhancer region when combined with the (−80 to +7) proximal promoter (MSEC-297a) in differentiated skeletal muscle cells, but not in cardiomyocytes (PMID: 8617727). This implies that synthetic enhancers with multiple Six/4 CEs would likely have elevated skeletal muscle expression without concomitant increases in cardiac expression (see Exemplary Embodiments).

Identification of Control Elements (CEs) in the CKM Gene Promoter. As discovered in the mouse CKM gene 5′-enhancer, basic studies of the gene's proximal promoter also disclose clusters of highly conserved sequences. These are located within the (−356 to +7) region (FIG. 9); and as in the 5′-enhancer some conserved sequences could be identified by their close similarities to the consensus sequences of known TFs; e.g., E-box, CArG/SRF, SP1, AP2 and TATA-box sequences. Among these, mutational analysis of the conserved proximal promoter E-box was particularly important due to the high transcriptional activity of E-boxes in the CKM 5′-enhancer. Mutation of the proximal promoter E-box in the context of the entire (−1256 to +7) region (MSEC-1263c) as well as deletion of the E-box region within the (−358 to +7) region when this was ligated to the 206-bp 5′enhancer (MSEC-466) led to significantly reduced transcriptional activity in transgenic mice (see FIG. 1 and Table 1 in PMID:8756664). Deletion of a proximal promoter region containing the conserved CArG/SRF CE was also examined and found to decrease transcriptional activity when this version of the proximal promoter was ligated to the 206-bp enhancer (MSEC-476) (see FIG. 1 and Table 2 in PMID:8756664).

Several other highly conserved sequence regions within the (−358 to +7) mouse CKM proximal promoter region did not correspond to the DNA-binding sites of any known muscle transcription factors (FIG. 9). These conserved regions were thus studied via the same mutation and differential quantitative proteomics strategies that proved successful for identifying the 5′-enhancer Six/4 CE and its TF (PMID: 14966291). These studies identified MAZ and KLF CEs that bind the MAZ and KLF3 TFs (PMID: 18710939; PMID: 20404088). The mouse CKM gene MAZ CE is located in the (−81 to −67) region, and its mutation causes a more than several-fold reduction in transcriptional activity in both skeletal and cardiac muscle cultures (FIG. 1 in PMID: 18710939). The mouse CKM gene contains 2 KLF3 CEs located in the (−136 to −130) and (50 to −44) regions, and mutation or deletion of these elements causes a 2-3-fold reduction in transcriptional activity in skeletal muscle cultures (FIG. 3 in PMID: 20404088).

Putative CKM Gene Control Elements (CEs). In addition to highly conserved CKM gene 5′-enhancer and promoter regions that have already been identified with respect to their TF binding characteristics, both regions contain examples of highly conserved sequences whose binding factors have not yet been identified. The enhancer contains a 25 bp region between the Right E-box and 3′-MEF2 CE with 80% sequence conservation that is very likely to provide one or more regulatory functions (FIG. 8), yet deletion of this entire region has little to no effect in any in vitro or in vivo physiological context yet tested in terms of MSEC expression levels. The CKM (−358 to +7) proximal promoter contains 7 unidentified regions with very high sequence conservation and no known binding factors (FIG. 9), yet only one of these putative CEs can be mutated or deleted without substantially decreasing transcriptional activity (see MSEC Miniaturization Paragraph below).

Generalization of CKM Gene Control Elements (CEs) to Analogous Elements in other Muscle Genes. An important aspect of identifying all of the mouse CKM gene CEs described above is that sequence searches for similar CEs in other skeletal and cardiac muscle gene control regions disclosed that these elements, albeit with sometimes slightly altered sequences, are present in many other muscle genes. Thus mutating or multimerizing these elements within the enhancers and promoters of other vertebrate muscle genes is predicted to have similar effects on the transcriptional activities of these regulatory regions (see Exemplary Embodiments). Furthermore, discovery that the same CEs were present in different skeletal muscle enhancers and promoters suggested that totally synthetic muscle regulatory regions could be designed by combining libraries of muscle gene CEs in different linear orders (see below, Synthetic MSECs and see Exemplary Embodiments).

Miniaturization of CKM Gene 5′-Enhancer and Promoter Sequences to Facilitate Packaging in AdenoAssociated Virus and Other Small Vectors. Sequence alignments of enhancer and promoter regions of the CKM genes from different vertebrates also disclose regions between identified CEs that exhibit much less sequence conservation. While some portions of these regions may provide critical spatial distances between contiguous CEs, it was hypothesized that it would be possible to delete some bp between neighboring CEs without decreasing transcriptional activity. If so, this would create smaller MSECs that could then be packaged more efficiently in AAV vectors in conjunction with large cDNAs. Additionally, and when the cDNA component size will not result in vector packaging problems, creation of MSECs containing miniaturized CKM enhancers and promoters provides the opportunity for adding additional CEs within the miniaturized regulatory regions, as well as adding multimerized CKM enhancers and enhancers from other muscle genes (see sections below, and see Exemplary Embodiments). The miniaturization concept was tested by progressively deleting incrementally larger portions of non-conserved sequence regions between neighboring CEs in both the (−1256 to −1050) CKM 5′-enhancer region (FIG. 8), and the (−358 to +7) promoter region (FIG. 9). Regions that were tested for deletion without appreciable decreases in transcriptional activity are illustrated by “X'd Out” sequence regions in these FIGs., but deletions within only some tested regions caused no losses in transcriptional activity. The largest of these within the 5′-enhancer was removal of 63 bp between the Right E-box and 3′-MEF2 site; the largest deletion within the proximal promoter was removal of 104 bp from the 5′-end to the −254 position. Beginning from an original CKM-based MSEC containing 605 bp this iterative process initially resulted in the design of a MSEC-571 (similar to the 580-bp cassette in PMID: 17235310), and subsequently to numerous MSECs in the 400-500 bp size range that have very high muscle-specific expression levels in cell cultures as well as in vivo; e.g. MSEC-400.

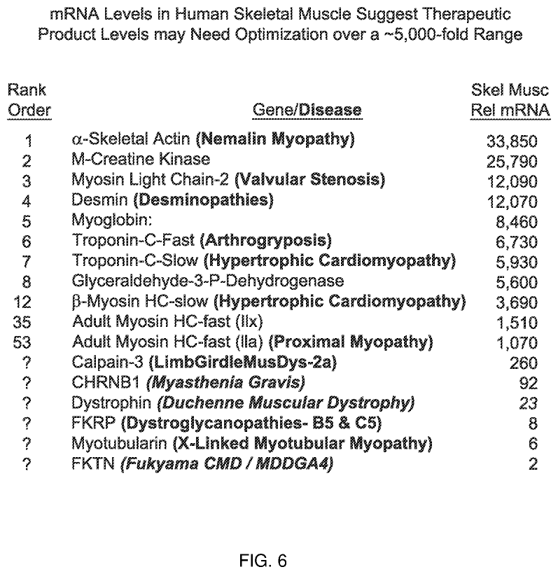

Identification of Regulatory Regions and Control Elements (CEs) in the CKM Gene Intron-1 Modulatory Region. The CKM Intron-1 Modulatory Region (MR1) was discovered fortuitously during early studies of CKM gene expression when a 1 kb restriction fragment within intron-1 was inserted 5′ of the gene's proximal promoter and observed to increase expression in skeletal muscle cultures and transgenic mice, and to have enhancer-like properties (PMID: 3336366; PMID: 2796990). Further analysis of MR1 began after finding that numerous MSECs with high overall expression levels were much less active in slow than in fast muscle fibers (FIG. 6 in PMID: 17235310); and yet that a native 6.5 kb mouse CKM gene restriction fragment encompassing the (−3349 to +3236) region was capable of expressing full-length dystrophin in all fiber types within transgenic mice (PMID:8355788). It was thus hypothesized that an enhancer within the MR1 region might be required for full CKM expression in slow fibers (PMID: 21797989). Sequence alignments of the mouse CKM MR1 region with intron-1 sequences from 5 other mammals disclosed several sub-regions containing conserved bp-clusters (Fig. S1 in PMID: 21797989). A 95-bp cluster in the (+901 to +995) region was of particular interest due to its content of 4 known muscle gene CEs: 2 E-boxes, 1 MEF2, and 1 overlapping MAF and AP1 sequence (FIG. 1 in PMID: 21797989). Subsequent cell culture studies of the 95-bp region in various MSECs (e.g., MSEC-732; MSEC-734; MSEC-804) proved that it behaved like an enhancer (it was thus named the Small Intronic Enhancer, SIE), and that it exhibited high transcriptional activity when combined with the (−358 to +7) mouse CKM gene proximal promoter (FIG. 2 in PMID: 21797989). CE mutation studies then showed that the 2 E-box CEs and the MEF2 CE are critical for full SIE transcriptional activity in skeletal muscle cultures, but that deleting bps within the overlapping MAF and AP1 half-site sequence caused no loss of activity (FIG. 3 in PMID: 21797989). Although this deletion result does not indicate whether the highly conserved half-site may play functional roles in vivo, it suggested that the SIE could be further miniaturized by deleting part of this sequence. This strategy led to the design of a 74-bp sequence (MSEC-74) that exhibits high transcriptional activity in skeletal muscle cultures, as well as additive activity when ligated to other MSECs such as MSEC-571; e.g., MSECs: 732, 734, 804, 809, 892a/b, 966, 970, and 1204 (see sections below, and see Exemplary Embodiments).

MSECs Containing Slow Muscle Fiber Gene Regulatory Components. As mentioned above, it was discovered that numerous CKM-based MSECs with high overall expression levels are much less active in slow than in fast muscle fibers (FIG. 6 in PMID: 17235310). This fiber type-transcriptional bias would not be optimal for MSEC applications in which equal product expression is required in all skeletal muscle fiber types. As a potential strategy for overcoming this issue, enhancer regions from slow muscle-specific genes such as Slow Muscle Troponin I (TNNI1) were ligated to CKM-based MSECs (e.g., MSECs-550, -563 and -757), or to TNNT2-based MSECs (e.g., MSEC-587).

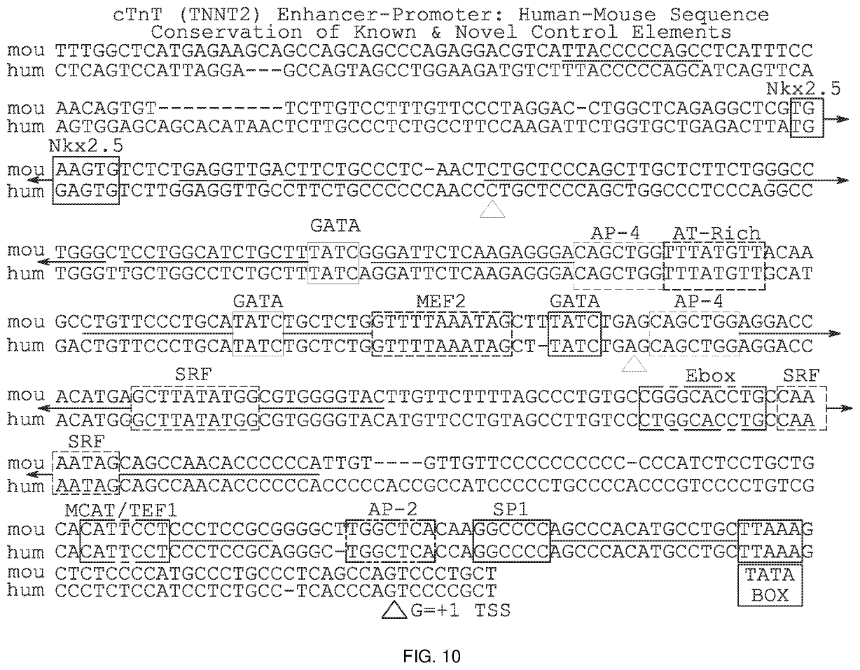

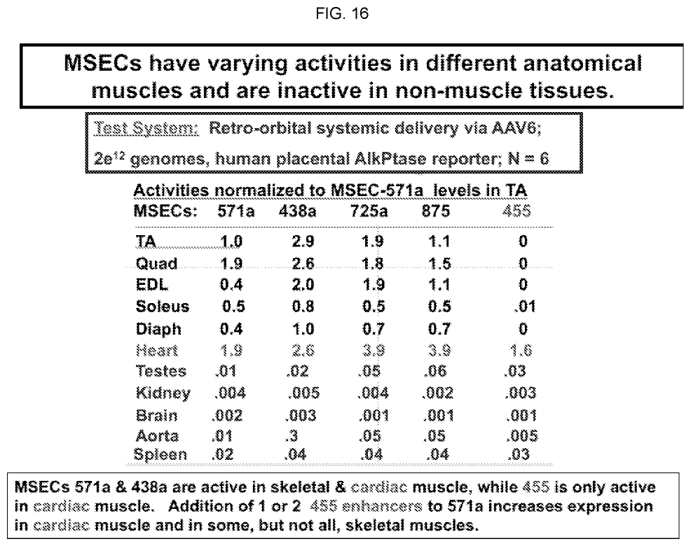

Identification of Regulatory Regions and Control Elements (CEs) in the Human Cardiac Troponin T (TNNT2) Gene. Although the CKM gene is expressed at high levels in both skeletal and cardiac muscle, CKM's high skeletal muscle expression properties means that CKM-based MSECs would not be optimal for treating medical problems restricted to cardiac muscle. For this reason it is useful to design MSECs that exhibit cardiac muscle-specific expression. Since transcription from the cardiac troponin T gene (TNNT2) is known to be cardiac-specific (PMID: 26774798), and since studies by other investigators had identified regulatory regions of avian and rodent TNNT2 genes (PMID: 2993302; PMID: 7982978; PMID:18951515, PMID: 9689598) analogous regions of the human TNNT2 gene were cloned. A 495-bp construct (MSEC-495) (FIG. 10) was tested in fetal rat cardiomyocyte cell cultures and it had high transcriptional activity. MSEC-495 was then miniaturized, as described above for the (−1256 to +7) CKM 5′-enhancer-promoter genomic fragment via the iterative deletion of poorly conserved sequence regions to create MSEC-320, and the miniaturized 130-bp enhancer region was then multimerized to create the highly active MSEC-455a. As anticipated, MSEC-455a had high expression in cardiomyocytes and only background expression in non-muscle cells; however it also exhibited expression in skeletal muscle cultures during the onset of differentiation. This behavior is consistent with the fact that an initial step in skeletal muscle differentiation entails activation of numerous cardiac muscle genes, followed by their repression as muscle fibers mature (PMID: 1728592). Consistent with this fact, MSEC-455a exhibits only background transcriptional activity when systemically delivered to adult mice via AAV (FIG. 16). The miniaturized TNNT2 enhancer was also shown to synergize with MSECs containing CKM enhancer and promoter components such as MSEC-571a by increasing expression levels in heart muscle, and in some, but not all, skeletal muscles e.g., MSEC-725a and MSEC-875 (FIG. 16). These TNNT2-based MSECs are highly relevant to (see Exemplary Embodiments).

Multimerization of Enhancers in MSECs. MSEC transcriptional activities can be increased by ligating additional enhancers 5′ of the promoter or 3′ of the cDNA and the enhancer sequences can be in either orientation relative to the TSS. The added enhancers can be identical or differ from each other, and they do not need to be derived from the same native gene as the promoter. For example, MSECs 455a, 590, and 725a have 2, 3 and 4 miniaturized TNNT2 enhancers ligated to a miniaturized TNNT2 promoter; MSEC-1204 has 8 74-bp CKM SIEs ligated to the CKM 5′-enhancer, and proximal CKM regions composing MSEC-571, MSEC-770 has a MYH6 enhancer ligated to a miniaturized CKM enhancer ligated to a CKM promoter; and MSEC-518 has a TNNI1 enhancer ligated to a Synthetic enhancer ligated to a miniaturized ACTA1 promoter.

MSECs Containing ACTA1 Promoter Regions. Because CKM enhancers and promoters exhibit higher expression in fast fibers, a strategy for circumventing this issue is to substitute miniaturized versions of the ACTA1 gene promoter (because alpha-skeletal actin is produced at approximately equal levels in all fiber types) for the CKM promoter, and then to combine the ACTA1 promoters with synthetic enhancers; e.g., MSECs 285, 319b and 429a (see Exemplary Embodiments). Transcriptional activity can be increased further by ligation of the (ACTA1 “Distal Regulatory Element/DRE” located within the enhancer-like (−1282 to −1177) region of the human ACTA1 gene (PMID:1633435).

Modification of Control Element (CE) Types in MSECs. Previous analysis indicates that enhancers and promoters from different muscle genes contain different and variable numbers of CEs that confer positive transcriptional activity to the regulatory fragment; and these CEs exhibit a wide variety of spatial locations with respect to adjacent CEs. This implies that the addition or substitution of virtually any muscle gene CEs and/or CEs with ubiquitous activities within many locations in native or miniaturized enhancer and promoter regions can modify transcriptional activities in beneficial fashions; e.g., insertion of additional MEF2 and E-box CEs within the CKM proximal promoter causes increased activity of the parental MSEC (see FIG. 9). These CE-mediated modifications can increase or decrease MSEC activities in all striated muscle types, can increase or decrease relative MSEC activities in selected muscle types, or can increase “leaky” expression in non-muscle cell types such that low product levels are achieved in these cells as well as much higher levels in striated muscle cells.

Hormone and Vitamin Responsive Control Elements (CEs). Additional types of CE modifications to MSEC enhancer and promoter regions can include insertion of DNA response elements for glucocorticoids and steroid hormones that function via DNA Glucocorticoid/Steroid-Response Elements (GREs/SREs) (PMID:32822588), for thyroid hormone that functions via DNA Thyroid Response Elements (TREs) (PMID: 1318069), and for Vitamin D that functions via DNA VDREs (PMID: 31203824). One therapeutic advantage of including CEs of these types in MSECs is their capacity to respond to external/pharmacological signals, thereby activating transcriptional activity of the MSEC when this would be beneficial.

Artificial Control Elements (CEs) for Muscle-Specific and Externally Regulatable Product Expression. Based on the feasibility of designing proteins containing DNA binding domains for any DNA sequence as well as domains that facilitate drug-mediated dimerization with partner proteins (PMID: 25989233, PMID: 19933107, PMID: 22753599), MSECs targeted by these proteins can be designed so as to contain one or more unique DNA binding sequences (i.e., sequences not present in the genome that will be uniquely bound by the designed protein), in addition to other essential CEs. MSECs of this type are then fused to any cDNA encoding the desired product. Transcription from MSECs of this type is then achieved in a muscle-specific fashion by co-expression of the unique DNA-binding protein as well as a second protein containing both a transcription activation domain and a dimerization domain that permits functional interaction with the DNA binding subunit only in the presence of a pharmacological agent. Since the artificial TF components are only made in muscle cells, and since dimerization of the two TF subunits only occurs in response to the drug, transcription of the product can be externally regulated by drug levels. An externally regulatable system of this general type has been designed and extensively tested by Ariad Pharmaceuticals (PMID: 22753599), but it was not formulated in a tightly regulated muscle-specific fashion. Parts of the Ariad system were adapted for the muscle-specific expression and secretion of parathyroid hormone in cell culture and in mice (Figures in: Salva, M. Z., 2007, PhD Thesis, University of Washington).

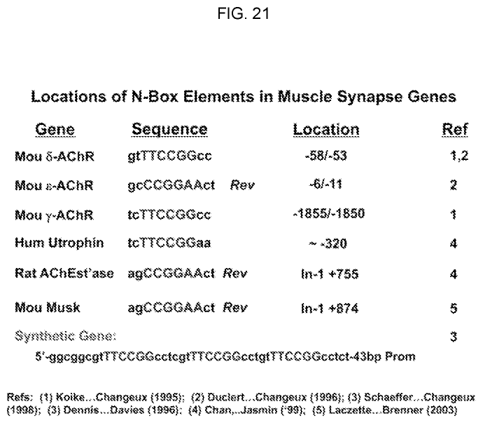

N-Box Control Elements (CEs) for Synapse-Specific Product Expression. N-box CEs occur in the promoters and enhancers of muscle genes that are transcribed at high levels in muscle fiber myonuclei located near neuromuscular synapses. N-boxes are thus a special category of MSEC CE insertions that can selectively enhance transcription in skeletal muscle fiber myonuclei within the synaptic region and repress MSEC expression in non-synaptic regions. These short CEs (e.g., multimers of -TTCCGG- as found in the enhancer and promoter regions of Acetylcholine esterase (ACHE) and Acetylycholine receptor subunits such as (CHRM1, CHRND, CHRNG), acetylcholine receptor genes), respond to nerve-mediated signals such as agrin and neuregulin that stimulate the productive binding of transcription factors such as GABP to N-Box CEs (PMID: 11498047; PMID: 7479853, and Refs listed in FIGs.). N-boxes are thus beneficial for localizing expression of therapeutic products to myonuclei in the subsynaptic region for the treatment of various myogenic neuromuscular junction diseases (PMID: 27112691), as well as neuromuscular junction-mediated secretion of neurotrophic factors that potentiate motor neuron survival, as in Amyotrophic Lateral Sclerosis (ALS) (PMID: 8860837). A particular MSEC design advantage of N-box CEs is that they are known to function both 5′- and 3-′ of the TSS and at a wide range of distances (FIG. 21), thereby facilitating their insertion in diverse locations that can enhance or prevent steric interactions of N-box TFs with other MSEC-associated TFs. This implies that N-boxes can function when inserted in many different MSEC locations, either as miniaturized N-box-containing enhancers isolated from muscle genes such as Acetylcholine esterase (ACHE) and Acetylycholine receptor subunits such as (CHRM1, CHRND, CHRNG), or as multimerized N-box clusters such as the 43-bp cluster of 3 N-boxes shown in FIG. 21. In vitro assays prove the function of such N-box-containing MSECs in differentiated skeletal muscle cells in response to the addition of specific ligands such as agrin and neuregulin to the culture media (PMID: 11498047; and Refs listed in fig). Optimal N-box locations within MSECs are thus determined by quantitative comparisons of expression levels of N-box-containing MSECs in response to externally added nerve-mediated factors (PMID: 11498047).

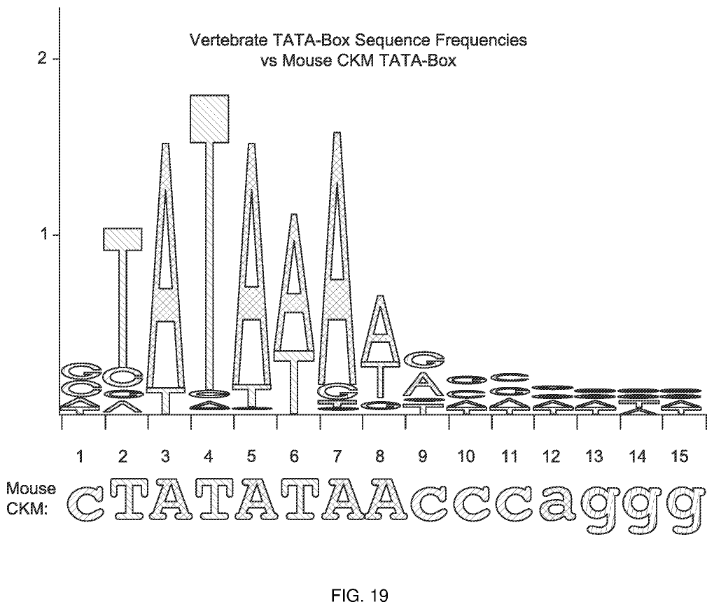

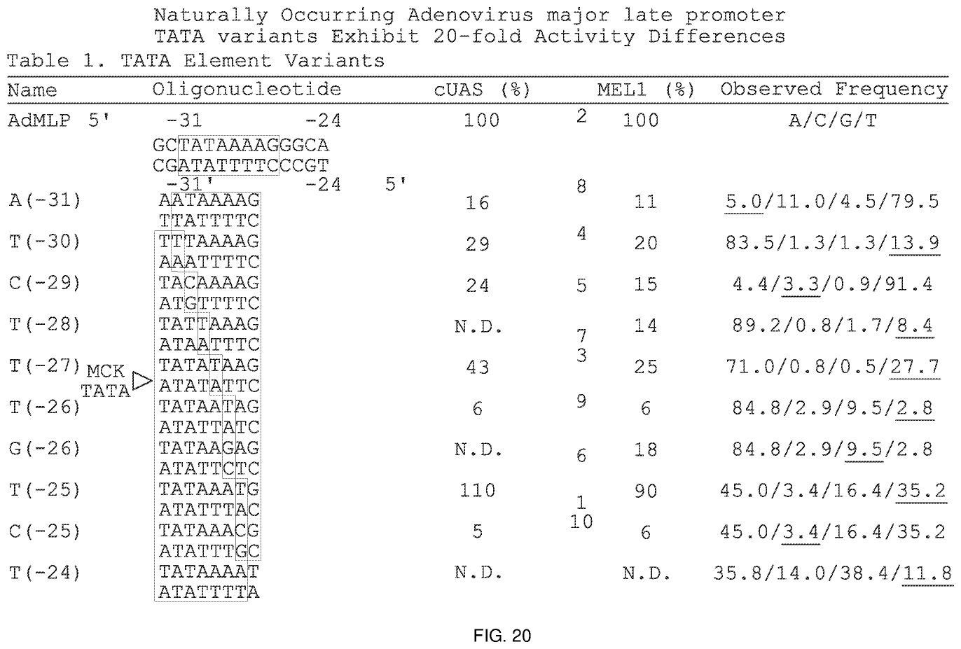

TATA-Box Modifications to Facilitate Graded MSEC Expression Levels. A particularly useful characteristic for MSECs would be enhancer/promoter designs that had near-identical signal transduction responses, but that expressed products at different levels in response to the same sets of environmental inputs. Since TATA-box DNA sequences are known to affect transcription rates (see FIGS. 19 and 20) (PMID: 10617571), identical MSECs containing TATA-boxes with different sequences will exhibit different transcription rates. This attribute not only provides MSECs that produce products over a range of concentrations, but the uniform signal response of such MSECs—because the common MSEC-set contains identical enhancer/promoter regions—facilitates evaluation of their safety parameters for gene therapy applications. The consensus vertebrate TATA-box core sequence is: gTATAAAA, and the TATA-box of the most highly transcribed human skeletal muscle gene (FIG. 6) ACTA1 is a perfect match to the vertebrate consensus TATA sequence (see MSEC-239 sequence); whereas the mouse CKM TATA-box sequence differs from the “consensus” vertebrate TATA sequence (see FIG. 19). These comparisons are consistent with the hypothesis that the TATA-box sequence is an important determinant of MSEC transcriptional activities, and that different TATA-box modifications of the same MSEC can produce MSECs with different transcriptional activities.

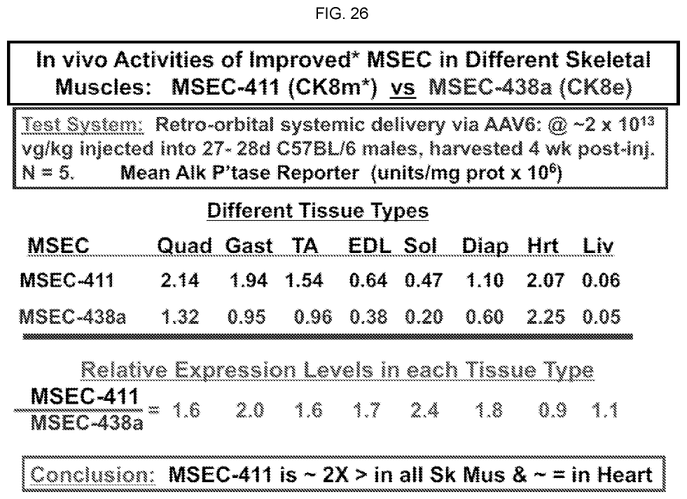

MSEC-Exon-1 Variations Affect MSEC-Mediated Product Levels. Many of the CKM-based MSECs described herein have 3′-termini at the +7 position relative to the +1 transcription start site (TSS). However, because the short Exon-1 sequence of CKM genes is highly conserved, and because both transcriptional and translational regulatory mechanisms are known to occur 3′ of the TSS, effects of extending MSEC sequences to the +50 mouse CKM genomic location were investigated. This provided a nearly 2-fold increase in MSEC-mediated product levels in skeletal muscle cultures and a 5-fold increase in cardiomyocyte cultures (PMID: 17235310). Later studies investigated how much of the (+1 to +50) region was required for maximal activity of MSEC-438a. Successive truncations of the sequence from its 3′-end disclosed that the (+1 to +12) CKM Exon-1 sequence was optimal and that it increased transcriptional activity of the otherwise identical MSEC-400 by about 2-fold in skeletal muscle cultures and also by about 2-fold in vivo following systemic delivery, while causing no change in heart muscle expression (see FIG. 26).

Human Versions of MSECs: Although at least two thirds of the sequence entries in the MSEC library contain partial and modified sequences from the mouse CKM gene, Human CKM gene sequence versions of several MSECs have been designed for comparison purposes, and as a general rule the transcriptional activities of these MSECs—when tested in rodent skeletal and cardiac muscle cultures—is roughly equivalent. This is anticipated because the sequences of mouse and human muscle gene CEs are very similar, thus facilitating the binding of human TFs to mouse CEs. Furthermore, since MSECs composed of mouse CKM sequences appear to be functioning well in human clinical trials, species differences between mouse-based MSEC sequences and the DNA-recognition capacities of human TFs appear to be minimal. Many other modified sequences in the MSEC library are derived from human genes. Sequences for cardiac-specific MSECs are derived from human TNNT2, sequences for enhancing expression in slow muscle fibers are derived from human TNNI1, and many of the minimal promoter sequences used in conjunction with synthetic MSEC enhancers are derived from human ACTA1. However, and regardless of the species origins of each MSEC component, it is important to emphasize that all of the MSECs are artificial in comparison to the entire DNA regulatory components of their genes of origin.

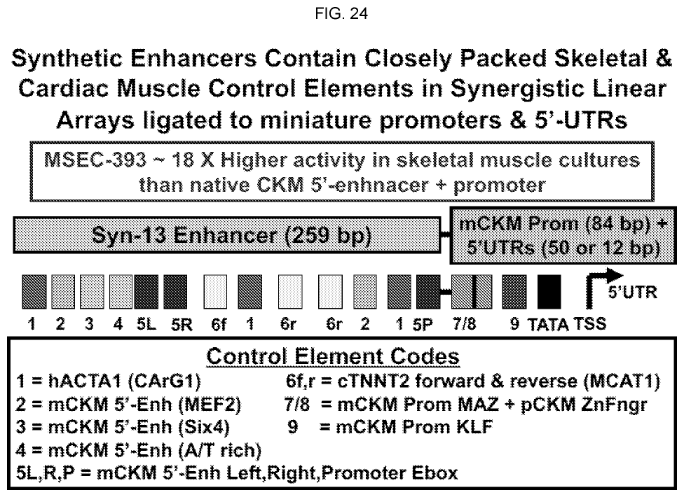

Synthetic MSECs. The numerous variations observed in CE types, numbers, and spacing observed in muscle gene enhancers and promoters, makes it possible to create rationally-designed Synthetic MSECs (see FIGS. 2 and 24). A particularly advantageous design advantage of synthetic MSECs over the stepwise modification of native MSECs, is that CEs whose TFs are known to interact (e.g., E-box and MEF2 TFs, TEF1 and MEF2 TFs, NKX2.5 and MEF2c TFs) as well as TFs whose dimerization is facilitated by adjacent DNA binding sites (e.g., TEF1-TEF1 interactions separated by 3-bp distances (PMID: 10975468), can be positioned adjacent to each other and appropriate distances apart to maximize protein-protein interactions. Synthetic MSECs can be composed of synthetic enhancers ligated to native or modified/miniaturized gene promoters (e.g., CKM, ACTA1 promoters: MSEC-503 and MSECs-285, -322, -361), native or modified/miniaturized enhancers ligated to synthetic promoters, or the enhancer and promoter regions can both be synthetic. The enhancer portions of synthetic MSECs can also be multimerized and placed 5′- and 3′- of the TSS.

Combinatorial Use of MSECs with cDNAs Containing miRNA Target Sequences. Because gene therapy strategies for treating most Neuromuscular and Cardiac diseases entail systemic delivery of gene delivery vectors, skeletal and cardiac muscle cells are both transduced. When the particular disease affects both muscle tissue types, treatments using a single MSEC with optimal expression characteristics in each muscle type is feasible. However, in situations in which only one muscle type is affected by the disease, and thus requires therapy, the unaffected muscle type can be affected deleteriously by production of the therapeutic product. This issue can be circumvented if MSECs with sufficient skeletal- or cardiac-specific expression characteristics exist; for example, MSEC-455a is highly cardiac muscle specific (see FIG. 16). However, if even very low-level expression of a therapeutic product in skeletal muscle, or cardiac muscle is toxic, this can preclude using an MSEC whose expression characteristics in the diseased tissue are otherwise beneficial. Although this issue can be partially addressed via improvements in tissue type-specific vector targeting, such improvements may not be sufficient to solve the problem of toxic effects due to very low level product expression in the inappropriate muscle type.

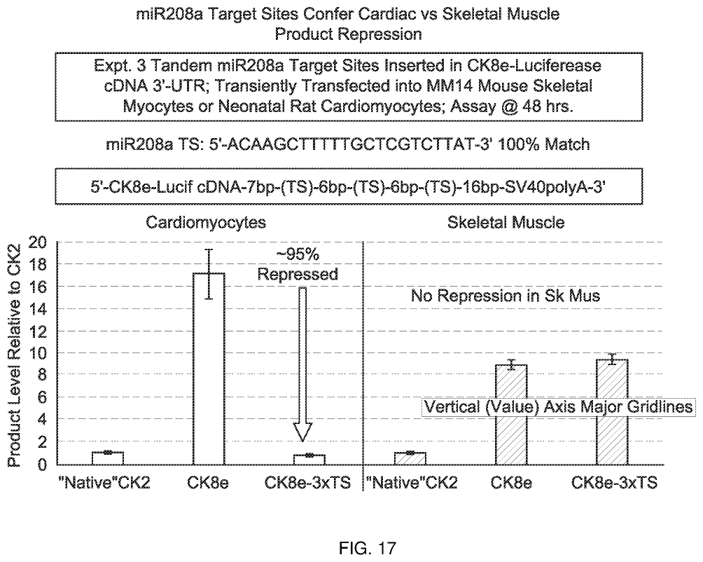

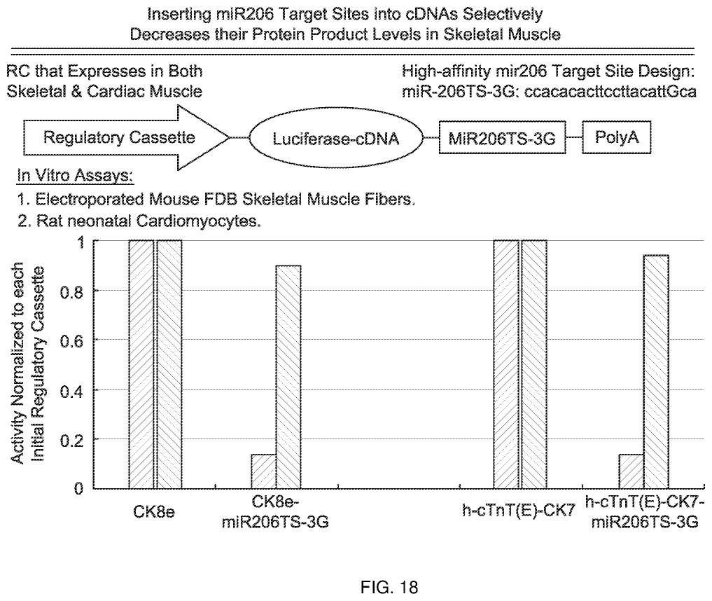

Therapeutic product toxicity problems of this type can be ameliorated by the combinatorial use of MSECs in conjunction with cDNAs in which muscle type-specific miRNA target sequences have been inserted, typically, but not necessarily in the cDNA 3′-region. Product mRNAs containing the miR target sequences are then preferentially degraded in muscle cell types that express miRs complementary to the target sites but not in muscle types that do not express the target-type miR. This process occurs naturally in skeletal muscle via it is expression of miR-206 (PMID: 25678853; PMID: 32620696), and in cardiac muscle via its expression of miR-208a (PMID: 25678853; PMID: 32620696; PMID: 19726871). This hypothesis was tested and verified in skeletal and cardiac muscle cultures by inserting multimerized miR-206 or mir-208a target sites (see miR target site sequences, FIG. 27, page-1) in the 3′end of reporter gene cDNAs. 90-95% reductions in product levels were achieved in a muscle type-specific manner (see FIGS. 17 and 18). Combinatorial use of muscle type-specific miR target sites with MSECs will thus work in vivo and be applicable to muscle type-specific therapies. With respect to utilizing miR-208a target sites to reduce potentially toxic product levels in cardiac muscle, it is of interest that various types of cardiac stress such as Ischemia and Diabetes increase heart muscle mir-208a levels (PMID: 30696455), thereby implying that mir-208a target site function will be enhanced in response to cardiac toxicity problems.

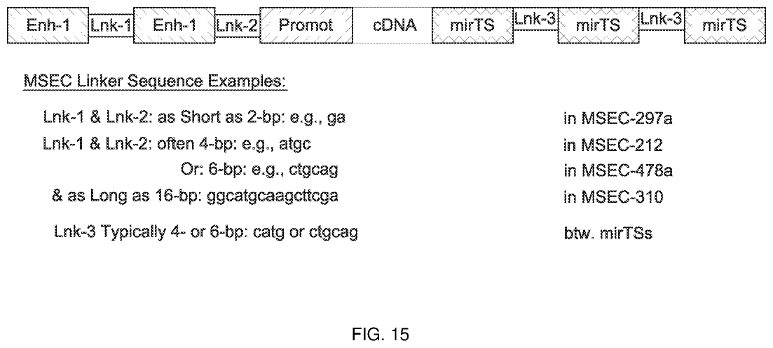

Linker Sequences are Transcriptionally Functional Components of MSECs. DNA linkers of varying sizes and sequences are included in many MSECs as inserted sequences between enhancer and promoter regions, between multiple enhancers, and between multimerized miRNA target sites. FIG. 15 shows examples of short and long linker sequences to illustrate the size range and sequence varieties that are present in different MSECs. Three fundamental aspects of linker inclusion within MSEC sequences are that: (1.) they introduce artificial sequences between the ligated components; (2.) they introduce different spatial distances between the ligated components; and (3.) these two factors can both affect each MSEC's transcriptional activity independently of the intrinsic activities of the MSEC's enhancer and promoter sequences. Linker sequences can thus be considered as functional components within each MSEC in relation to its degree of translational activity. This does not extend to selectivity of expression within muscle versus non-muscle cell types.

Overall Value of the Entire MSEC Library. The current MSEC library contains roughly 350 MSEC sequences that exhibit muscle-specific expression over a several thousand-fold range in muscle cultures. While MSECs with very low expression levels in muscle cultures have not been tested in vivo, the expectation is that an analogous 3-orders of magnitude product expression range would also be detected in vivo if all MSECs were tested under identical vector-dose protocols. Thus when very low in vivo expression levels are required MSECs are already available for that purpose. Furthermore, when in vivo dose-response evaluations of therapeutic products are necessary, the use of MSECs that exhibit an about 30-fold transcription activity range in skeletal and cardiac muscle provides much less ambiguous results than are obtained by the common practice of simply varying the vector dose over a 30-fold level. The reason for this is that the vector-dose protocol contains 2 variables: percent cells transduced plus the product level in each transduced cell; whereas the MSEC-dose protocol contains only 1 variable: product level in each transduced cell. Since safety issues preclude excessive vector doses, and since low vector doses result in many non-transduced cells, the availability of MSECs that will produce optimal product levels at vector doses that transduce sufficient cell numbers is critical for effective gene therapy development.

Methods to Select an MSEC for a Specific Use. Selecting optimal MSECs for treating different NMDs from among the MSECs currently available in the disclosed library entails a multistep process that will differ for each disease, and possibly among different alleles for each disease. This is due to the fact that each NMD affects a different protein, and the normal concentrations of these proteins may differ by 3 or more orders of magnitude; (e.g., some proteins such as -skeletal and -cardiac actin are present at much higher levels than others such as myosin and titin, and at even higher levels than those of regulatory proteins such as kinases and phosphatases). Moreover, since each mRNA and its encoded protein have different intrinsic half-lives, the amounts of mRNA required to produce normal amounts of each therapeutic protein will differ for each NMD.

A further complexity is the possibility that different disease gene alleles among different patients with the same NMD may produce non-functional or poorly functioning mutant proteins that compete with the therapeutic protein for binding to partner proteins, thereby necessitating higher levels of the therapeutic protein than found in normal muscle cells in order to “outcompete” the deleterious protein. An additional complexity is that until biomarker or functional improvement assays demonstrate efficacy in pilot studies that test graded vector doses or MSEC activity levels, it is challenging to predict how much therapeutic product is needed for optimal benefits. In this regard, it is important to recognize that while suboptimal therapeutic protein levels can be overcome via graded vector dose and MSEC activity increases, toxic levels will not necessarily be detected by the same biomarker or functional assays. Rather, toxicity may only be detected via assays that focus on predicted problems that could be logically associated with excess therapeutic product levels. Furthermore, these toxicity phenotypes may require extended time periods to be observed. Patient safety is thus at risk by assuming that high levels of a therapeutic protein will be inconsequential simply because functional benefits are observed.

Step-1. MSEC Selection Based on cDNA Size. Stepwise strategies for identifying optimal MSECs can begin with determining the size of the therapeutic product's cDNA, including the size of any 5′- and/or 3′-untranslated regions, as well as introns that may be necessary for obtaining high levels of the functional protein. This information, together with the packaging size limits of the vector can then be used to determine the size range of MSECs that can be efficiently packaged with the particular cDNA.

For AAV vectors that have a 4.8 kb efficient packaging limit, and that require the presence of two Inverted Terminal Repeat (ITR) sequences of about 145 bases for genomic packaging, the combined MSEC plus cDNA size limit is about 4.5 kb. Thus, for cDNAs smaller than 0.5, 1, 2, 3, and 4 kb, the compatible MSEC sizes would need to be less than 4, 3.5, 2.5, 1.5, and 0.5 kb. Since many MSECs have been purposely miniaturized so as to be compatible with packaging large cDNAs, this means that multiple MSECs are available for expressing almost all NMD proteins. Use of MSECs for expressing even larger proteins is not, however, precluded, because AAV vectors can package constructs as large as 5.2 kb with much lower efficiencies, and also because the cDNAs encoding larger proteins can be subdivided into 2 or more fragments whose encoded proteins will associate in the correct N-terminal to C-terminal order via appropriate intein technology (PMID: 32251274).

Step-2. MSEC Selection Based on Muscle Type Expression. The second step in identifying the most appropriate MSECs for a particular NMD therapy is knowledge concerning which muscle types require therapy; e.g., Skeletal and Cardiac muscle, Skeletal muscle only, Cardiac muscle only, as well as whether expression of the therapeutic product may be beneficial in one muscle type and toxic in others. Further narrowing of MSEC choices would come from knowledge regarding which human skeletal or cardiac muscles are most seriously affected by the disease (e.g., Duchenne Muscular Dystrophy affects essentially all striated muscles, while Limb Girdle Muscular Dystrophies affect only a subset of anatomical muscles, and Facioscapulohumeral Muscular Dystrophy affects a predominantly different muscle group). Similarly, some NMDs have greater relative effects among certain human skeletal muscle fiber types: I, IIa, and IIx.

Although no MSECs exhibit identical transcriptional activity in all striated muscles, many have several times higher expression in skeletal muscle than cardiac muscle or vice versa, and some exhibit 10 to several hundred times greater activity in one or the other muscle type. Except for skeletal versus cardiac muscle expression differences, no current MSECs yet exhibit expression differences greater than several fold between different anatomical skeletal muscles in mice, and this observation will probably be true for human anatomical muscles. However, some MSECs have different expression levels among different skeletal muscle fiber types (e.g. MSEC-438a produces 8:4:2:1 relative levels of reporter protein in mouse muscle fiber types IIb, IIx, IIa, and I respectively). As a consequence, human skeletal muscles containing primarily fast fibers (Type IIx and IIa) would be expected to contain higher therapeutic product levels than muscles containing primarily slow fibers (Type I) if they were transduced with AAV vectors containing an MSEC-438a-Therapeutic cDNA construct. Although MSEC-438a is currently the only MSEC for which fiber type-specific transcriptional activities have been measured, studies of this type are in progress with newly designed MSECs.

Step-3. MSEC Selection Based on Functional Product Concentration Levels. After narrowing MSEC choices based on the previous two criteria, it is necessary to estimate the concentration of therapeutic protein required to provide functional benefits for the particular NMD; and, if data is available, to know whether excessive levels of the therapeutic protein may be toxic. Unfortunately, conclusive data regarding the concentrations of many NMD proteins, the translational efficiency of their encoding mRNAs, and their steady-state half-lives in different human striated muscles and fiber types are not well established (Steed, et al., 2020. PMID: 32403418); and information pertaining to toxic effects of therapeutic proteins is also limited. Nevertheless, relative mRNA levels obtained from the gtexportal.org web site and other RNA seq studies can be useful starting points since these indicate at least a 5,000-fold difference between the most and least abundant muscle gene mRNAs; i.e., relative therapeutic mRNA levels in the thousands, hundreds, or tens of transcripts per million (TPMs) are likely to be at least somewhat proportional to each NMD protein's normal steady-state levels. Analogous “high, medium, and low” relative protein levels based on biochemical and immuno-microscopic data, as well as knowledge of each NMD protein's function are also worth considering, since this information can suggest whether to focus screening efforts on MSECs with “high, medium, or low” relative transcriptional activities. Information regarding each NMD mRNA's and protein's transport and diffusion parameters from transduced myonuclei are also important for selecting MSECs with transcriptional activity needed for the optimal treatment of each NMD.

Unless it is obvious that an NMD will require the highest possible therapeutic protein expression levels, the strategy of simply testing the most active MSEC at the highest permissible vector dose is not a sensible starting point. This may yield initial therapeutic responses that appear beneficial, but this positive outcome may mask better responses that would have been obtained at lower protein levels and it may well cause toxic effects that are not evident within initial short-term screening periods.

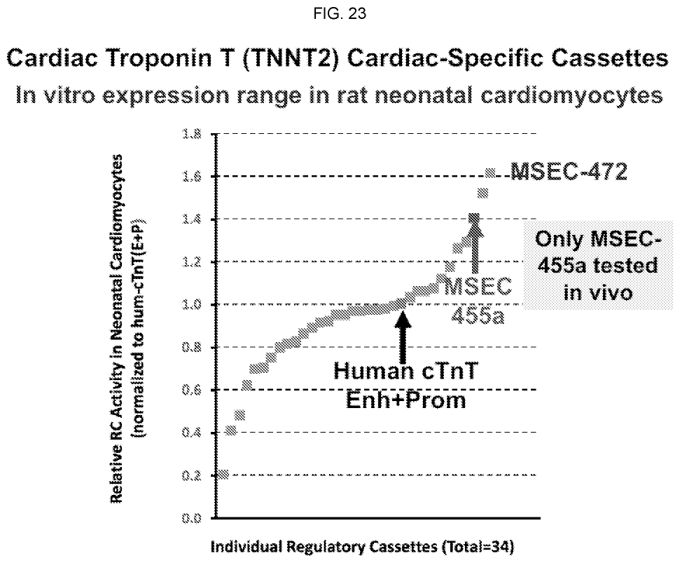

A more informative strategy for identifying MSECs with optimal transcriptional activities for each NMD is to bracket the “best estimate” of mRNA levels needed by testing MSECs that seem likely to provide 5-, 20-, 100- and 500-fold greater mRNA levels than the native NMD transcript levels when delivered at a safe mid-range vector dose (e.g., 7e13 vg/kg, as explained above). Selection of these MSECs can be performed based on the MSEC transcriptional activity tables in conjunction with the MSEC packaging size and muscle-type expression level criteria relative to that of the native M-creatine kinase enhancer-promoter MSEC (transcriptional activity designated as 1.0 in skeletal and cardiac muscle cell culture studies (see FIGS. 5, 7, and 23).

In practice, since mRNA transcripts from the native CKM gene in adult human skeletal muscle are 25,000 TPM, initial screening choices for NMDs whose mRNAs are in the range of 50, 500, and 5,000 TPM, would then be MSECs whose 5×, 20×, 100×, and 500× transcriptional activities relative to the native CKM enhancer-promoter activities would be: (0.01, 0.04, 0.2, 1), (0.1, 0.4, 2.0, 10), and (1, 4, 20, highest available), respectively. Careful data analyses from these pilot studies should then identify the approximate MSEC expression level range most likely to be beneficial, and possibly free of toxicity. Subsequent screening rounds would then test MSECs with transcriptional activities between those of the “best” initial MSECs.

### Exemplary Embodiments—Set 1

1. In some embodiments, the MSEC is based on the (−3,356 to +7) sequence of the native mouse muscle creatine kinase (CKM) gene (MSEC-3363), as well as smaller genomic segments located within this region. Examples of these are (−1,256 to +7): MSEC-1263a that contains the mouse CKM gene 5′-enhancer region located within the (−1256 to −1051) region, as well as a variety of enhancerless mouse CKM proximal promoters: (−1050 to +7): MSEC-1057; (−1020 to +7): MSEC-1027; (−776 to +7): MSEC-783; (−723 to +7): MSEC-730; (−358 to +7): MSEC-365; as well as a basal mouse CKM promoter: (−80 to +7): MSEC-87 (PMID: 3990682; PMID: 3785216; PMID: 3336366). These and other analogous mouse CKM enhancerless proximal promoters exhibit varying low levels of muscle-specific expression when they are used as stand-alone MSECs, whereas the basal promoter exhibits only background level expression. Nevertheless, the basal promoters such as MSEC-87 and others that are somewhat larger are useful MSEC components because of their small size, and because they function well with CKM and other muscle gene promoters and enhancers that convey varying levels of muscle-specific transcriptional activity.

2. In some embodiments, all of the enhancerless mouse CKM promoters listed in Embodiment-1 as well as any other promoters fashioned from sequences within the (−1050 to +7) region can be ligated to a mouse CKM 5′-enhancer in either its native (e.g., MSEC-584 and MSEC-297a) or modified forms (e.g., MSEC-297b through MSEC-297k) (PMID: 3336366; PMID: 8474439).

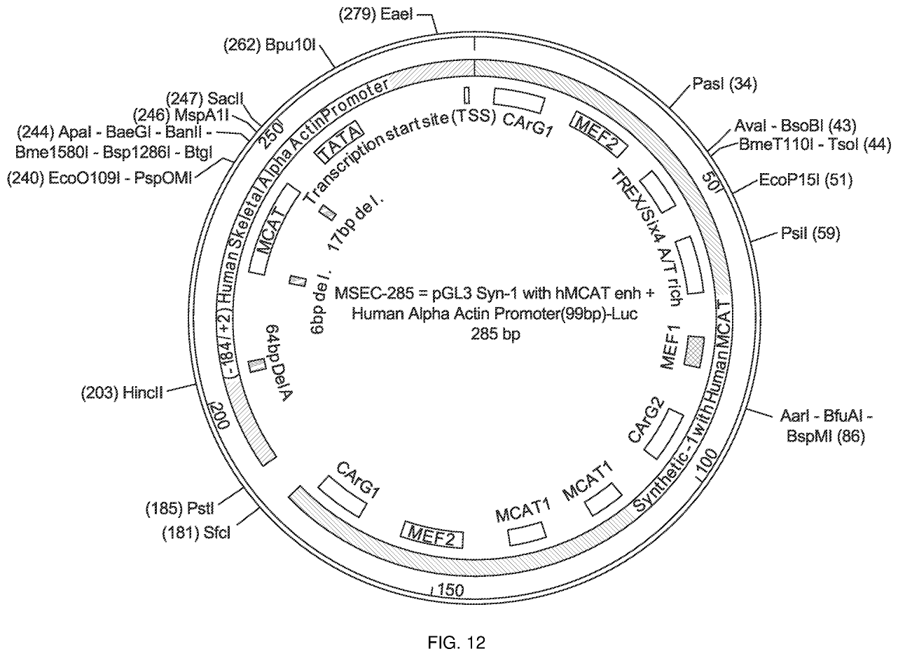

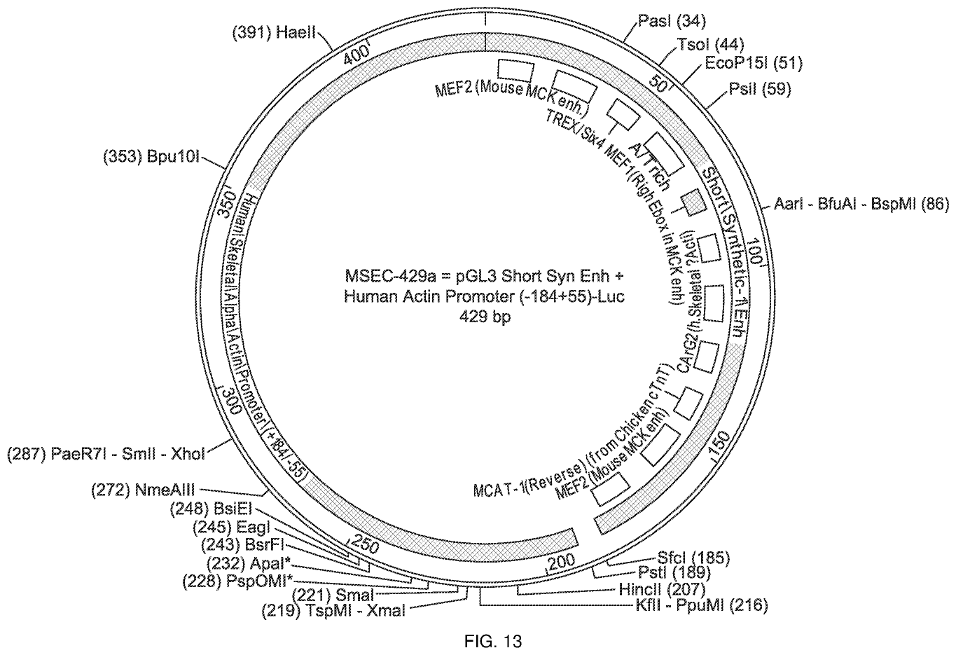

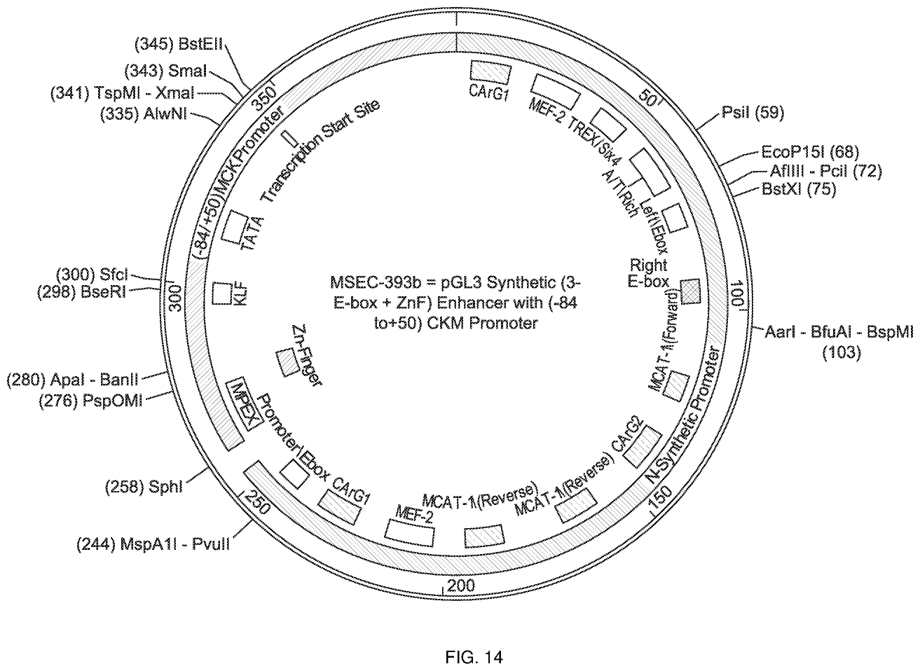

It is important to note that the majority of MSECs described in this disclosure contain short non-genomic linker DNA segments of varying lengths between enhancers and promoters due to the molecular biology technology of ligating the two components together (see FIG. 15 and FIGS. 12-14). For this reason, the MSEC bp lengths provided in the FIG. 27 sequences do not necessarily correspond to the exact predicted bp length of the components that are ligated together. For example, MSEC-297a in Embodiment-2 is 297 bp long, whereas its “predicted length” based on the ligation of a 206-bp 5′-enhancer segment to the 87-bp basal promoter segment would be only 293 bp. The seeming discrepancy is due to the 2-bp linker sequence “GA” inserted between the enhancer and promoter in order to ligate the two regulatory regions (PMID: 8474439). Importantly, since the linker sequence can be considered an integral part of the MSEC's overall sequence, it should not be assumed that the linker sequences in each MSEC are transcriptionally “neutral;” they could have either positive, negative, or no effects on the MSEC's transcriptional activity. These effects would be due to either the specific linker base pairs having either intrinsic transcriptional activities by themselves or in association with contiguous sequences 5- and/or 3′ of the linker sequence, or due to creating a more or less efficient association between transcription factors bound to sequences 5- and 3′ of the linker. Linker sequences could be removed to make slightly smaller MSECs via the simple expedient of synthesizing the multi-component MSECs denovo based on an entire sequence that lacks the linkers. However, these versions would not necessarily exhibit identical transcriptional activities to the analogous linker-containing MSECs. Linker sequences are underlined in some, but not all, MSECs in FIG. 27.

An additional bp-length aspect of the MSEC descriptions is that many MSECs were assembled from genomic fragments obtained from different restriction enzyme digests. Thus, depending on the enzymes used, slightly different fragment lengths were obtained; e.g. proximal promoter fragments: [(−1020 to +7), MSEC-1030; (−776 to +7;) MSEC-783; and (−723 to +7), MSEC-730]; and basal promoters [(−117 to +1) MSEC-118; and (−80 to +7) MSEC-87]. Many of the proximal promoters were subsequently miniaturized a 318 bp size spanning the region mouse CKM region (−268 to +50), or 280 bp size (−268 to +12), and these are used in many of the MSECs described below.

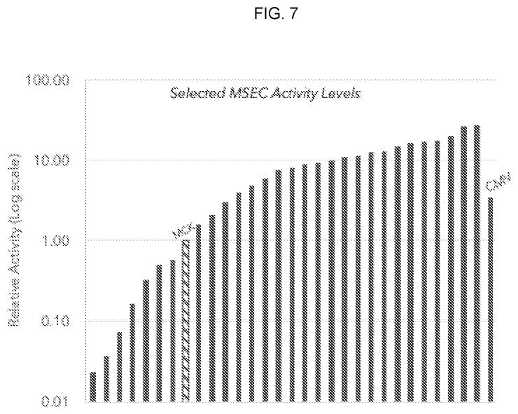

3. In some embodiments, the enhancer component described in Embodiment-2 may have one or more of its CEs mutated or deleted. The individual CKM enhancer and promoter CEs are depicted in the FIGS. 3, 8, and 9, Depending on the CE that is mutated, as well as on the resulting mutated sequence, this typically leads to MSECs with lower transcriptional activities ranging from about 1% to 95% of the native MSEC's activity (e.g., FIG. 7 and Tables and Figs in PMIDs 8474439 and 876664); however, some mutations such as those of the 5′-enhancer AP2 CE, may cause elevated activity. (PMID: 8474439). MSECs with these ranges of lower as well as higher activities are potentially useful for expressing lower as well as higher product levels when this is needed for particular applications (FIG. 7).

4. In some embodiments of the types described in Embodiments-2 and 3, the CKM 5′-native or modified enhancer can be ligated in reverse orientation relative to the MSEC's TSS (see many examples in PMID: 3336366 as well as in PMID: 8474439. e.g., MSEC-310). Depending on the enhancer and its modifications, this manipulation may increase or decrease the MSEC's transcriptional activity by different relative amounts in skeletal vs cardiac muscle cultures, without necessarily changing the overall MSEC's size (PMID: 8474439). Analogous activity changes would be anticipated for such MSECs in vivo.

5. In some embodiments of the types described in Embodiments 2, 3 and 4, single copies of the CKM 5′-native or modified enhancer can be ligated at the 3′-end of the cDNA in either its forward or reverse orientation relative to the MSEC's TSS (PMID: 3336366, and FIG. 2 in (PMID: 8474439). Depending on the enhancer and its modifications, this manipulation may increase or decrease the MSEC's transcriptional activity (PMID: 8474439). Based on Embodiment-6 plus the data in (PMID: 8474439), placing single enhancers in both 5′- and 3′-locations is also anticipated to increase activity.

6. In some embodiments of the types described in Embodiments-2, 3 and 4 the CKM 5′-native or modified enhancer can be multimerized so that the MSEC contains 2, 3, 4 or more tandem enhancers located 5′ of the promoter region e.g., MSEC-507; MSEC 746; MSEC-749). Furthermore, within each group of multimerized enhancers, their orientation relative to the TSS can be in either forward or reversed orientations (see FIG. 27 sequences). Depending on the enhancer and its modifications, these manipulations typically increase the MSECs transcriptional activity several fold. Based on Embodiment-5 and the data in (PMID: 8474439), placing multimerized enhancers 3′ of the cDNA, or in both 5′- and 3′-locations is also anticipated to increase activity.

7. Based on Embodiments-5 and 6, and the data in (PMID: 8474439), placing multimerized enhancers in both 5′- and 3′-locations, as well as in either forward or reverse orientations within each contiguous group of enhancers is also anticipated to increase activity.

8. In some embodiments, analogous regions of the human as well as other vertebrate CKM proximal and basal promoters (Embodiment-1) can be designed and used in the same ways as in Embodiments-2, 3, 4, 5 and 6 as a means of providing CEs whose DNA binding sites may provide improved affinity for transcription factors from the same species (e.g., MSEC-463; MSEC-464a; MSEC-463b).

9. In some embodiments of the types described in Embodiments-2 through 8, any CKM 5′-native or modified enhancer can be ligated to a heterologous promoter, (e.g., Thymidine Kinase and SV40 promoters) in either its forward or reverse orientation relative to the MSEC's TSS (see multiple examples in PMID: 33363666), and FIG. 7 in (PMID: 8474439). Depending on the enhancer, the heterologous promoter, and its modifications, this manipulation may increase or decrease the MSEC's transcriptional activity (PMID: 8474439). However, combining CKM enhancers with heterologous promoters may also decrease the MSEC's muscle specificity such that the combinations have higher relative activities in non-muscle cells.

10. In some embodiments, the mouse CKM intron-1 (3′-enhancer, also called Modulatory Region 1/MR1) located in the (+740 to +1,721) genomic region of the mouse CKM gene, (see FIG. 3), and FIG. 2 in PMID: 21797989 is used together with a CKM proximal promoter segment such as the (−358 to +7) region (e.g., MSEC-365). This large MSEC [(+740 to +1,721)+(−358 to +7) exhibits in vitro activity in differentiated skeletal muscle cultures and also exhibits high transcriptional activity in vivo in a transgenic mouse context (FIG. 5 in PMID: 21797989).

11a. In some embodiments, the mouse CKM intron-1 MR1 enhancer region is miniaturized to a 95-bp Small Intronic Enhancer (SIE) sequence located in the (+904 to +998) region of the native mouse CKM gene, (see FIG. 3), and FIG. 2 in PMID: 21797989. When the SIE (MSEC-95) is used together with a CKM proximal promoter segment such as the (−358 to +7) region (e.g., MSEC-365) the resulting MSEC-476b exhibits high in vitro activity in skeletal muscle cultures that is equivalent to that of the CKM 5′-enhancer (FIG. 2 in PMID: 21797989.

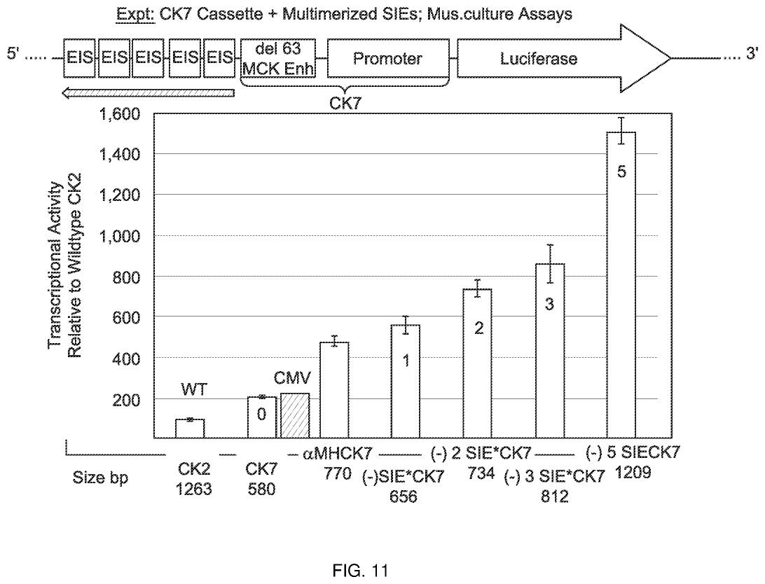

11b. The miniaturized SIE can also be multimerized and combined with various other MSEC promoters to provide further incremental increases in activity (see FIG. 5, “blue data entries”, FIG. 11, and sequences for MSECs-681, 804 and 805.

12. In some embodiments, enhancer and/or promoters from vertebrate muscle genes other than CKM can be designed and used in the same ways as in Embodiments-2 through 8. For example, many MSECs have been constructed from enhancer and promoter regions of the human cardiac-Troponin T gene (TNNT2), (see FIG. 10 and MSEC-495). These TNNT2-based MSECs are particularly useful for any purposes in which cardiac muscle-specific gene expression is desirable, and skeletal muscle expression is undesirable; and MSECs-320, -455a, and -590 that contain 1, 2 and 3 copies of the miniaturized TNNT2 enhancer, together with a miniaturized TNNT2 promoter, can be used to obtain increasing levels of cardiac muscle-specific expression.

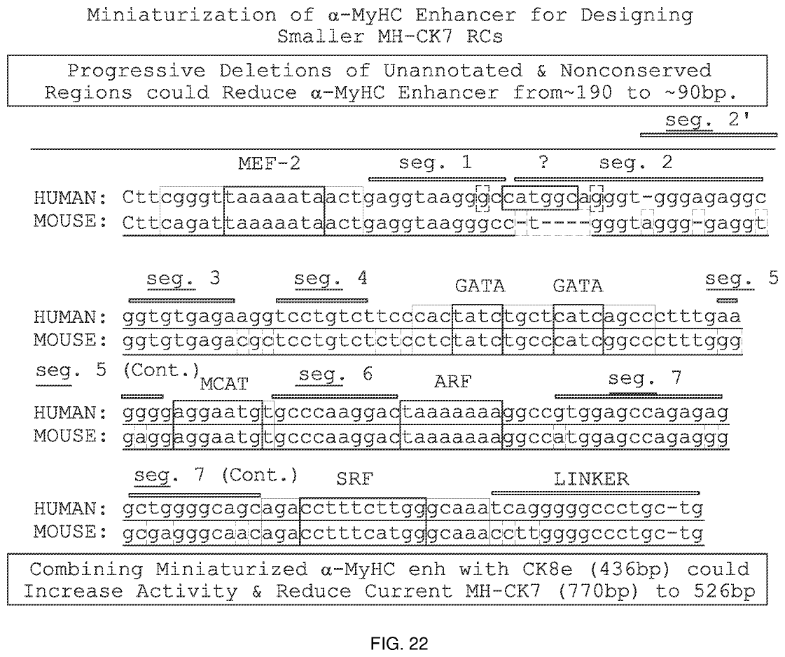

13. In some embodiments, enhancer and/or promoters from different vertebrate muscle genes can be combined in the same MSEC. This was first done in an MSEC called MH-CK7 (MSEC-770) in which the human alpha-Myosin heavy chain enhancer was combined with modified versions of the mouse CKM 5′-enhancer and proximal promoter (PMID: 17235310). This created an MSEC with higher transcriptional activity in both skeletal and cardiac muscle. The alpha_Myosin heavy chain and CK7 portions original MH-CK7/MSEC-770 construct can also be further miniaturized to create smaller versions with similar activities; e.g., MSECs-745, 720, 714, 697, 689, and 672 (see FIG. 22). Other combinatorial MSECs have combined enhancers from the Slow and Fast skeletal muscle Troponin-I (TNNI1 and TNNI2) genes (PMID: 8900050), with enhancers and promoters from the mouse CKM gene; e.g., MSECs-481, 518, 541a, 550, 555, 563, 569, 587, 726, 757, 758, 768, 773, 778, 855, 960.

14. In some embodiments the (−1,256 to +7) region of the native mouse CKM gene has been mutated in one or more of its conserved E-boxes. Depending on the mutation and the test system, this differentially decreases the MSEC's relative expression in cardiac muscle by as much as 1000-fold in transgenic mice, and to a lesser degree in skeletal muscles containing relatively more type-I muscle fibers, while having little effect on muscles with primarily fast fibers (PMID: 8756664; 12968024). MSECs such as MSEC-1263f, as well as analogous MSEC constructs that could be made by making similar E-box mutations or deletions within the 5′-enhancer regions of any other MSECs containing these control elements. MSECs of these types could be useful for treating diseases such as Limb Girdle Muscular Dystrophy 2A, X-Linked Myotubularian, Nemalin myopathies and other NMDs in which skeletal muscle expression of the therapeutic product is desirable, while cardiac muscle expression may be toxic.

15. In some embodiments the CArG/SRF control element within the 5′-enhancer region of MSEC-1263a has been mutated. This differentially decreases the resulting MSEC's relative expression in cardiac muscle by about 1000-fold relative to skeletal muscles in transgenic mice (PMID: 8657140). Analogous MSEC constructs could be made by making similar CArG/SRF mutations or deletions within the 5′-enhancer regions of any other MSECs containing such CEs, e.g., CArG/SRF mutations in the CKM 206 bp enhancer almost totally abolish in vitro activity in cardiomyocytes while having much lesser effects in skeletal muscle (PMID: 8474439). MSECs of these types could be useful for developing treatments for diseases such as Limb Girdle Muscular Dystrophy 2A, X-Linked Myotubularian, Nemalin myopathies and other NMDs in which skeletal muscle expression of the therapeutic product is desirable, while cardiac muscle expression may be toxic.

16. In some embodiments the AT-rich control element within the 5′-enhancer region of MSEC-1263a has been mutated. This differentially decreases the resulting MSEC's relative expression in cardiac muscle by about 10-fold relative to skeletal muscles (PMID: 8657140). As described in Embodiment “A” above, (some exemplary MSECs are provided in the Sequence List), as well as Analogous MSEC constructs could be made by making similar CArG/SRF CE mutations within the 5′-enhancer regions of any other MSECs containing such CEs, and could be potentially useful for developing treatments for diseases such as Limb Girdle Muscular Dystrophy 2A, X-Linked Myotubularian, Nemalin myopathies and other NMDs in which cardiac toxicity is an issue.

17. In some embodiments the Six4 control element within the 5′-enhancer region of MSEC-1263a has been mutated. This differentially decreases the resulting MSEC's relative expression in cardiac muscle by about 20-fold relative to skeletal muscles (PMID: 12779122). As described in Embodiment “A” above, (some exemplary MSECs are provided in the Sequence Listing), as well as Analogous MSEC constructs could be made by making similar Six4 CE mutations within the 5′-enhancer regions of any other MSECs containing such CEs, and could be potentially useful for developing treatments for diseases such as Limb Girdle Muscular Dystrophy 2A, X-Linked Myotubularian, Nemalin myopathies and other NMDs in which cardiac toxicity is an issue.

18. In some embodiments, the MSEC is miniaturized by decreasing the number of intervening bp between functional control elements. Modifications of this type are illustrated in FIGS. 8 and 9. In some cases these MSEC design changes have the additional advantage of facilitating protein-protein interactions between the transcription factors bound to adjacent control elements and thereby increasing an MSEC's transcriptional activity. An example of this MSEC design attribute is illustrated by deleting most of the intervening bp between the CKM 5′-enhancer Right E-Box and 5′-MEF2 control element to create the CK7 regulatory cassette (MSEC-580). Miniaturization can also permit the insertion of additional control elements within native enhancers and promoters that increase MSEC activities, as illustrated by the addition of adjacent MEF2 and E-Box elements in the miniaturized CK9 promoter (FIG. 9) Additional MSEC miniaturizations can be achieved by deleting control elements such as those in Embodiments 15 through 18 whose activities are relatively more important for producing therapeutic products in cardiac than in skeletal muscle. As long as skeletal muscle expression levels remain high enough to achieve beneficial outcomes, this strategy has the advantage of creating smaller MSECs that can be used in conjunction with therapeutic cDNAs whose sizes are incompatible with efficient AAV packaging; e.g., natural cDNAs or cDNAs encoding Cas9 plus any additional functional domains, whose sizes are too large for efficient AAV packaging.

19. In some embodiments, the MSEC is modified by addition of the first 50 bp of the mouse CKM Exon-1 region (+1 to +50) (see FIGS. 3 and 9), and FIG. 1 in PMID: 17235310) so as to increase transcriptional activity above that of MSECs containing only the (+1 to +7) sequence of CKM Exon-1. However, subsequent studies of the (+1 to +50) Exon-1 region found that inclusion of only the (+1 to +12) Exon-1 region is optimal.

20. In some embodiments the MSEC is modified by removing one or more of the less active CKM enhancer(s) or promoter control elements so as to further miniaturize the MSECs size without sacrificing necessary transcriptional activity. Examples of this are deletion of the CKM 5′-enhancer AP2 control element. Interestingly, this strategy is also likely to increase overall activity of the CKM enhancer activity (see PMID: 8474439, FIG. 5). Additionally, when the AP2 and CArG/SRF control elements, plus flanking and intervening sequences, are deleted, and the Exon-1 region (Embodiment-19) is reduced to the (+1 to +12) overall size of the already miniaturized CK8e MSEC-438 to ˜ 340 bp.

21. In some embodiments the MSEC is modified by removing one or more of the less active CKM enhancer(s)' or promoter's control elements so as to further miniaturize the MSECs size without sacrificing necessary transcriptional activity, and then inserting the sequence of a more active CE e.g, insertion of a MEF2 CE (FIG. 9), MSECs-336a, 336b, 336c.

22. [Reserved]

23. In some embodiments, the MSEC enhancer and/or promoter regions contain totally novel arrangements of control elements. These “Synthetic” MSECs have the important attributes of both high transcriptional activities and small sizes, and they can also be used in combination with miniaturized enhancers and promoters from multiple muscle genes (see FIGS. 12, 13, 14), and MSECs-259, 285, 319b, 322, 341, 361, 367, 383, 386, 388, 339a,b, 405a, 425, 474, 529a, 531a,b, 586, 562, 812a,b), as well as with promoters from non-muscle genes; e.g., MSECs using the Thymidine Kinase promoter FIG. 7 in PMID: 8474439).

24. In some embodiments MSECs can contain inserted N-box control elements so as to enhance transcriptional activity in myonuclei located in the muscle nerve synapse region, while decreasing transcriptional activity in non-synaptic regions (see FIG. 21). These MSECs would contain 1 to 5 enhancer-like 43-bp clusters of N-Box consensus sequences ligated 5′- and/or 3′- of permissive muscle gene promoters such as MSEC-118 or MSEC-239, and would be useful for treating a subset of NMDs in which the defect is due to genes that are selectively expressed within myonuclei localized in the neuromuscular junction region; examples of these are: Acetylcholine receptor subunits (multiple CHRN genes), Acetylcholinesterase (ACHE), Agrin (AGRN), Muscle-Associated receptor tyrosine kinase (MUSK), Collagen-Like Tail subunit (COLQ), Utrophin (UTRN). Localized expression of essential trophic factors for nerves such as BDNF could also be beneficial for treating Spinal Bulbar Muscular Atrophy (SBMA) (PMID: 308775922).

25. In some embodiments MSECs can contain TATA-boxes with sequences that differ from the native gene's TATA-box for the purposes of changing MSEC transcriptional activities without modifying the MSEC's reception of transcriptional signals impinging on all other enhancer and promoter regulatory elements (see FIGS. 19 and 20). TATA-Box sequences conveying a 20- or greater-fold range of transcriptional activities (e.g., MSEC-438a and MSEC-455a with either cTATAAAAg or cTATAAAGg TATA-Boxes will have the therapeutic advantage of producing quantitatively different levels of therapeutic products within qualitatively identical muscle types.

26. In some embodiments MSECs can contain TATA-boxes whose position relative to the TSS is changed so as to modify the efficiency of transcription initiation and thereby modify the MSEC's overall transcriptional activity and product production levels without modifying the MSEC's reception of transcriptional signals impinging on all other enhancer and promoter regulatory elements; e.g., variants of MSEC-438a and MSEC-455a containing 27-34 bp distances between the 5′T in the TATA-Box sequence and the TSS will conveying a 50- or greater-fold range of transcriptional activities (PMID: 16916456). As in Embodiment-25 these MSECs will have the therapeutic advantage of producing quantitatively different levels of therapeutic products within qualitatively identical muscle types. Furthermore, MSECs containing both TATA-box AND distance variations Embodiments 25 and 26) will exhibit even greater quantitative differences.

27. In some embodiments, MSECs can be used in conjunction with miRNA target sites that are ligated 3′ of whatever cDNA component is to be expressed as an RNA or protein product. When constructs of this type are expressed in vitro or in vivo in cell types in which the appropriate miRNAs are present, these bind to their target sites within the transcribed RNA and cause its degradation, thereby reducing product levels in that particular cell type by as much as 20-fold. In contrast, RNA transcripts produced in cell types that do not express miRNAs capable of binding to the miRNA target sites are not degraded. This strategy facilitates the expression of high in vivo product levels in skeletal muscle, and greatly reduced product levels in cardiac muscle (because, cardiac muscle contains miR208a (PMID: 34957257); in contrast, this strategy facilitates the expression of high in vivo product levels in cardiac muscle, and greatly reduced product levels in skeletal muscle because skeletal muscle contains miR206 (PMID:23439498). The use of this strategy in conjunction with MSEC-438a together with 3 tandem miRNA208a target sites yields high product levels in skeletal muscle and repressed levels in cardiac muscle (illustrated in FIG. 17); and its use in conjunction with MSEC-438a or MSEC-455 together with 3 tandem miRNA206 target sites (in this case slightly modified to provide greater specificity) yields high product levels in cardiac muscle and repressed levels in skeletal muscle (illustrated in FIG. 18). Importantly, these two differential targeting strategies can be used with ANY MSEC simply by ligating the appropriate tandem arrays of mir208a or miR206 target sites to whatever cDNA encodes a therapeutic product that would be beneficial to express in one striated muscle type and not the other.

28. In relation to all of the embodiments above, it is important to emphasize that an overall attribute of the entire MSEC library is the greater-than-3-orders-of-magnitude-expression-level-range between the least and most active MSECs (FIG. 7). Subject to packaging size constraints that could restrict which MSECs can be used in conjunction with a particular vector this means that one or more MSECs in the Library would probably be suitable for almost any use for which a specific product level was required in skeletal and/or cardiac muscle tissue. This attribute of the entire MSEC Library is important because the natural mRNA expression levels of genes whose mutations cause NMDs spans more than 3 orders of magnitude. The library thus contains MSECs whose activities will likely match those required for obtaining near normal levels of each NMD therapeutic product.

29. In relation to all of the embodiments above, it is likewise important to emphasize that an overall attribute of the entire MSEC library is that transcriptional activity can be made more or less responsive to signal transduction pathways that impinge on the transcription factors that bind to one or more control elements within each MSEC. As but one example, increasing the number of MEF2 CEs within an MSEC would be anticipated to increase an MSEC's responsiveness to external signal transductions that function via the MEF2 signal transduction pathways. As more information related to how various skeletal and cardiac muscle signal transduction pathways impinge on specific transcription factors, it should thus be feasible to modify different MSECs or to construct synthetic MSECs to be more or less responsive to external signals.

30. A general embodiment of all of the MSECs described above is that each of them can be used to produce any protein, RNA product, metabolite, or synthetic product derived from the expression of any enzyme or synthetic protein. These products can either remain within the differentiated muscle fibers or can be engineered with secretory signals so that they are secreted and have access to the extracellular matrix region and/or the circulatory system. Evidence for this is based upon the fact that to date more than 30 different cDNAs have been ligated to different MSECs and these have encoded and produced: full-length mouse dystrophin, Dp260-dystrophin, mini-dystrophin, and more than 10 different micro-dystrophins, and micro-utrophins, hormones and cytokines such as human parathyroid hormone, human growth hormone, human placental alkaline phosphatase, mouse Inerleukin-10, clotting factors such as Factor IX, human TDP43, and other proteins that remain and function within the muscle fiber such as mouse Ribonucleotide Reductase subunit-1, mouse Ribonucleotide Reductase subunit 2, mouse alpha-Gluocosidase, mouse Glycogen Synthase, and human liver arginase. The latter is of particular interest because it has the capacity of using skeletal muscle to carry out biochemical functions that typically occur in the liver. MSECs have also been used to express a wide variety of bacterial and invertebrate proteins such as: Cas9, Chloramphenicol Acetyl Transferase, Beta-galactosidase, Luciferase, Renilla, Green Fluorescent protein, M-cherry fluorescent protein, and mTmG-2a-puromycin-resistance fusion protein. Importantly, no cDNA ever ligated to an MSEC has failed to exhibit muscle-specific expression of the appropriate product.

### Exemplary Embodiments—Set 2

In some embodiments the MSEC is based on smaller segments of the sequence of the native mouse muscle creatine kinase (CKM) gene located within the (−3,300 to +7) region relative to the gene's Transcription Start Site (TSS), as well as within the (+740 to +1721) intron-1 region within mouse CKM.

In some embodiments the MSEC is based on a 1,260 bp 5′-portion of the (−1,256 to +7) region of the native mouse CKM gene that contains both a 5′-enhancer and “proximal” promoter region.

In some embodiments the MSEC internal portions of the (−1256 to +7) region of the native mouse CKM gene are removed to bring the 5′-enhancer closer to the “proximal” promoter region.

In some embodiments the 206 bp 5′-enhancer region (−1256 to −1050) is ligated directly to proximal promoter regions of different lengths; e.g., (776 to +7), (356 to +7), (an 220 bp portion of the (356 to +7) promoter following removal of many internal sequences), (−117 to +7), and (−80 to +7).

In some embodiments the MSEC's 5′-enhancer region is placed in a reversed orientation relative to a promoter fragment.

In some embodiments the MSEC's 5′-enhancer region is placed at different linear distances from the promoter.

In some embodiments the native or modified creatine kinase gene Intron-1 enhancer is added to MSECs.

In some embodiments the MSEC is used in conjunction with a modified array of miRNA target sites to repress expression in selected muscle or other tissue types.

In some embodiments the MSEC is modified to change the number of nucleotides between functional CEs.

In some embodiments the MSEC is modified with an insertion of additional CEs.

In some embodiments the MSEC is modified by removing one or more CEs.

In some embodiments the MSEC is modified by first deleting the sequence of a specific CE and then inserting the sequence of a different CE.

In some embodiments the MSEC is modified with the rearrangement of CE linear orders within enhancer and promoter regions of the MSEC.

In some embodiments the MSEC 3′-promoter region is ligated to different portions of CKM Exon-1 to provide MSECs with different expression levels in skeletal and cardiac muscles.

In some embodiments MSECs with modified enhancers or promoters derived from one or more muscle genes also contain one or more CEs that have been identified in the enhancers or promoters of other muscle genes.

For example, in some embodiments MSECs contain inserted N-box CEs so as to enhance transcriptional activity in myonuclei located in the muscle nerve synapse region, while decreasing transcriptional activity in non-synaptic regions.

In some embodiments MSECs contain TATA-boxes with sequences that differ from the native gene's TATA-box for the purposes of changing MSEC transcriptional activities without modifying the MSEC's reception of transcriptional signals impinging on all other regulatory components.

In some embodiments MSECs contain TATA-boxes whose position relative to the TSS is changed so as to modify the efficiency of transcription initiation and thereby modify the MSEC's overall transcriptional activity and product production levels.

In some embodiments the MSEC contains modified portions of the enhancer and/or promoter sequences of other mouse and human skeletal or cardiac muscle genes: e.g., the human alpha Myosin Heavy Chain gene (hMYH6) and the human cardiac troponin T gene (hTNNT2).

In some embodiments, MSECs include hormone and vitamin responsive control elements.

In MSECs of the types described above, all of the embodiments described for modifying MSECs designed from CKM components would also be applicable.

In some embodiments the MSEC contains a miniaturized version of the human TNNT2 enhancer region (MSEC-130).

The current disclosure describes MSECs that can express equal high, medium, or low product levels in all muscle fiber types, as well as MSECs whose transcription rates are high, medium, low, or even “off” in different fiber types (e.g., high in Type I, medium in Type IIa, and low or “off’ in Type IIx), as well as all combinations of these activity levels. Analogous TF differences are responsible for differential gene expression in atrial, ventricular, and conducting cardiomyocytes in the heart. produce graded product levels in different skeletal and cardiac muscles. The wide range of MSEC transcriptional activities in the disclosed MSEC library has been created by modifying muscle gene enhancers, promoters, and CE types within these, sequences, as well as the linear order of CEs and spacing between them and adjacent CEs to create synthetic MSECs that respond differently to the wide variety of physiological signals associated with different muscle types as well as changes in workloads. MSECs have also been miniaturized by deleting DNA sequences between enhancers and promoters and between CEs for the purpose of increasing the cDNA length that can be efficiently packaged in AdenoAssociated Virus and other vectors. As described below, optimal MSECs for different therapeutic goals will differ due to unique attributes of each therapeutic product, as well as pathological differences between diseases.

Relevant experimental methods are contained in many dozens of publications by Stephen D. Hauschka. When not described in detail, methods were always based upon standard Cell and Molecular Biology, Biochemical, and Immunological protocols used during the particular period.

MSEC Design and Construction: MSEC sequences were designed based on partial genomic sequences from different skeletal and cardiac muscle genes in which basic regulatory regions had been identified. These native sequences were tested for muscle-specific expression in skeletal and cardiac muscle cultures and in non-muscle cultures. Sequences that exhibited muscle-specific transcriptional activity were then subjected to iterative deletion-mediated miniaturization protocols to reduce MSEC sequence sizes. Miniaturized enhancer and promoter regions were then ligated together in many different gene-identity combinations to create MSECs containing components from different muscle genes. At all iterative steps MSECs were sequenced by standard protocols and sequences were entered in the MSEC Sequence Library (FIG. 27).

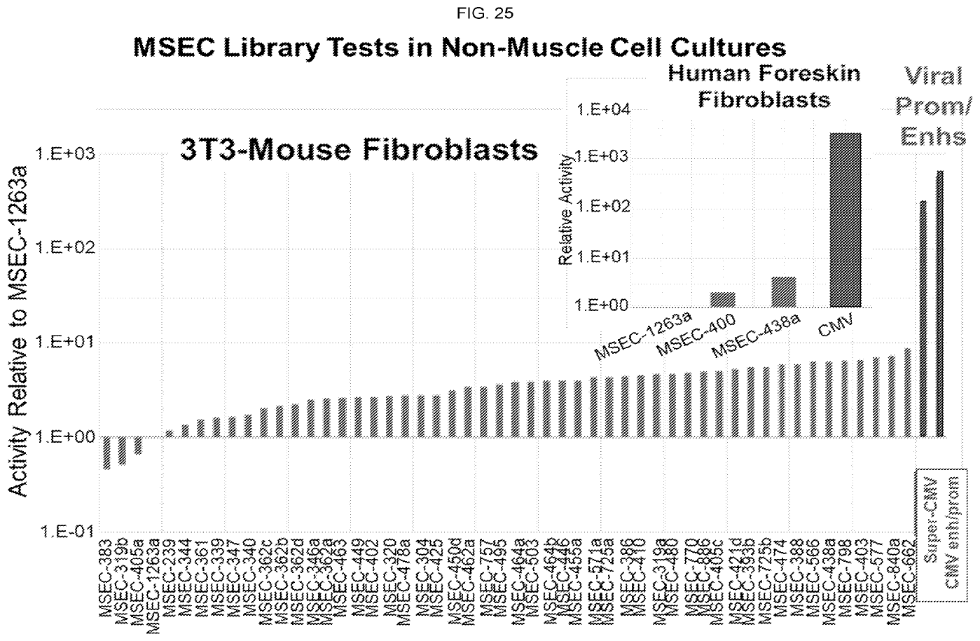

In Vitro MSEC Assays: MSECs were tested for transcriptional activity via transient transfection assays using standard transfection techniques. Skeletal muscle cultures typically used the MM14 mouse myoblast cell line, and Cardiac muscle cultures typically used newborn rat cardiomyocytes (PMID: 8474439), and protocols for normalizing data to adjust for different transfection efficiencies and levels of differentiation are illustrated in FIG. 4. Other in vitro skeletal muscle MSEC assays used isolated mouse FDB muscle fibers, and transfections were accomplished via electroporation. Many types of Non-muscle cells (FIG. 25) were also used for testing MSEC expression; cultures were transiently transduced by standard methods and reporter gene expression levels were determined by the same methods used for skeletal and cardiac muscle cultures, except that activities were not normalized for muscle differentiation levels.

In Vitro Data Quantitation: All MSEC comparisons were done by transfecting 4 or more culture dishes with the same MSEC, and mean reporter gene levels were calculated. MSEC-1263a (containing the native mouse CKM (−1263 to +7) genomic region) was included in all experiments, and its mean activity level was set to 1.0 and used as a basis for normalizing expression levels from all MSECs tested in the same experiment. Depending on preliminary data, MSEC analyses were repeated one or more times and the normalized mean data values from each repetition were averaged. This provided a basis for comparing all MSEC transcriptional activities even though the hundreds of different MSECs could not be compared in a single experiment.

In Vivo MSEC Assays: MSECs were tested for in vivo transcriptional activity in normal and dystrophic mice via both transgenic mouse, intramuscular vector injection, and systemic vector delivery protocols. Standard protocols were used in all studies, and these are described in publications by Stephen D. Hauschka. Recipient mice were weighed prior to systemic injections, and vectors were delivered at the same vector genome/kg body weight to all mice in each experimental cohort. Vectors were typically AAV6, but other AAV serotypes were also tested and MSECs were found to exhibit analogous transcriptional activity levels, albeit that individual AAV serotypes have different relative transduction efficiencies in different tissue types.

Vector Production and Quantification: Vectors were made and titered in the University of Washington Wellstone Center Vector Core under standard procedures.

Promoters. Preferred promoters for use in MSEC are provided in the disclosed MSEC library (e.g., within each MSEC listed in FIG. 27). However, the disclosure is not limited to these preferred promoters. MSECs or components from MSECs may be combined with promoter components from viral, artificial, or essentially any other vertebrate gene promoter to provide desired product expression levels in striated as well as in all or in specific other tissue types; e.g., striated muscle and smooth muscle, or liver, or connective tissue, etc., or in subsets of muscle tissue cells such as Satellite Cells in which standard MSECs are inactive.

As indicated, particular embodiments utilize promoter sequences included within MSECs presented in the FIG. 27 library. Certain examples utilize a basal promoter or a proximal promoter from either CKM or from other muscle genes such as ACTA1 or TNNI1, or non-muscle genes such as Thymidine kinase. Promoters selected for use within an MSEC can optionally include TATA box mutations (FIGS. 19 and 20) and/or N-box ligations (FIG. 21). Examples of TATA Box mutations that affect transcriptional activity can be found in PMIDs: 2342467 and 10617571 and FIG. 20).

When a gene is selectively expressed in targeted muscle cells and is not substantially expressed in non-targeted cells, the product of the coding sequence is preferentially expressed in the targeted cell type. In particular embodiments, selective expression is greater than 50% expression as compared to a reference cell type; greater than 60% expression as compared to a reference cell type; greater than 70% expression as compared to a reference cell type; greater than 80% expression as compared to a reference cell type; or greater than 90% expression as compared to a reference cell type. In particular embodiments, a reference cell type refers to non-muscle cells or a non-targeted muscle cell type. In particular embodiments, a reference cell type is within an anatomical structure that is adjacent to an anatomical structure that includes the targeted muscle cell type.

In particular embodiments, the product of the coding sequence may be expressed at low levels in non-selected and/or reference cell types, for example at less than 1% or 1%, 2%, 3%, 5%, 10%, 15% or 20% of the levels at which the product is expressed in targeted cells. In particular embodiments, the targeted muscle cell type is the only cell type that expresses the right combination of transcription factors that bind an enhancer disclosed herein to drive gene expression. Thus, in particular embodiments, expression occurs exclusively within the targeted cell type.

In certain examples, CK7 (e.g., MSEC-770) and CK8 (e.g., MSEC-438a) enhancers lead to selective expression in skeletal and cardiac muscle, as compared to non-muscle cell types. In other examples, cTnT (e.g., MSEC-455a) enhancers lead to selective expression in cardiac muscle, as compared to skeletal muscle and other non-muscle cell types.

Additional exemplary enhancers can be derived from slow muscle gene enhancers, TNNC1, TNNT1, TNNI1, and CKM slow muscle intronic enhancer (SIE) or from fast muscle gene enhancers, TNNI2 and TNNC2, as well as from genes such as ACTA1 that is thought to be expressed at equivalent levels in all skeletal muscle fiber types.

Within the current disclosure, enhancers can be multimerized. In certain examples, active segments of enhancers can be multimerized. Multimerized enhancers can include 2, 3, 4, 5, 6, 7, 8, 9, or 10 copies of an enhancer or active segment thereof.

Genes and Gene Products. A nucleic acid sequence (e.g., cDNA) encoding a protein or RNA of interest can be prepared and assembled into a complete coding sequence by standard techniques of molecular cloning (genomic library screening, polymerase chain reaction (PCR), primer-assisted ligation, libraries from yeast and bacteria, site-directed mutagenesis, etc.). The resulting coding region can be inserted into an expression vector as described herein.

The term “gene” refers to a nucleic acid sequence (used interchangeably with polynucleotide or nucleotide sequence) that encodes a protein or RNA of interest. This definition includes various sequence polymorphisms, mutations, and/or sequence variants wherein such alterations do not substantially affect the function of the encoded product. The term “gene” may include not only coding sequences but also regulatory regions such as promoters, enhancers, and termination regions. The term further can include all introns and other DNA sequences spliced from an mRNA transcript, along with variants resulting from alternative splice sites. The sequences can also include degenerate codons of the native sequence or sequences that may be introduced to provide codon preference in a specific cell type. Codon-optimized gene sequences can also be used.

“Encoding” refers to the property of specific sequences of nucleotides in a gene, such as a complementary DNA (cDNA), or a messenger RNA (mRNA), to serve as templates for synthesis of other macromolecules such as a defined sequence of amino acids. Thus, a gene codes for a protein if transcription and translation of mRNA corresponding to that gene produces the protein in a cell or other biological system. A “gene sequence encoding a protein” includes all nucleotide sequences that are degenerate versions of each other and that code for the same amino acid sequence or amino acid sequences of substantially similar form and function.

Particular embodiments of MSECs are used to selectively express proteins or RNA. Exemplary proteins include dystrophin, actin (skeletal or cardiac), kinases, phosphatases (e.g., P13P phosphatase (myotubularin)), proteases (e.g., Calpain 3), reporter proteins such as Luciferase, Alkaline phosphatase, Chloramphenicol acetyltransferase, GFP, mCherry, and others, gene-editing nucleases (e.g., Cas9, Cpf1), hormones, cytokines, extracellular matrix proteins, enzymes, antibodies, viral antigens for vaccines, and clotting factors, as well as any smaller versions of such proteins. Exemplary RNA types include gene-editing guide RNA (e.g., CRISPR RNA), microRNAs, and long non-coding RNAs.

Additional examples of expression products include dystrophins (e.g., full-length mouse dystrophin, Dp260-dystrophin, mini-dystrophins and micro-dystrophins, as well as dystrophin fragments containing intein assembly sequences), human liver arginase, human parathyroid hormone, human growth hormone, human placental alkaline phosphatase, clotting factor iX, human TDP43, mouse Ribonucleotide Reductase subunit-1, mouse Ribonucleotide Reductase subunit 2, mouse Interleukin-10, mouse alpha-Gluocosidase, mouse Glycogen Synthase, bacterial Cas/9, bacterial Chloramphenicol Acetyl Transferase, bacterial Beta-galactosidase, bacterial Luciferase, bacterial Renilla, Green Fluorescent protein, M-cherry fluorescent protein, and mTmG-2a-puromycin-resistance fusion protein.

microRNA Target Sites. An additional level of gene product control is exerted by the numerous microRNAs (miRNAs) expressed in proliferating skeletal muscle myoblasts, differentiated fibers types, and cardiomyocyte types. miRNAs typically affect the extent to which specific mRNAs are translated into proteins by enhancing degradation rates of the mRNAs to which they bind. The selectivity of which mRNAs are degraded is due to qualitative and concentration differences among the miRNAs produced by each cell type, and on the miRNA binding affinities for the slightly differing miRTS sequences that reside within different muscle gene mRNAs. By inserting different numbers of miRTs with high-to-low binding affinities into the non-coding regions of any desired product's cDNA, it is thus possible to adjust product levels in specific muscle subtypes with even greater precision than can be achieved via transcriptional controls alone. Finer tuning of product levels between muscle types can thus be obtained by selecting appropriate MSECs and miRTSs for each disease therapy. As one example, microRNA208a can reduce product levels in cardiac muscle cells. Additional examples of relevant microRNA target sites are provided in FIG. 27, page-1 and FIGS. 17 and 18.

Barcodes. In certain examples, MSEC can include or encode barcodes linked to cDNAs. MSEC-linked barcodes will generally be used to track particular MSEC locations in research studies following transfection of the MSECs. cDNA-linked barcodes will generally be used to for comparing the transcriptional activities of 2 or more MSECs ligated to cDNAs labeled with different barcodes. A mixture of AAVs or other vectors containing the indirectly barcoded cDNAs is then administered to cell cultures or tissue, and MSEC transcriptional activity is subsequently determined by Next Generation Sequencing of the resulting mRNAs produced by each MSEC type.

Barcodes are well known to those of skill in the art. In particular embodiments, barcodes refer to DNA sequences that can utilized to identify an MSEC. In particular embodiments, these barcodes can be designed to be unique. In particular embodiments, DNA barcodes can include standardized short sequences of DNA. See, for example, Kress and Erickson, Proc. Natl. Acad. Sci. USA, 105(8): 2761-2762; Savolainen et al., Trans R Soc London Ser B. 2005; 360:1805-1811.

In certain examples, different MSEC include or encode different barcodes. In other examples, MSECs are grouped by inclusion of a common feature, and those MSECs within the group share a common barcode. An exemplary common features is identity of enhancer. In this example, MSEC with the same enhancer would all share a common barcode while MSEC with a different enhancer would have a different barcode.

Barcodes of a great variety of lengths can be used. Longer sequences generally accommodate a larger number and variety of barcodes. In certain examples, all barcoded MSEC can have the same length barcode (albeit with different sequences), but it is also possible to use different length barcodes in different MSECs. A barcode sequence can be at least 2, 4, 6, 8, 10, 12, 15, 20 or more nucleotides in length (e.g., 3-6 nucleotides). In particular embodiments, the length of the barcode sequence can be at most 20, 15, 12, 10, 8, 6, 4 or fewer nucleotides. In particular embodiments, a barcode sequence may have a length in range of from 4 to 36 nucleotides, or from 6 to 30 nucleotides, or from 8 to 20 nucleotides. Barcode sequences are described in, for example: U.S. Pat. No. 5,635,400; Brenner et al., Proc. Natl. Acad. Sci., 97:1665-1670, 2000; Shoemaker et al., Nature Genetics 14: 450-456, 1996; EP0799897; U.S. Pat. No. 5,981,179; US20140342921; and U.S. Pat. No. 8,460,865.

MSEC Delivery. In particular embodiments, an MSEC ligated to cDNA encoding a protein or RNA of interest can be introduced into cells in a vector. A “vector” is a nucleic acid molecule that is capable of transporting another nucleic acid.

Many viral vector types are available and being developed for gene therapy delivery (PMID: 29883422). All of the MSECs described in this patent are compatible with incorporation into and delivery with one or more of these viruses, the only constraints being each vector's genomic packaging size and the cDNA size of interest. This MSEC attribute applies not only to currently available viral vectors, but will also apply to all newly developed viral vectors. A particular advantage of the MSEC technology is that muscle-specific MSECs as small as several hundred base pairs are available for use in viruses with small packaging size constraints, while larger MSECs are compatible with vectors with greater packaging capacities. An advantage of the latter vector types is that much larger cDNA sizes can also be accommodated. This means that full length cDNAs for even the largest known natural proteins as well as cDNAs encoding multiple proteins and/or RNAs, as well as very large artificial proteins can be packaged and expressed at appropriate levels using different MSECs and delivery vector types.

In other embodiments MSEC-cDNA constructs can be delivered via different formulations of “naked DNA” (e.g., Froehner, March 2021 MDA Clinical & Scientific Conference: Non-viral Delivery in Neuromuscular Disease), and by artificial lipid, and/or protein nanoparticles which encapsulate the MSEC-cDNA, and that contain various modifications such as tissue-specific ligands or antibodies to enhance binding to and uptake by muscle cells, and/or to facilitate transport of the internalized MSEC-cDNA complex to the cell nucleus (PMID: 30186185).

MSECs can also be used to express any protein and RNA components needed for genome modifications as well as for activating or repressing gene expression from targeted loci.

Compositions. Vectors described herein can be formulated into compositions for administration to subjects. Compositions include a therapeutically effective amount of MSEC (e.g., in vector form) and a pharmaceutically acceptable carrier.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants.

A “prophylactic treatment” includes a treatment administered to a subject who does not display signs or symptoms of a muscle-related disorder or displays only early signs or symptoms of a muscle-related disorder such that treatment is administered for the purpose of diminishing or decreasing the risk of developing the muscle-related disorder further. Thus, a prophylactic treatment functions as a preventative treatment against a muscle-related disorder. In particular embodiments, prophylactic treatments reduce, delay, or prevent the worsening of a muscle-related disorder.

A “therapeutic treatment” includes a treatment administered to a subject who displays symptoms or signs of a muscle-related disorder and is administered to the subject for the purpose of diminishing or eliminating those signs or symptoms of the muscle-related disorder. The therapeutic treatment can reduce, control, or eliminate the presence or activity of the muscle-related disorder and/or reduce control or eliminate side effects of the muscle-related disorder.

Function as a prophylactic treatment or therapeutic treatment are not mutually exclusive, and in particular embodiments, administered dosages may accomplish more than one treatment type.

Skeletal and cardiac muscles are affected by hundreds of genetic diseases, are subject to many types of physical injury, undergo progressive functional weakness during disuse and aging, and skeletal muscle also undergoes debilitating catabolic degradation in conjunction with cancers. Since all skeletal muscle fibers are innervated, and since the function of muscle cell synaptic regions where neuronal axons stimulate muscle contraction depends on neuronal interactions, muscle cells also exhibit a variety of Neuromuscular Junction (NMJ) diseases. Additionally, since the maintenance of innervating neurons is partially dependent on muscle-mediated signals, muscles also play important roles in the normal function of their innervating neurons. MSECs can play major roles in therapeutic strategies for combating all of these medical issues, as well as analogous issues in veterinary medicine.

Age-related sarcopenia, cancer cachexia, and spinal cord injuries tend to affect Type II fibers more than Type I fibers. Duchenne (DMD) and Facioscapulohumeral (FSHD) muscular dystrophies also exhibit greater effects on Type II fibers, while FKRP-mediated Dystroglycanopathies (MDDGA5, MDDGB5 and MDDGC5), exhibit relative increases in Type I fibers, possibly due to gradual Type II- to Type I transitions. In contrast, Myotonic dystrophy and some Limb Girdle Muscular Dystrophies (e.g., LGMD2A due to Calpain-3 deficiency) are associated with reduced Type I fibers. NMJ diseases also exhibit skeletal muscle fiber type changes; e.g., infants with the most severe forms of Spinal Muscular Atrophy (SMA) have many fewer Type II fibers and an associated increase in Type I fibers; and patients with advanced Amyotrophic Lateral Sclerosis (ALS) exhibit a transition from Type II to Type I fibers. Some striated muscle diseases such as DMD effect both skeletal and cardiac muscles, whereas others primarily affect skeletal or cardiac muscle, and some cardiac muscle diseases have their most pronounced effects on either ventricular, atrial, or conduction components. These disease-specific muscle type differences underscore the importance of MSECs that are optimized for each disease type so as to focus gene therapies to the most affected muscles and fiber types.

Particular muscle-related disorders that can be treated include cardiac muscle disease (e.g., Hypertrophic Cardiomyopathy) and Striated muscle diseases including dystrophies and dystroglycanopathies. Dystrophies include muscular dystrophies and Myotonic dystrophies. Examples of muscular dystrophies include Limb-girdle muscular dystrophies (LGMD), LGMD2A due to calpain-3 deficiency, MTM1, ACTA1 LGMD, Duchenne Muscular Dystrophy (DMD), and Facioscapulohumeral muscular dystrophy (FSHD). Examples of Dystroglycanopathies include MDC1A, MDDGA5, MDDGB5, MDDGC5, and MDDGC14 (GMPPB disease). Neuromuscular disorders (NMD) and Neuromuscular junction disorders (NMJ) can also be treated. These include Spinal Muscular Atrophy (SMA), amyotrophic lateral sclerosis (ALS), and Myasthenic NMDs (e.g., Congenital myasthenic (those affecting acetylcholine receptor subunits (CHRNA1; CHRNB1; CHRND; CHRNE), COLQ or DOK7), LGMD2A and MTM1. Additional examples of disorders that can be treated include amyopathies, Nemalin myopathies (e.g., Nemaline Myopathy-2), Myofibrillar Myopathy-5, Miyoshi Myopathy, Scapuloperoneal Myopathy, X-linked myotubular myopathy, Central Core Disease, Paramyotonia, Pompe Disease, Cancer cachexia, and aging diseases (age-related sarcopenia), among other diseases or disorders described elsewhere herein.

The term “muscle” means a structure, which is composed of myoblasts, myotubes, myofibers, stem cells that could produce myoblasts, and proteins that support those structures. The muscle includes skeletal, cardiac, and smooth muscles.

The term “muscle injury” refers to the condition that muscle does not function normally. The injury could be caused by excessive impact to a muscle where muscle fibers compressed in this manner can become irritated and even torn, caused when a muscle is stretched beyond its capacity and caused when intense and rapid contraction is demanded of a muscle.

The terms “muscle atrophy” and “muscle loss” refer to the condition which is caused by disuse of muscles, e.g. a lack of physical activity. For example, a subject under the medical conditions that limit their movement can lose muscle tone and develop atrophy.

The term “muscle strength” means the amount of the force that muscle can produce with maximal efforts.

The term “cancer-associated cachexia” and “infection-induced cachexia” mean an ongoing loss of skeletal muscle mass that cannot be reversed by conventional nutritional support and leads to progressive functional impairment. Cachexia caused by cancer refers “cancer-associated cachexia” and induced by infection is defined as “infection-induced cachexia”.

The term “aging” means the physiological process, which associates a progressive functional decline, or a gradual deterioration of physiological function with age.

The term “cardiovascular disease” refers to disease of the circulatory system including the heart and blood vessels. There are four main types of cardiovascular disease: coronary heart disease, stroke, peripheral arterial disease, and aortic disease.

The term “regeneration” means the repair of cells, tissues, or organs. In the present disclosure, the term regeneration refers to the repair of myoblasts, myofibers, and muscular environment, which could provide an optimal environment to generate myofibers.

As indicated previously, the most direct use of MSEC is in the development of treatments for muscle-related disorders. However, there are numerous other uses for MSEC as well. Examples include in the development of treatments for disorders in which skeletal muscle tissue can be used to produce beneficial secreted products such as hormones, clotting factors, antibodies, or other beneficial proteins or metabolites. MSECs could also be used for immunization against virtually any antigen, for a wide variety of veterinary and animal agricultural purposes, as well as for cell-based meat production.

### Exemplary Embodiments—Set 3

1. An artificial muscle-specific expression cassette (MSEC) that, when administered to a heterogenous cell population, results in selective expression of a first coding sequence in a muscle cell within the heterogenous cell population.

2. An MSEC of embodiment 1, wherein the MSEC is disclosed herein.

3. An MSEC of embodiment 1, wherein the MSEC comprises a promoter.

4. An MSEC of embodiment 2, wherein the promoter is a CKM proximal promoter, a TNNT2 promoter, a TNNT1 promoter, a thymidine kinase promoter, an SV40 promoter, or a CMV promoter.

5. An MSEC of embodiment 3 or 4, wherein the promoter is a miniaturized promoter.

6. An MSEC of any of embodiments 3-5, wherein the promoter is multimerized.

7. An MSEC of embodiment 5, wherein the multimerized promoter has 2, 3, 4, 5, 6, 7, 8, 9, or 10 copies of the promoter and/or miniaturized promoter.

8. An MSEC of any of embodiments 3-7, wherein the promoter includes a TATA box mutation.

9. An MSEC of any of embodiments 3-8, wherein the promoter has an N box ligation.

10. An MSEC of any of embodiments 1-9, wherein the MSEC further includes an enhancer.

11. An MSEC of embodiment 10, wherein the enhancer is a CKM intron-1 MR1 enhancer, a TNNT2 enhancer, a TNNT1 enhancer, a mouse CKM 5′ enhancer, or an alpha-myosin heavy chain enhancer.

12. An MSEC of embodiment 10 or 11, wherein the enhancer is a miniaturized enhancer.

13. An MSEC of embodiment 12, wherein the miniaturized enhancer is a miniaturized CKM intron-1 MR1 enhancer.

14. An MSEC of embodiment 12, wherein the miniaturized enhancer includes +904 to +998 of the native mouse CKM gene.

15. An MSEC of any of embodiments 10-14, wherein the enhancer is multimerized.

16. An MSEC of embodiment 15, wherein the multimerized enhancer has 2, 3, 4, 5, 6, 7, 8, 9, or 10 copies of the enhancer and/or miniaturized enhancer.

17. An MSEC of any of embodiments 1-16, wherein the first nucleotide coding sequence includes cDNA.

18. An MSEC of any of embodiments 1-16, wherein the first nucleotide coding sequence or cDNA encodes a protein or RNA.

19. An MSEC of any of embodiments 1-18, further including a second nucleotide coding sequence.

20. An MSEC of embodiment 19, wherein the second nucleotide coding sequence encodes a microRNA target site.

21. An MSEC of embodiment 20, wherein the second nucleotide coding sequence encodes 1, 2, 3, 4, 5, 6, 7, 8, 9, or 10 copies the microRNA target site

22. An MSEC of embodiment 20 or 21, wherein the microRNA target site reduces expression of the first nucleotide coding sequence in cardiac muscle or skeletal muscle.

23. An MSEC of any of embodiments 20-22, wherein the microRNA target site is a microRNA target site disclosed herein.

24. An MSEC of any of embodiments 16-23, wherein the second nucleotide coding sequence encodes at least two different microRNA target sites.

25. A sequence as set forth in any one of SEQ ID NOs. 1-372 or a sequence having at least 90%, 91%, 92%, 93%, 94%, 95%, 96%, 97%, 98%, or 99% sequence identity to a sequence as set forth in any one of SEQ ID NOs. 1-372.

26. An MSEC or sequence of any of embodiments 1-25, formulated for administration to a subject.

27. An MSEC of any of embodiments 1-26, within a vector for delivery to a subject.

28. An MSEC of embodiment 27, wherein the vector for delivery is a viral vector.

29. An MSEC of embodiment 28, wherein the viral vector is an adeno-associated (AAV) viral vector.

30. Use of an MSEC or sequence of any of embodiments 1-29, to selectively drive gene expression in a muscle cell.

31. A use of embodiment 30, wherein the muscle cell is a striated muscle, a skeletal muscle, or a cardiac muscle.

32. A use of embodiment 30, wherein the muscle is a skeletal muscle or a cardiac muscle.

33. A use of embodiment 30, wherein the use is to develop a treatment for a muscle-related disorder.

Section headings within the disclosure are provided for organization purposes only and do not limit the scope or interpretation of the disclosure.

The nucleic acid and/or amino acid sequences described herein are shown using standard letter abbreviations, as defined in 37 C.F.R. § 1.822. In certain examples, only one strand of each nucleic acid sequence is shown, but the complementary strand is understood as included in embodiments where it would be appropriate.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of’ excludes any element, step, ingredient or component not specified. The transition phrase “consisting essentially of’ limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. An artificial muscle-specific expression cassette (MSEC) having a sequence as set forth in FIG. 27 for MSEC-3363, MSEC-74, MSEC-87, MSEC-95, MSEC-118, MSEC-144, MSEC-206A, MSEC-206B, MSEC-212, MSEC-220, MSEC-239, MSEC-259, MSEC-283, MSEC-285, MSEC-288, MSEC-290, MSEC-297A, MSEC-297B, MSEC 297C, MSEC 297D, MSEC 297E, MSEC 297F, MSEC 297G, MSEC 297H, MSEC 2971, MSEC 297J, MSEC 297K, MSEC 297L, MSEC-302A, MSEC-302B, MSEC-302C, MSEC-303, MSEC-304, MSEC-310A, MSEC-310B, MSEC 310C, MSEC 310D, MSEC 310E, MSEC 310F, MSEC 310G, MSEC 310H, MSEC 3101, MSEC 310J, MSEC 310K, MSEC 310L, MSEC-315A, MSEC-315B, MSEC-315C, MSEC-318, MSEC-319B, MSEC-320, MSEC-322, MSEC-327, MSEC-330, MSEC-335A, MSEC-335B, MSEC-336A, MSEC-336B, MSEC-336C, MSEC-336D, MSEC-339, MSEC-340, MSEC-341, MSEC-344, MSEC-345, MSEC-346A, MSEC-347, MSEC-348, MSEC-350, MSEC-351, MSEC-352A, MSEC-352B, MSEC-356, MSEC-360A, MSEC-360B, MSEC-361, MSEC-362A, MSEC-362B, MSEC-362C, MSEC-362D, MSEC-362E, MSEC-362F, MSEC-365A, MSEC-365B, MSEC-367, MSEC-378, MSEC-382, MSEC-383, MSEC-384, MSEC-386, MSEC-388, MSEC-393A, MSEC-393B, MSEC-395, MSEC-395B, MSEC-400, MSEC-402, MSEC-403, MSEC-403B, MSEC-405A, MSEC-405B, MSEC-405C, MSEC-406, MSEC-410, MSEC-411, MSEC-413, MSEC-417, MSEC-420A, MSEC-420B, MSEC-421A, MSEC-421B, MSEC-421C, MSEC-421D, MSEC-423, MSEC-425, MSEC-427A, MSEC-427B, MSEC-427C, MSEC-429A, MSEC-429B, MSEC-433, MSEC-438A, MSEC-438B, MSEC-438C, MSEC-438D, MSEC-438E, MSEC-438F, MSEC-438G, MSEC-438H, MSEC-4381, MSEC-439A, MSEC-439B, MSEC-440, MSEC-443, MSEC-444, MSEC-446, MSEC-449, MSEC-450A, MSEC-450B, MSEC-450C, MSEC-450D, MSEC-455A, MSEC-455B, MSEC-455C, MSEC-457, MSEC-461, MSEC-462A, MSEC-462B, MSEC-463, MSEC-464A, MSEC-464B, MSEC-466, MSEC-472, MSEC-474, MSEC-476, MSEC-476B, MSEC-478A, MSEC-478B, MSEC-478C, MSEC-480, MSEC-481, MSEC-486A, MSEC-486B, MSEC-486C, MSEC-487A, MSEC-487B, MSEC-493A, MSEC-493B, MSEC-495, MSEC-501, MSEC-503, MSEC-508, MSEC-510, MSEC-513, MSEC-517, MSEC-518, MSEC-523, MSEC-527A, MSEC-527B, MSEC-529A, MSEC-529B, MSEC-529C, MSEC-531A, MSEC-531B, MSEC-538A, MSEC-538B, MSEC-539A, MSEC-539B, MSEC-540, MSEC-541A, MSEC-541B, MSEC-546, MSEC-547, MSEC-550, MSEC-553, MSEC-555, MSEC-556A, MSEC-556B, MSEC-556C, MSEC-563, MSEC-566, MSEC-569, MSEC-571A, MSEC-571B, MSEC-577, MSEC-581, MSEC-582A, MSEC-582B, MSEC-584, MSEC-586, MSEC-587, MSEC-588A, MSEC-588B, MSEC-589A, MSEC-589B, MSEC-590, MSEC-594, MSEC-601, MSEC-602A, MSEC-602B, MSEC-602C, MSEC-602D, MSEC-602E, MSEC-602F, MSEC-602G, MSEC-602H, MSEC-605, MSEC-607, MSEC-608, MSEC-610, MSEC-611, MSEC-617, MSEC-623, MSEC-625, MSEC-626, MSEC-633, MSEC-638, MSEC-640, MSEC-643, MSEC-652, MSEC-653, MSEC-656A, MSEC-656B, MSEC-658, MSEC-662, MSEC-672, MSEC-673, MSEC-674, MSEC-679A, MSEC-679B, MSEC-681, MSEC-685, MSEC-686, MSEC-689, MSEC-697, MSEC-701, MSEC-714, MSEC-718, MSEC-720, MSEC-721, MSEC-723A, MSEC-723B, MSEC-725A, MSEC-725B, MSEC-725C, MSEC-725D, MSEC-726, MSEC-730, MSEC-732, MSEC-734, MSEC-736, MSEC-745, MSEC-746, MSEC-749, MSEC-754, MSEC-757, MSEC-758, MSEC-764, MSEC-767, MSEC-768, MSEC-770, MSEC-771, MSEC-773, MSEC-778, MSEC-783, MSEC-786, MSEC-793, MSEC-794, MSEC-798, MSEC-804, MSEC-806, MSEC-809, MSEC-812A, MSEC-812B, MSEC-836, MSEC-840A, MSEC-840B, MSEC-855, MSEC-872, MSEC-875, MSEC-879, MSEC-886, MSEC-890, MSEC-892A, MSEC-892B, MSEC-899, MSEC-935, MSEC-952, MSEC-960, MSEC-964, MSEC-966, MSEC-970, MSEC-988, MSEC-995, MSEC-1016, MSEC-1031, MSEC-1204, MSEC-1207, MSEC-1225, MSEC-1240, MSEC-1263B, MSEC-1263C, MSEC-1263D, MSEC-1263E, MSEC-1263F, MSEC-1263G, MSEC-1263H, MSEC-12631, MSEC-1263J, SEC-1263K, MSEC-1263L, MSEC-1263M, MSEC-1263N, MSEC-12630, MSEC-1263P, MSEC-1263S, MSEC-1263T, MSEC-1263U, MSEC-1263V, MSEC-1279, MSEC-1360, MSEC-1370A, MSEC-1370B, MSEC-1465, MSEC-1666, MSEC-1755, MSEC-1263A, or MSEC-1027;
or an MSEC having at least 90%, 91%, 92%, 93%, 94%, 95%, 96%, 97%, 98%, or 99% sequence identity to MSEC-74, MSEC-87, MSEC-95, MSEC-118, MSEC-144, MSEC-206A, MSEC-206B, MSEC-212, MSEC-220, MSEC-239, MSEC-259, MSEC-283, MSEC-285, MSEC-288, MSEC-290, MSEC-297A, MSEC-297B, MSEC 297C, MSEC 297D, MSEC 297E, MSEC 297F, MSEC 297G, MSEC 297H, MSEC 2971, MSEC 297J, MSEC 297K, MSEC 297L, MSEC-302A, MSEC-302B, MSEC-302C, MSEC-303, MSEC-304, MSEC-310A, MSEC-310B, MSEC 310C, MSEC 310D, MSEC 310E, MSEC 310F, MSEC 310G, MSEC 310H, MSEC 3101, MSEC 310J, MSEC 310K, MSEC 310L, MSEC-315A, MSEC-315B, MSEC-315C, MSEC-318, MSEC-319B, MSEC-320, MSEC-322, MSEC-327, MSEC-330, MSEC-335A, MSEC-335B, MSEC-336A, MSEC-336B, MSEC-336C, MSEC-336D, MSEC-339, MSEC-340, MSEC-341, MSEC-344, MSEC-345, MSEC-346A, MSEC-347, MSEC-348, MSEC-350, MSEC-351, MSEC-352A, MSEC-352B, MSEC-356, MSEC-360A, MSEC-360B, MSEC-361, MSEC-362A, MSEC-362B, MSEC-362C, MSEC-362D, MSEC-362E, MSEC-362F, MSEC-365A, MSEC-365B, MSEC-367, MSEC-378, MSEC-382, MSEC-383, MSEC-384, MSEC-386, MSEC-388, MSEC-393A, MSEC-393B, MSEC-395, MSEC-395B, MSEC-400, MSEC-402, MSEC-403, MSEC-403B, MSEC-405A, MSEC-405B, MSEC-405C, MSEC-406, MSEC-410, MSEC-411, MSEC-413, MSEC-417, MSEC-420A, MSEC-420B, MSEC-421A, MSEC-421B, MSEC-421C, MSEC-421D, MSEC-423, MSEC-425, MSEC-427A, MSEC-427B, MSEC-427C, MSEC-429A, MSEC-429B, MSEC-433, MSEC-438A, MSEC-438B, MSEC-438C, MSEC-438D, MSEC-438E, MSEC-438F, MSEC-438G, MSEC-438H, MSEC-4381, MSEC-439A, MSEC-439B, MSEC-440, MSEC-443, MSEC-444, MSEC-446, MSEC-449, MSEC-450A, MSEC-450B, MSEC-450C, MSEC-450D, MSEC-455A, MSEC-455B, MSEC-455C, MSEC-457, MSEC-461, MSEC-462A, MSEC-462B, MSEC-463, MSEC-464A, MSEC-464B, MSEC-466, MSEC-472, MSEC-474, MSEC-476, MSEC-476B, MSEC-478A, MSEC-478B, MSEC-478C, MSEC-480, MSEC-481, MSEC-486A, MSEC-486B, MSEC-486C, MSEC-487A, MSEC-487B, MSEC-493A, MSEC-493B, MSEC-495, MSEC-501, MSEC-503, MSEC-508, MSEC-510, MSEC-513, MSEC-517, MSEC-518, MSEC-523, MSEC-527A, MSEC-527B, MSEC-529A, MSEC-529B, MSEC-529C, MSEC-531A, MSEC-531B, MSEC-538A, MSEC-538B, MSEC-539A, MSEC-539B, MSEC-540, MSEC-541A, MSEC-541B, MSEC-546, MSEC-547, MSEC-550, MSEC-553, MSEC-555, MSEC-556A, MSEC-556B, MSEC-556C, MSEC-563, MSEC-566, MSEC-569, MSEC-571A, MSEC-571B, MSEC-577, MSEC-581, MSEC-582A, MSEC-582B, MSEC-584, MSEC-586, MSEC-587, MSEC-588A, MSEC-588B, MSEC-589A, MSEC-589B, MSEC-590, MSEC-594, MSEC-601, MSEC-602A, MSEC-602B, MSEC-602C, MSEC-602D, MSEC-602E, MSEC-602F, MSEC-602G, MSEC-602H, MSEC-605, MSEC-607, MSEC-608, MSEC-610, MSEC-611, MSEC-617, MSEC-623, MSEC-625, MSEC-626, MSEC-633, MSEC-638, MSEC-640, MSEC-643, MSEC-652, MSEC-653, MSEC-656A, MSEC-656B, MSEC-658, MSEC-662, MSEC-672, MSEC-673, MSEC-674, MSEC-679A, MSEC-679B, MSEC-681, MSEC-685, MSEC-686, MSEC-689, MSEC-697, MSEC-701, MSEC-714, MSEC-718, MSEC-720, MSEC-721, MSEC-723A, MSEC-723B, MSEC-725A, MSEC-725B, MSEC-725C, MSEC-725D, MSEC-726, MSEC-730, MSEC-732, MSEC-734, MSEC-736, MSEC-745, MSEC-746, MSEC-749, MSEC-754, MSEC-757, MSEC-758, MSEC-764, MSEC-767, MSEC-768, MSEC-770, MSEC-771, MSEC-773, MSEC-778, MSEC-783, MSEC-786, MSEC-793, MSEC-794, MSEC-798, MSEC-804, MSEC-806, MSEC-809, MSEC-812A, MSEC-812B, MSEC-836, MSEC-840A, MSEC-840B, MSEC-855, MSEC-872, MSEC-875, MSEC-879, MSEC-886, MSEC-890, MSEC-892A, MSEC-892B, MSEC-899, MSEC-935, MSEC-952, MSEC-960, MSEC-964, MSEC-966, MSEC-970, MSEC-988, MSEC-995, MSEC-1016, MSEC-1031, MSEC-1204, MSEC-1207, MSEC-1225, MSEC-1240, MSEC-1263B, MSEC-1263C, MSEC-1263D, MSEC-1263E, MSEC-1263F, MSEC-1263G, MSEC-1263H, MSEC-12631, MSEC-1263J, SEC-1263K, MSEC-1263L, MSEC-1263M, MSEC-1263N, MSEC-12630, MSEC-1263P, MSEC-1263S, MSEC-1263T, MSEC-1263U, MSEC-1263V, MSEC-1279, MSEC-1360, MSEC-1370A, MSEC-1370B, MSEC-1465, MSEC-1666, MSEC-1755, MSEC-3363, MSEC-1263A, or MSEC-1027.

2. A muscle-specific expression cassette (MSEC) comprising an enhancer and a promoter operably linked to a first coding sequence, wherein, when administered to a heterogenous cell population, the MSEC results in selective expression of the first coding sequence in a muscle cell within the heterogenous cell population.

3. The MSEC of claim 2, wherein the promoter is a an ACTA1 promoter, a CKM proximal promoter, a TNNT2 promoter, a TNNT1 promoter, a thymidine kinase promoter, an SV40 promoter, or a CMV promoter.

4. The MSEC of claim 2, wherein the promoter is a miniaturized promoter.

5. The MSEC of claim 4, wherein the miniaturized promoter is multimerized.

6. The MSEC of claim 5, wherein the multimerized miniaturized promoter has 2, 3, 4, 5, 6, 7, 8, 9, or 10 copies of the miniaturized promoter.

7. The MSEC of claim 2, wherein the promoter includes a TATA box mutation.

8. The MSEC of claim 2, wherein the promoter has an N box ligation.

9. The MSEC of claim 2, wherein the enhancer is a CKM intron-1 MR1 enhancer, a TNNT2 enhancer, a TNNT1 enhancer, a mouse CKM 5′ enhancer, or an alpha-myosin heavy chain enhancer, or an ACTA1 enhancer.

10. The MSEC of claim 2, wherein the enhancer is a miniaturized enhancer.

11. The MSEC of claim 10, wherein the miniaturized enhancer is a miniaturized CKM intron-1 MR1 enhancer.

12. An MSEC of claim 2 or a combination thereof, formulated for administration to a subject.

13. An MSEC of claim 2 or a combination thereof, within a vector for delivery to a subject.

14. The MSECs of claim 13, wherein the vector for delivery is a viral vector.

15. The MSEC of claim 14, wherein the viral vector is an adeno-associated (AAV) viral vector.

16. The MSEC of claim 2, wherein the first coding sequence comprises cDNA.

17. The MSEC of claim 16, wherein the first coding sequence encodes a protein or RNA.

18. The MSEC of claim 2, further comprising a second coding sequence.

19. The MSEC of claim 18, wherein the second coding sequence encodes a microRNA target site.

20. The MSEC of claim 19, wherein the second coding sequence encodes 1, 2, 3, 4, 5, 6, 7, 8, 9, or 10 copies the microRNA target site

21. The MSEC of claim 20, wherein the microRNA target site reduces expression of the first coding sequence in cardiac muscle or skeletal muscle.

22. The MSEC of claim 20, wherein the second coding sequence encodes at least two different microRNA target sites.

23. Use of an MSEC of claim 1, to selectively drive gene expression in a muscle cell.

24. The use of claim 23, wherein the muscle cell is a striated muscle, a skeletal muscle, or a cardiac muscle.

25. The use of claim 23, wherein the muscle is a skeletal muscle or a cardiac muscle.

26. The use of claim 23, wherein the use is to develop a treatment for a muscle-related disorder.


SEQUENCE LISTING (372 sequences)

SEQ ID NO: 1 - DNA, 74 bp, Mus musculus
gcccacctgtcccaatgctgacttagtgcaaggcgagccagaggacaggtggcagagcatctaaaaatagccac

SEQ ID NO: 2 - DNA, 87 bp, Mus musculus
cctccctggggacagcccctcctggctagtcacaccctgtaggctcctctatataacccaggggcacaggggctgcccccgggtcac

SEQ ID NO: 3 - DNA, 95 bp, Mus musculus
gcccacctgtcccaatgctgacttagtgcaaggcgagccagcaaggagggaggacaggtggcagtggggggtgaggagcatctaaaaatagccac

SEQ ID NO: 4 - DNA, 118 bp, Mus musculus
gggcaacgagctgaaagctcatctgctctcaggggcccctccctggggacagcccctcctggctagtcacaccctgtaggctcctctatataacccaggggcacaggggctgcccccg

SEQ ID NO: 5 - skipped

SEQ ID NO: 6 - DNA, 206 bp, Mus musculus
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggat

SEQ ID NO: 7 - DNA, 206 bp, Artificial Sequence
  MSEC-206B
atgcctggttataattaacccagacatgtggctgcccccccccccccaacacctgctgcctgagcctcacccccaccccggtgcctgggtcttaggctctgtacaccatgtcgacggcccctccctggggacagcccctcctggctagtcacaccctgtaggctcctctatataacccaggggcacaggggctgcccccgggtcac

SEQ ID NO: 8 - DNA, 212 bp, Artificial Sequence
  MSEC-212
gctgcccatgtaaggaggcctggttataattaacccagacatgtggctgcccccccccccccaacacctgctgcatgcggcccctccctggggacagcccctcctggctagtcacaccctgtaggctcctctatataacccaggggcacaggggctgcccccgggtcaccaccacctccacagcacagacagacactcaggagccagccagc

SEQ ID NO: 9 - DNA, 220 bp, Artificial Sequence
  MSEC-220
aatcaaggctgtgggggactgagggcaggctgtaacaggcttgggggccagggcttatacgtgcctgggactcccaaagtattactgttcgtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 10 - DNA, 239 bp, Homo sapiens
ccgctccttctttggtcaacgcaggggacccgggcgggggcccaggccgcgaaccggccgagggagggggctctagtgcccaacacccaaatatggctcgagaagggcagcgacattcctgcggggtggcgcggagggaatcgcccgcgggctatataaaacctgagcagagggacaagcggccaccgcagcggacagcgccaagtgaagcctcgcttcccctccgcggcgaccagggc

SEQ ID NO: 11 - DNA, 259 bp, Artificial Sequence
  MSEC-259
acacccaaatatggctcgcgctctaaaaataaccctgggacacccgagatgcctggttataattaacccacccaacacctgctgcccccgctccttctttggtcagtcgcacattcctccctgtcgcacattcctcccttgccgctctaaaaataaccctgaacacccaaatatggctcgctgcagcattcctgggggccgggcggtgctcccgcccgcctcgataaaaggctccggggccggcggcggcccacgagct

SEQ ID NO: 12 - DNA, 283 bp, Artificial Sequence
  MSEC-283
ctgctcccagctggccctcccagtctgctttatcaggtttatgttgcatgactgttccctgcatatctgctctggttttaaatagcttatctgagcagctggaggaccacatgggcttatatggggcacctgccaaaatagcagccaacacccccccctgtcgcacattcctccctggctcaccaggccccagcccacatgcctgcttaaagccctctccatcctctgcctcacccagtccccgctgagactgagcagacgcctccaggatctgtcggcagct

SEQ ID NO: 13 - DNA, 285 bp, Artificial Sequence
  MSEC-285
acacccaaatatggctcgcgctctaaaaataaccctgggacacccgagatgcctggttataattaacccacccaacacctgctgcccccgctccttctttggtcagtcgcacattcctccctgtcgcacattcctcccttgccgctctaaaaataaccctgaacacccaaatatggctcgctgcagccgctccttctttggtcaacacccaaatatggctcgacagcgacattcctgcggggcccgcgggctatataaaacctgagcagagggacaagcggccac

SEQ ID NO: 14 - DNA, 288 bp, Artificial Sequence
  MSEC-288
gctgcccatgtaaggaggcctggttataattaacccagacatgtggctgcccccccccccccaacacctgctgcatgctgcccatgtaaggaggcctggttataattaacccagacatgtggctgcccccccccccccaacacctgctgcatgcggcccctccctggggacagcccctcctggctagtcacaccctgtaggctcctctatataacccaggggcacaggggctgcccccgggtcaccaccacctccacagcacagacagacactcaggagccagccagc

SEQ ID NO: 15 - DNA, 290 bp, Artificial Sequence
  MSEC-290
ctgctcccagctggccctcccagtctgctttatcagacagctggtttatgttgcatgactgttccctgcatatctgctctggttttaaatagcttatctgagcagctggaggaccacatgggcttatatggggcacctgccaaaatagcagccaacacccccccctgtcgcacattcctccctggctcaccaggccccagcccacatgcctgcttaaagccctctccatcctctgcctcacccagtccccgctgagactgagcagacgcctccaggatctgtcggcagct

SEQ ID NO: 16 - DNA, 297 bp, Mus musculus
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccgacctccctggggacagcccctcctggctagtcacaccctgtaggctcctctatataacccaggggcacaggggctgcccccgggtcac

SEQ ID NO: 17 - DNA, 297 bp, Artificial Sequence
  MSEC-297B
ccactacgggtctaggctgcccatgtaaggaggcaagatctagagacacagctgatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccgacctccctggggacagcccctcctggctagtcacaccctgtaggctcctctatataacccaggggcacaggggctgcccccgggtcac

SEQ ID NO: 18 - DNA, 297 bp, Artificial Sequence
  MSEC-297C
ccactacgggtctaggctgcgcaggtaccgaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccgacctccctggggacagcccctcctggctagtcacaccctgtaggctcctctatataacccaggggcacaggggctgcccccgggtcac

SEQ ID NO: 19 - DNA, 297 bp, Artificial Sequence
  MSEC-297D
ccactacgggtctaggctgcccatgtaaggaggcaagatctagagacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccgacctccctggggacagcccctcctggctagtcacaccctgtaggctcctctatataacccaggggcacaggggctgcccccgggtcac

SEQ ID NO: 20 - DNA, 297 bp, Artificial Sequence
  MSEC-297E
ccactacgggtctaggctgcccatgtaaggaggcaaggcctggggacacccgagatgcctggctagcatgcacccagacatgtggctgcccccccccccccaacacctgctgcctgagcctcacccccaccccggtgcctgggtcttaggctctgtacaccatggaggagaagctcgctctaaaaataaccctgtccctggtggatccgacctccctggggacagcccctcctggctagtcacaccctgtaggctcctctatataacccaggggcacaggggctgcccccgggtcac

SEQ ID NO: 21 - DNA, 297 bp, Artificial Sequence
  MSEC-297F
ccactacgggtctaggctgcccatgtaaggaggcaaggcctggggacacccgagatgcctggttctagatctcccagacatgtggctgcccccccccccccaacacctgctgcctgagcctcacccccaccccggtgcctgggtcttaggctctgtacaccatggaggagaagctcgctctaaaaataaccctgtccctggtggatccgacctccctggggacagcccctcctggctagtcacaccctgtaggctcctctatataacccaggggcacaggggctgcccccgggtcac

SEQ ID NO: 22 - DNA, 297 bp, Artificial Sequence
  MSEC-297G
ccactacgggtctaggctgcccatgtaaggaggcaaggcctggggacacccgagatgcctggttataattaacccatctagatggctgcccccccccccccaacacctgctgcctgagcctcacccccaccccggtgcctgggtcttaggctctgtacaccatggaggagaagctcgctctaaaaataaccctgtccctggtggatccgacctccctggggacagcccctcctggctagtcacaccctgtaggctcctctatataacccaggggcacaggggctgcccccgggtcac

SEQ ID NO: 23 - DNA, 297 bp, Artificial Sequence
  MSEC-297H
ccactacgggtctaggctgcccatgtaaggaggcaaggcctggggacacccgagatgcctggttataattaacccagacatgtggctgcccccccccccccaagatctgctgcctgagcctcacccccaccccggtgcctgggtcttaggctctgtacaccatggaggagaagctcgctctaaaaataaccctgtccctggtggatccgacctccctggggacagcccctcctggctagtcacaccctgtaggctcctctatataacccaggggcacaggggctgcccccgggtcac

SEQ ID NO: 24 - DNA, 297 bp, Artificial Sequence
  MSEC-297I
ccactacgggtctaggctgcccatgtaaggaggcaaggcctggggacacccgagatgcctggttataattaacccatctagatggctgcccccccccccccaacacggtaaccctgagcctcacccccaccccggtgcctgggtcttaggctctgtacaccatggaggagaagctcgctctaaaaataaccctgtccctggtggatccgacctccctggggacagcccctcctggctagtcacaccctgtaggctcctctatataacccaggggcacaggggctgcccccgggtcac

SEQ ID NO: 25 - DNA, 297 bp, Artificial Sequence
  MSEC-297J
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctgggcccaaccctgtccctggtggatccgacctccctggggacagcccctcctggctagtcacaccctgtaggctcctctatataacccaggggcacaggggctgcccccgggtcac

SEQ ID NO: 26 - DNA, 297 bp, Artificial Sequence
  MSEC-297K
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctctagatctccctgtccctggtggatccgacctccctggggacagcccctcctggctagtcacaccctgtaggctcctctatataacccaggggcacaggggctgcccccgggtcac

SEQ ID NO: 27 - DNA, 297 bp, Artificial Sequence
  MSEC-297L
ccactacgggtctaggctgcccatgtaaggaggcaaggcctggggacacccgagatgcctggttataattaacccagacatgtggctgcccccccccccccaacacggtaaccctgagcctcacccccaccccggtgcctgggtcttaggctctgtacaccatggaggagaagctcgctctaaaaataaccctgtccctggtggatccgacctccctggggacagcccctcctggctagtcacaccctgtaggctcctctatataacccaggggcacaggggctgcccccgggtcac

SEQ ID NO: 28 - DNA, 302 bp, Artificial Sequence
  MSEC-302A
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccgacctccctggggacagcccctcctggctagtcacaccctgtaggctcctctatataacccaggggcacaggggctgcccccgggtcac

SEQ ID NO: 29 - DNA, 302 bp, Artificial Sequence
  MSEC-302B
ccactacgggtctaggctgcccatgtaaggaggcaaggcctggggacacagctgatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccgacctccctggggacagcccctcctggctagtcacaccctgtaggctcctctatataacccaggggcacaggggctgcccccgggtcac

SEQ ID NO: 30 - DNA, 302 bp, Artificial Sequence
  MSEC-302C
ccactacgggtctaggctgcccatgtaaggaggcaagatctagagacacagctgatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccgacctccctggggacagcccctcctggctagtcacaccctgtaggctcctctatataacccaggggcacaggggctgcccccgggtcac

SEQ ID NO: 31 - DNA, 303 bp, Artificial Sequence
  MSEC-303
gctgcccatgtaaggaggggacacccgagacctggttataattaacccagacatgtggctgccccccccccaacacctgctgcctctaaaaataaccctgcatgcgggccagctgtcccccgccagcactcagcacttagtgcccatacaaggccatggggctgggcgcctgggtccggggtgggtgcctctaaaaataacccaagggccagctgtcccccgtccctggggacagcccctcctggcatcacaccctaggctcctctatataacccaggggcacaggggctgcccccgggtcac

SEQ ID NO: 32 - DNA, 304 bp, Artificial Sequence
  MSEC-304
ctgctcccagctggccctcccagtctgctttatcaggattctcaagagggacagctggtttatgttgcatgactgttccctgcatatctgctctggttttaaatagcttatctgagcagctggaggaccacatgggcttatatggggcacctgccaaaatagcagccaacacccccccctgtcgcacattcctccctggctcaccaggccccagcccacatgcctgcttaaagccctctccatcctctgcctcacccagtccccgctgagactgagcagacgcctccaggatctgtcggcagct

SEQ ID NO: 33 - DNA, 310 bp, Artificial Sequence
  MSEC-310A
gatccaccagggacagggttatttttagagcgagcttctcctccatggtgtacagagcctaagacccaggcaccggggtgggggtgaggctcaggcagcaggtgttgggggggggggggcagccacatgtctgggttaattataaccaggcatctcgggtgtccccaggccttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 34 - DNA, 310 bp, Artificial Sequence
  MSEC-310B
gatccaccagggacagggttatttttagagcgagcttctcctccatggtgtacagagcctaagacccaggcaccggggtgggggtgaggctcaggcagcaggtgttgggggggggggggcagccacatgtctgggttaattataaccaggcatcagctgtgtctctagatcttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 35 - DNA, 310 bp, Artificial Sequence
  MSEC-310C
gatccaccagggacagggttatttttagagcgagcttctcctccatggtgtacagagcctaagacccaggcaccggggtgggggtgaggctcaggcagcaggtgttgggggggggggggcagccacatgtctgggttaattataaccaggcatctcgggtgtccccaggccttgcctcggtacctgcgcagcctagacccgtagtggggcatgcaagcttcgacctccctggggacagcccctcctggctagtcacaccctgtaggctcctctatataacccaggggcacaggggctgcccccgggtcac

SEQ ID NO: 36 - DNA, 310 bp, Artificial Sequence
  MSEC-310D
gatccaccagggacagggttatttttagagcgagcttctcctccatggtgtacagagcctaagacccaggcaccggggtgggggtgaggctcaggcagcaggtgttgggggggggggggcagccacatgtctgggttaattataaccaggcatctcgggtgtctctagatcttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 37 - DNA, 310 bp, Artificial Sequence
  MSEC-310E
gatccaccagggacagggttatttttagagcgagcttctcctccatggtgtacagagcctaagacccaggcaccggggtgggggtgaggctcaggcagcaggtgttgggggggggggggcagccacatgtctgggtgcatgctagccaggcatctcgggtgtccccaggccttgcctccttacatgggcagcctaaacccgtagtggggcatgcaagcttcgacctccctggggacagcccctcctggctagtcacaccctgtaggctcctctatataacccaggggcacaggggctgcccccgggtcac

SEQ ID NO: 38 - DNA, 310 bp, Artificial Sequence
  MSEC-310F
gatccaccagggacagggttatttttagagcgagcttctcctccatggtgtacagagcctaagacccaggcaccggggtgggggtgaggctcaggcagcaggtgttgggggggggggggcagccacatgtctgggagatctagaaccaggcatctcgggtgtccccaggccttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 39 - DNA, 310 bp, Artificial Sequence
  MSEC-310G
gatccaccagggacagggttatttttagagcgagcttctcctccatggtgtacagagcctaagacccaggcaccggggtgggggtgaggctcaggcagcaggtgttgggggggggggggcagccatctagatgggttaattataaccaggcatctcgggtgtccccaggccttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 40 - DNA, 310 bp, Artificial Sequence
  MSEC-310H
gatccaccagggacagggttatttttagagcgagcttctcctccatggtgtacagagcctaagacccaggcaccggggtgggggtgaggctcagggttaccgtgttgggggggggggggcagccacatgtctgggttaattataaccaggcatctcgggtgtccccaggccttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 41 - DNA, 310 bp, Artificial Sequence
  MSEC-310I
gatccaccagggacagggttatttttagagcgagcttctcctccatggtgtacagagcctaagacccaggcaccggggtgggggtgaggctcaggcagcagatcttgggggggggggggcagccacatgtctgggttaattataaccaggcatctcgggtgtccccaggccttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 42 - DNA, 310 bp, Artificial Sequence
  MSEC-310J
gatccaccagggacagggttatttttagagcgagcttctcctccatggtgtacagagcctaagacccaggcaccggggtgggggtgaggctcagggttaccgtgttgggggggggggggcagccatctagatgggttaattataaccaggcatctcgggtgtccccaggccttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 43 - DNA, 310 bp, Artificial Sequence
  MSEC-310K
gatccaccagggacagggttgggcccagagcgagcttctcctccatggtgtacagagcctaagacccaggcaccggggtgggggtgaggctcaggcagcaggtgttgggggggggggggcagccacatgtctgggttaattataaccaggcatctcgggtgtccccaggccttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 44 - DNA, 310 bp, Artificial Sequence
  MSEC-310L
gatccaccagggacagggagatctagagagcgagcttctcctccatggtgtacagagcctaagacccaggcaccggggtgggggtgaggctcaggcagcaggtgttgggggggggggggcagccacatgtctgggttaattataaccaggcatctcgggtgtccccaggccttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 45 - DNA, 315 bp, Artificial Sequence
  MSEC-315A
gatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatctcgggtgtccccaggccttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 46 - DNA, 315 bp, Artificial Sequence
  MSEC-315B
gatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatcagctgtgtccccaggccttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 47 - DNA, 315 bp, Artificial Sequence
  MSEC-315C
gatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatcagctgtgtctctaaatcttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 48 - skipped

SEQ ID NO: 49 - DNA, 319 bp, Artificial Sequence
  MSEC-319B
acacccaaatatggctcgcgctctaaaaataaccctgggacacccgagatgcctggttataattaacccacccaacacctgctgcccccgctccttctttggtcagtcgcacattcctccctgtcgcacattcctcccttgccgctctaaaaataaccctgaacacccaaatatggctcgctgcagccgctccttctttggtcaacacccaaatatggctcgacccgcgggctatataaaacctgagcagagggacaagcggccaccgcagcggacagcgccaagtgaagcctcgcttcccctccgcggcgaccagggc

SEQ ID NO: 50 - DNA, 320 bp, Artificial Sequence
  MSEC-320
ctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggggcacctgccaaaatagcagccaacacccccccctgtcgcacattcctccctggctcaccaggccccagcccacatgcctgcttaaagccctctccatcctctgcctcacccagtccccgctgagactgagcagacgcctccaggatctgtcggcagct

SEQ ID NO: 51 - DNA, 322 bp, Artificial Sequence
  MSEC-322
acacccaaatatggctcgcgctctaaaaataaccctgggacacccgagatgcctggttataattaacccacccaacacctgctgcccccgctccttctttggtcagtcgcacattcctccctgtcgcacattcctcccttgccgctctaaaaataaccctgaacacccaaatatggctcgctgcagccgctccttctttggtcaacacccaaatatggctcgacagcgacattcctgcggggcccgcgggctatataaaacctgagcagagggacaagcggccaccggacaagcctcgcttcccctccgcggcgaccagggc

SEQ ID NO: 52 - DNA, 327 bp, Artificial Sequence
  MSEC-327
caccattcctcacggccgacacccaaatatggcgacggccgtgaggaatggtgcggccgggagttatttttagagcggccgtgaggaatggtgggcaggcagcaggtgttggcggccgctctaaaaataactcccggccgggagttatttttagagcggccgtgaggaatggtggacacccaaatatggcgacggggacacccgagatgcctggttataattaacccaccgtcgccatatttgggtgtctgcagcattcctgggggccgggcggtgctcccgcccgcctcgataaaaggctccggggccggcggcggcccacgagct

SEQ ID NO: 53 - DNA, 330 bp, Artificial Sequence
  MSEC-330
ctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggggcacctgccaaaatagcagccaacacccccccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 54 - DNA, 335 bp, Artificial Sequence
  MSEC-335A
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatctcgactctaggggcaacgagctgaaagctcatctgctctcaggggcccctccctggggacagcccctcctggctagtcacaccctgtaggctcctctatataacccaggggcacaggggctgcccccg

SEQ ID NO: 55 - DNA, 335 bp, Artificial Sequence
  MSEC-335B
ccactacgggtctaggctgcccatgtaaggaggcaaggcctggggacacccgagatgcctggttataattaacccagacatgtggctgcccccccccccccaacacggtaaccctgagcctcacccccaccccggtgcctgggtcttaggctctgtacaccatggaggagaagctcgctctaaaaataaccctgtccctggtggatctcgactctaggggcaacgagctgaaagctcatctgctctcaggggcccctccctggggacagcccctcctggctagtcacaccctgtaggctcctctatataacccaggggcacaggggctgcccccg

SEQ ID NO: 56 - DNA, 336 bp, Artificial Sequence
  MSEC-336A
gctgcccatgtaaggaggggacacccgagacctggttataattaacccagacatgtggctgccccccccccaacacctgctgcctctaaaaataaccctgcatgcgggccagctgtcccccgccagctagactcagcacttagtcagcccttggggcagcccatacaaggccatggggctgggcgcctgggtccggggtgggcacggtgcctctaaaaataacccaagggccagctgtcccccggggcccctccctggggagggcccctcctggctagtcacaccctgtaggctcctctatataacccaggggcacaggggctgcccccgggtcac

SEQ ID NO: 57 - DNA, 336 bp, Artificial Sequence
  MSEC-336B
gctgcccatgtaaggaggggacacccgagacctggttataattaacccagacatgtggctgccccccccccaacacctgctgcctctaaaaataaccctgcatgcgggccagctgtcccccgccagctagactcagcacttagtcagcccttggggcagcccatacaaggccatggggctgggcgcctgggtccggggtgggcacggtgcctctaaaaataacccaagggccagctgtcccccggggcccctccctggggacagcccctcctggctagtcacaccctgtaggctcctctatataacccaggggcacaggggctgcccccgggtcac

SEQ ID NO: 58 - DNA, 336 bp, Artificial Sequence
  MSEC-336C
gctgcccatgtaaggaggggacacccgagacctggttataattaacccagacatgtggctgccccccccccaacacctgctgcatctaaaaatagccacgcatgcgggccagctgtcccccgccagctagactcagcacttagtcagcccttggggcagcccatacaaggccatggggctgggcgcctgggtccggggtgggcacggtgcatctaaaaatagccaaagggccagctgtcccccggggcccctccctggggacagcccctcctggctagtcacaccctgtaggctcctctatataacccaggggcacaggggctgcccccgggtcac

SEQ ID NO: 59 - DNA, 336 bp, Artificial Sequence
  MSEC-336D
gctgcccatgtaaggaggggacacccgagacctggttataattaacccagacatgtggctgccccccccccaacacctgctgcatctaaaaatagccacgcatgcgggccagctgtcccccgccagctagactcagcacttagtcagcccttggggcagcccatacaaggccatggggctgggcgcctgggtccggggtgggcacggtgcctctaaaaataacccaagggccagctgtcccccggggcccctccctggggacagcccctcctggctagtcacaccctgtaggctcctctatataacccaggggcacaggggctgcccccgggtcac

SEQ ID NO: 60 - DNA, 339 bp, Artificial Sequence
  MSEC-339
gcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacacccccccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 61 - DNA, 340 bp, Artificial Sequence
  MSEC-340
gctgcccttgtaaggaggggacacccgagacgcggttataattaaccaggacacgtggcgaccccccccccaacacctgccccctctaaaaataactgcagccgctccttctttggtcaacgcaggggacccgggcgggggcccaggccgcgaaccggccgagggagggggctctagtgcccaacacccaaatatggctcgagaagggcagcgacattcctgcggggtggcgcggagggaatcgcccgcgggctatataaaacctgagcagagggacaagcggccaccgcagcggacagcgccaagtgaagcctcgcttcccctccgcggcgaccagggc

SEQ ID NO: 62 - DNA, 341 bp, Artificial Sequence
  MSEC-341
acacccaaatatggctcgcgctctaaaaataaccctgggacacccgagatgcctggttataattaacccacccaacacctgctgcccccgctccttctttggtcagtcgcacattcctccctgtcgcacattcctcccttgccgctctaaaaataaccctgaacacccaaatatggctcgctgcagccgctccttctttggtcaacacccaaatatggctcgagaagggcagcgacattcctgcggggtggcgcggagggacccgcgggctatataaaacctgagcagagggacaagcggccaccggacaagcctcgcttcccctccgcggcgaccagggc

SEQ ID NO: 63 - DNA, 344 bp, Artificial Sequence
  MSEC-344
ctggttataattaaccccccaacacctgctgccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 64 - DNA, 346 bp, Artificial Sequence
  MSEC-346A
ctgctcccagctggccctcccagtctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacacccccccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 65 - skipped

SEQ ID NO: 66 - DNA, 347 bp, Artificial Sequence
  MSEC-347
ctgctcccagctggccctcccaggcctgggttgctggcctctgctttatcaggattctcaagagggacagctggtttatgttgcatatctgctctggttttaaatagcttatctgagcagctggaggaccacatgggcttatatggcgtggggtacatgttcctgtagccttgtccctggcacctgccaaaatagcagccaacacccccccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 67 - DNA, 348 bp, Artificial Sequence
  MSEC-348
ctgctcccagctggccctcccaggcctgggttgctggcctctgctttatcagacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacacccccccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 68 - DNA, 350 bp, Artificial Sequence
  MSEC-350
tctagaaaatctgaaaggcatagccccatatatcagtgatataaatagaacctgcagcaggctctggtaaatgatgactacaaggtggactgggaggcagcccggcgctagccgctccttctttggtcaacgcaggggacccgggcgggggcccaggccgcgaaccggccgagggagggggctctagtgcccaacacccaaatatggctcgagaagggcagcgacattcctgcggggtggcgcggagggaatcgcccgcgggctatataaaacctgagcagagggacaagcggccaccgcagcggacagcgccaagtgaagcctcgcttcccctccgcggcgaccagggc

SEQ ID NO: 69 - DNA, 351 bp, Artificial Sequence
  MSEC-351
gctgcccatgtaaggaggcaaggcctggggacacccgagatgcctggttataattaacccagacatgtggctgcccccccccccccaacacctgctgcctctaaaaataaccctgcatgcgggccagctgtcccccgccagctagactcagcacttagtcagcccttggggcagcccatacaaggccatggggctgggcgcctgggtccggggtgggcacggtgcatctaaaaatagccaaagggccagctgtcccccggggcccctccctggggacagcccctcctggctagtcacaccctgtaggctcctctatataacccaggggcacaggggctgcccccgggtcac

SEQ ID NO: 70 - DNA, 352 bp, Artificial Sequence
  MSEC-352A
ctgctcccagctggccctcccaggcctgggttgctggcctctgctttatcaggattctcaagagggacagctggtttatgttgcatgactgttccctgcatatctgctctggttttaaatagcttatctgagcagctggaggcttatatggcgtggggtacatgttcctgtagccttgtccctggcacctgccaaaatagcagccaacacccccccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 71 - DNA, 352 bp, Artificial Sequence
  MSEC-352B
ctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacacccccccctgtcgcacattcctccctggctcaccaggccccagcccacatgcctgcttaaagccctctccatcctctgcctcacccagtccccgctgagactgagcagacgcctccaggatctgtcggcagct

SEQ ID NO: 72 - DNA, 356 bp, Artificial Sequence
  MSEC-356
gctgcccatgtaaggaggctgcatatctgctctggacacccgagacctggttataattaacccagacatgtggctgccccccccccaacacctgctgcctctaaaaataaccctgcatgcaacacccaaatatggctcgagaagggccagctgtcccccgccagcactcagcacttagtctgcatatctgctctgcccatacaaggccatggggctgggcgcctgggtccggggtgggtgcctctaaaaataacccaagggccagctgtcccccgtccctggggacagcccctcctggcatcacaccctaggctcctctatataacccaggggcacaggggctgcccccgggtcac

SEQ ID NO: 73 - DNA, 360 bp, Artificial Sequence
  MSEC-360A
acacccaaatatggctcgcgctctaaaaataaccctgggacacccgagatgcctggttataattaacccacccaacacctgctgccgctcacaagtgttgcattcctctctgcccgctccttctttggtcacagagaggaatgcaacacttgtgagccagagaggaatgcaacacttgtgagccgctctaaaaataaccctgaacacccaaatatggctcgcatgcggcccctccctggggacagcccctcctggctagtcacaccctgtaggctcctctatataacccaggggcacaggggctgcccccgggtcaccaccacctccacagcacagacagacactcaggagccagccagc

SEQ ID NO: 74 - DNA, 360 bp, Artificial Sequence
  MSEC-360B
gagccatatttgggtgttcagggttatttttagagcggctcacaagtgttgcattcctctctggctcacaagtgttgcattcctctctgtgaccaaagaaggagcgggcagagaggaatgcaacacttgtgagcggcagcaggtgttgggtgggttaattataaccaggcatctcgggtgtcccagggttatttttagagcgcgagccatatttgggtgtgcatgcgccccctccctggggacagcccctcctggctagtcacaccctgtaggctcctctatataacccaggggcacaggggctgcccccgggtcaccaccacctccacagcacagacagacactcaggagccagccagc

SEQ ID NO: 75 - DNA, 361 bp, Artificial Sequence
  MSEC-361
acacccaaatatggctcgcgctctaaaaataaccctgggacacccgagatgcctggttataattaacccacccaacacctgctgcccccgctccttctttggtcagtcgcacattcctccctgtcgcacattcctcccttgccgctctaaaaataaccctgaacacccaaatatggctcgctgcagccgctccttctttggtcaacacccaaatatggctcgagaagggcagcgacattcctgcggggtggcgcggagggaatcgcccgcgggctatataaaacctgagcagagggacaagcggccaccgcagcggacagcgccaagtgaagcctcgcttcccctccgcggcgaccagggc

SEQ ID NO: 76 - DNA, 362 bp, Artificial Sequence
  MSEC-362A
ctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacacccccccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 77 - DNA, 362 bp, Artificial Sequence
  MSEC-362B
ctgctcccagctggccctcccaggcctgggttgctggcctctgcttcttaaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacacccccccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 78 - DNA, 362 bp, Artificial Sequence
  MSEC-362C
ctcgagctcactggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacacccccccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 79 - DNA, 362 bp, Artificial Sequence
  MSEC-362D
ctgctcccagctggccctcccaggcctgggttgctggcctctgctttatcaggattctcaagagggacagctggtttatgttcagtgaaaactctctgcatatctgctctggttttaaatagcttatctgagcagctggaggaccacatgggcttatatggcgtggggtacatgttcctgtagccttgtccctggcacctgccaaaatagcagccaacacccccccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 80 - DNA, 362 bp, Artificial Sequence
  MSEC-362E
ctagacccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacacccccccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 81 - DNA, 362 bp, Artificial Sequence
  MSEC-362F
ctgctcccagcgtgccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacacccccccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 82 - DNA, 365 bp, Artificial Sequence
  MSEC-365A
aatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 83 - DNA, 365 bp, Artificial Sequence
  MSEC-365B
aatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaagtctagggtgctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 84 - DNA, 367 bp, Artificial Sequence
  MSEC-367
acacccaaatatggctcgcgctctaaaaataaccctgggacacccgagatgcctggttataattaacccacccaacacctgctgcccccgctccttctttggtcagtcgcacattcctccctgtcgcacattcctcccttgccgctctaaaaataaccctgaacacccaaatatggctcgctgcagccgctccttctttggtcaacgcaggggacccgggcgggggcccaggccgcgaaccggccgagggagggggctctagtgcccaacacccaaatatggctcgacccgcgggctatataaaacctgagcagagggacaagcggccaccggacaagcctcgcttcccctccgcggcgaccagggc

SEQ ID NO: 85 - DNA, 378 bp, Artificial Sequence
  MSEC-378
gctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 86 - DNA, 382 bp, Artificial Sequence
  MSEC-382
gctgcccatgtaaggaggctgcatatctgctctggacacccgagacctggttataattaacccagacatgtggctgccccccccccaacacctgctgcctctaaaaataaccctgcatgcaacacccaaatatggctcgagaagggccagctgtcccccgccagctagactcagcacttagtctgcatatctgctctcagcccttggggcagcccatacaaggccatggggctgggcgcctgggtccggggtgggcacggtgcctctaaaaataacccaagggccagctgtcccccgtccctggggacagcccctcctggctagtcacaccctgtaggctcctctatataacccaggggcacaggggctgcccccgggtcac

SEQ ID NO: 87 - DNA, 383 bp, Artificial Sequence
  MSEC-383
acacccaaatatggctcgcgctctaaaaataaccctgggacacccgagatgcctggttataattaacccacccaacacctgctgcccccgctccttctttggtcagtcgcacattcctccctgtcgcacattcctcccttgccgctctaaaaataaccctgaacacccaaatatggctcgctgcagccgctccttctttggtcaacgcaggggacccgggcgggggcccaggccgcgaaccggccgagggagggggctctagtgcccaacacccaaatatggctcgacccgcgggctatataaaacctgagcagagggacaagcggccaccgcagcggacagcgccaagtgaagcctcgcttcccctccgcggcgaccagggc

SEQ ID NO: 88 - DNA, 384 bp, Artificial Sequence
  MSEC-384
gctgcccatgtaaggaggcctgcatatctgctctggacacccgagatgcctggttataattaacccagacatgtggctgcccccccccccccaacacctgctgcctctaaaaataaccctgcatgccatgttcccggcgaagggccagctgtcccccgccagctagactcagcacttagtctgcatatctgctctcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 89 - DNA, 386 bp, Artificial Sequence
  MSEC-386
ctggttataattaaccccccaacacctgctgccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccag

SEQ ID NO: 90 - DNA, 388 bp, Artificial Sequence
  MSEC-388
cagggttatttttagaggcagcaggtgttggggcagcaggtgttggggggttaattataaccaggcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 91 - DNA, 393 bp, Artificial Sequence
  MSEC-393A
acacccaaatatggctcgcgctctaaaaataaccctgggacacccgagatgcctggttataattaacccagacatgtggctgccccccccaacacctgctgccgctcacaagtgttgcattcctctctgcccgctccttctttggtcacagagaggaatgcaacacttgtgagccagagaggaatgcaacacttgtgagccgctctaaaaataaccctgaacacccaaatatggctcgggccagctgtcccccgcatgcggcccctccctggggacagcccctcctggctagtcacaccctgtaggctcctctatataacccaggggcacaggggctgcccccgggtcaccaccacctccacagcacagacagacactcaggagccagccagc

SEQ ID NO: 92 - DNA, 393 bp, Artificial Sequence
  MSEC-393B
acacccaaatatggctcgcgctctaaaaataaccctgggacacccgagatgcctggttataattaacccagacatgtggctgccccccccaacacctgctgccgctcacaagtgttgcattcctctctgcccgctccttctttggtcacagagaggaatgcaacacttgtgagccagagaggaatgcaacacttgtgagccgctctaaaaataaccctgaacacccaaatatggctcgggccagctgtcccccgcatgcggcccctccctggggagggcccctcctggctagtcacaccctgtaggctcctctatataacccaggggcacaggggctgcccccgggtcaccaccacctccacagcacagacagacactcaggagccagccagc

SEQ ID NO: 93 - DNA, 395 bp, Artificial Sequence
  MSEC-395
gctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 94 - DNA, 400 bp, Artificial Sequence
  MSEC-400
gctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccacca

SEQ ID NO: 95 - DNA, 402 bp, Artificial Sequence
  MSEC-402
ctgctcccagctggccctcccaggcctgggttgctggcctctgctttatcaggattctcaagagggacagctggtttatgttgcatgactgttccctgcatatctgctctggttttaaatagcttatctgagcacctgcatatctgctctggttttaaatagcttatctgagcagctggaggaccacatgggcttatatggcgtggggtacatgttcctgtagccttgtccctggcacctgccaaaatagcagccaacacccccccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 96 - DNA, 403 bp, Artificial Sequence
  MSEC-403
aggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 97 - DNA, 405 bp, Artificial Sequence
  MSEC-405A
acacccaaatatggctcgcgctctaaaaataaccctgggacacccgagatgcctggttataattaacccacccaacacctgctgcccccgctccttctttggtcagtcgcacattcctccctgtcgcacattcctcccttgccgctctaaaaataaccctgaacacccaaatatggctcgctgcagccgctccttctttggtcaacgcaggggacccgggcgggggcccaggccgcgaaccggccgagggagggggctctagtgcccaacacccaaatatggctcgagaagggcagcgacattcctgcggggtggcgcggagggacccgcgggctatataaaacctgagcagagggacaagcggccaccggacaagcctcgcttcccctccgcggcgaccagggc

SEQ ID NO: 98 - DNA, 405 bp, Artificial Sequence
  MSEC-405B
caaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagc

SEQ ID NO: 99 - DNA, 405 bp, Artificial Sequence
  MSEC-405C
gctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctcc

SEQ ID NO: 100 - DNA, 406 bp, Artificial Sequence
  MSEC-406
gctgcccatgtaaggaggcctgcatatctgctctggacacccgagatgcctggttataattaacccagacatgtggctgcccccccccccccaacacctgctgcctctaaaaataaccctgcatgccatgttcccaacacccaaatatggctcgagaaggcgaagggccagctgtcccccgccagctagactcagcacttagtctgcatatctgctctcagcccatacaaggccatggggctgggcaagctgcacgcctgggtccggggtgggcacggtgcctctaaaaataacccaacacctgctgctctcaggggcccctccctggggacagcccctcctggctagtcacaccctgtaggctcctctatataacccaggggcacaggggctgcccccgggtcac

SEQ ID NO: 101 - DNA, 410 bp, Artificial Sequence
  MSEC-410
ttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 102 - DNA, 411 bp, Artificial Sequence
  MSEC-411
gctgcccatgtaaggaggcctgcatatctgctctggacacccgagatgcctggttataattaacccagacatgtggctgcccccccccccccaacacctgctgcctctaaaaataaccctgcatgccatgttcccaacacccaaatatggctcgagaaggcgaagggccagctgtcccccgccagctagactcagcacttagtctgcatatctgctctcagcccatacaaggccatggggctgggcaagctgcacgcctgggtccggggtgggcacggtgcctctaaaaataacccaacacctgctgctctcaggggcccctccctggggacagcccctcctggctagtcacaccctgtaggctcctctatataacccaggggcacaggggctgccctcattctaccacca

SEQ ID NO: 103 - DNA, 413 bp, Artificial Sequence
  MSEC-413
gataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccatccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacc

SEQ ID NO: 104 - DNA, 417 bp, Artificial Sequence
  MSEC-417
ctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggggcacctgccaaaatagcagccaacacccccccctgtcgcacattcctccctggctcaccaggccccagcccacatgcctgcttaaagccctctccatcctctgcctcacccagtccccgc

SEQ ID NO: 105 - DNA, 420 bp, Artificial Sequence
  MSEC-420A
gctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccccagcccaacagaca

SEQ ID NO: 106 - DNA, 420 bp, Artificial Sequence
  MSEC-420B
gctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacaacggttg

SEQ ID NO: 107 - DNA, 421 bp, Artificial Sequence
  MSEC-421A
gctgcccatgtaaggaggcaagatctagagacacccgagatgcctggttataattaacccagacatgtggctgcccccccccccccaacacctgctgcctctaaaaataaccctgcatgccatgttcccggcgaagggccagctgtcccccgccagctagactcagcacttagtagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 108 - DNA, 421 bp, Artificial Sequence; identical to SEQ ID NO: 107
  MSEC-421B

SEQ ID NO: 109 - DNA, 421 bp, Artificial Sequence
  MSEC-421C
gctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagc

SEQ ID NO: 110 - DNA, 421 bp, Artificial Sequence
  MSEC-421D
gctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagaca

SEQ ID NO: 111 - DNA, 423 bp, Artificial Sequence
  MSEC-423
gctgcccatgtaaggaggggacacccgagacctggttataattaacccagacatgtggctgccccccccccaacacctgctgcatctaaaaatagccacgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 112 - DNA, 425 bp, Artificial Sequence
  MSEC-425
acacccaaatatggctcgcgctctaaaaataaccctgggacacccgagatgcctggttataattaacccacccaacacctgctgcccccgctccttctttggtcagtcgcacattcctccctgtcgcacattcctcccttgccgctctaaaaataaccctgaacacccaaatatggctcgctgcagccgctccttctttggtcaacgcaggggacccgggcgggggcccaggccgcgaaccggccgagggagggggctctagtgcccaacacccaaatatggctcgagaagggcagcgacattcctgcggggtggcgcggagggaatcgcccgcgggctatataaaacctgagcagagggacaagcggccaccgcagcggacagcgccaagtgaagcctcgcttcccctccgcggcgaccagggc

SEQ ID NO: 113 - DNA, 427 bp, Artificial Sequence
  MSEC-427A
gctgcccatgtaaggaggcctgcatatctgctctggacacccgagatgcctggttataattaacccagacatgtggctgcccccccccccccaacacctgctgcctctaaaaataaccctgcatgccatgttcccggcgaagggccagctgtcccccgccagctagactcagcacttagtctgcatatctgctctcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 114 - DNA, 427 bp, Artificial Sequence
  MSEC-427B
gctgcccatgtaaggaggcctgcatatctgctctggacacccgagatgcctggttataattaacccagacatgtggctgcccccccccccccaacacctgctgcctctaaaaataaccctgcatgccatgttcccggcgaagggccagctgtcccccgccagctagactcagcacttagtctgcatatctgctctcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagc

SEQ ID NO: 115 - DNA, 427 bp, Artificial Sequence
  MSEC-427C
gctgcccatgtaaggaggcctgcatatctgctctggacacccgagatgcctggttataattaacccagacatgtggctgcccccccccccccaacacctgctgcctctaaaaataaccctgcatgccatgttcccggcgaagggccagctgtcccccgccagctagactcagcacttagtagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 116 - DNA, 429 bp, Artificial Sequence
  MSEC-429A
acacccaaatatggctcgcgctctaaaaataaccctgggacacccgagatgcctggttataattaacccacccaacacctgctgccttgcattcctctctccgctccttctttggtcagagaggaatgcaacagagaggaatgcaacgctctaaaaataaccctgaacacccaaatatggctcgctgcagccgctccttctttggtcaacgcaggggacccgggcgggggcccaggccgcgaaccggccgagggagggggctctagtgcccaacacccaaatatggctcgagaagggcagcgacattcctgcggggtggcgcggagggaatcgcccgcgggctatataaaacctgagcagagggacaagcggccaccgcagcggacagcgccaagtgaagcctcgcttcccctccgcggcgaccagggc

SEQ ID NO: 117 - skipped

SEQ ID NO: 118 - DNA, 433 bp, Artificial Sequence
  MSEC-433
gctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcagtcgcgatcgaacactcgagccgagcagacgtgcctacggaccg

SEQ ID NO: 119 - DNA, 438 bp, Artificial Sequence
  MSEC-438A
gctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 120 - DNA, 438 bp, Artificial Sequence
  MSEC-438B
gctgcccatgtaaggaggcaaggcctggggacacccgagatgcctggttataattaacccagacatgtggctgcccccccccccccaacacctgctgcctctaaaaataaccctgcatgccatgttcccggcgaagggccagggtaaccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 121 - DNA, 438 bp, Artificial Sequence
  MSEC-438C
gctgcccatgtaaggaggcaaggcctggggacacccgagatgcctggttataattaacccatctagatggctgcccccccccccccaacacggtaaccctctaaaaataaccctgcatgccatgttcccggcgaagggccagggtaaccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 122 - DNA, 438 bp, Artificial Sequence
  MSEC-438D
gctgcccatgtaaggaggcaagatctagagacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 123 - DNA, 438 bp, Artificial Sequence
  MSEC-438E
gctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagc

SEQ ID NO: 124 - DNA, 438 bp, Artificial Sequence
  MSEC-438F
gctgcccatgtaaggaggcaaggcctggggacacccgagatgcctggttataattaacccatctagatggctgcccccccccccccaacacggtaaccctctaaaaataaccctgcatgccatgttcccggcgaagggccagggtaaccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcagggtaactcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 125 - DNA, 438 bp, Artificial Sequence
  MSEC-438G
gctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaagagcacaggggctgccctcattctaccaccacctccacagcacagacagacactcaggagccagccagc

SEQ ID NO: 126 - DNA, 438 bp, Artificial Sequence
  MSEC-438H
gctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacgtaggggcacaggggctgccctcattctaccaccacctccacagcacagacagacactcaggagccagccagc

SEQ ID NO: 127 - DNA, 439 bp, Artificial Sequence
  MSEC-439A
ctgctcccagctggccctcccaggcctgggttgctggcccctgggttgctggcctctgctttatcaggatcctgggttgctggcctctgctttatcaggatcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacacccccccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 128 - DNA, 439 bp, Artificial Sequence
  MSEC-439B
gctgcccatgtaaggaggggacacccgagacctggttataattaacccagacatgtggctgccccccccccaacacctgctgcatctaaaaatagccacgcatgctgcccatgtaaggaggggacacccgagacctggttataattaacccagacatgtggctgccccccccccaacacctgctgcatctaaaaatagccacgcatgcgggccagctgtcccccgccagctagactcagcacttagtcagcccttggggcagcccatacaaggccatggggctgggcgcctgggtccggggtgggcacggtgcatctaaaaatagccaaagggccagctgtcccccggggcccctccctggggacagcccctcctggctagtcacaccctgtaggctcctctatataacccaggggcacaggggctgcccccgggtcac

SEQ ID NO: 129 - DNA, 440 bp, Artificial Sequence
  MSEC-440
gctgcccttgtaaggaggggacacccgagacgcggttataattaaccaggacacgtggcgaccccccccccaacacctgccccctctaaaaataactgcagctgcccttgtaaggaggggacacccgagacgcggttataattaaccaggacacgtggcgaccccccccccaacacctgccccctctaaaaataactgcagccgctccttctttggtcaacgcaggggacccgggcgggggcccaggccgcgaaccggccgagggagggggctctagtgcccaacacccaaatatggctcgagaagggcagcgacattcctgcggggtggcgcggagggaatcgcccgcgggctatataaaacctgagcagagggacaagcggccaccgcagcggacagcgccaagtgaagcctcgcttcccctccgcggcgaccagggc

SEQ ID NO: 130 - DNA, 443 bp, Artificial Sequence
  MSEC-443
ccctgtcaaattatttataggagatctaagctagcatgctgcccatgtaaggaggcctgcatatctgctctggacacccgagatgcctggttataattaacccagacatgtggctgcccccccccccccaacacctgctgcctctaaaaataaccctgcatgccatgttcccaacacccaaatatggctcgagaaggcgaagggccagctgtcccccgccagctagactcagcacttagtctgcatatctgctctcagcccatacaaggccatggggctgggcaagctgcacgcctgggtccggggtgggcacggtgcctctaaaaataacccaacacctgctgctctcaggggcccctccctggggacagcccctcctggctagtcacaccctgtaggctcctctatataacccaggggcacaggggctgcccccgggtcac

SEQ ID NO: 131 - DNA, 444 bp, Artificial Sequence
  MSEC-444
gctgcccatgtaaggaggcctgcatatctgctctggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 132 - DNA, 446 bp, Artificial Sequence
  MSEC-446
gctgcccttgtaaggaggggacacccgagacgcggttataattaaccaggacacgtggcgaccccccccccaacacctgccccctctaaaaataactgcagccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggccctctaaaaataactccttcagagaggaatgcaacacttgtgagcctcctccctggggacaacccctcccagccaatagcacagcctaggtccccctatataaggccacggctgctggcccttccttcattctagtgtcacctccaggatacagacagccccccttcagcccagcccagccag

SEQ ID NO: 133 - DNA, 449 bp, Artificial Sequence
  MSEC-449
gctgcccatgtaaggaggcctgcatatctgctctggacacccgagatgcctggttataattaacccagacatgtggctgcccccccccccccaacacctgctgcctctaaaaataaccctgcatgccatgttcccaacacccaaatatggctcgagaaggcgaagggccagctgtcccccgccagctagactcagcacttagtctgcatatctgctctcagcccatacaaggccatggggctgggcaagctgcacgcctgggtccggggtgggcacggtgcctctaaaaataacccaacacctgctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 134 - DNA, 450 bp, Artificial Sequence
  MSEC-450A
gctgcccatgtaaggaggcctgcatatctgctctggacacccgagatgcctggttataattaacccagacatgtggctgcccccccccccccaacacctgctgcctctaaaaataaccctgcatgccatgttcccaacacccaaatatggctcgagaaggcgaagggccagctgtcccccgccagctagactcagcacttagtctgcatatctgctctcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 135 - DNA, 450 bp, Artificial Sequence
  MSEC-450B
gctgcccatgtaaggaggcctgcatatctgctctggacacccgagatgcctggttataattaacccagacatgtggctgcccccccccccccaacacctgctgcctctaaaaataaccctgcatgccatgttcccaacacccaaatatggctcgagaaggcgaagggccagctgtcccccgccagctagactcagcacttagtctgcatatctgctctcagcccatacaaggccatggggctgggcaagctgcacgcctgggtccggggtgggcacggtgcctctaaaaataacccaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 136 - DNA, 450 bp, Artificial Sequence
  MSEC-450C
gctgcccatgtaaggaggcctgcatatctgctctggacacccgagatgcctggttataattaacccagacatgtggctgcccccccccccccaacacctgctgcctctaaaaataaccctgcatgccatgttcccaacacccaaatatggctcgagaaggcgaagggccagctgtcccccgccagctagactcagcacttagtctgcatatctgctctcagcccatacaaggccatggggctgggcaagctgcacgcctgggtccggggtgggcacggtgcatctaaaaatagccaaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 137 - DNA, 450 bp, Artificial Sequence
  MSEC-450D
gctgcccatgtaaggaggcctgcatatctgctctggacacccgagatgcctggttataattaacccagacatgtggctgcccccccccccccaacacctgctgcctctaaaaataaccctgcatgccatgttcccaacacccaaatatggctcgagaaggcgaagggccagctgtcccccgccagctagactcagcacttagtctgcatatctgctctcagcccatacaaggccatggggctgggcaagctgcacgcctgggtccggggtgggcacggtgcctctaaaaataacccaagggccagctgtcccccggggcccctccctggggacagcccctcctggctagtcacaccctgtaggctcctctatataacccaggggcacaggggctgccctcattctaccaccacctccacagcacagacagacactcaggagccagccagc

SEQ ID NO: 138 - DNA, 455 bp, Artificial Sequence
  MSEC-455A
ctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggggcacctgccaaaatagcagccaacacccccccctgtcgcacattcctccctggctcaccaggccccagcccacatgcctgcttaaagccctctccatcctctgcctcacccagtccccgctgagactgagcagacgcctccaggatctgtcggcagct

SEQ ID NO: 139 - DNA, 455 bp, Artificial Sequence
  MSEC-455B
ctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggggcacctgccaaaatagcagccaacacccccagagagaaatgcaacacttgtggctcaccaggccccagcccacatgcctgcttaaagccctctccatcctctgcctcacccagtccccgctgagactgagcagacgcctccaggatctgtcggcagct

SEQ ID NO: 140 - DNA, 455 bp, Artificial Sequence
  MSEC-455C
ctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggggcacctgccaaaatagcagccaacacccccagggaggaatgtgcgacagggggctcaccaggccccagcccacatgcctgcttaaagccctctccatcctctgcctcacccagtccccgctgagactgagcagacgcctccaggatctgtcggcagct

SEQ ID NO: 141 - DNA, 457 bp, Artificial Sequence
  MSEC-457
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcgaagatcttcgaacatgcccaatcaaggctgtgggggactgagggcaggctgtaacaggcttgggggccagggcttatacgtgcctgggactcccaaagtattactgttcgtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 142 - DNA, 461 bp, Artificial Sequence
  MSEC-461
cagataagctatttaaaaccagagcagatatgcagggaacagtcatgcaacataaaccagctgtccctcttgagaatcctgataaagcagaggccagcaacccaggcctgggagggccagctgggagcaggctagcagataagctatttaaaaccagagcagatatgcagggaacagtcatgcaacataaaccagctgtccctcttgagaatcctgataaagcagaggccagcaacccaggcctgggagggccagctgggagcaggagctcagcagctggaggaccacatgggcttatatggggcacctgccaaaatagcagccaacacccccccctgtcgcacattcctccctggctcaccaggccccagcccacatgcctgcttaaagccctctccatcctctgcctcacccagtccccgctgagactgagcagacgcctccaggatctgtcggcagct

SEQ ID NO: 143 - DNA, 462 bp, Artificial Sequence
  MSEC-462A
agttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacacccccccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 144 - DNA, 462 bp, Artificial Sequence
  MSEC-462B
ctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgagcagctggaggttatcagaccacatgggcttatatggggcacctgccaaaatagcagccaacacccccccctgtcgcacattcctccctggctcaccaggccccagcccacatgcctgcttaaagccctctccatcctctgcctcacccagtccccgctgagactgagcagacgcctccaggatctgtcggcagct

SEQ ID NO: 145 - DNA, 463 bp, Artificial Sequence
  MSEC-463
aaggagttatttttagagggggcaggtgttggaggggggttcgccacgtgtcctggttaattataaccgggcgtctcgggtgtcctcgggcctcgcctccttacaagggcagctgcagccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggccctctaaaaataactccttcagagaggaatgcaacacttgtgagcctcctccctggggacaacccctcccagccaatagcacagcctaggtccccctatataaggccacggctgctggcccttccttcattctagtgtcacctccaggatacagacagccccccttcagcccagcccagccag

SEQ ID NO: 146 - DNA, 464 bp, Artificial Sequence
  MSEC-464A
gctgcccttgtaaggaggcgaggcccgaggacacccgagacgcccggttataattaaccaggacacgtggcgaacccccctccaacacctgccccctctaaaaataactccttctgcagccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgccggagcagggaggtgagaggctcagctgccctccagaactcctccctggggacaacccctcccagccaatagcacagcctaggtccccctatataaggccacggctgctggcccttccttcattctagtgtcacctccaggatacagacagccccccttcagcccagcccagccag

SEQ ID NO: 147 - DNA, 464 bp, Artificial Sequence
  MSEC-464B
gctgcccttgtaaggaggcgaggcccgaggacacccgagacgcccggttataattaaccaggacacgtggcgaacccccctccaacacctgccccctctaaaaataactccttctgcagccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggccctctaaaaataactccttcagagaggaatgcaacacttgtgagcctcctccctggggacaacccctcccagccaatagcacagcctaggtccccctatataaggccacggctgctggcccttccttcattctagtgtcacctccaggatacagacagccccccttcagcccagcccagccag

SEQ ID NO: 148 - DNA, 466 bp, Artificial Sequence
  MSEC-466
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcccaatcaaggctgtgggggactgagggcaggctgtaacaggcttgggggccagggcttatacgcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 149 - DNA, 472 bp, Artificial Sequence
  MSEC-472
ctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgagcagctggaggttatcagaccacatgggcttatatggggtttatcaggacacctgccaaaatagcagccaacacccccccctgtcgcacattcctccctggctcaccaggccccagcccacatgcctgcttaaagccctctccatcctctgcctcacccagtccccgctgagactgagcagacgcctccaggatctgtcggcagct

SEQ ID NO: 150 - DNA, 474 bp, Artificial Sequence
  MSEC-474
cagggttatttttagaggcagcaggtgttggggcagcaggtgttggggggttaattataaccaggcatgcaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggccccctccctggggacagcccctcctggctagtcacaccctgtaggctcctctatataacccaggggcacaggggctgccctcattctaccaccacctccacagcacagacagacactcaggagccagccagc

SEQ ID NO: 151 - DNA, 476 bp, Artificial Sequence
  MSEC-476
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagccccctccctggggacagcccctcctggctagtcacaccctgtaggctcctctatataacccaggggcacaggggctgcccccgggtcac

SEQ ID NO: 152 - DNA, 478 bp, Artificial Sequence
  MSEC-478A
caccattcctcacggccgacacccaaatatggcgacggccgtgaggaatggtgcggccgggagttatttttagagcggccgtgaggaatggtgggcaggcagcaggtgttggcggccgctctaaaaataactcccggccgggagttatttttagagcggccgtgaggaatggtggacacccaaatatggcgacggccgcaccattcctcacggccgtcgccatatttgggtgtctgcagccgctccttctttggtcaacgcaggggacccgggcgggggcccaggccgcgaaccggccgagggagggggctctagtgcccaacacccaaatatggctcgagaagggcagcgacattcctgcggggtggcgcggagggaatcgcccgcgggctatataaaacctgagcagagggacaagcggccaccgcagcggacagcgccaagtgaagcctcgcttcccctccgcggcgaccagggc

SEQ ID NO: 153 - DNA, 478 bp, Artificial Sequence
  MSEC-478B
ctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggggcacctgccaaaatagcagccaacacccccagagaggaatgcaacacttgtgcagagaggaatgcaacacttgtggctcaccaggccccagcccacatgcctgcttaaagccctctccatcctctgcctcacccagtccccgctgagactgagcagacgcctccaggatctgtcggcagct

SEQ ID NO: 154 - DNA, 478 bp, Artificial Sequence
  MSEC-478C
ctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggggcacctgccaaaatagcagccaacacccccagggaggaatgtgcgacaggggcagggaggaatgtgcgacagggggctcaccaggccccagcccacatgcctgcttaaagccctctccatcctctgcctcacccagtccccgctgagactgagcagacgcctccaggatctgtcggcagct

SEQ ID NO: 155 - DNA, 480 bp, Artificial Sequence
  MSEC-480
agttcaagtggagcagcacataactcttgccctctgccttccaagattctggtgctgagacttatggagtgtcttggaggttgccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 156 - DNA, 486 bp, Artificial Sequence
  MSEC-486A
ccgagatgcctggttataattaaccccaacacctgctgcccccccccccccaacacctgctgcctgagcctcacccccaccccggtgcctgggtcttaggctctgtacaccatgtcgacccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 157 - DNA, 486 bp, Artificial Sequence
  MSEC-486B
catggtgtacagagcctaagacccaggcaccggggtgggggtgaggctcaggcagcaggtgttgggggggggggggcagcaggtgttggggttaattataaccaggcatctcggtcgacccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 158 - DNA, 486 bp, Artificial Sequence
  MSEC-486C
ccctgtcaaattatttataggagatctaagctagcatgctgcccatgtaaggaggcctgcatatctgctctggacacccgagatgcctggttataattaacccagacatgtggctgcccccccccccccaacacctgctgcctctaaaaataaccctgcatgccatgttcccaacacccaaatatggctcgagaaggcgaagggccagctgtcccccgccagctagactcagcacttagtctgcatatctgctctcagcccatacaaggccatggggctgggcaagctgcacgcctgggtccggggtgggcacggtgcctctaaaaataacccaacacctgctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 159 - DNA, 487 bp, Artificial Sequence
  MSEC-487A
ccgagatgcctggttataattaacccagacatgtggctgcccccccccccccaacacctgctgcctgagcctcacccccaccccggtgcctgggtcttaggctctgtacaccatgtcgacccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 160 - DNA, 487 bp, Artificial Sequence
  MSEC-487B
catggtgtacagagcctaagacccaggcaccggggtgggggtgaggctcaggcagcaggtgttgggggggggggggcagccacatgtctgggttaattataaccaggcatctcggtcgacccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 161 - DNA, 493 bp, Artificial Sequence
  MSEC-493A
ccgagatgcctggttataattaacccagacatgtggctgcccccccccccccaacacctgctgcctgagcctgagcggttaccccaccccggtgcctgggtcttaggctctgtacaccatgtcgacccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 162 - DNA, 493 bp, Artificial Sequence
  MSEC-493B
catggtgtacagagcctaagacccaggcaccggggtgggggtaaccgctcaggctcaggcagcaggtgttgggggggggggggcagccacatgtctgggttaattataaccaggcatctcggtcgacccaatcaaggctgtgggggactgagggcaggctgtaacaggcttggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 163 - DNA, 495 bp, Artificial Sequence
  MSEC-495
agttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagct

SEQ ID NO: 164 - DNA, 501 bp, Artificial Sequence
  MSEC-501
ctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggggcacctgccaaaatagcagccaacacccccagagaggaatgcaacacttgtgcagagaggaatgcaacacttgtgcagagaggaatgcaacacttgtggctcaccaggccccagcccacatgcctgcttaaagccctctccatcctctgcctcacccagtccccgctgagactgagcagacgcctccaggatctgtcggcagct

SEQ ID NO: 165 - DNA, 503 bp, Artificial Sequence
  MSEC-503
acacccaaatatggctcgcgctctaaaaataaccctgggacacccgagatgcctggttataattaacccacccaacacctgctgcccccgctccttctttggtcagtcgcacattcctccctgtcgcacattcctcccttgccgctctaaaaataaccctgaacacccaaatatggctcgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 166 - skipped

SEQ ID NO: 167 - DNA, 510 bp, Artificial Sequence
  MSEC-510
gctgcccatgtaaggaggcaaggcctggggacacccgagatgcctggttataattaacccagacatgtggctgcccccccccccccaacacctgctgcctctaaaaataaccctgcatgcgaagatcttcgaacatgcccgcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagc

SEQ ID NO: 168 - DNA, 513 bp, Artificial Sequence
  MSEC-513
gctgcccatgtaaggaggcaaggcctggggacacccgagatgcctggttataattaacccagacatgtggctgcccccccccccccaacacctgctgcctctaaaaataaccctgcatgcgaagatcttcgaacaaggctgtgggggactgagggcaggctgtaacaggcttgggggccagggcttatacgtgcctgggactccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagc

SEQ ID NO: 169 - DNA, 517 bp, Artificial Sequence
  MSEC-517
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcagcttgcatgccccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatctcgacctccctggggacagcccctcctggctagtcacaccctgtaggctcctctatataacccaggggcacaggggctgcccccgggtcac

SEQ ID NO: 170 - DNA, 518 bp, Artificial Sequence
  MSEC-518
cccatgagatgacagactataatagccacaggattaacatagcaggcattgtctttctctgactatagggtgggtattatgtgttcatcaaccatcctaaaaatacccggtaaacaggtgcagcccgagctcctggttataattaaccccccaacacctgctgccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccag

SEQ ID NO: 171 - DNA, 523 bp, Artificial Sequence
  MSEC-523
gctgcccatgtaaggaggcaaggcctggggacacccgagatgcctggttataattaacccagacatgtggctgcccccccccccccaacacctgctgcctctaaaaataaccctgcatgcaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 172 - DNA, 527 bp, Artificial Sequence
  MSEC-527A
ccactacgggtctaggctgcccatgtaaggaggcaaggcctggggacacagctgatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatcagcttgcatgccccactacgggtctaggctgcccatgtaaggaggcaaggcctggggacacagctgatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatctcgacctccctggggacagcccctcctggctagtcacaccctgtaggctcctctatataacccaggggcacaggggctgcccccgggtcac

SEQ ID NO: 173 - DNA, 527 bp, Artificial Sequence
  MSEC-527B
ccactacgggtctaggctgcccatgtaaggaggcaagatctagagacacagctgatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatcagcttgcatgccccactacgggtctaggctgcccatgtaaggaggcaagatctagagacacagctgatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatctcgacctccctggggacagcccctcctggctagtcacaccctgtaggctcctctatataacccaggggcacaggggctgcccccgggtcac

SEQ ID NO: 174 - DNA, 529 bp, Artificial Sequence
  MSEC-529A
ctggttataattaaccccccaacacctgctgccccaacacctgctgcctctaaaaataaccctgcatgcctggttataattaaccccccaacacctgctgccccaacacctgctgcctctaaaaataaccctgcatgcatgcctggttataattaaccccccaacacctgctgccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 175 - DNA, 529 bp, Artificial Sequence
  MSEC-529B
gatccaccagggacagggttatttttagagcgagcttctcctccatggtgtacagagcctaagacccaggcaccggggtgggggtgaggctcaggcagcaggtgttgggggggggggggcagccacatgtctgggttaattataaccaggcatctcgggtgtccccaggccttgcctccttacatgggcagcctagacccgtagtggggcatgcaagctgatccaccagggacagggttatttttagagcgagcttctcctccatggtgtacagagcctaagacccaggcaccggggtgggggtgaggctcaggcagcaggtgttgggggggggggggcagccacatgtctgggttaattataaccaggcatctcgggtgtccccaggccttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 176 - DNA, 529 bp, Artificial Sequence
  MSEC-529C
ccactacgggtctaggctgcccatgtaaggaggcaaggcctggggacacccgagatgcctggttataattaaccccaacacctgctgcccccccccccccaacacctgctgcctgagcctgagcggttacccccaccccggtgcctgggtcttaggctctgtacaccatggaggagaagctcgctctaaaaataaccctgtccctggtggatcagcttgcatgccccactacgggtctaggctgcccatgtaaggaggcaaggcctggggacacccgagatgcctggttataattaaccccaacacctgctgcccccccccccccaacacctgctgcctgagcctgagcggttacccccaccccggtgcctgggtcttaggctctgtacaccatggaggagaagctcgctctaaaaataaccctgtccctggtggatctcgacctccctggggacagcccctcctggctagtcacaccctgtaggctcctctatataacccaggggcacaggggctgcccccgggtcac

SEQ ID NO: 177 - DNA, 531 bp, Artificial Sequence
  MSEC-531A
acacccaaatatggctcgcgctctaaaaataaccctgggacacccgagatgcctggttataattaacccacccaacacctgctgcccccgctccttctttggtcagtcgcacattcctccctgtcgcacattcctcccttgccgctctaaaaataaccctgaacacccaaatatggctcgctgcagccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggccctctaaaaataactccttcagagaggaatgcaacacttgtgagcctcctccctggggacaacccctcccagccaatagcacagcctaggtccccctatataaggccacggctgctggcccttccttcattctagtgtcacctccaggatacagacagccccccttcagcccagcccagccag

SEQ ID NO: 178 - DNA, 531 bp, Artificial Sequence
  MSEC-531B
gctgcccatgtaaggaggcaaggcctggggacacccgagatgcctggttataattaacccagacatgtggctgcccccccccccccaacacctgctgcctctaaaaataaccctgcatgcgaagatcttcgaacatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagc

SEQ ID NO: 179 - DNA, 538 bp, Artificial Sequence
  MSEC-538A
gctgcccatgtaaggaggcaaggcctggggacacccgagatgcctggttataattaacccagacatgtggctgcccccccccccccaacacctgctgcctctaaaaataaccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 180 - DNA, 538 bp, Artificial Sequence
  MSEC-538B
ccactacgggtctaggctgcccatgtaaggaggcaaggcctggggacacccgagatgcctggttataattaacccagacatgtggctgcccccccccccccaacacctgctgcctctaaaaataaccctgcatgcaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 181 - DNA, 539 bp, Artificial Sequence
  MSEC-539A
gatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatcagctgtgtccccaggccttgcctccttacatgggcagcctagacccgtagtggggcatgcaagctgatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatcagctgtgtccccaggccttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 182 - DNA, 539 bp, Artificial Sequence
  MSEC-539B
gatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatcagctgtgtctctaaatcttgcctccttacatgggcagcctagacccgtagtggggcatgcaagctgatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatcagctgtgtctctaaatcttgcctccttacatgggcagcctagacccgtagtggggcatgcaagcttcgacctccctggggacagcccctcctggctagtcacaccctgtaggctcctctatataacccaggggcacaggggctgcccccgggtcac

SEQ ID NO: 183 - DNA, 540 bp, Artificial Sequence
  MSEC-540
gatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatctcgggtgtccccaggccttgcctccttacatgggcagcctagacccgtagtggggcatgcaagctgatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatctcgggtgtccccaggccttgcctccttacatgggcagcctagacccgtagtggtggcatgcaagcttcgacctccctggggacagcccctcctggctagtcacaccctgtaggctcctctatataacccaggggcacaggggctgcccccgggtcac

SEQ ID NO: 184 - DNA, 483 bp, Artificial Sequence
  MSEC-541A
cccatgagatgacagactataatagccacaggattaacatagcaggcattgtctttctctgactatagggtgggtattatgtgttcatcaaccatcctaaaaatacccggtaaacaggtgcagcccgagctctgcccatgtaaggaggcctgcatatctgctctggacacccgagatgcctggttataattaacccagacatgtggctgcccccccccccccaacacctgctgcctctaaaaataaccctgcatgccatgttcccaacacccaaatatggctcgagaaggcgaagggccagctgtcccccgccagctagactcagcacttagtctgcatatctgctctcagcccatacaaggccatggggctgggcaagctgcacgcctgggtccggggtgggcacggtgcctctaaaaataacccaacacctgctgctctcaggggcccctccctggggacagcccctcctggctagtcaca

SEQ ID NO: 185 - DNA, 541 bp, Artificial Sequence
  MSEC-541B
gctgcccatgtaaggaggcaaggcctggggacacccgagatgcctggttataattaacccagacatgtggctgcccccccccccccaacacctgctgcctctaaaaataaccctgtccctggtggatcccctgcatgcaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 186 - DNA, 546 bp, Artificial Sequence
  MSEC-546
gctgcccttgtaaggaggggacacccgagacgcggttataattaaccaggacacgtggcgaccccccccccaacacctgccccctctaaaaataactgcagctgcccttgtaaggaggggacacccgagacgcggttataattaaccaggacacgtggcgaccccccccccaacacctgccccctctaaaaataactgcagccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggccctctaaaaataactccttcagagaggaatgcaacacttgtgagcctcctccctggggacaacccctcccagccaatagcacagcctaggtccccctatataaggccacggctgctggcccttccttcattctagtgtcacctccaggatacagacagccccccttcagcccagcccagccag

SEQ ID NO: 187 - DNA, 547 bp, Artificial Sequence
  MSEC-547
gttatttttagagggggcaggtgttggggggggggtcgccacgtgtcctggttaattataaccgcgtctcgggtgtcccctccttacaagggcagctgcaggttatttttagagggggcaggtgttggggggggggtcgccacgtgtcctggttaattataaccgcgtctcgggtgtcccctccttacaagggcagctgcagccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggccctctaaaaataactccttcagagaggaatgcaacacttgtgagcctcctccctggggacaacccctcccagccaatagcacagcctaggtccccctatataaggccacggctgctggcccttccttcattctagtgtcacctccaggatacagacagccccccttcagcccagcccagccag

SEQ ID NO: 188 - DNA, 550 bp, Artificial Sequence
  MSEC-550
ggggctgcacctgtttaccgggtatttttaggatggttgatgaacacataatacccaccctatagtcagagaaagacaatgcctgctatgttaatcctgtggctattatagtctgtcatctcatgggttggggcaggacactgaccctctcagaggcccagatcatcagatcagcttgcatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 189 - DNA, 553 bp, Artificial Sequence
  MSEC-553
ccactacgggtctaggctgcccatgtaaggaggcaaggcctggggacacccgagatgcctggttataattaacccagacatgtggctgcccccccccccccaacacctgctgcctctaaaaataaccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 190 - DNA, 555 bp, Artificial Sequence
  MSEC-555
ggcctctgagagggtcagtgtcctgccccaacccatgagatgacagactataatagccacaggattaacatagcaggcattgtctttctctgactatagggtgggtattatgtgttcatcaaccatcctaaaaatacccggtaaacaggtgcagccccagatcatcatcgataagctagcttgcatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 191 - DNA, 556 bp, Artificial Sequence
  MSEC-556A
gctgcccatgtaaggaggcaaggcctggggacacccgagatgcctggttataattaacccagacatgtggctgcccccccccccccaacacctgctgcctctaaaaataaccctgcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 192 - DNA, 556 bp, Artificial Sequence
  MSEC-556B
gctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 193 - DNA, 556 bp, Artificial Sequence
  MSEC-556C
ccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 194 - DNA, 563 bp, Artificial Sequence
  MSEC-563
cccatgagatgacagactataatagccacaggattaacatagcaggcattgtctttctctgactatagggtgggtattatgtgttcatcaaccatcctaaaaatacccggtaaacaggtgcagcccgagctcttacgcgtgctagctagcatgctgcccatgtaaggaggcctgcatatctgctctggacacccgagatgcctggttataattaacccagacatgtggctgcccccccccccccaacacctgctgcctctaaaaataaccctgcatgccatgttcccaacacccaaatatggctcgagaaggcgaagggccagctgtcccccgccagctagactcagcacttagtctgcatatctgctctcagcccatacaaggccatggggctgggcaagctgcacgcctgggtccggggtgggcacggtgcctctaaaaataacccaacacctgctgctctcaggggcccctccctggggacagcccctcctggctagtcacaccctgtaggctcctctatataacccaggggcacaggggctgccctcattctaccacca

SEQ ID NO: 195 - DNA, 566 bp, Artificial Sequence
  MSEC-566
ccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcagtcgcgatcgaacactcgagccgagcagacgtgcctacggaccg

SEQ ID NO: 196 - DNA, 569 bp, Artificial Sequence
  MSEC-569
gctgcccatgtaaggaggcaaggcctggggacacccgagatgcctggttataattaacccagacatgtggctgcccccccccccccaacacctgctgcctctaaaaataaccctgctgcagcccacaagatgactgactataatagccactggattaacatagcaggcattgtctttctctgactatagggtgggtattatgtgttcatcaacagccctaaaaatactcaggaaacaggtgtggctcgagccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 197 - DNA, 571 bp, Artificial Sequence
  MSEC-571A
ccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 198 - DNA, 571 bp, Artificial Sequence
  MSEC-571B
ccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagc

SEQ ID NO: 199 - DNA, 577 bp, Artificial Sequence
  MSEC-577
acacccaaatatggctcgcgctctaaaaataaccctgggacacccgagatgcctggttataattaacccagacatgtggctgccccccccaacacctgctgccgctcacaagtgttgcattcctctctgcccgctccttctttggtcacagagaggaatgcaacacttgtgagccagagaggaatgcaacacttgtgagccgctctaaaaataaccctgaacacccaaatatggctcgggccagctgtcccccgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 200 - DNA, 581 bp, Artificial Sequence
  MSEC-581
aaggagttatttttagagggggcaggtgttggaggggggttcgccacgtgtcctggttaattataaccgggcgtctcgggtgtcctcgggcctcgcctccttacaagggcagctgcagaaggagttatttttagagggggcaggtgttggaggggggttcgccacgtgtcctggttaattataaccgggcgtctcgggtgtcctcgggcctcgcctccttacaagggcagctgcagccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggccctctaaaaataactccttcagagaggaatgcaacacttgtgagcctcctccctggggacaacccctcccagccaatagcacagcctaggtccccctatataaggccacggctgctggcccttccttcattctagtgtcacctccaggatacagacagccccccttcagcccagcccagccag

SEQ ID NO: 201 - DNA, 582 bp, Artificial Sequence
  MSEC-582A
gctgcccttgtaaggaggcgaggcccgaggacacccgagacgcccggttataattaaccaggacacgtggcgaacccccctccaacacctgccccctctaaaaataactccttctgcagctgcccttgtaaggaggcgaggcccgaggacacccgagacgcccggttataattaaccaggacacgtggcgaacccccctccaacacctgccccctctaaaaataactccttctgcagccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggccctctaaaaataactccttcagagaggaatgcaacacttgtgagcctcctccctggggacaacccctcccagccaatagcacagcctaggtccccctatataaggccacggctgctggcccttccttcattctagtgtcacctccaggatacagacagccccccttcagcccagcccagccag

SEQ ID NO: 202 - DNA, 582 bp, Artificial Sequence
  MSEC-582B
ggatccaccagggacagggttatttttagagcgagcttctcctccatggtgtacagagcctaagacccaggcaccggggtgggggtgaggctcaggcagcaggtgttgggggggggggggcagccacatgtctgggttaattataaccaggcatctcgggtgtccccaggccttgcctccttacatgggcagcctagacccgtagtggggcatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 203 - DNA, 584 bp, Homo sapiens
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 204 - DNA, 586 bp, Artificial Sequence
  MSEC-586
acacccaaatatggctcgcgctctaaaaataaccctgggacacccgagatgcctggttataattaacccacccaacacctgctgccgctcacaagtgttgcattcctctctgcccgctccttctttggtcacagagaggaatgcaacacttgtgagccagagaggaatgcaacacttgtgagccgctctaaaaataaccctgaacacccaaatatggctcgcatgcacacccaaatatggctcgcgctctaaaaataaccctgggacacccgagatgcctggttataattaacccacccaacacctgctgccgctcacaagtgttgcattcctctctgcccgctccttctttggtcacagagaggaatgcaacacttgtgagccagagaggaatgcaacacttgtgagccgctctaaaaataaccctgaacacccaaatatggctcgcatgcggcccctccctggggacagcccctcctggctagtcacaccctgtaggctcctctatataacccaggggcacaggggctgcccccgggtcaccaccacctccacagcacagacagacactcaggagccagccagc

SEQ ID NO: 205 - DNA, 587 bp, Artificial Sequence
  MSEC-587
cccatgagatgacagactataatagccacaggattaacatagcaggcattgtctttctctgactatagggtgggtattatgtgttcatcaaccatcctaaaaatacccggtaaacaggtgcagcccgagctcctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggggcacctgccaaaatagcagccaacacccccccctgtcgcacattcctccctggctcaccaggccccagcccacatgcctgcttaaagccctctccatcctctgcctcacccagtccccgctgagactgagcagacgcctccaggatctgtcggcagct

SEQ ID NO: 206 - DNA, 588 bp, Artificial Sequence
  MSEC-588A
ctgccccccaaccctgctcccagctggccctcccaggcctgggttgctggcctctgctttatcaggattctcaagagggacagctggtttatgttgcatgactgttccctgcatatctgctctggttttaaatagcttatctgctagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 207 - DNA, 588 bp, Artificial Sequence
  MSEC-588B
ggatccaccagggacagggttatttttagagcgagcttctcctccatggtgtacagagcctaagacccaggcaccggggtgggggtaaccgctcaggctcaggcagcaggtgttgggggggggggggcagcaggtgttggggttaattataaccaggcatcagctgtgtctctagatcttgcctccttacatgggcagcctagacccgtagtggggcatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 208 - DNA, 589 bp, Artificial Sequence
  MSEC-589A
ccactacgggtctaggctgccaccattcctcacgacacccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 209 - DNA, 589 bp, Artificial Sequence
  MSEC-589B
ccactacgggtctaggctgcccatgtaaggaggcaagatctagagacacagctgatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatcccctgcatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 210 - DNA, 590 bp, Artificial Sequence
  MSEC-590
ctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggggcacctgccaaaatagcagccaacacccccccctgtcgcacattcctccctggctcaccaggccccagcccacatgcctgcttaaagccctctccatcctctgcctcacccagtccccgctgagactgagcagacgcctccaggatctgtcggcagct

SEQ ID NO: 211 - DNA, 594 bp, Artificial Sequence
  MSEC-594
ccactacgggtctaggctgccagagaggaatgcaacacttgcaccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 212 - DNA, 601 bp, Artificial Sequence
  MSEC-601
ccacccagggccccgtggctgcccttgtaaggaggcgaggcccgaggacacccgagacgcccggttataattaaccaggacacgtggcgaacccccctccaacacctgccccctctaaaaataactcctttcccggcgaccgagactgcagaagatcttcggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttccttcattctagtgtcacctccaggatacagacagccccccttcagcccagcccagccag

SEQ ID NO: 213 - DNA, 602 bp, Artificial Sequence
  MSEC-602A
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcgaagatcttcgaacatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 214 - DNA, 602 bp, Artificial Sequence
  MSEC-602B
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcgaagatcttcgaacatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggtctagggtctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 215 - DNA, 602 bp, Artificial Sequence
  MSEC-602C
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcgaagatcttcgaacatgcccaatcaaggctgtgggggactgagggcaggctgtaacaggcttgggggccagggcttatacgtgcctgggactcccaaagtattactgttccatgttcccggcgaagggccagggtaaccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 216 - DNA, 602 bp, Artificial Sequence
  MSEC-602D
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcgaagatcttcgaacatgcccaatcaaggctgtgggggactgagggcaggctgtaacaggcttgggggccagggcttatacgtgcctgggactcccaaagtattactgttccatgttcccggcgaagggcgagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 217 - DNA, 602 bp, Artificial Sequence
  MSEC-602E
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcgaagatcttcgaacatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcagggtaactcaggggcccctccctggggacagcccctcctggctagtcacaccctgtaggctcctctatataacccaggggcacaggggctgcccccgggtcac

SEQ ID NO: 218 - DNA, 602 bp, Artificial Sequence
  MSEC-602F
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcgaagatcttcgaacatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctgatctgctctcaggggcccctccctggggacagcccctcctggctagtcacaccctgtaggctcctctatataacccaggggcacaggggctgcccccgggtcac

SEQ ID NO: 219 - DNA, 602 bp, Artificial Sequence
  MSEC-602G
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcgaagatcttcgaacatgcccaatcaaggctgtgggggactgagggcaggctgtaacaggcttgggggccagggcttatacgtgcctgggactcccaaagtattactgttccatgttcccggcgaagggcgagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctgatctgctctcaggggcccctccctggggacagcccctcctggctagtcacaccctgtaggctcctctatataacccaggggcacaggggctgcccccgggtcac

SEQ ID NO: 220 - DNA, 602 bp, Artificial Sequence
  MSEC-602H
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcgaagatcttcgaacatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttggtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 221 - DNA, 605 bp, Artificial Sequence
  MSEC-605
ggatccaccagggacagggttatttttagagcgagcttctcctccatggtgtacagagcctaagacccaggcaccggggtgggggtgaggctcaggcagcaggtgttgggggggggggggcagccacatgtctgggttaattataaccaggcatctcgggtgtccccaggccttgcctccttacatgggcagcctagacccgtagtggggcatgcgaagatcttcgaacatgcccgcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagc

SEQ ID NO: 222 - DNA, 607 bp, Artificial Sequence
  MSEC-607
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatcccctgcatgcgaagatcttcgaacatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 223 - DNA, 608 bp, Artificial Sequence
  MSEC-608
ggatccaccagggacagggttatttttagagcgagcttctcctccatggtgtacagagcctaagacccaggcaccggggtgggggtgaggctcaggcagcaggtgttgggggggggggggcagccacatgtctgggttaattataaccaggcatctcgggtgtccccaggccttgcctccttacatgggcagcctagacccgtagtggggcatgcgaagatcttcgaacaaggctgtgggggactgagggcaggctgtaacaggcttgggggccagggcttatacgtgcctgggactccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagc

SEQ ID NO: 224 - DNA, 610 bp, Artificial Sequence
  MSEC-610
ccactacgggtctaggctgccaacacctgctgccaacacctgctgccaacacctgctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 225 - DNA, 611 bp, Artificial Sequence
  MSEC-611
ccctgtcaaattatttataggagatctaagctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 226 - DNA, 617 bp, Artificial Sequence
  MSEC-617
ccactacgggtctaggctgccagagaggaatgcaacacttgcacagagaggaatgcaacacttgcaccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 227 - DNA, 623 bp, Artificial Sequence
  MSEC-623
ccactacgggtctaggctgccaacacctgctgccaacacctgctgccaacacctgctgccaacacctgctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 228 - DNA, 625 bp, Artificial Sequence
  MSEC-625
ccactacgggtctaggctgcgagaggaatgcaacacttgagaggaatgcaacacttgagaggaatgcaacacttccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 229 - DNA, 626 bp, Artificial Sequence
  MSEC-626
ggatccaccagggacagggttatttttagagcgagcttctcctccatggtgtacagagcctaagacccaggcaccggggtgggggtgaggctcaggcagcaggtgttgggggggggggggcagccacatgtctgggttaattataaccaggcatctcgggtgtccccaggccttgcctccttacatgggcagcctagacccgtagtggggcatgcgaagatcttcgaacatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagc

SEQ ID NO: 230 - DNA, 633 bp, Artificial Sequence
  MSEC-633
ggatccaccagggacagggttatttttagagcgagcttctcctccatggtgtacagagcctaagacccaggcaccggggtgggggtgaggctcaggcagcaggtgttgggggggggggggcagccacatgtctgggttaattataaccaggcatctcgggtgtccccaggccttgcctccttacatgggcagcctagacccgtagtggggcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 231 - DNA, 638 bp, Artificial Sequence
  MSEC-638
ccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagctagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 232 - DNA, 640 bp, Artificial Sequence
  MSEC-640
ccactacgggtctaggctgccagagaggaatgcaacacttgcacagagaggaatgcaacacttgcacagagaggaatgcaacacttgcaccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 233 - DNA, 643 bp, Artificial Sequence
  MSEC-643
ggatccaccagggacagggttatttttagagcgagcttctcctccatggtgtacagagcctaagacccaggcaccggggtgggggtgaggctcaggcagcaggtgttgggggggggggggcagccacatgtctgggttaattataaccaggcatctcgggtgtccccaggccttgcctccttacatgggcagcctagacccgtagtggggcatgcgaagatcttcgaacatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagc

SEQ ID NO: 234 - DNA, 652 bp, Artificial Sequence
  MSEC-652
ttttgctattttaaacccgtcggacggagatacgtgagtgcccgaggggctgacacaagccagccagctagctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 235 - DNA, 653 bp, Artificial Sequence
  MSEC-653
ctggctggcttgtgtcagcccctcgggcactcacgtatctccgtccgacgggtttaaaatagcaaaagctagctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 236 - DNA, 656 bp, Artificial Sequence
  MSEC-656A
gcccacctgtcccaatgctgacttagtgcaaggcgagccagaggacaggtggcagagcatctaaaaatagccacgctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 237 - DNA, 656 bp, Artificial Sequence
  MSEC-656B
gctgcccatgtaaggaggcaaggcctggggacacccgagatgcctggttataattaacccagacatgtggctgcccccccccccccaacacctgctgcctctaaaaataaccctgcatgctgcccatgtaaggaggcaaggcctggggacacccgagatgcctggttataattaacccagacatgtggctgcccccccccccccaacacctgctgcctctaaaaataaccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 238 - DNA, 658 bp, Artificial Sequence
  MSEC-658
gtggctatttttagatgctctgccacctgtcctctggctcgccttgcactaagtcagcattgggacaggtgggctagctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 239 - DNA, 662 bp, Artificial Sequence
  MSEC-662
acacccaaatatggctcgcgctctaaaaataaccctgggacacccgagatgcctggttataattaacccagacatgtggctgccccccccaacacctgctgccgctcacaagtgttgcattcctctctgcccgctccttctttggtcacagagaggaatgcaacacttgtgagccagagaggaatgcaacacttgtgagccgctctaaaaataaccctgaacacccaaatatggctcgggccagctgtcccccgcatgcaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 240 - DNA, 673 bp, Artificial Sequence
  MSEC-673
tctgccccccaaccctgctcccagctggccctcccaggcctgggttgctggcctctgctttatcaggattctcaagagggacagctggtttatgttgcatgactgttccctgcatatctgctctggttttaaatagcttatctgctagcatgctgcccatgtaaggaggcaaggcctggggacacccgagatgcctggttataattaacccagacatgtggctgcccccccccccccaacacctgctgcctctaaaaataaccctgcatgaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 241 - DNA, 674 bp, Artificial Sequence
  MSEC-674
gctgcccatgtaaggaggcaaggcctggggacacccgagatgcctggttataattaacccagacatgtggctgcccccccccccccaacacctgctgcctctaaaaataaccctgcatgctgcccatgtaaggaggcaaggcctggggacacccgagatgcctggttataattaacccagacatgtggctgcccccccccccccaacacctgctgcctctaaaaataaccctgcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 242 - DNA, 679 bp, Artificial Sequence
  MSEC-679A
ctgtcgcacattcctccctccgcctgtcgcacattcctccctccgcctgtcgcacattcctccctccgcctgtcgcacattcctccctccgcctgtcgcacattcctccctccgcgctagcatgctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 243 - DNA, 679 bp, Artificial Sequence
  MSEC-679B
gcggagggaggaatgtgcgacaggcggagggaggaatgtgcgacaggcggagggaggaatgtgcgacaggcggagggaggaatgtgcgacaggcggagggaggaatgtgcgacaggctagcatgctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 244 - DNA, 681 bp, Artificial Sequence
  MSEC-681
gcccacctgtcccaatgctgacttagtgcaaggcgagccagcaaggagggaggacaggtggcagtggggggtgaggagcatctaaaaatagccacttcgaatagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 245 - DNA, 686 bp, Artificial Sequence
  MSEC-686
ccactacgggtctaggctgccagagaggaatgcaacacttgcacagagaggaatgcaacacttgcacagagaggaatgcaacacttgcacagagaggaatgcaacacttgcacagagaggaatgcaacacttgcaccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 246 - skipped

SEQ ID NO: 247 - DNA, 701 bp, Artificial Sequence
  MSEC-701
gcggagggaggaatgtgcgacaggcggagggaggaatgtgcgacaggcggagggaggaatgtgcgacaggcggagggaggaatgtgcgacaggcggagggaggaatgtgcgacaggctagctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 248 - DNA, 718 bp, Artificial Sequence
  MSEC-718
ctggctggcttgtgtcagcccctcgggcactcacgtatctccgtccgacgggtttaaaatagcaaaagctagcatgccccactacgggtctaggctgccagagaggaatgcaacacttgcacagagaggaatgcaacacttgcacagagaggaatgcaacacttgcaccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 249 - DNA, 721 bp, Artificial Sequence
  MSEC-721
ttttgctattttaaacccgtcggacggagatacgtgagtgcccgaggggctgacacaagccagccagctagctagcatgccccactacgggtctaggctgccagagaggaatgcaacacttgcacagagaggaatgcaacacttgcacagagaggaatgcaacacttgcaccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 250 - DNA, 722 bp, Artificial Sequence
  MSEC-723A
ccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagctagcatgctgcccatgtaaggaggcaaggcctggggacacccgagatgcctggttataattaacccagacatgtggctgcccccccccccccaacacctgctgcctctaaaaataaccctgcatgaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 251 - DNA, 723 bp, Artificial Sequence
  MSEC-723B
ggatccaccagggacagggttatttttagaggcagcaggtgttgggggggggggggcagccacatgtctgggttaattataaccaggcatctcgggtgtccccaggccttgcctccttacatgggcagcctagacccgtagtggggcatgcaggggatccaccagggacagggttatttttagaggcagcaggtgttgggggggggggggcagccacatgtctgggttaattataaccaggcatctcgggtgtccccaggccttgcctccttacatgggcagcctagacccgtagtggggcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 252 - DNA, 725 bp, Artificial Sequence
  MSEC-725A
tctgccccccaaccctgctcccagctggccctcccaggcctgggttgctggcctctgctttatcaggattctcaagagggacagctggtttatgttgcatgactgttccctgcatatctgctctggttttaaatagcttatctgctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 253 - DNA, 725 bp, Artificial Sequence
  MSEC-725B
tctgccccccaaccctgctcccagctggccctcccaggcctgggtttctggcctctgctttatcaggattctcaagagggacagctggtttatgttgcatgactgttccctgcatatctgctctggttttaaatagcttatctgctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 254 - DNA, 725 bp, Artificial Sequence
  MSEC-725C
ctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggggcacctgccaaaatagcagccaacacccccccctgtcgcacattcctccctggctcaccaggccccagcccacatgcctgcttaaagccctctccatcctctgcctcacccagtccccgctgagactgagcagacgcctccaggatctgtcggcagct

SEQ ID NO: 255 - DNA, 725 bp, Artificial Sequence
  MSEC-725D
ccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 256 - DNA, 726 bp, Artificial Sequence
  MSEC-726
cccatgagatgacagactataatagccacaggattaacatagcaggcattgtctttctctgactatagggtgggtattatgtgttcatcaaccatcctaaaaatacccggtaaacaggtgcagcccgagctcttacgcgtgctagctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 257 - DNA, 730 bp, Artificial Sequence
  MSEC-730
catggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 258 - DNA, 732 bp, Artificial Sequence
  MSEC-732
gtggctatttttagatgctctgccacctgtcctctggctcgccttgcactaagtcagcattgggacaggtgggctagcgtggctatttttagatgctctgccacctgtcctctggctcgccttgcactaagtcagcattgggacaggtgggctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 259 - DNA, 734 bp, Artificial Sequence
  MSEC-734
gcccacctgtcccaatgctgacttagtgcaaggcgagccagaggacaggtggcagagcatctaaaaatagccacgctagcccacctgtcccaatgctgacttagtgcaaggcgagccagaggacaggtggcagagcatctaaaaatagccacgctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 260 - DNA, 736 bp, Artificial Sequence
  MSEC-736
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcagcttgcatgccccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcagcttgcatgccccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatctcgacctccctggggacagcccctcctggctagtcacaccctgtaggctcctctatataacccaggggcacaggggctgcccccgggtcac

SEQ ID NO: 261 - DNA, 746 bp, Artificial Sequence
  MSEC-746
gctgcccatgtaaggaggcaaggcctggggacacccgagatgcctggttataattaacccagacatgtggctgcccccccccccccaacacctgctgcctctaaaaataaccctgcatgctgcccatgtaaggaggcaaggcctggggacacccgagatgcctggttataattaacccagacatgtggctgcccccccccccccaacacctgctgcctctaaaaataaccctgcatgctgcccatgtaaggaggcaaggcctggggacacccgagatgcctggttataattaacccagacatgtggctgcccccccccccccaacacctgctgcctctaaaaataaccctgcatgcgaagatcttcgaacatgcccgcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagc

SEQ ID NO: 262 - DNA, 749 bp, Artificial Sequence
  MSEC-749
gctgcccatgtaaggaggcaaggcctggggacacccgagatgcctggttataattaacccagacatgtggctgcccccccccccccaacacctgctgcctctaaaaataaccctgcatgctgcccatgtaaggaggcaaggcctggggacacccgagatgcctggttataattaacccagacatgtggctgcccccccccccccaacacctgctgcctctaaaaataaccctgcatgctgcccatgtaaggaggcaaggcctggggacacccgagatgcctggttataattaacccagacatgtggctgcccccccccccccaacacctgctgcctctaaaaataaccctgcatgcgaagatcttcgaacaaggctgtgggggactgagggcaggctgtaacaggcttgggggccagggcttatacgtgcctgggactccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagc

SEQ ID NO: 263 - DNA, 754 bp, Artificial Sequence
  MSEC-754
ccactacgggtctaggctgcccatgtaaggaggcaaggcctggggacacccgagatgcctggttataattaaccccaacacctgctgcccccccccccccaacacctgctgcctgagcctgagcggttacccccaccccggtgcctgggtcttaggctctgtacaccatggaggagaagctcgctctaaaaataaccctgtccctggtggatcagcttgcatgccccactacgggtctaggctgcccatgtaaggaggcaaggcctggggacacccgagatgcctggttataattaaccccaacacctgctgcccccccccccccaacacctgctgcctgagcctgagcggttacccccaccccggtgcctgggtcttaggctctgtacaccatggaggagaagctcgctctaaaaataaccctgtccctggtggatcagcttgcatgccccactacgggtctaggctgcccatgtaaggaggcaaggcctggggacacccgagatgcctggttataattaaccccaacacctgctgcccccccccccccaacacctgctgcctgagcctgagcggttacccccaccccggtgcctgggtcttaggctctgtacaccatggaggagaagctcgctctaaaaataaccctgtccctggtggatctcgacctccctggggacagcccctcctggctagtcacaccctgtaggctcctctatataacccaggggcacaggggctgcccccgggtcac

SEQ ID NO: 264 - DNA, 757 bp, Artificial Sequence
  MSEC-757
ggcctctgagagggtcagtgtcctgccccaacccatgagatgacagactataatagccacaggattaacatagcaggcattgtctttctctgactatagggtgggtattatgtgttcatcaaccatcctaaaaatacccggtaaacaggtgcagcccgagctcttacgcgtgctagctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 265 - DNA, 764 bp, Artificial Sequence
  MSEC-764
gatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatctcgggtgtccccaggccttgcctccttacatgggcagcctagacccgtagtggggcatgcaagctgatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatctcgggtgtccccaggccttgcctccttacatgggcagcctagacccgtagtggggcatgcaagctgatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatctcgggtgtccccaggccttgcctccttacatgggcagcctagacccgtagtggtggcatgcaagcttcgacctccctggggacagcccctcctggctagtcacaccctgtaggctcctctatataacccaggggcacaggggctgcccccgggtcac

SEQ ID NO: 266 - DNA, 767 bp, Artificial Sequence
  MSEC-767
gctgcccatgtaaggaggcaaggcctggggacacccgagatgcctggttataattaacccagacatgtggctgcccccccccccccaacacctgctgcctctaaaaataaccctgcatgctgcccatgtaaggaggcaaggcctggggacacccgagatgcctggttataattaacccagacatgtggctgcccccccccccccaacacctgctgcctctaaaaataaccctgcatgctgcccatgtaaggaggcaaggcctggggacacccgagatgcctggttataattaacccagacatgtggctgcccccccccccccaacacctgctgcctctaaaaataaccctgcatgcgaagatcttcgaacatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagc

SEQ ID NO: 267 - DNA, 768 bp, Artificial Sequence
  MSEC-768
ggggctgcacctgtttaccgggtatttttaggatggttgatgaacacataatacccaccctatagtcagagaaagacaatgcctgctatgttaatcctgtggctattatagtctgtcatctcatgggttggggcaggacactgaccctctcagaggcccagatcatcagatcagcttgcatgccccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 268 - DNA, 770 bp, Artificial Sequence
  MSEC-770
ccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 269 - DNA, 771 bp, Artificial Sequence
  MSEC-771
cagcagggccccaaggtttgcccatgaaaggtctgttgccctcgcccctctggctccatggcctttttttagtccttgggcacattcctcctccccaaagggccgatgggcagatagaggagagacaggagcgtctcacaccacctcccctacccaggcccttacctcagttatttttaatctgaagggtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 270 - DNA, 773 bp, Artificial Sequence
  MSEC-773
ggcctctgagagggtcagtgtcctgccccaacccatgagatgacagactataatagccacaggattaacatagcaggcattgtctttctctgactatagggtgggtattatgtgttcatcaaccatcctaaaaatacccggtaaacaggtgcagccccagatcatcatcgataagctagcttgcatgcaggggatccaccagggacagggttatttttagagcgagcttctcctccatggtgtacagagcctaagacccaggcaccggggtgggggtgaggctcaggcagcaggtgttgggggggggggggcagccacatgtctgggttaattataaccaggcatctcgggtgtccccaggccttgcctccttacatgggcagcctagacccgtagtggggcatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 271 - DNA, 778 bp, Artificial Sequence
  MSEC-778
ggcctctgagagggtcagtgtcctgccccaacccatgagatgacagactataatagccacaggattaacatagcaggcattgtctttctctgactatagggtgggtattatgtgttcatcaaccatcctaaaaatacccggtaaacaggtgcagccccagatcatcatcgataagctagcttgcatgccccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcccaatcaaggctgtgggggactgagggcaggctgtaacaggcttgggggccagggcttatacgtgatctgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 272 - DNA, 783 bp, Mus musculus
caggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 273 - DNA, 786 bp, Artificial Sequence
  MSEC-786
ctgccccccaaccctgctcccagctggccctcccaggcctgggttgctggcctctgctttatcaggattctcaagagggacagctggtttatgttgcatgactgttccctgcatatctgctctggttttaaatagcttatctgctagcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagctagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 274 - DNA, 793 bp, Artificial Sequence
  MSEC-793
ccactacgggtctaggctgccagagaggaatgcaacacttgcacagagaggaatgcaacacttgcacagagaggaatgcaacacttgcaccatgtaaggaggcaaggcctggggacacccgagatgcctggttataattaacccagacatgtggctgcccccccccccccaacacctgctgcctctaaaaataaccctgtccctggtggatcccctgcatgcgaagagctctgccccccaaccctgctcccagctggccctcccaggcctgggttgctggcctctgctttatcaggattctcaagagggacagctggtttatgttgcatgactgttccctgcatatctgctctggttttaaatagcttatctgctagctagcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 275 - DNA, 794 bp, Artificial Sequence
  MSEC-794
tctgccccccaaccctgctcccagctggccctcccaggcctgggtttctggcctctgctttatcaggattctcaagagggacagctggtttatgttgcatgactgttccctgcatatctgctctggttttaaatagcttatctgctagcatgccccactacgggtctaggctgccagagaggaatgcaacacttgcacagagaggaatgcaacacttgcacagagaggaatgcaacacttgcaccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 276 - DNA, 798 bp, Artificial Sequence
  MSEC-798
tctgccccccaaccctgctcccagctggccctcccaggcctgggttgctggcctctgctttatcaggattctcaagagggacagctggtttatgttgcatgactgttccctgcatatctgctctggttttaaatagcttatctgctagctagcatgccccactacgggtctaggctgccagagaggaatgcaacacttgcacagagaggaatgcaacacttgcacagagaggaatgcaacacttgcaccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 277 - DNA, 804 bp, Artificial Sequence
  MSEC-804
gtggctatttttagatgctctgccacctgtcctctggctcgccttgcactaagtcagcattgggacaggtgggctagcgtggctatttttagatgctctgccacctgtcctctggctcgccttgcactaagtcagcattgggacaggtgggtatttttagatgctctgccacctgtcctctggctcgccttgcactaagtcagcattgggacaggtgggctagctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 278 - DNA, 806 bp, Artificial Sequence
  MSEC-806
ccctgtcaaattatttataggagatctaagctagacagcagggccccaaggtttgcccatgaaaggtctgttgccctcgcccctctggctccatggcctttttttagtccttgggcacattcctcctccccaaagggccgatgggcagatagaggagagacaggagcgtctcacaccacctcccctacccaggcccttacctcagttatttttaatctgaagggtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 279 - DNA, 809 bp, Artificial Sequence
  MSEC-809
gcgtccaatgctgacttagtgcaaggcgagccagaggacaggtggcagagcatctaaaaatagccacgctagcccacctgtcccaatgctgacttagtgcaaggcgagccagaggacaggtggcagagcatctaaaaatagccacgctagcccacctgtcccaatgctgacttagtgcaaggcgagccagaggacaggtggcagagcatctaaaaatagccacgctagctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 280 - DNA, 812 bp, Artificial Sequence
  MSEC-812A
acacccaaatatggctcgcgctctaaaaataaccctgggacacccgagatgcctggttataattaacccacccaacacctgctgccgctcacaagtgttgcattcctctctgcccgctccttctttggtcacagagaggaatgcaacacttgtgagccagagaggaatgcaacacttgtgagccgctctaaaaataaccctgaacacccaaatatggctcgcatgcacacccaaatatggctcgcgctctaaaaataaccctgggacacccgagatgcctggttataattaacccacccaacacctgctgccgctcacaagtgttgcattcctctctgcccgctccttctttggtcacagagaggaatgcaacacttgtgagccagagaggaatgcaacacttgtgagccgctctaaaaataaccctgaacacccaaatatggctcgcatgcacacccaaatatggctcgcgctctaaaaataaccctgggacacccgagatgcctggttataattaacccacccaacacctgctgccgctcacaagtgttgcattcctctctgcccgctccttctttggtcacagagaggaatgcaacacttgtgagccagagaggaatgcaacacttgtgagccgctctaaaaataaccctgaacacccaaatatggctcgcatgcggcccctccctggggacagcccctcctggctagtcacaccctgtaggctcctctatataacccaggggcacaggggctgcccccgggtcaccaccacctccacagcacagacagacactcaggagccagccagc

SEQ ID NO: 281 - DNA, 812 bp, Artificial Sequence
  MSEC-812B
gagccatatttgggtgttcagggttatttttagagcggctcacaagtgttgcattcctctctggctcacaagtgttgcattcctctctgtgaccaaagaaggagcgggcagagaggaatgcaacacttgtgagcggcagcaggtgttgggtgggttaattataaccaggcatctcgggtgtcccagggttatttttagagcgcgagccatatttgggtgtgcatgcgagccatatttgggtgttcagggttatttttagagcggctcacaagtgttgcattcctctctggctcacaagtgttgcattcctctctgtgaccaaagaaggagcgggcagagaggaatgcaacacttgtgagcggcagcaggtgttgggtgggttaattataaccaggcatctcgggtgtcccagggttatttttagagcgcgagccatatttgggtgtgcatgcgagccatatttgggtgttcagggttatttttagagcggctcacaagtgttgcattcctctctggctcacaagtgttgcattcctctctgtgaccaaagaaggagcgggcagagaggaatgcaacacttgtgagcggcagcaggtgttgggtgggttaattataaccaggcatctcgggtgtcccagggttatttttagagcgcgagccatatttgggtgtgcatgcgccccctccctggggacagcccctcctggctagtcacaccctgtaggctcctctatataacccaggggcacaggggctgcccccgggtcaccaccacctccacagcacagacagacactcaggagccagccagc

SEQ ID NO: 282 - DNA, 836 bp, Artificial Sequence
  MSEC-836
acacccaaatatggctcgcgctctaaaaataaccctgggacacccgagatgcctggttataattaacccagacatgtggctgccccccccaacacctgctgccgctcacaagtgttgcattcctctctgcccgctccttctttggtcacagagaggaatgcaacacttgtgagccagagaggaatgcaacacttgtgagccgctctaaaaataaccctgaacacccaaatatggctcgggccagctgtcccccgcatgcacacccaaatatggctcgcgctctaaaaataaccctgggacacccgagatgcctggttataattaacccagacatgtggctgccccccccaacacctgctgccgctcacaagtgttgcattcctctctgcccgctccttctttggtcacagagaggaatgcaacacttgtgagccagagaggaatgcaacacttgtgagccgctctaaaaataaccctgaacacccaaatatggctcgggccagctgtcccccgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 283 - DNA, 840 bp, Artificial Sequence
  MSEC-840A
tctgccccccaaccctgctcccagctggccctcccaggcctgggttgctggcctctgctttatcaggattctcaagagggacagctggtttatgttgcatgactgttccctgcatatctgctctggttttaaatagcttatctgctagcatgccccactacgggtctaggctgccagagaggaatgcaacacttgcacagagaggaatgcaacacttgcacagagaggaatgcaacacttgcacagagaggaatgcaacacttgcacagagaggaatgcaacacttgcaccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 284 - DNA, 840 bp, Artificial Sequence
  MSEC-840B
tctgccccccaaccctgctcccagctggccctcccaggcctgggtttctggcctctgctttatcaggattctcaagagggacagctggtttatgttgcatgactgttccctgcatatctgctctggttttaaatagcttatctgctagcatgccccactacgggtctaggctgccagagaggaatgcaacacttgcacagagaggaatgcaacacttgcacagagaggaatgcaacacttgcacagagaggaatgcaacacttgcacagagaggaatgcaacacttgcaccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 285 - skipped

SEQ ID NO: 286 - DNA, 875 bp, Artificial Sequence
  MSEC-875
ctgccccccaaccctgctcccagctggccctcccaggcctgggttgctggcctctgctttatcaggattctcaagagggacagctggtttatgttgcatgactgttccctgcatatctgctctggttttaaatagcttatctgctagctgccccccaaccctgctcccagctggccctcccaggcctgggttgctggcctctgctttatcaggattctcaagagggacagctggtttatgttgcatgactgttccctgcatatctgctctggttttaaatagcttatctgctagctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 287 - DNA, 879 bp, Artificial Sequence
  MSEC-879
ccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 288 - DNA, 886 bp, Artificial Sequence
  MSEC-886
ggcctctgagagggtcagtgtcctgccccaacccatgagatgacagactataatagccacaggattaacatagcaggcattgtctttctctgactatagggtgggtattatgtgttcatcaaccatcctaaaaatacccggtaaacaggtgcagcccgagctctgccccccaaccctgctcccagctggccctcccaggcctgggttgctggcctctgctttatcaggattctcaagagggacagctggtttatgttgcatgactgttccctgcatatctgctctggttttaaatagcttatctgctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 289 - DNA, 890 bp, Artificial Sequence
  MSEC-890
ccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagctagagcttgcatgtctaagctagctgccccccaaccctgctcccagctggccctcccaggcctgggttgctggcctctgctttatcaggattctcaagagggacagctggtttatgttgcatgactgttccctgcatatctgctctggttttaaatagcttatctgctagcatgctgcccatgtaaggaggcaaggcctggggacacccgagatgcctggttataattaacccagacatgtggctgcccccccccccccaacacctgctgcctctaaaaataaccctgcatgcaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 290 - DNA, 892 bp, Artificial Sequence
  MSEC-892A
gtggctatttttagatgctctgccacctgtcctctggctcgccttgcactaagtcagcattgggacaggtgggcatgcgtggctatttttagatgctctgccacctgtcctctggctcgccttgcactaagtcagcattgggacaggtgggcatgcgtggctatttttagatgctctgccacctgtcctctggctcgccttgcactaagtcagcattgggacaggtgggcatgcgtggctatttttagatgctctgccacctgtcctctggctcgccttgcactaagtcagcattgggacaggtgggctagctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 291 - DNA, 892 bp, Artificial Sequence
  MSEC-892B
gcccacctgtcccaatgctgacttagtgcaaggcgagccagaggacaggtggcagagcatctaaaaatagccacgcatgcccacctgtcccaatgctgacttagtgcaaggcgagccagaggacaggtggcagagcatctaaaaatagccacgcatgcccacctgtcccaatgctgacttagtgcaaggcgagccagaggacaggtggcagagcatctaaaaatagccacgcatgcgcccacctgtcccaatgctgacttagtgcaaggcgagccagaggacaggtggcagagcatctaaaaatagccacgctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 292 - DNA, 899 bp, Artificial Sequence
  MSEC-899
gcccacctgtcccaatgctgacttagtgcaaggcgagccagcaaggagggaggacaggtggcagtggggggtgaggagcatctaaaaatagccacttcgaatagcatgcgcccacctgtcccaatgctgacttagtgcaaggcgagccagcaaggagggaggacaggtggcagtggggggtgaggagcatctaaaaatagccacttcgaatagcatgcgcccacctgtcccaatgctgacttagtgcaaggcgagccagcaaggagggaggacaggtggcagtggggggtgaggagcatctaaaaatagccacttcgaatagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 293 - DNA, 935 bp, Artificial Sequence
  MSEC-935
ccacccagggccccgtggctgcccttgtaaggaggcgaggcccgaggacacccgagacgcccggttataattaaccaggacacgtggcgaacccccctccaacacctgcccccgaacccccccatacccagcgcctcgggtctcggcctttgcggcagaggagacagcaaagcgccctctaaaaataactcctttcccggcgaccgagaccctccctgtcccccgcacagcggaaatctcccagtggcaccgagggggcgagggttaagtgggggggagggtgaccaccgcctcccacccttgccctgagtttgaatctctccaactcagccagcctcagtttcccctccactcagtccctaggaggaaggggcgcccaagcgcgggtttctggggttagactgccctccattgcaattggtccttctcccggcctctgcttcctccagctcacagggtatctgctcctcctggagccacaccttggttccccgaggtgccgctgggactcgggtaggggtgagggcccagggggcacagggggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgccggagcagggaggtgagaggctcagctgccctccagaactcctccctggggacaacccctcccagccaatagcacagcctaggtccccctatataaggccacggctgctggcccttcctttgggtcag

SEQ ID NO: 294 - DNA, 952 bp, Artificial Sequence
  MSEC-952
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 295 - DNA, 960 bp, Artificial Sequence
  MSEC-960
ggggctgcacctgtttaccgggtatttttaggatggttgatgaacacataatacccaccctatagtcagagaaagacaatgcctgctatgttaatcctgtggctattatagtctgtcatctcatgggttggggcaggacactgaccctctcagaggcccagatcatcagatcagcattatcgatgatgatctggggctgcacctgtttaccgggtatttttaggatggttgatgaacacataatacccaccctatagtcagagaaagacaatgcctgctatgttaatcctgtggctattatagtctgtcatctcatgggttggggcaggacactgaccctctcagaggcccagatcatcagatcagcttgcatgccccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 296 - DNA, 966 bp, Artificial Sequence
  MSEC-966
gtggctatttttagatgctctgccacctgtcctctggctcgccttgcactaagtcagcattgggacaggtgggcatgcgtggctatttttagatgctctgccacctgtcctctggctcgccttgcactaagtcagcattgggacaggtgggcatgcgtggctatttttagatgctctgccacctgtcctctggctcgccttgcactaagtcagcattgggacaggtgggcatgcgtggctatttttagatgctctgccacctgtcctctggctcgccttgcactaagtcagcattgggacaggtgggcatgcgtggctatttttagatgctctgccacctgtcctctggctcgccttgcactaagtcagcattgggacaggtgggctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 297 - DNA, 970 bp, Artificial Sequence
  MSEC-970
gcccacctgtcccaatgctgacttagtgcaaggcgagccagaggacaggtggcagagcatctaaaaatagccacgcatgcccacctgtcccaatgctgacttagtgcaaggcgagccagaggacaggtggcagagcatctaaaaatagccacgcatgcccacctgtcccaatgctgacttagtgcaaggcgagccagaggacaggtggcagagcatctaaaaatagccacgcatgcccacctgtcccaatgctgacttagtgcaaggcgagccagaggacaggtggcagagcatctaaaaatagccacgcatgcgcccacctgtcccaatgctgacttagtgcaaggcgagccagaggacaggtggcagagcatctaaaaatagccacgctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 298 - skipped

SEQ ID NO: 299 - DNA, 988 bp, Artificial Sequence
  MSEC-988
gatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatctcgggtgtccccaggccttgcctccttacatgggcagcctagacccgtagtggggcatgcaagctgatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatctcgggtgtccccaggccttgcctccttacatgggcagcctagacccgtagtggggcatgcaagctgatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatctcgggtgtccccaggccttgcctccttacatgggcagcctagacccgtagtggggcatgcaagctgatccaccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatctcgggtgtccccaggccttgcctccttacatgggcagcctagacccgtagtggtggcatgcaagcttcgacctccctggggacagcccctcctggctagtcacaccctgtaggctcctctatataacccaggggcacaggggctgcccccgggtcac

SEQ ID NO: 300 - DNA, 995 bp, Artificial Sequence
  MSEC-995
ctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgctagcctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggggcacctgccaaaatagcagccaacacccccccctgtcgcacattcctccctggctcaccaggccccagcccacatgcctgcttaaagccctctccatcctctgcctcacccagtccccgctgagactgagcagacgcctccaggatctgtcggcagct

SEQ ID NO: 301 - DNA, 1016 bp, Artificial Sequence
  MSEC-1016
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 302 - DNA, 1027 bp, Artificial Sequence
  MSEC-1027
cttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 303 - DNA, 1031 bp, Artificial Sequence
  MSEC-1031
ggatccaccagggacagggttatttttagaggcagcaggtgttgggggggggggggcagccacatgtctgggttaattataaccaggcatctcgggtgtccccaggccttgcctccttacatgggcagcctagacccgtagtggggcatgcaggggatccaccagggacagggttatttttagaggcagcaggtgttgggggggggggggcagccacatgtctgggttaattataaccaggcatctcgggtgtccccaggccttgcctccttacatgggcagcctagacccgtagtggggcatgcaggggatccaccagggacagggttatttttagaggcagcaggtgttgggggggggggggcagccacatgtctgggttaattataaccaggcatctcgggtgtccccaggccttgcctccttacatgggcagcctagacccgtagtggggcatgcaggggatccaccagggacagggttatttttagaggcagcaggtgttgggggggggggggcagccacatgtctgggttaattataaccaggcatctcgggtgtccccaggccttgcctccttacatgggcagcctagacccgtagtggggcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 304 - DNA, 1204 bp, Artificial Sequence
  MSEC-1204
gcccacctgtcccaatgctgacttagtgcaaggcgagccagaggacaggtggcagagcatctaaaaatagccacgcatgcccacctgtcccaatgctgacttagtgcaaggcgagccagaggacaggtggcagagcatctaaaaatagccacgcatgcccacctgtcccaatgctgacttagtgcaaggcgagccagaggacaggtggcagagcatctaaaaatagccacgcatgcccacctgtcccaatgctgacttagtgcaaggcgagccagaggacaggtggcagagcatctaaaaatagccacgctagcccacctgtcccaatgctgacttagtgcaaggcgagccagaggacaggtggcagagcatctaaaaatagccacgcatgcccacctgtcccaatgctgacttagtgcaaggcgagccagaggacaggtggcagagcatctaaaaatagccacgcatgcccacctgtcccaatgctgacttagtgcaaggcgagccagaggacaggtggcagagcatctaaaaatagccacgcatgcgcccacctgtcccaatgctgacttagtgcaaggcgagccagaggacaggtggcagagcatctaaaaatagccacgctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 305 - skipped

SEQ ID NO: 306 - DNA, 1225 bp, Artificial Sequence
  MSEC-1225
agctttctgcttctgcgtcttgcctcggtgtcctgtctcctggggtgcccctgagggagggggtagcagaggatacagaaccttctgaagggagagatctgggctgggagcccggggtgtccttgaggcccagagcctggctgtgtgtcctcctggccaccccagcccacctgtcccaatgctgacttagtgcaaggcgagccagcaaggagggaggacaggtggcagtggggggtgaggagcatctaaaaatagccacaaagtagcagcttcaagggctttgggtctctgtctgccccacactcttctctcagcttggtccaccttccctctcaccttcctctgaggcccccttccagccccgatggaggcctgatgtcccccatggtcagtgcttcagggatctagtcaataaaattaataatgaaaaacaacagtaataaaatacacgtgacgtgactggggcagcttagggcttagttcaaatcccagtgttcacaccctttaaaagacaagacaaaacaaaacagctggctgtgggggagaacatcagaatccccctggggaggtggggacaggggatctgtggggctccatggccaggcagcctagctccaggcctgcgagagaccctacctcaagataaaaataaaataaaataaaataaaataaaataaatatataaaataacaatgttgcagcacctgaggtcaccactggaatgtgcacacctgtgcacatacatgagcctgcactacaaacaaaaatattaacagtaactgttagaatcccagctgcaacttcatgccaggtgccaggtccatgctcatcagtcagggactggaactcagagatctcctgggaaagcttcagtctcacagattcaaaagccagagagatctagtcacagcctggggcccagagcagtgacttaggagagccgtgccttttaaagtggaccttgtagacagccagaggtggagggactgggattcgaacatgcccaatcaaggctgtgggggactgagggcaggctgtaacaggcttgggggccagggcttatacgtgcctgggactcccaaagtattactgttcgtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 307 - DNA, 1240 bp, Artificial Sequence
  MSEC-1240
agctttctgcttctgcgtcttgcctcggtgtcctgtctcctggggtgcccctgagggagggggtagcagaggatacagaaccttctgaagggagagatctgggctgggagcccggggtgtccttgaggcccagagcctggctgtgtgtcctcctggccaccccagcccacctgtcccaatgctgacttagtgcaaggcgagccagcaaggagggaggacaggtggcagtggggggtgaggagcatctaaaaatagccacaaagtagcagcttcaagggctttgggtctctgtctgccccacactcttctctcagcttggtccaccttccctctcaccttcctctgaggcccccttccagccccgatggaggcctgatgtcccccatggtcagtgcttcagggatctagtcaataaaattaataatgaaaaacaacagtaataaaatacacgtgacgtgactggggcagcttagggcttagttcaaatcccagtgttcacaccctttaaaagacaagacaaaacaaaacagctggctgtgggggagaacatcagaatccccctggggaggtggggacaggggatctgtggggctccatggccaggcagcctagctccaggcctgcgagagaccctacctcaagataaaaataaaataaaataaaataaaataaaataaatatataaaataacaatgttgcagcacctgaggtcaccactggaatgtgcacacctgtgcacatacatgagcctgcactacaaacaaaaatattaacagtaactgttagaatcccagctgcaacttcatgccaggtgccaggtccatgctcatcagtcagggactggaactcagagatctcctgggaaagcttcagtctcacagattcaaaagccagagagatctagtcacagcctggggcccagagcagtgacttaggagagccgtgccttttaaagtggaccttgtagacagccagaggtggagggactgggattcgaacatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacg

SEQ ID NO: 308 - DNA, 1263 bp, Mus musculus
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 309 - DNA, 1263 bp, Artificial Sequence
  MSEC-1263B
ccactacgggtctaggctgcccatgtaaggaggcaaggcctggggacacccgagatgcctggttataattaacccagacatgtggctgcccccccccccccaacacggtaaccctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagggtaaccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 310 - DNA, 1263 bp, Artificial Sequence
  MSEC-1263C
ccactacgggtctaggctgcccatgtaaggaggcaaggcctggggacacccgagatgcctggttataattaacccatctagatggctgcccccccccccccaacacggtaaccctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagggtaaccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 311 - DNA, 1263 bp, Artificial Sequence
  MSEC-1263D
ccactacgggtctaggctgcccatgtaaggaggcaaggcctggggacacccgagatgcctggttataattaacccatctagatggctgcccccccccccccaacacggtaaccctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 312 - DNA, 1263 bp, Artificial Sequence
  MSEC-1263E
ccactacgggtctaggctgcccatgtaaggaggcaaggcctggggacacccgagatgcctggttataattaacccagacatgtggctgcccccccccccccaacacggtaaccctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 313 - DNA, 1263 bp, Artificial Sequence
  MSEC-1263F
ccactacgggtctaggctgcccatgtaaggaggcaaggcctggggacacccgagatgcctggttataattaacccatctagatggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagggtaaccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 314 - DNA, 1263 bp, Artificial Sequence
  MSEC-1263G
ccactacgggtctaggctgcccatgtaaggaggcaaggcctggggacacccgagatgcctggttataattaacccatctagatggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 315 - DNA, 1263 bp, Artificial Sequence
  MSEC-1263H
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagggtaaccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 316 - DNA, 1263 bp, Artificial Sequence
  MSEC-1263I
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactcgccctgtccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 317 - DNA, 1263 bp, Artificial Sequence
  MSEC-1263J
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttcgccctgcaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 318 - DNA, 1263 bp, Artificial Sequence
  MSEC-1263K
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttcgccctgcaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactcgccctgtccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 319 - DNA, 1263 bp, Artificial Sequence
  MSEC-1263L
ccactacgggtctaggctgcgcaggtaccgaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 320 - DNA, 1263 bp, Artificial Sequence
  MSEC-1263M
ccactacgggtctaggctgcagatctaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 321 - DNA, 1263 bp, Artificial Sequence
  MSEC-1263N
ccactacgggtctaggctgcccatgtaaggaggcaagatctagagacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 322 - DNA, 1263 bp, Artificial Sequence
  MSEC-1263O
ccactacgggtctaggctgcccatgtaaggaggcaaggcctggggacacccgagatgcctggctagcatgcacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 323 - DNA, 1263 bp, Artificial Sequence
  MSEC-1263P
ccactacgggtctaggctgcccatgtaaggaggcaaggcctggggacacccgagatgcctggttctagatctcccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 324-325 - skipped (2 entries)

SEQ ID NO: 326 - DNA, 1263 bp, Artificial Sequence
  MSEC-1263S
ccactacgggtctaggctgcccatgtaaggaggcaaggcctggggacacccgagatgcctggttataattaacccagacatgtggctgcccccccccccccaagatctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 327 - DNA, 1263 bp, Artificial Sequence
  MSEC-1263T
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctgggcccaaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 328 - DNA, 1263 bp, Artificial Sequence
  MSEC-1263U
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctctagatctccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 329 - DNA, 1263 bp, Artificial Sequence
  MSEC-1263V
ccactacgggtctaggctgcccatgtaaggaggcaaggcctggggacaggtaaccactctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 330 - DNA, 1279 bp, Artificial Sequence
  MSEC-1279
agctttctgcttctgcgtcttgcctcggtgtcctgtctcctggggtgcccctgagggagggggtagcagaggatacagaaccttctgaagggagagatctgggctgggagcccggggtgtccttgaggcccagagcctggctgtgtgtcctcctggccaccccagcccacctgtcccaatgctgacttagtgcaaggcgagccagcaaggagggaggacaggtggcagtggggggtgaggagcatctaaaaatagccacaaagtagcagcttcaagggctttgggtctctgtctgccccacactcttctctcagcttggtccaccttccctctcaccttcctctgaggcccccttccagccccgatggaggcctgatgtcccccatggtcagtgcttcagggatctagtcaataaaattaataatgaaaaacaacagtaataaaatacacgtgacgtgactggggcagcttagggcttagttcaaatcccagtgttcacaccctttaaaagacaagacaaaacaaaacagctggctgtgggggagaacatcagaatccccctggggaggtggggacaggggatctgtggggctccatggccaggcagcctagctccaggcctgcgagagaccctacctcaagataaaaataaaataaaataaaataaaataaaataaatatataaaataacaatgttgcagcacctgaggtcaccactggaatgtgcacacctgtgcacatacatgagcctgcactacaaacaaaaatattaacagtaactgttagaatcccagctgcaacttcatgccaggtgccaggtccatgctcatcagtcagggactggaactcagagatctcctgggaaagcttcagtctcacagattcaaaagccagagagatctagtcacagcctggggcccagagcagtgacttaggagagccgtgccttttaaagtggaccttgtagacagccagaggtggagggactgggattcgaacatgcccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 331 - DNA, 1360 bp, Artificial Sequence
  MSEC-1360
gtggctatttttagatgctctgccacctgtcctctggctcgccttgcactaagtcagcattgggacaggtgggcatgcgtggctatttttagatgctctgccacctgtcctctggctcgccttgcactaagtcagcattgggacaggtgggcatgcgtggctatttttagatgctctgccacctgtcctctggctcgccttgcactaagtcagcattgggacaggtgggcatgcgtggctatttttagatgctctgccacctgtcctctggctcgccttgcactaagtcagcattgggacaggtgggcatgcgtggctatttttagatgctctgccacctgtcctctggctcgccttgcactaagtcagcattgggacaggtgggctagcgtggctatttttagatgctctgccacctgtcctctggctcgccttgcactaagtcagcattgggacaggtgggcatgcgtggctatttttagatgctctgccacctgtcctctggctcgccttgcactaagtcagcattgggacaggtgggcatgcgtggctatttttagatgctctgccacctgtcctctggctcgccttgcactaagtcagcattgggacaggtgggcatgcgtggctatttttagatgctctgccacctgtcctctggctcgccttgcactaagtcagcattgggacaggtgggcatgcgtggctatttttagatgctctgccacctgtcctctggctcgccttgcactaagtcagcattgggacaggtgggctagctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 332 - DNA, 1370 bp, Artificial Sequence
  MSEC-1370A
agctttctgcttctgcgtcttgcctcggtgtcctgtctcctggggtgcccctgagggagggggtagcagaggatacagaaccttctgaagggagagatctgggctgggagcccggggtgtccttgaggcccagagcctggctgtgtgtcctcctggccaccccagcccacctgtcccaatgctgacttagtgcaaggcgagccagcaaggagggaggacaggtggcagtggggggtgaggagcatctaaaaatagccacaaagtagcagcttcaagggctttgggtctctgtctgccccacactcttctctcagcttggtccaccttccctctcaccttcctctgaggcccccttccagccccgatggaggcctgatgtcccccatggtcagtgcttcagggatctagtcaataaaattaataatgaaaaacaacagtaataaaatacacgtgacgtgactggggcagcttagggcttagttcaaatcccagtgttcacaccctttaaaagacaagacaaaacaaaacagctggctgtgggggagaacatcagaatccccctggggaggtggggacaggggatctgtggggctccatggccaggcagcctagctccaggcctgcgagagaccctacctcaagataaaaataaaataaaataaaataaaataaaataaatatataaaataacaatgttgcagcacctgaggtcaccactggaatgtgcacacctgtgcacatacatgagcctgcactacaaacaaaaatattaacagtaactgttagaatcccagctgcaacttcatgccaggtgccaggtccatgctcatcagtcagggactggaactcagagatctcctgggaaagcttcagtctcacagattcaaaagccagagagatctagtcacagcctggggcccagagcagtgacttaggagagccgtgccttttaaagtggaccttgtagacagccagaggtggagggactgggattcgaacatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 333 - DNA, 1370 bp, Artificial Sequence
  MSEC-1370B
agctttctgcttctgcgtcttgcctcggtgtcctgtctcctggggtgcccctgagggagggggtagcagaggatacagaaccttctgaagggagagatctgggctgggagcccggggtgtccttgaggcccagagcctggctgtgtgtcctcctggccaccccagcccacctgtcccaatgctgacttagtgcaaggcgagccagcaaggagggaggacaggtggcagtggggggtgaggagcatctaaaaatagccacaaagtagcagcttcaagggctttgggtctctgtctgccccacactcttctctcagcttggtccaccttccctctcaccttcctctgaggcccccttccagccccgatggaggcctgatgtcccccatggtcagtgcttcagggatctagtcaataaaattaataatgaaaaacaacagtaataaaatacacgtgacgtgactggggcagcttagggcttagttcaaatcccagtgttcacaccctttaaaagacaagacaaaacaaaacagctggctgtgggggagaacatcagaatccccctggggaggtggggacaggggatctgtggggctccatggccaggcagcctagctccaggcctgcgagagaccctacctcaagataaaaataaaataaaataaaataaaataaaataaatatataaaataacaatgttgcagcacctgaggtcaccactggaatgtgcacacctgtgcacatacatgagcctgcactacaaacaaaaatattaacagtaactgttagaatcccagctgcaacttcatgccaggtgccaggtccatgctcatcagtcagggactggaactcagagatctcctgggaaagcttcagtctcacagattcaaaagccagagagatctagtcacagcctggggcccagagcagtgacttaggagagccgtgccttttaaagtggaccttgtagacagccagaggtggagggactgggattcgaacatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggtctagggtctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 334 - DNA, 1465 bp, Artificial Sequence
  MSEC-1465
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcacctagaggatctgcagaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcaattcccg

SEQ ID NO: 335 - skipped

SEQ ID NO: 336 - DNA, 1755 bp, Mus musculus
gatcttactggaggtggtgtggcagaaacccattccttttagtgtcttgggctaaaagtaaaaggcccagaggaggcctttgctcatctgaccatgctgacaaggaacacgggtgccaggacagaggctggaccccaggaacaccttaaacacttcttcccttctccgccccctagagcaggctcccctcaccagcctgggcagaaatgggggaagatggagtgaagccatactggctactccagaatcaacagagggagccgggggcaatactggagaagctggtctccccccaggggcaatcctggcacctcccaggcagaagaggaaacttccacagtgcatctcacttccatgaatcccctcctcggactctgaggtccttggtcacagctgaggtgcaaaaggctcctgtcatattgtgtcctgctctggtctgccttccacagcttgggggccacctagcccacctctccctagggatgagagcagccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 337-342 - skipped (6 entries)

SEQ ID NO: 343 - DNA, 22 bp, Artificial Sequence
  MIR206TS-3G
ccacacacttccttacattgca

SEQ ID NO: 344 - skipped

SEQ ID NO: 345 - DNA, 78 bp, Artificial Sequence
  MIR206TS-3GX3
ccacacacttccttacattgcactgcagccacacacttccttacattgcactgcagccacacacttccttacattgca

SEQ ID NO: 346 - skipped

SEQ ID NO: 347 - DNA, 162 bp, Artificial Sequence
  MIR206TS-3GX6
ccacacacttccttacattgcactgcagccacacacttccttacattgcactgcagccacacacttccttacattgcatctagaccacacacttccttacattgcactgcagccacacacttccttacattgcactgcagccacacacttccttacattgca

SEQ ID NO: 348 - skipped

SEQ ID NO: 349 - DNA, 78 bp, Artificial Sequence
  MIR208ATSX3
acaagctttttgctcgtcttatctgcagacaagctttttgctcgtcttatctgcagacaagctttttgctcgtcttat

SEQ ID NO: 350 - DNA, 144 bp, Mus musculus
tctgccccccaaccctgctcccagctggccctcccaggcctgggttgctggcctctgctttatcaggattctcaagagggacagctggtttatgttgcatgactgttccctgcatatctgctctggttttaaatagcttatctg

SEQ ID NO: 351 - DNA, 318 bp, Artificial Sequence
  MSEC-318
catgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 352 - DNA, 345 bp, Artificial Sequence
  MSEC-345
gctgcccatgtaaggaggggacacccgagacctggttataattaacccagacatgtggctgccccccccccaacacctgctgcctctaaaaataaccctgcatgcgggccagctgtcccccgccagcactcagcacttagtgcccatacaaggccatggggctgggcgcctgggtccggggtgggtgcctctaaaaataacccaagggccagctgtccccctccctggggacagcccctcctggcatcacaccctaggctcctctatataacccaggggcacaggggctgcccccgggtcaccaccacctccacagcacagacagacactcaggagccagccagc

SEQ ID NO: 353 - DNA, 395 bp, Artificial Sequence
  MSEC-395B
gctgcccttgtaaggaggggacacccgagacgcggttataattaaccaggacacgtggcccccaacacctgccccctctaaaaataactgcagccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggccctctaaaaataactccttcagagaggaatgcaacacttgtgagcctcctccctggggacaacccctcccagccaatagcacagcctaggtccccctatataaggccacggctgctggcccttccttcattctagtgtca

SEQ ID NO: 354 - DNA, 403 bp, Artificial Sequence
  MSEC-403B
gctgcccttgtaaggaggggacacccgagacgcggttataattaaccaggacacgtggcgaccccccccccaacacctgccccctctaaaaataactgcagccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggccctctaaaaataactccttcagagaggaatgcaacacttgtgagcctcctccctggggacaacccctcccagccaatagcacagcctaggtccccctatataaggccacggctgctggcccttccttcattctagtgtca

SEQ ID NO: 355 - DNA, 438 bp, Artificial Sequence
  MSEC-438I
gctgcccttgtaaggaggggacacccgagacgcggttataattaaccaggacacgtggcccccaacacctgccccctctaaaaataactgcagccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggccctctaaaaataactccttcagagaggaatgcaacacttgtgagcctcctccctggggacaacccctcccagccaatagcacagcctaggtccccctatataaggccacggctgctggcccttccttcattctagtgtcacctccaggatacagacagccccccttcagcccagcccagccag

SEQ ID NO: 356 - DNA, 476 bp, Artificial Sequence
  MSEC-476B
gcccacctgtcccaatgctgacttagtgcaaggcgagccagcaaggagggaggacaggtggcagtggggggtgaggagcatctaaaaatagccacttcgaatagcatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 357 - DNA, 481 bp, Artificial Sequence
  MSEC-481
cccatgagatgacagactataatagccacaggattaacatagcaggcattgtctttctctgactatagggtgggtattatgtgttcatcaaccatcctaaaaatacccggtaaacaggtgcagcccgagctcctggttataattaaccccccaacacctgctgccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccacca

SEQ ID NO: 358 - DNA, 508 bp, Artificial Sequence
  MSEC-508
gctgcccatgtaaggaggcctgcatatctgctctggacacccgagatgcctggttataattaacccagacatgtggctgcccccccccccccaacacctgctgcctctaaaaataaccctgcatgctgcccatgtaaggaggcctgcatatctgctctggacacccgagatgcctggttataattaacccagacatgtggctgcccccccccccccaacacctgctgcctctaaaaataaccctgcatgccatgttcccggcgaagggccagctgtcccccgccagctagactcagcacttagtctgcatatctgctctcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 359 - DNA, 672 bp, Artificial Sequence
  MSEC-672
cagattaaaaataactcctctatctgcccatcggccctttggggaggaggaatgtgcccaaggactaaaaaaaggccagacctttcatgggcaaactgcagccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 360 - DNA, 685 bp, Artificial Sequence
  MSEC-685
ctgctcccagctggccctcccagtctgctttatcagacagctggtttatgttgcatgactgttccctgcatatctgctctggttttaaatagcttatctgctagctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 361 - DNA, 689 bp, Artificial Sequence
  MSEC-689
cagattaaaaataactgaggtaagggcctgggtcctctatctgcccatcggccctttggggaggaggaatgtgcccaaggactaaaaaaaggccagacctttcatgggcaaactgcagccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 362 - DNA, 697 bp, Artificial Sequence
  MSEC-697
cagattaaaaataactcctctatctgcccatcggccctttggggaggaggaatgtgcccaaggactaaaaaaaggccatggagccagaggggcgagggcaacagacctttcatgggcaaactgcagccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 363 - DNA, 714 bp, Artificial Sequence
  MSEC-714
cagattaaaaataactgaggtaagggcctgggtcctctatctgcccatcggccctttggggaggaggaatgtgcccaaggactaaaaaaaggccatggagccagaggggcgagggcaacagacctttcatgggcaaactgcagccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 364 - DNA, 720 bp, Artificial Sequence
  MSEC-720
cagattaaaaataactgaggtaagggcctgggtaggggaggtggtgtgagacgctcctgtctctcctctatctgcccatcggccctttggggaggaggaatgtgcccaaggactaaaaaaaggccagacctttcatgggcaaactgcagccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 365 - DNA, 745 bp, Artificial Sequence
  MSEC-745
cagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaactgcagccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 366 - DNA, 758 bp, Artificial Sequence
  MSEC-758
cccatgagatgacagactataatagccacaggattaacatagcaggcattgtctttctctgactatagggtgggtattatgtgttcatcaaccatcctaaaaatacccggtaaacaggtgcagcccgagctcttacgcgtgctagcatgccccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatcccctgcatgcgaagatcttcgaacatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 367 - DNA, 855 bp, Artificial Sequence
  MSEC-855
cccatgagatgacagactataatagccacaggattaacatagcaggcattgtctttctctgactatagggtgggtattatgtgttcatcaaccatcctaaaaatacccggtaaacaggtgcagcccgagctcttacgcgtgctagcatgccccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatcccctgcatgcgaagatcttcgaacatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcactgcagaagaggtaagggtttaagggatggttggttggtggggtattaatgtttaattacctggagcacctgcctgaaatcactttttttcaggttgg

SEQ ID NO: 368 - DNA, 872 bp, Artificial Sequence
  MSEC-872
tctgccccccaaccctgctcccagctggccctcccaggcctgggttgctggcctctgctttatcaggattctcaagagggacagctggtttatgttgcatgactgttccctgcatatctgctctggttttaaatagcttatctgctagcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagctagcatgctgcccatgtaaggaggcaaggcctggggacacccgagatgcctggttataattaacccagacatgtggctgcccccccccccccaacacctgctgcctctaaaaataaccctgcatgcaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 369 - DNA, 964 bp, Artificial Sequence
  MSEC-964
agctttctgcttctgcgtcttgcctcggtgtcctgtctcctggggtgcccctgagggagggggtagcagaggatacagaaccttctgaagggagagatctgggctgggagcccggggtgtccttgaggcccagagcctggctgtgtgtcctcctggccaccccagcccacctgtcccaatgctgacttagtgcaaggcgagccagcaaggagggaggacaggtggcagtggggggtgaggagcatctaaaaatagccacaaagtagcagcttcaagggctttgggtctctgtctgccccacactcttctctcagcttggtccaccttccctctcaccttcctctgaggcccccttccagccccgatggaggcctgatgtcccccatggtcagtgcttcagggatctagtcaataaaattaataatgaaaaacaacagtaataaaatacacgtgacgtgactggggcagcttagggcttagttcaaatcccagtgttcacaccctttaaaagacaagacaaaacaaaacagctggctgtgggggagaacatcagaatccccctggggaggtggggacaggggatctgtggggctccatggccaggcagcctagctccaggcctgcgagagaccctacctcaagataaaaataaaataaaataaaataaatatataaaataacaatgttgcagcacctgaggtcaccactggaatgtgcacacctgtgcacatacatgagcctgcactacaaacaaaaatattaacagtaactgttagaatcccagctgcaacttcatgccaggtgccaggtccatgctcatcagtcagggactggaactcagagatctcctgggaaagcttgcatgcctgcaggtcgacctccctggggacagcccctcctggctagtcacaccctgtaggctcctctatataacccaggggcacaggggctgcccccgggtcac

SEQ ID NO: 370 - DNA, 1207 bp, Artificial Sequence
  MSEC-1207
gtggctatttttagatgctcctcaccccccactgccacctgtcctccctccttgctggctcgccttgcactaagtcagcattgggacaggtgggcgcatgctagcttagacatgcaagctcgaagtggctatttttagatgctcctcaccccccactgccacctgtcctccctccttgctggctcgccttgcactaagtcagcattgggacaggtgggcgcatgctagcttagacatgcaagctcgaagtggctatttttagatgctcctcaccccccactgccacctgtcctccctccttgctggctcgccttgcactaagtcagcattgggacaggtgggcgcatgctagcttagacatgcaagctcgaagtggctatttttagatgctcctcaccccccactgccacctgtcctccctccttgctggctcgccttgcactaagtcagcattgggacaggtgggcgcatgctagcttagacatgcaagctcgaagtggctatttttagatgctcctcaccccccactgccacctgtcctccctccttgctggctcgccttgcactaagtcagcattgggacaggtgggcgcatgctagcttagacatgcaagctcgcgtgctagctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 371 - DNA, 1666 bp, Artificial Sequence
  MSEC-1666
agctttctgcttctgcgtcttgcctcggtgtcctgtctcctggggtgcccctgagggagggggtagcagaggatacagaaccttctgaagggagagatctgggctgggagcccggggtgtccttgaggcccagagcctggctgtgtgtcctcctggccaccccagcccacctgtcccaatgctgacttagtgcaaggcgagccagcaaggagggaggacaggtggcagtggggggtgaggagcatctaaaaatagccacaaagtagcagcttcaagggctttgggtctctgtctgccccacactcttctctcagcttggtccaccttccctctcaccttcctctgaggcccccttccagccccgatggaggcctgatgtcccccatggtcagtgcttcagggatctagtcaataaaattaataatgaaaaacaacagtaataaaatacacgtgacgtgactggggcagcttagggcttagttcaaatcccagtgttcacaccctttaaaagacaagacaaaacaaaacagctggctgtgggggagaacatcagaatccccctggggaggtggggacaggggatctgtggggctccatggccaggcagcctagctccaggcctgcgagagaccctacctcaagataaaaataaaataaaataaaataaatatataaaataacaatgttgcagcacctgaggtcaccactggaatgtgcacacctgtgcacatacatgagcctgcactacaaacaaaaatattaacagtaactgttagaatcccagctgcaacttcatgccaggtgccaggtccatgctcatcagtcagggactggaactcagagatctcctgggaaagcttgcatgcctgcaggtcgactctagcaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 372 - DNA, 3355 bp, Mus musculus
atcctggtctatagagagagttccagaacagccagggctacagataaacccatctggaaaaacaaagttgaatgacccaagaggggttctcagagggtggcgtgtgctccctggcaagcctatgacatggccggggcctgcctctctctgcctctgaccctcagtggctcccatgaactccttgcccaatggcatctttttcctgcgctccttgggttattccagtctcccctcagcattccttcctcagggcctcgctcttctctctgctccctccttgcacagctggctctgtccacctcagatgtcacagtgctctctcagaggaggaaggcaccatgtaccctctgtttcccaggtaagggttcaatttttaaaaatggttttttgtttgtttgtttgtttgtttgtttgtttgtttttcaagacagggctcctctgtgtagtcctaactgtcttgaaactccctctgtagaccaggtcgacctcgaactcttgaaacctgccacggaccacccagtcaggtatggaggtccctggaatgagcgtcctcgaagctaggtgggtaagggttcggcggtgacaaacagaaacaaacacagaggcagtttgaatctgagtgtattttgcagctctcaagcaggggattttatacataaaaaaaaaaaaaaaaaaaaaaccaaacattacatctcttagaaactatatccaatgaaacaatcacagataccaaccaaaaccattgggcagagtaaagcacaaaaatcatccaagcattacaactctgaaaccatgtattcagtgaatcacaaacagaacaggtaacatcattattaatataaatcaccaaaatataacaattctaaaaggatgtatccagtgggggctgtcgtccaaggctagtggcagatttccaggagcaggttagtaaatcttaaccactgaactaactctccagccccatggtcaattattatttagcatctagtgcctaatttttttttataaatcttcactatgtaatttaaaactattttaattcttcctaattaaggctttctttaccatataccaaaattcacctccaatgacacacgcgtagccatatgaaattttattgttgggaaaatttgtacctatcataatagttttgtaaatgatttaaaaagcaaagtgttagccgggcgtggtggcacacgcctttaatccctgcactcgggaggcaggggcaggaggatttctgagtttgaggccagcctggtctacagagtgagttccaggacagccagggctacacagagaaaccctgtctcgaaccccccaccccccaaaaaaagcaaagtgttggtttccttggggataaagtcatgttagtggcccatctctaggcccatctcacccattattctcgcttaagatcttggcctaggctaccaggaacatgtaaataagaaaaggaataagagaaaacaaaacagagagattgccatgagaactacggctcaatattttttctctccggcgaagagttccacaaccatctccaggaggcctccacgttttgaggtcaatggcctcagtctgtggaacttgtcacacagatcttactggaggtggtgtggcagaaacccattccttttagtgtcttgggctaaaagtaaaaggcccagaggaggcctttgctcatctgaccatgctgacaaggaacacgggtgccaggacagaggctggaccccaggaacaccttaaacacttcttcccttctccgccccctagagcaggctcccctcaccagcctgggcagaaatgggggaagatggagtgaagccatactggctactccagaatcaacagagggagccgggggcaatactggagaagctggtctccccccaggggcaatcctggcacctcccaggcagaagaggaaacttccacagtgcatctcacttccatgaatcccctcctcggactctgaggtccttggtcacagctgaggtgcaaaaggctcctgtcatattgtgtcctgctctggtctgccttccacagcttgggggccacctagcccacctctccctagggatgagagcagccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac